(19) United States
(12) Patent Application Publication
(10) Pub. No.: US 2017/0050980 A1
(43) Pub. Date: Feb. 23, 2017

(54) OXADIAZINE COMPOUNDS AND METHODS OF USE THEREOF

(71) Applicant: FORUM Pharmaceuticals Inc., Waltham, WA (US)

(72) Inventors: Duane A. Burnett, Wayland, MA (US); Matthew Gregory Bursavich, Needham, MA (US); Bryce Alden Harrison, Framingham, MA (US)

(21) Appl. No.: 15/240,419

(22) Filed: Aug. 18, 2016

Related U.S. Application Data
(60) Provisional application No. 62/311,228, filed on Mar. 21, 2016.
(60) Provisional application No. 62/279,391, filed on Jan. 15, 2016.
(60) Provisional application No. 62/206,577, filed on Aug. 18, 2015.

(51) Int. Cl. C07D 498/04; C07D 413/14; C07D 471/04; C07D 413/04

(52) U.S. Cl. CPC C07D 498/04; C07D 413/04; C07D 413/14; C07D 471/04

(57) ABSTRACT

The present disclosure relates to oxadiazine compounds, pharmaceutical compositions comprising an effective amount of an oxadiazine compound and methods for using an oxadiazine compound in the treatment of a neurodegenerative disease, comprising administering to a subject in need thereof an effective amount of an oxadiazine compound.

This application claims priority to U.S. Provisional Application Ser. No. 62/311,228, filed Mar. 21, 2016; U.S. Provisional Application Ser. No. 62/279,391, filed Jan. 15, 2016; and U.S. Provisional Application Ser. No. 62/206,577, filed Aug. 18, 2015, the contents of each of which are herein incorporated by reference in their entirety.

### 1. FIELD

This disclosure relates generally to oxadiazine compounds. More specifically, the disclosure relates to the use of the oxadiazine compounds for the treatment of neurological disease.

### 2. BACKGROUND

Alzheimer's disease (AD) is the most prevalent form of dementia. It is a neurodegenerative disease that is associated (though not exclusively) with aging. The disease is clinically characterized by a progressive loss of memory, cognition, reasoning and judgment that leads to an extreme mental deterioration and ultimately death. The disease is pathologically characterized by the deposition of extracellular plaques and the presence of neurofibrillary tangles. The plaques are considered to play an important role in the pathogenesis of the disease. They mainly consist of fibrillar aggregates of β-amyloid peptide (Aβ), which are products of the amyloid precursor protein (APP). APP is initially processed by β-secretase forming a secreted peptide and a membrane bound C99 fragment. The C99 fragment is subsequently processed by the proteolytic activity of γ-secretase. Multiple sites of proteolysis on the C99 fragment lead to the production of a range of smaller peptides (Aβ 37-42 amino acids). N-terminal truncations can also be found e.g., Aβ (4-42). For convenience, notations Aβ40 and Aβ42, as used herein, include these N-terminal truncated peptides. Upon secretion, the Aβ peptides initially form soluble aggregates which ultimately lead to the formation of insoluble deposits and plaques. Aβ42 is believed to be the most neurotoxic; the shorter peptides have less propensity to aggregate and form plaques. Aβ plaques in the brain are also associated with cerebral amyloid angiopathy, hereditary cerebral hemorrhage with amyloidosis, multi infarct dementia, dementia pugilistica and Down's Syndrome.

γ-secretase is an association of four proteins: Aph1, nicastrin, presenilin and Pen-2 (review De Strooper, Neuron 38:9-12 (2003)). Subjects carrying particular mutations in one of these components, presenilin, show increased Aβ42/Aβ40 ratio. These mutations are correlated with early onset familial AD. Inhibition of γ-secretase resulting in the lowering of Aβ42 has been investigated by the pharmaceutical community, and numerous inhibitors have been found. See, e.g., Thompson et al. (Bioorg. Med. Chem. Lett. 2006, 16, 2357-63), Shaw et al. (Bioorg. Med. Chem. Lett. 2006, 17, 511-16) and Asberom et al. (Bioorg. Med. Chem. Lett. 2007, 15, 2219-2223). Inhibition of γ-secretase, though, is not without side-effects, some of which are due to the γ-secretase complex processing substrates other than C99, e.g., Notch. A more desirable approach is to modulate the proteolytic activity of the γ-secretase complex in a manner that lowers Aβ42 in favor of shorter peptides without significantly affecting the activity of γ-secretase on substrates such as Notch.

Compounds that have shown modulation of γ-secretase include certain non-steroidal, anti-inflammatory drugs (NSAIDs), for example Flurbiprofen, (Stock et al., Bioorg. Med. Chem. Lett. 2006, 16, 2219-2223). Other publications that disclose agents said to reduce Aβ42 through the modulation of γ-secretase include: WO 2004/074232, WO 2005/054193, Perreto et al., Journal of Medicinal Chemistry 2005, 48, 5705-20, WO 2005/108362, WO 2006/008558, WO 2006/021441, WO 2006/041874, WO 2006/045554, WO 2004/110350, WO 2006/043964, WO 2005/115990, EP 1847524, WO 2007/116228, WO 2007/110667, WO 2007/124394, EP 184752, EP 1849762, WO 2007/125364, WO 2009/086277 and others.

### 3. SUMMARY

It is understood that any of the embodiments described below can be combined in any desired way, and that any embodiment or combination of embodiments can be applied to each of the aspects described below, unless the context indicates otherwise.

In one aspect, the invention provides a compound of Formula (I)

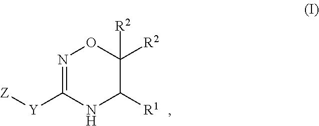

or a pharmaceutically acceptable salt thereof, wherein: R¹ is phenyl, 5- to 6-membered aromatic heterocycle, 8- to 10-membered bicyclic heterocycle or 11- to 14-membered tricyclic heterocycle, each of which is unsubstituted or substituted with one or more substituents independently selected from the group consisting of -halo, —C₁-C₄ alkyl, —C₃-C₈ monocyclic cycloalkyl, halo-substituted C₁-C₄ alkyl, —CN, —OH, —C₁-C₄ alkoxy, —O—C₃-C₈ monocyclic cycloalkyl, halo-substituted C₁-C₄ alkoxy and 3- to 7-membered monocyclic heterocycle; each R² is independently hydrogen, —C₁-C₄ alkyl or —C₃-C₆ monocyclic cycloalkyl with the proviso that both R² are not hydrogen, or both R² together with the carbon atom they are attached to form a C₃-C₆ monocyclic cycloalkyl, wherein each —C₁-C₄ alkyl and —C₃-C₆ monocyclic cycloalkyl is unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄ alkoxy; Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; and Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄ alkoxy and —OCF₃.

In some embodiments, R¹ is phenyl, 5- to 6-membered aromatic heterocycle, or 8- to 10-membered bicyclic heterocycle, each of which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more -halo; or a pharmaceutically acceptable salt thereof.

In some embodiments, R¹ is phenyl, each of which is unsubstituted or substituted with one or more —Cl or —F; or a pharmaceutically acceptable salt thereof.

In some embodiments, R¹ is phenyl substituted with one —Cl; or a pharmaceutically acceptable salt thereof.

In some embodiments, R¹ is 5- to 6-membered aromatic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 5- to 6-membered aromatic heterocycle which is unsubstituted or substituted with one or more -halo; or a pharmaceutically acceptable salt thereof.

In some embodiments, R¹ is 5- to 6-membered aromatic heterocycle which is unsubstituted or substituted with one or more —Cl or —F; or a pharmaceutically acceptable salt thereof.

In some embodiments, R¹ is 5- to 6-membered aromatic heterocycle substituted with one —Cl; or a pharmaceutically acceptable salt thereof.

In some embodiments, R¹ is 8- to 10-membered bicyclic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 8- to 10-membered bicyclic heterocycle which is unsubstituted or substituted with one or more -halo or —C₁-C₄ alkyl; or a pharmaceutically acceptable salt thereof.

In some embodiments, R¹ is 8- to 10-membered bicyclic heterocycle which is unsubstituted or substituted with one or more —C₃-C₈ monocyclic cycloalkyl or halo-substituted C₁-C₄ alkyl; or a pharmaceutically acceptable salt thereof.

In some embodiments, R¹ is 11- to 14-membered tricyclic heterocycle which is unsubstituted or substituted with one or more -halo or —C₁-C₄ alkyl; or a pharmaceutically acceptable salt thereof.

In some embodiments, 1, wherein R¹ is 11- to 14-membered tricyclic heterocycle substituted with one -halo; or a pharmaceutically acceptable salt thereof.

In some embodiments, R is

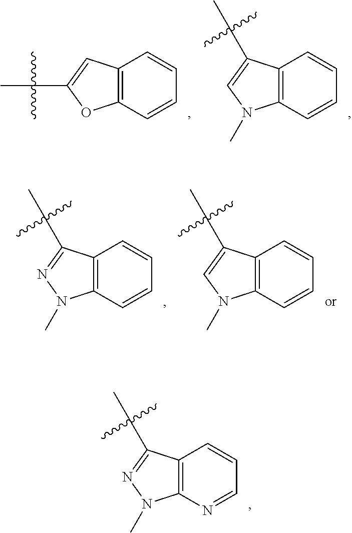 [SMILES: *c1cc2ccccc2o1 ; *c1cn(C)c2ccccc12 ; *c1nn(C)c2ccccc12 ; *c1cn(C)c2ccccc12 ; *c1nn(C)c2ncccc12]

each of which is unsubstituted or substituted with one or more -halo or halo-substituted C₁-C₄ alkyl; or a pharmaceutically acceptable salt thereof.

In some embodiments, R is selected from the group consisting of

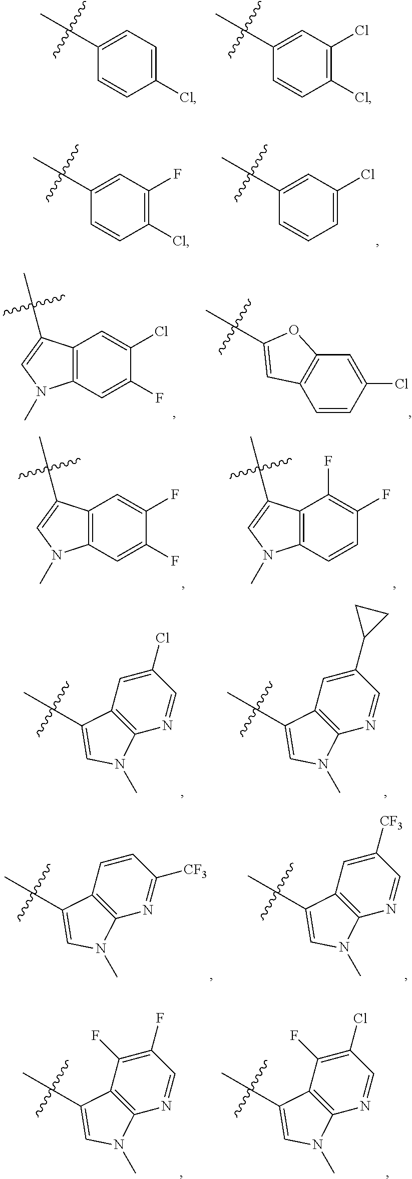

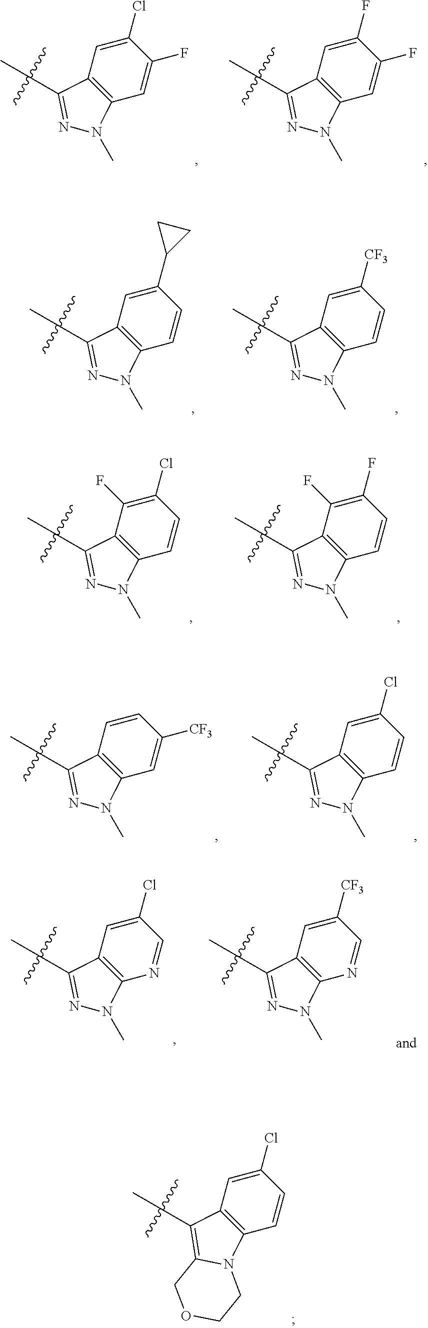

or a pharmaceutically acceptable salt thereof.

In some embodiments, R¹ is 8- to 10-membered bicyclic heterocycle substituted with —Cl, —F, —CF₃, -cyclopropyl or -methyl; or a pharmaceutically acceptable salt thereof.

In some embodiments, both R² together with the carbon atom they are attached to form a C₃-C₆ monocyclic cycloalkyl; or a pharmaceutically acceptable salt thereof.

In some embodiments, both R² together with the carbon atom they are attached to form cyclopropyl; or a pharmaceutically acceptable salt thereof.

In some embodiments, each R² is independently hydrogen, —C₁-C₄ alkyl or cyclopropyl with the proviso that both R² are not hydrogen; or a pharmaceutically acceptable salt thereof.

In some embodiments, one R² is hydrogen and the other R² is —C₁-C₄ alkyl or cyclopropyl; or a pharmaceutically acceptable salt thereof.

In some embodiments, one R² is hydrogen and the other R² is methyl or cyclopropyl; or a pharmaceutically acceptable salt thereof.

In some embodiments, one R² is hydrogen and the other R² is methyl; or a pharmaceutically acceptable salt thereof.

In some embodiments, one R² is hydrogen and the other R² is cyclopropyl; or a pharmaceutically acceptable salt thereof.

In some embodiments, each R² is —C₁-C₄ alkyl; or a pharmaceutically acceptable salt thereof.

In some embodiments, each R² is methyl; or a pharmaceutically acceptable salt thereof.

In some embodiments, one R² is hydrogen and wherein R¹ and the R² which is not hydrogen are cis; or a pharmaceutically acceptable salt thereof.

In some embodiments, one R² is hydrogen and wherein R¹ and the R² which is not hydrogen are trans; or a pharmaceutically acceptable salt thereof.

In some embodiments, Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more —C₁-C₄ alkoxy; or a pharmaceutically acceptable salt thereof.

In some embodiments, Y is pyridinyl which is substituted with one methoxy; or a pharmaceutically acceptable salt thereof.

In some embodiments, Y is

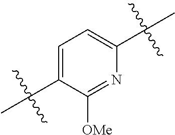 [SMILES: *c1ccc(*)c(OC)n1]

wherein the left most radical is connected to the Z group in Formula (I); or a pharmaceutically acceptable salt thereof.

In some embodiments, Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄ alkoxy and —OCF₃; or a pharmaceutically acceptable salt thereof.

In some embodiments, Z is nitrogen-containing 3- to 7-membered monocyclic heterocycle which is unsubstituted or substituted with one or more —C₁-C₄ alkyl; or a pharmaceutically acceptable salt thereof.

In some embodiments, Z is nitrogen-containing 3- to 7-membered monocyclic heterocycle which is substituted with one methyl; or a pharmaceutically acceptable salt thereof.

In some embodiments, wherein Z is imidazolyl which is unsubstituted or substituted with one methyl; or a pharmaceutically acceptable salt thereof.

In some embodiments, Z is imidazolyl which is substituted with one methyl; or a pharmaceutically acceptable salt thereof.

In some embodiments, Z is

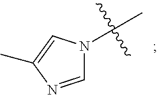 [SMILES: *n1cnc(C)c1]

or a pharmaceutically acceptable salt thereof.

In some embodiments, the compound of Formula (I) is selected from the group consisting of: (+)-(5S,6R)-5-(4-chlorophenyl)-3-[6-methoxy-5-(4-methyl-1H-imidazol-1-yl)pyridin-2-yl]-6-methyl-5,6-dihydro-4H-1,2,4-oxadiazine; (+)-cis-5-(3,4-dichlorophenyl)-3-[6-methoxy-5-(4-methyl-1H-imidazol-1-yl)pyridin-2-yl]-6-methyl-5,6-dihydro-4H-1,2,4-oxadiazine; (+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazin; (−)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-3-[6-methoxy-5-(4-methyl-1H-imidazol-1-yl)pyridin-2-yl]-6,6-dimethyl-5-phenyl-5,6-dihydro-4H-1,2,4-oxadiazin; (+)-5-(4-chlorophenyl)-3-[6-methoxy-5-(4-methyl-1H-imidazol-1-yl)pyridin-2-y]-6,6-dimethyl-5,6-dihydro-4H-1,2,4-oxadiazine; (+)-trans-5-(4-chlorophenyl)-3-[6-methoxy-5-(4-methyl-1H-imidazol-1-yl)pyridin-2-yl]-6-methyl-5,6-dihydro-4H-1,2,4-oxadiazine; and (+)-8-(4-chlorophenyl)-6-[6-methoxy-5-(4-methyl-1H-imidazol-1-yl)pyridin-2-yl]-4-oxa-5,7-diazaspiro[2.5]oct-5-ene; or a pharmaceutically acceptable salt thereof.

In one embodiment, the compound of Formula (I) is selected from the group consisting of: (+)-(5S,6R)-5-(4-chlorophenyl)-3-[6-methoxy-5-(4-methyl-1H-imidazol-1-yl)pyridin-2-yl]-6-methyl-5,6-dihydro-4H-1,2,4-oxadiazine; (+)-cis-5-(3,4-dichlorophenyl)-3-[6-methoxy-5-(4-methyl-1H-imidazol-1-yl)pyridin-2-yl]-6-methyl-5,6-dihydro-4H-1,2,4-oxadiazine; (+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazin; (−)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; (+)-5-(4-chlorophenyl)-3-[6-methoxy-5-(4-methyl-1H-imidazol-1-yl)pyridin-2-yl]-6,6-dimethyl-5,6-dihydro-4H-1,2,4-oxadiazine; (+)-trans-5-(4-chlorophenyl)-3-[6-methoxy-5-(4-methyl-1H-imidazol-1-yl)pyridin-2-yl]-6-methyl-5,6-dihydro-4H-1,2,4-oxadiazine; and (+)-8-(4-chlorophenyl)-6-[6-methoxy-5-(4-methyl-1H-imidazol-1-yl)pyridin-2-yl]-4-oxa-5,7-diazaspiro[2.5]oct-5-ene; or a pharmaceutically acceptable salt thereof.

In some embodiments, the compound is (+)-(5S,6R)-5-(4-chlorophenyl)-3-[6-methoxy-5-(4-methyl-1H-imidazol-1-yl)pyridin-2-yl]-6-methyl-5,6-dihydro-4H-1,2,4-oxadiazine; or a pharmaceutically acceptable salt thereof.

In some embodiments, the compound is (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; or a pharmaceutically acceptable salt thereof.

In some embodiments, the compound is (+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazine; or a pharmaceutically acceptable salt thereof.

In one aspect, the invention provides a compound of Formula (II)

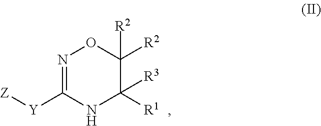

or a pharmaceutically acceptable salt thereof, wherein: R¹ is phenyl, 5- to 6-membered aromatic heterocycle, 8- to 10-membered bicyclic heterocycle or 11- to 14-membered tricyclic heterocycle, each of which is unsubstituted or substituted with one or more substituents independently selected from the group consisting of -halo, —C₁-C₄ alkyl, —C₃-C₈ monocyclic cycloalkyl, halo-substituted C₁-C₄ alkyl, —CN, —OH, —C₁-C₄ alkoxy, —O—C₃-C₈ monocyclic cycloalkyl, halo-substituted C₁-C₄ alkoxy and 3- to 7-membered monocyclic heterocycle; each R² is independently hydrogen, —C₁-C₄ alkyl or —C₃-C₆ monocyclic cycloalkyl, or both R² together with the carbon atom they are attached to form a C₃-C₆ monocyclic cycloalkyl, wherein each —C₁-C₄ alkyl and —C₃-C₆ monocyclic cycloalkyl is unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄ alkoxy; R³ is —C₁-C₄ alkyl or —C₃-C₆ monocyclic cycloalkyl, each of which is unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄ alkoxy; Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; and Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄alkoxy and —OCF₃.

In some embodiments, R¹ is phenyl, 5- to 6-membered aromatic heterocycle, or 8- to 10-membered bicyclic heterocycle, each of which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more -halo; or a pharmaceutically acceptable salt thereof.

In some embodiments, R¹ is phenyl, each of which is unsubstituted or substituted with one or more —Cl or —F; or a pharmaceutically acceptable salt thereof.

In some embodiments, R¹ is phenyl substituted with one —Cl; or a pharmaceutically acceptable salt thereof.

In some embodiments, R¹ is 5- to 6-membered aromatic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 5- to 6-membered aromatic heterocycle which is unsubstituted or substituted with one or more -halo; or a pharmaceutically acceptable salt thereof.

In some embodiments, R¹ is 5- to 6-membered aromatic heterocycle which is unsubstituted or substituted with one or more —Cl or —F; or a pharmaceutically acceptable salt thereof.

In some embodiments, R¹ is 5- to 6-membered aromatic heterocycle substituted with one —Cl; or a pharmaceutically acceptable salt thereof.

In some embodiments, R¹ is 8- to 10-membered bicyclic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 8- to 10-membered bicyclic heterocycle which is unsubstituted or substituted with one or more -halo or —C₁-C₄ alkyl; or a pharmaceutically acceptable salt thereof.

In some embodiments, R¹ is 8- to 10-membered bicyclic heterocycle which is unsubstituted or substituted with one or more —C₃-C₈ monocyclic cycloalkyl or halo-substituted C₁-C₄ alkyl; or a pharmaceutically acceptable salt thereof.

In some embodiments, R¹ is 11- to 14-membered tricyclic heterocycle which is unsubstituted or substituted with one or more -halo or —C₁-C₄ alkyl; or a pharmaceutically acceptable salt thereof.

In some embodiments, R¹ is 11- to 14-membered tricyclic heterocycle substituted with one -halo; or a pharmaceutically acceptable salt thereof.

In some embodiments, R is

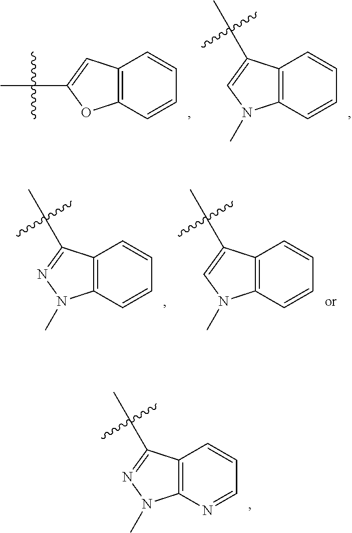 [SMILES: *c1cc2ccccc2o1 ; *c1cn(C)c2ccccc12 ; *c1nn(C)c2ccccc12 ; *c1cn(C)c2ccccc12 ; *c1nn(C)c2ncccc12]

each of which is unsubstituted or substituted with one or more -halo or halo-substituted C₁-C₄ alkyl; or a pharmaceutically acceptable salt thereof.

In some embodiments, R¹ is selected from the group consisting of

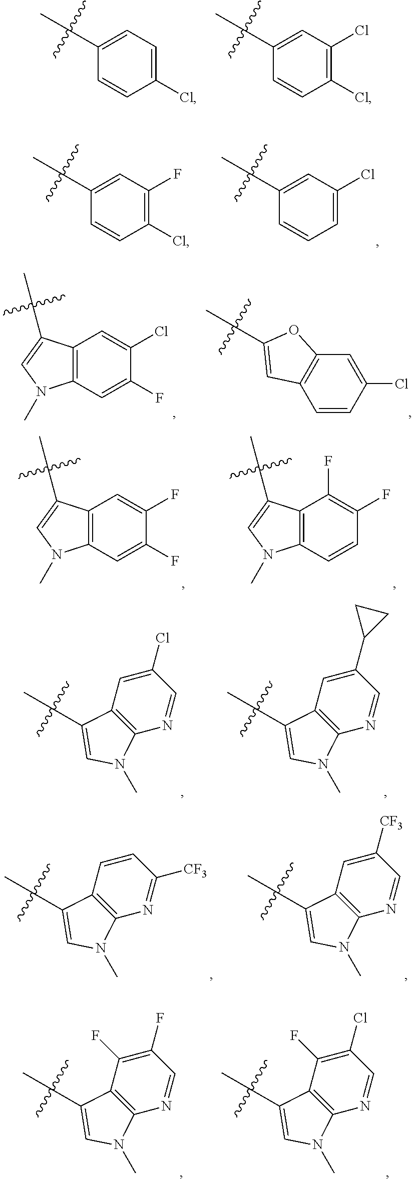

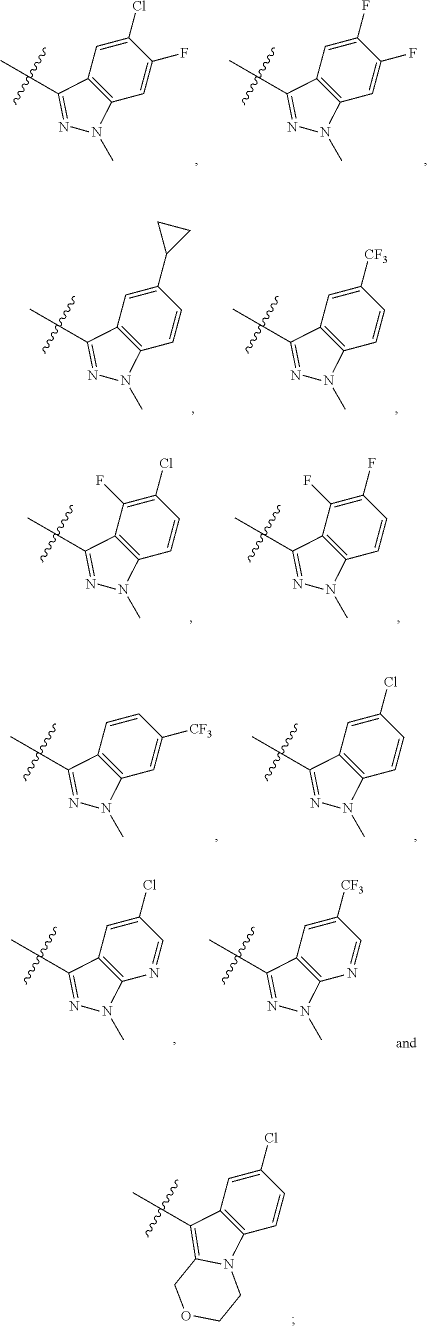

or a pharmaceutically acceptable salt thereof.

In some embodiments, R¹ is 8- to 10-membered bicyclic heterocycle substituted with —Cl, —F, —CF₃, -cyclopropyl or -methyl; or a pharmaceutically acceptable salt thereof.

In some embodiments, both R² together with the carbon atom they are attached to form a C₃-C₆ monocyclic cycloalkyl; or a pharmaceutically acceptable salt thereof.

In some embodiments, both R² together with the carbon atom they are attached to form cyclopropyl; or a pharmaceutically acceptable salt thereof.

In some embodiments, each R² is independently hydrogen, —C₁-C₄ alkyl or cyclopropyl; or a pharmaceutically acceptable salt thereof.

In some embodiments, one R² is hydrogen and the other R² is —C₁-C₄ alkyl or cyclopropyl; or a pharmaceutically acceptable salt thereof.

In some embodiments, one R² is hydrogen and the other R² is methyl or cyclopropyl; or a pharmaceutically acceptable salt thereof.

In some embodiments, one R² is hydrogen and the other R² is methyl; or a pharmaceutically acceptable salt thereof.

In some embodiments, one R² is hydrogen and the other R² is cyclopropyl; or a pharmaceutically acceptable salt thereof.

In some embodiments, each R² is —C₁-C₄ alkyl; or a pharmaceutically acceptable salt thereof.

In some embodiments, each R² is methyl; or a pharmaceutically acceptable salt thereof.

In some embodiments, one R² is hydrogen and wherein R¹ and the R² which is not hydrogen are cis; or a pharmaceutically acceptable salt thereof.

In some embodiments, one R² is hydrogen and wherein R¹ and the R² which is not hydrogen are trans; or a pharmaceutically acceptable salt thereof.

In some embodiments, R³ is —C₁-C₄ alkyl unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄alkoxy; or a pharmaceutically acceptable salt thereof.

In some embodiments, R³ is methyl; or a pharmaceutically acceptable salt thereof.

In some embodiments, R³ is —C₃-C₆ monocyclic cycloalkyl, unsubstituted or substituted with one or more substituents independently selected from the group consisting of -halo, —OH, —C₁-C₄alkoxy, —O—C₃-C₈ monocyclic cycloalkyl which is unsubstituted or substituted with halo, halo-substituted C₁-C₄ alkyl or halo-substituted C₁-C₄ alkoxy; or a pharmaceutically acceptable salt thereof.

In some embodiments, Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more —C₁-C₄ alkoxy; or a pharmaceutically acceptable salt thereof.

In some embodiments, Y is pyridinyl which is substituted with one methoxy; or a pharmaceutically acceptable salt thereof.

In some embodiments, Y is

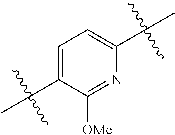 [SMILES: *c1ccc(*)c(OC)n1]

wherein the left most radical is connected to the Z group in Formula (II); or a pharmaceutically acceptable salt thereof.

In some embodiments, Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄ alkoxy and —OCF₃; or a pharmaceutically acceptable salt thereof.

In some embodiments, Z is nitrogen-containing 3- to 7-membered monocyclic heterocycle which is unsubstituted or substituted with one or more —C₁-C₄ alkyl; or a pharmaceutically acceptable salt thereof.

In some embodiments, Z is nitrogen-containing 3- to 7-membered monocyclic heterocycle which is substituted with one methyl; or a pharmaceutically acceptable salt thereof.

In some embodiments, Z is imidazolyl which is unsubstituted or substituted with one methyl; or a pharmaceutically acceptable salt thereof.

In some embodiments, Z is imidazolyl which is substituted with one methyl; or a pharmaceutically acceptable salt thereof.

In some embodiments, Z is

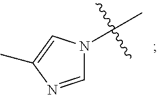 [SMILES: *n1cnc(C)c1]

or a pharmaceutically acceptable salt thereof.

In some embodiments, the compound is (+)-3-[3-[6-methoxy-5-(4-methyl-1H-imidazol-1-yl)pyridin-2-yl]-5-methyl-5,6-dihydro-4H-1,2,4-oxadiazin-5-yl]-1-methyl-1H-indole; or a pharmaceutically acceptable salt thereof.

In some embodiments, the compound is (−)-3-[3-[6-methoxy-5-(4-methyl-1H-imidazol-1-yl)pyridin-2-yl]-5-methyl-5,6-dihydro-4H-1,2,4-oxadiazin-5-yl]-1-methyl-1H-indole; or a pharmaceutically acceptable salt thereof.

In some embodiments, the compound is (5S,6S)-5-(4-chlorophenyl)-3-(6-methoxy-5-(4-methyl-1H-imidazol-1-yl)pyridin-2-yl)-5,6-dimethyl-5,6-dihydro-4H-1,2,4-oxadiazine; or a pharmaceutically acceptable salt thereof.

In some embodiments, the compound is (5S,6R)-5-(4-chlorophenyl)-3-(6-methoxy-5-(4-methyl-1H-imidazol-1-yl)pyridin-2-yl)-5,6-dimethyl-5,6-dihydro-4H-1,2,4-oxadiazine or a pharmaceutically acceptable salt thereof.

In some embodiments, the compound is (5S,6S)-5-(4-chlorophenyl)-6-cyclopropyl-3-(6-methoxy-5-(4-methyl-1H-imidazol-1-yl)pyridin-2-yl)-5-methyl-5,6-dihydro-4H-1,2,4-oxadiazine; or a pharmaceutically acceptable salt thereof.

In some embodiments, the compound is (5S,6R)-5-(4-chlorophenyl)-6-cyclopropyl-3-(6-methoxy-5-(4-methyl-1H-imidazol-1-yl)pyridin-2-yl)-5-methyl-5,6-dihydro-4H-1,2,4-oxadiazine; or a pharmaceutically acceptable salt thereof.

In some embodiments, the invention provides a levorotatory isomer of the compound of Formula (I) or Formula (II); or a pharmaceutically acceptable salt thereof.

In some embodiments, the invention provides a dextrorotatory isomer of the compound of Formula (I) or Formula (II); or a pharmaceutically acceptable salt thereof.

In one aspect, the invention provides a pharmaceutical composition comprising a pharmaceutically acceptable carrier or vehicle and an effective amount of a compound of Formula (I) or Formula (II); or a pharmaceutically acceptable salt thereof.

In one aspect, the invention provides a method for treating a neurodegenerative disease, comprising administering to a subject in need thereof an effective amount of a compound of Formula (I) or Formula (II); or a pharmaceutically acceptable salt thereof.

In some embodiments, the neurodegenerative disease is panic disorder, obsessive compulsive disorder, delusional disorder, drug-induced psychosis, post-traumatic stress disorder, age-related cognitive decline, attention deficit/hyperactivity disorder, personality disorder of the paranoid type, personality disorder of the schizoid type, dyskinesia, choreiform condition, psychosis associated with Parkinson's disease, psychotic symptoms associated with Alzheimer's disease, mood disorder, or dementia.

In some embodiments, the neurodegenerative disease is Alzheimer's disease, probable Alzheimer's disease, mild cognitive impairment, age-related cognitive decline, or another neurodegenerative disease with co-existing symptoms of Alzheimer's disease (eg. Lewy body disease or Parkinsons's disease with Alzheimer's disease).

In some embodiments, the neurodegenerative results from injuries which increase amyloid with or without cognitive impairment including post-traumatic encephalopathy and traumatic brain injury.

In some embodiments, the invention provides a method for treating Alzheimer's disease, comprising administering to a subject in need thereof an effective amount of a compound of Formula (I) or Formula (II); or a pharmaceutically acceptable salt thereof.

In some embodiments, the invention provides a method for improving an impaired cognitive function, comprising administering to a subject having impaired cognitive function an effective amount of a compound of Formula (I) or Formula (II); or a pharmaceutically acceptable salt thereof.

In some embodiments, the cognitive function impaired is one or more of attention, learning, delayed memory, working memory, visual learning, speed of processing, vigilance, verbal learning, visual motor function, social cognition, long term memory or executive function, aphasias, apraxias or frontal lobe symptoms.

In some embodiments, the invention provides a method for ameliorating a symptom of Alzheimer's disease, comprising administering to a subject in need thereof an effective amount of a compound of Formula (I) or Formula (II); or a pharmaceutically acceptable salt thereof.

In some embodiments, the symptom is progressive loss of memory, progressive loss of cognition, progressive loss of reasoning and/or progressive loss of judgment.

In some embodiments, the compound of Formula (I) or Formula (II) is a compound selected from the list of compounds in Table I or a pharmaceutically acceptable salt thereof.

In some embodiments, Alzheimer's disease is early onset Alzheimer's disease.

In some embodiments, the subject is a human.

In some embodiments, the subject is 65 years old or older. In other embodiments, the subject is 55 years old or older. In still other embodiments, the subject is 55 years old or younger, or 50 years old or younger.

A compound of Formula (I) or Formula (II) or a pharmaceutically acceptable salt thereof (also referred to herein as an “Oxadiazine”) is useful for treating, preventing or ameliorating one or more symptoms of a neurodegenerative disease.

A pharmaceutical composition comprising an effective amount of an Oxadiazine Compound and a pharmaceutically acceptable carrier or vehicle is useful for treating, preventing or ameliorating one or more symptoms of a neurodegenerative disease.

The details of the invention are set forth in the accompanying description below.

All patents and publications cited in this specification are hereby incorporated by reference in their entirety.

### 4. BRIEF DESCRIPTION OF THE DRAWINGS

FIG. 1 is a graphic representation of the single crystal structure of the compound of Example 10B.

### 5. DETAILED DESCRIPTION

### 5.1 Definitions and Abbreviations

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “C₁-C₄ alkyl” as used herein, refers to a straight chain or branched non-cyclic hydrocarbon having from 1 to 4 carbon atoms, wherein one of the hydrocarbon's hydrogen atoms has been replaced by a single bond. Representative straight chain C₁-C₄ alkyls include -methyl, -ethyl, -n-propyl and -n-butyl. Representative branched C₁-C₄ alkyls include -isopropyl, -sec-butyl, -isobutyl and -tert-butyl.

The term “C₁-C₄ alkoxy” as used herein, refers to a C₁-C₄ alkyl-O— group wherein the C₁-C₄ alkyl is as defined above. Examples of C₁-C₄ alkoxy include, but are not limited to methoxy, ethoxy, propoxy or butoxy.

The terms “halogen” or “halo” as used herein, refer to chlorine, bromine, fluorine or iodine.

The term “halo-substituted C₁-C₄ alkyl” as used herein, refers to a C₁-C₄ alkyl group, as defined above, wherein one or more of the C₁-C₄ alkyl group's hydrogen atoms have been replaced with —F, —Cl, —Br or —I. Examples of a halo-substituted C₁-C₄ alkyl include, but are not limited to, —CH₂F, —CCl₃, —CF₃, —CH₂Cl, —CH₂CH₂Br, —CH₂CH₂I, —CF₂CF₃, —CH₂CH₂CH₂F, —CH₂CH₂CH₂Cl, —CH₂CH₂CH₂CH₂Br, —CH₂CH₂CH₂CH₂I, —CH₂C H(Br)CH₃, —CH₂CH(Cl)CH₂CH₃, —CH(F)CH₂CH₃, —CH₂CF₃ and —C(CH₃)₂(CH₂Cl).

The term “halo-substituted C₁-C₄ alkoxy” as used herein, refers to a C₁-C₄ alkoxy group, as defined above, wherein one or more of the C₁-C₄ alkoxy group's hydrogen atoms have been replaced with —F, —Cl, —Br or —I. Examples of a halo-substituted C₁-C₄ alkoxy include, but are not limited to, —O—CH₂F, —O—CCl₃, —O—CF₃, —O—CH₂Cl, —O—CH₂CH₂Br, —O—CH₂CH₂I, —O—CF₂CF₃, —O—CH₂CH₂CH₂F, —O—CH₂CH₂CH₂Cl, —O—CH₂CH₂CH₂CH₂Br, —O—CH₂CH₂CH₂CH₂I, —O—CH₂CH(Br)CH₃, —O—CH₂CH(Cl)CH₂CH₃, —O—CH(F)CH₂CH₃, —OCH₂CF₃ and —O—C(CH₃)₂(CH₂Cl).

A “5- to 6-membered aromatic heterocycle” refers to a monocyclic 5- to 6-membered aromatic cycloalkyl group in which 1-4 of the cycloalkyl group's ring carbon atoms have been independently replaced with a N, O or S atom. The 5- to 6-membered aromatic heterocycles can be attached via a nitrogen or carbon atom. Representative examples of a 5- to 6-membered aromatic heterocycle group include, but are not limited to thiophenyl, furanyl, thiazolyl, isothiazolyl, oxazolyl, isoxazolyl, 1,2,4-oxadiazolyl, 1,3,4-oxadiazolyl, oxatriazolyl, pyrrazolyl, pyrrolyl, imidazolyl, tetrazolyl, 1,2,3-triazolyl, 1,3,4-triazolyl, thiadiazolyl, thiazolyl, pyridinyl, pyrimidinyl, pyrazinyl and pyridazinyl.

A “8- to 10-membered bicyclic heterocycle” refers to a bicyclic 8- to 10-membered bridged, aromatic or non-aromatic cycloalkyl group in which 1-4 of the cycloalkyl group's ring carbon atoms have been independently replaced with a N, O or S atom. The 8- to 10-membered bicyclic heterocycles can be attached via a nitrogen or carbon atom. Representative examples of a 8- to 10-membered bicyclic heterocycle group include, but are not limited to benzimidazolyl, benzothiophenyl, benzthiazolyl, benzoxazolyl, benzofuranyl, quinolinyl, isoquinolinyl, tetrahydroquinolinyl, tetrahydroisoquinolinyl, cinnolinyl, quinazolinyl, tetrahydroquinazolinyl, quinoxalinyl, tetrahydroquinoxazolinyl, indolyl, indolinyl, 1,5-naphthyridinyl, 1,2,3,4-tetrahydro-1,5-naphthyridine, 1,6-naphthyridinyl, 1,2,3,4-tetrahydro-1,6-naphthyridine, 1,7-naphthyridinyl, 1,2,3,4-tetrahydro-1,7-naphthyridine, 1,8-naphthyridinyl, 1,2,3,4-tetrahydro-1,8-naphthyridine, indazolyl, azaindazolyl and azaindolyl.

The term “3- to 7-membered monocyclic heterocycle” as used herein, refers to a monocyclic 3- to 7-membered aromatic or non-aromatic monocyclic cycloalkyl group in which 1-4 of the cycloalkyl group's ring carbon atoms have been independently replaced with a N, O or S atom. The 3- to 7- membered monocyclic heterocycles can be attached via a nitrogen or carbon atom. Representative examples of a 3- to 7-membered monocyclic heterocycle group include, but are not limited to, nitrogen-containing 3- to 7-membered monocyclic heterocycles discussed above, tetrahydrofuranyl, dihydrofuranyl, pyranyl, dihydropyranyl, tetrahydropyranyl, thiopyranyl, dihydrothiopyranyl, tetrahydrothiopyranyl, dioxanyl, dithianyl, trithianyl, dioxolanyl, furanyl and thiophenyl. In one embodiment, the 3- to 7-membered monocyclic heterocycle is a nitrogen-containing 3- to 7-membered monocyclic heterocycle. In another embodiment, the 3- to 7-membered monocyclic heterocycle is fully saturated or partially saturated.

The term “C₃-C₆ monocyclic cycloalkyl” as used herein, refers to a saturated cyclic hydrocarbon having from 3 to 6 carbon atoms. Representative C₃-C₆ monocyclic cycloalkyls include -cyclopropyl, -cyclobutyl, -cyclopentyl and -cyclohexyl.

The term “C₃-C₈ monocyclic cycloalkyl” as used herein, refers to a saturated cyclic hydrocarbon having from 3 to 8 carbon atoms. Representative C₃-C₈ monocyclic cycloalkyls include -cyclopropyl, -cyclobutyl, -cyclopentyl, -cyclohexyl, -cycloheptyl and -cyclooctyl.

The term “nitrogen-containing 5- to 6-membered aromatic monocyclic heterocycle” as used herein, refers to a 5- or 6-membered aromatic monocyclic cycloalkyl group in which from 1 to 4 of the cycloalkyl group's ring carbon atoms have been independently replaced with a nitrogen atom and O-4 of the cycloalkyl group's remaining ring carbon atoms have been independently replaced with an O or S atom. The nitrogen-containing 5- to 6-membered aromatic monocyclic heterocycle can be attached via a nitrogen or carbon atom. Representative examples of a 5- to 6-membered aromatic monocyclic heterocycles include, but are not limited to, imidazolyl, isothiazolyl, isoxazolyl, oxazolyl, oxadiazolyl, pyrimidinyl, pyrazolyl, pyrazinyl, pyridazinyl, pyridinyl, pyrrolyl, thiazolyl, thiadiazolyl, triazinyl and triazolyl. Unless otherwise indicated, the nitrogen-containing 5- to 6-membered aromatic monocyclic heterocycle is unsubstituted.

The term “C₃-C₈ monocyclic cycloalkoxy” as used herein, refers to a saturated cyclic hydrocarbon having from 3 to 8 carbon atoms that is attached through an O atom. Representative C₃-C₈ monocyclic cycloalkoxy groups include —O-cyclopropyl, —O-cyclobutyl, —O-cyclopentyl, —O-cyclohexyl, —O-cycloheptyl and —O-cyclooctyl.

The term “11- to 14-membered tricyclic heterocycle” as used herein, refers to a tricyclic 11- to 14-membered bridged and/or fused, aromatic and/or non-aromatic cycloalkyl group in which 1-6 of the ring carbon atoms have been independently replaced with a N, O or S atom. The 11- to 14-membered tricyclic heterocycle can be attached via a nitrogen or carbon atom. In some embodiments, a 11- to 14-membered tricyclic heterocycle group includes at least one 3- to 7-membered monocyclic heterocycle ring, as defined above, and the other rings may be aromatic or non-aromatic. Representative examples of a 11- to 14-membered tricyclic heterocycle group include, but are not limited to, 8-chloro-3,4-dihydro-1H-[1,4]oxazino[4,3-a]indole, 6,7,8,9-tetrahydropyrido[1,2-a]indole, 6,7,8,9-tetrahydropyrido[3,2-b]indolizine, 6,7,8,9-tetrahydropyrido[4,3-b]indolizine, 6,7,8,9-tetrahydropyrido[3,4-b]indolizine, 6,7,8,9-tetrahydropyrido[2,3-b]indolizine, 3,4-dihydro-1H-pyrido[3′,4′:4,5]pyrrolo[2,1-c][1,4]thiazine 2,2-dioxide, 3,4-dihydro-1H-[1,4]thiazino[4,3-a]indole 2,2-dioxide, 3,4-dihydro-1H-pyrido[4′,3′:4,5]pyrrolo[2,1-c][1,4]thiazine 2,2-dioxide, 8,9-dihydro-6H-pyrido[3′,2′:4,5]pyrrolo[2,1-c][1,4]thiazine 7,7-dioxide, 7,9-dihydro-6H-pyrido[2′,3′:4,5]pyrrolo[2,1-c][1,4]thiazine 8,8-dioxide, 2,3-dihydro-1H-pyrrolo[1,2-a]indole, 7,8-dihydro-6H-pyrido[4,3-b]pyrrolizine, 7,8-dihydro-6H-pyrido[3,2-b]pyrrolizine, 7,8-dihydro-6H-pyrido[3,4-b]pyrrolizine, 7,8-dihydro-6H-pyrido[2,3-b]pyrrolizine, 6-methyl-3,6-dihydro-2H-[1,4]dioxino[2,3-f]indole, 9-methyl-3,9-dihydro-2H-[1,4]dioxino[2,3-g]indole, 7-methyl-3,7-dihydro-2H-[1,4]dioxino[2,3-e]indole, 6-methyl-2,6-dihydro-1H-furo[3,2-e]indole, 6-methyl-3,6-dihydro-2H-furo[2,3-e]indole, 5-methyl-3,5-dihydro-2H-furo[2,3-f]indole, 7-methyl-3,7-dihydro-2H-furo[3,2-f]indole, 2,3-dihydro-[1,4]dioxino[2,3-f]benzofuran, 2,3-dihydro-[1,4]dioxino[2,3-g]benzofuran, 2,3-dihydro-[1,4]dioxino[2,3-e]benzofuran, 1,2-dihydrobenzo[1,2-b:4,3-b′]difuran, 2,3-dihydrobenzo[1,2-b:3,4-b′]difuran, 2,3-dihydrobenzo[1,2-b:4,5-b′]difuran, 2,3-dihydrothieno[3,2-f]benzofuran, 2,3-dihydrothieno[2,3-f]benzofuran, 2,3-dihydrothieno[3,2-f]benzofuran, 2,3-dihydrothieno[2′,3′:4,5]benzo[1,2-b][1,4]dioxine, 2,3-dihydrothieno[2′,3′:3,4]benzo[1,2-b][1,4]dioxine, 2,3-dihydrothieno[3′,2′:3,4]benzo[1,2-b][1,4]dioxine, 1,2-dihydrothieno[3,2-e]benzofuran and 2,3-dihydrothieno[2,3-g]benzofuran.

Unless otherwise indicated, any heteroatom with unsatisfied valences is assumed to have hydrogen atoms sufficient to satisfy the valences.

The phrase “pharmaceutically acceptable carrier or vehicle” as used herein, refers to a pharmaceutically acceptable material, composition or vehicle, such as a liquid or solid filler, diluent, excipient, solvent or encapsulating material, involved in carrying or transporting the Oxadiazine Compound from one organ, or portion of the body, to another organ, or portion of the body. Each carrier or vehicle must be “acceptable” in the sense of being compatible with the other ingredients of the formulation and not injurious to the subject. Some examples of materials which can serve as pharmaceutically acceptable carriers or vehicles include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The compounds described herein may form salts which are also within the scope of this invention. Reference to a compound described herein is understood to include reference to salts thereof, unless otherwise indicated. The term “salt(s)”, as employed herein, denotes acidic and/or basic salts formed with inorganic and/or organic acids and bases. In addition, when a compound described herein contains both a basic moiety, such as, but not limited to, amine, pyridine or imidazole and an acidic moiety, such as, but not limited to, a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds described herein may be formed, for example, by reacting a compound with an amount of acid or base, such as an equivalent amount, in a medium, such as one in which the salt precipitates or in an aqueous medium followed by lyophilization.

The compounds described herein which contain a basic moiety, such as, but not limited to, an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates, such as tosylates, undecanoates and the like.

The compounds described herein which contain an acidic moiety, such as, but not limited to, a carboxylic acid, may form salts with a variety of organic and inorganic bases. Exemplary basic salts include ammonium salts, alkali metal salts, such as sodium, lithium and potassium salts, alkaline earth metal salts, such as calcium and magnesium salts, salts with organic bases (for example, organic amines), such as benzathines, dicyclohexylamines, hydrabamines (formed with N,N-bis(dehydroabietyl) ethylenediamine), N-methyl-D-glucamines, N-methyl-D-glycamides, t-butyl amines and salts with amino acids, such as arginine, lysine and the like. Basic nitrogen-containing groups may be quaternized with agents, such as lower alkyl halides (e.g., methyl, ethyl, propyl and butyl chlorides, bromides and iodides), dialkyl sulfates (e.g., dimethyl, diethyl, dibutyl and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds described herein are also contemplated herein. The term “prodrug” as employed herein denotes a compound that, upon administration to a subject, undergoes chemical conversion by metabolic or chemical processes to yield a compound described herein, or a salt and/or solvate thereof. Solvates of the compounds described herein include, for example, hydrates.

Compounds described herein are, subsequent to their preparation, preferably isolated and purified to obtain a composition containing an amount by weight equal to or greater than 90%, for example, equal to or greater than 95%, equal to or greater than 97%, equal to or greater than 98%, or equal to or greater than 99% of the compounds (“substantially pure” compounds), which is then used or formulated as described herein. Such “substantially pure” compounds described herein are also contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds described herein may, for example, be substantially free of other isomers (e.g., as a pure or substantially pure optical isomer having a specified activity), or may be admixed, for example, as racemates or with all other, or other selected, stereoisomers. The chiral centers of the compounds described herein may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

All configurational isomers of the compounds described herein are contemplated, either in admixture or in pure or substantially pure form. Certain compounds described herein may exist in particular geometric or stereoisomeric forms. The present invention contemplates all such compounds, including cis- and trans-isomers, cis (Z) and trans (E) alkene isomers, R- and S-enantiomers, diastereomers, (D)-isomers, (L)-isomers, the racemic mixtures thereof, and other mixtures thereof, as falling within the scope of the invention. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

Throughout the specifications, groups and substituents thereof may be chosen to provide stable moieties and compounds. Compounds useful in the treatment, for example, are neurodegenerative disorders. The term “stable”, as used herein, refers to compounds which possess stability sufficient to allow manufacture and which maintain the integrity of the compound for a sufficient period of time to be detected and preferably for a sufficient period of time to be useful for the purposes detailed herein.

Definitions of specific functional groups and chemical terms are described in more detail above. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999, the entire contents of which are incorporated herein by reference.

In some embodiments, the present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl, respectively. Compounds described herein, or an enantiomer, diastereomer, tautomer, or pharmaceutically acceptable salt or solvate thereof, which contain the aforementioned isotopes and/or other isotopes of other atoms, are within the scope of this invention. Certain isotopically labeled compounds described herein, for example those into which radioactive isotopes, such as ³H and ¹⁴C are incorporated, are useful in drug and/or substrate tissue distribution assays. Tritiated, i.e., ³H, and carbon-14, i.e. ¹⁴C, isotopes are particularly preferred for their ease of preparation and detectability. Further, substitution with heavier isotopes, such as deuterium, i.e., ²H, can afford certain therapeutic advantages resulting from greater metabolic stability, for example increased in vivo half-life or reduced dosage requirements, and hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

When more than one position in any given structure may be substituted with more than one substituent selected from a specified group, the substituent may be either the same or different at every position.

As used herein, “effective amount” refers to any amount that is necessary or sufficient for achieving or promoting a desired outcome, e.g., for treating, preventing, or ameliorating a symptom of a neurodegenerative disease. In some instances an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition.

As used herein, “treat” or “treating” includes stopping the progression and/or reducing or ameliorating a symptom of a neurodegenerative disease, for example, improving cognitive function.

As used herein, the term “subject” refers to a vertebrate animal. In one embodiment the subject is a mammal. In one embodiment the subject is a human. In other embodiments the subject is a non-human vertebrate animal, including, without limitation, non-human primates, laboratory animals, livestock, domesticated animals and non-domesticated animals. Non-limiting examples of subject include a mammal, e g, a human, mouse, rat, guinea pig, dog, cat, horse, cow, pig, and non-human primate, such as a monkey, chimpanzee, baboon or rhesus. In one embodiment, the subject is a human.

Practitioners of the art will recognize that certain chemical groups may exist in multiple tautomeric forms (for example, as an amide or imino ether). The scope of this disclosure is meant to include all such tautomeric forms. For example, a tetrazole may exist in two tautomeric forms, 1-H tetrazole and a 2-H tetrazole. This is depicted in the figure below. This example is not meant to be limiting in the scope of tautomeric forms.

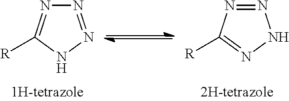

Practitioners of the art will recognize that certain electrophilic ketones, may exist in a hydrated form. The scope of this disclosure is to include all such hydrated forms. For example, a trifluoromethyl ketone may exist in a hydrated form via addition of water to the carbonyl group. This is depicted in the figure below. This example is not meant to be limiting in the scope of hydrated forms.

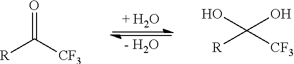

### ABBREVIATIONS

Abbreviations used in the following examples and preparations include:
Å Angstrom
Aβ Amyloid-beta
Ac Acyl (Me—C(O)—)
ACN Acetonitrile
AD Alzheimer's Disease
APP Amyloid Precursor Protein
Aq Aqueous
Bn Benzyl
Boc Tert-Butyloxycarbonyl
BSA Bovine Serum Albumin
Bu Butyl
BuLi Butyllithium
c Cyclo
cBu Cylcobutyl
cm Centimeter
Conc. Concentrated
cPr Cyclopropyl
CSF Cerebrospinal Fluid
d Day(s)
dba Dibenzylideneacetone
DCE 1, 2-Dichloroethane
DCM Dichloromethane
DEA Di-ethylamine
Dens. Density
DIAD Diisopropyl Azodicarboxylate
DIBALH Diisobutylaluminium Hydride
DIPEA N, N-Diisopropylethylamine
DMAP 4-Dimethylaminopyridine
DMF Dimethylformamide
DMS Dimethylsulfate
DMSO Dimethyl Sulfoxide
dppf 1,1′-Bis(diphenylphosphino)ferrocene
EDC 1-[3-(Dimethylamino)propyl]-3-ethylcarbodiimide
ELISA Enzyme-Linked Immuno Sorbent Assay
ESI Electrospray Ionization
Et Ethyl
Et3N Triethylamine
Eq. Equivalent
g Grams(s)
HATU 1-[Bis(dimethylamino)nethylenel-1H-1,2,3-triazolol4,5-b]pyridinium 3-oxid Hexafluorophosphate
HOAt 1-Hydroxy-7-azabenzotriazole
HPLC High Pressure Liquid Chromatography
h Hour(s)
IPA Isopropyl Alcohol
iPr Isopropyl
iOPr Isopropoxyl
i.v or IV. Intravenous
LAH Lithium Aluminum Hydride
LC-MS Liquid Chromatography-Mass Spectrometry
LC/MS Liquid Chromatography Mass Spectrometry
LG Leaving Group
LRMS Low Resolution Mass Spectrometry
m Multiplet
Max. Maximum
mCPBA Meta-Chloroperoxybenzoic Acid
Me Methyl
MeOH Methanol
Min. Minimum
min Minute(s)
mmol Millimoles
μl Microliter
ul Microliter
μm Micrometer
MS Mass Spectrometry
MW Molecular Weight (all values are ±0.05)
n Normal
N Normal
NaHMDS Sodium Hexamethyldisilazane
NB S N-Bromosuccinimide
NCS N-Chlorosuccinimide
NIS N-Iodosuccinimide
NMP 1-Methylpyrrolidin-2-one
NMR Nuclear Magnetic Resonance
Nref The Number of Reflections Used in the Refinement
Npar The Number of Refined Parameters
NSAIDS Non-Steroidal Anti-Inflammatory Drugs
o/n Overnight
PBS Phosphate Buffered Saline
PPA Poly Phosphoric Acid
Ph Phenyl
Phth Phthalimide
Phthal Phthalyl
Pr Propyl
PS-BEMP Polystyrene 2-Tert-butylimino-2-diethylamino-1,3-dimethyl-perhydro-1,3,2-diazaphosphorine
Py Pyridine
Ra-Ni Raney Nickel
Resd. Residential
Rf Retention Factor
RT (or rt) Room Temperature (about 20-25° C.) or Retention Time
RT Retention Time
s Singlet
sat. Saturated
SEM-Cl 2-(Trimethylsilyl)ethoxymethyl Chloride
t Triplet
TBAF Tetrabutylammonium Fluoride
t-Bu Tertiary Butyl
tert Tertiary
TFA Trifluoroacetic Acid
TFAA Trifluoroacetic Anhydride
THF Tetrahydrofuran
TLC Thin Layer Chromatography
TMS Trimethylsilyl
TPP Triphenylphosphine
UPLC Ultra Performance Liquid Chromatography
v/v Volume/volume
wt/v Weight/volume
XPhos 2-Dicyclohexylphosphino-2′,4′,6′-triisopropylbiphenyl

### 5.2 Oxadiazine Compounds

Described below are Oxadiazine Compounds, i.e., compounds according to Formula (I), Formula (II) or compounds in Table I-1, and pharmaceutically acceptable salts thereof, as well as methods for preparing the compounds and using the compounds to treat one or more neurodegenerative diseases, e.g., reducing a symptom of Alzheimer's disease (such as improving cognitive function). The compounds of the disclosure are believed to be gamma secretase modulators (GSMs),i.e., compounds that act to shift the relative levels of Aβ peptides produced by γ-secretase. In some embodiments, the compounds alter the relative levels of Aβ peptides produced by γ-secretase, for example the level of Aβ42 peptide, without significantly changing the total level of Aβ peptides produced.

In one aspect, Oxadiazine Compounds described herein are compounds according to Formula (I), below:

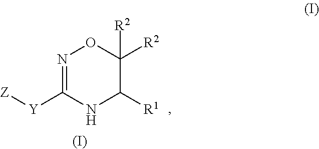

and pharmaceutically acceptable salts thereof,
wherein R¹, R², Y and Z are as defined above for the compounds of Formula (I).

In some embodiments, R¹ is phenyl, 5- to 6-membered aromatic heterocycle, or 8- to 10-membered bicyclic heterocycle, each of which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more -halo; or a pharmaceutically acceptable salt thereof.

In some embodiments, R¹ is phenyl, each of which is unsubstituted or substituted with one or more —Cl or —F; or a pharmaceutically acceptable salt thereof.

In some embodiments, R¹ is phenyl substituted with one —Cl; or a pharmaceutically acceptable salt thereof.

In some embodiments, R¹ is 5- to 6-membered aromatic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 5- to 6-membered aromatic heterocycle which is unsubstituted or substituted with one or more -halo; or a pharmaceutically acceptable salt thereof.

In some embodiments, R¹ is 5- to 6-membered aromatic heterocycle which is unsubstituted or substituted with one or more —Cl or —F; or a pharmaceutically acceptable salt thereof.

In some embodiments, R¹ is 5- to 6-membered aromatic heterocycle substituted with one —Cl; or a pharmaceutically acceptable salt thereof.

In some embodiments, R¹ is 8- to 10-membered bicyclic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 8- to 10-membered bicyclic heterocycle which is unsubstituted or substituted with one or more -halo or —C₁-C₄ alkyl; or a pharmaceutically acceptable salt thereof.

In some embodiments, R¹ is 8- to 10-membered bicyclic heterocycle which is unsubstituted or substituted with one or more —C₃-C₈ monocyclic cycloalkyl or halo-substituted C₁-C₄ alkyl; or a pharmaceutically acceptable salt thereof.

In some embodiments, R¹ is 11- to 14-membered tricyclic heterocycle which is unsubstituted or substituted with one or more -halo or —C₁-C₄ alkyl; or a pharmaceutically acceptable salt thereof.

In some embodiments, 1, wherein R¹ is 11- to 14-membered tricyclic heterocycle substituted with one -halo; or a pharmaceutically acceptable salt thereof.

In some embodiments, R¹ is

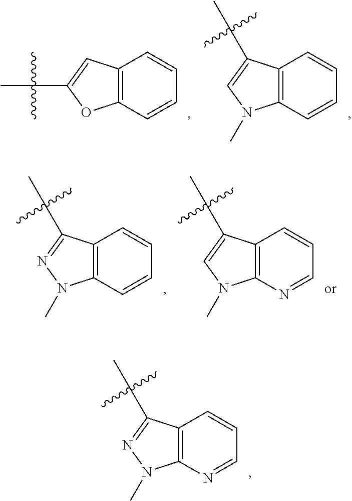 [SMILES: *c1cc2ccccc2o1 ; *c1cn(C)c2ccccc12 ; *c1nn(C)c2ccccc12 ; *c1cn(C)c2ncccc12 ; *c1nn(C)c2ncccc12]

each of which is unsubstituted or substituted with one or more -halo or halo-substituted C₁-C₄alkyl; or a pharmaceutically acceptable salt thereof.

In some embodiments, R¹ is selected from the group consisting of

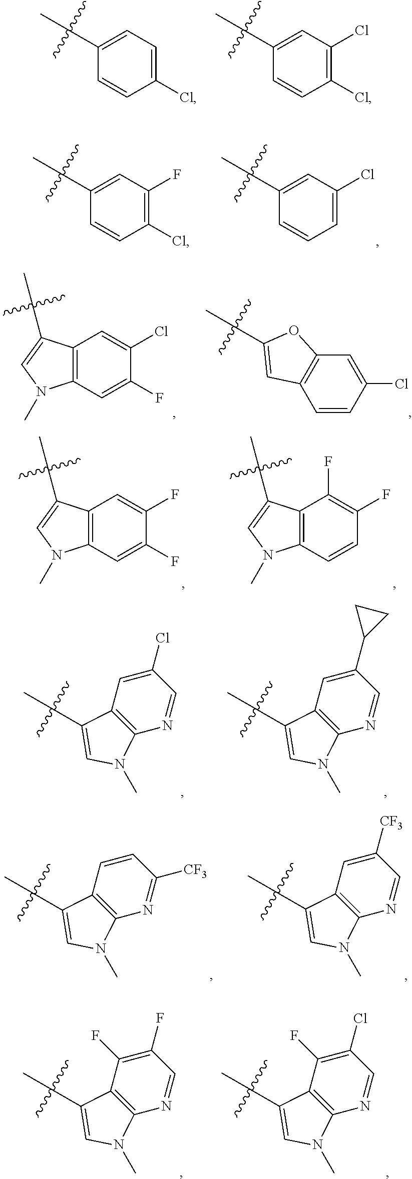

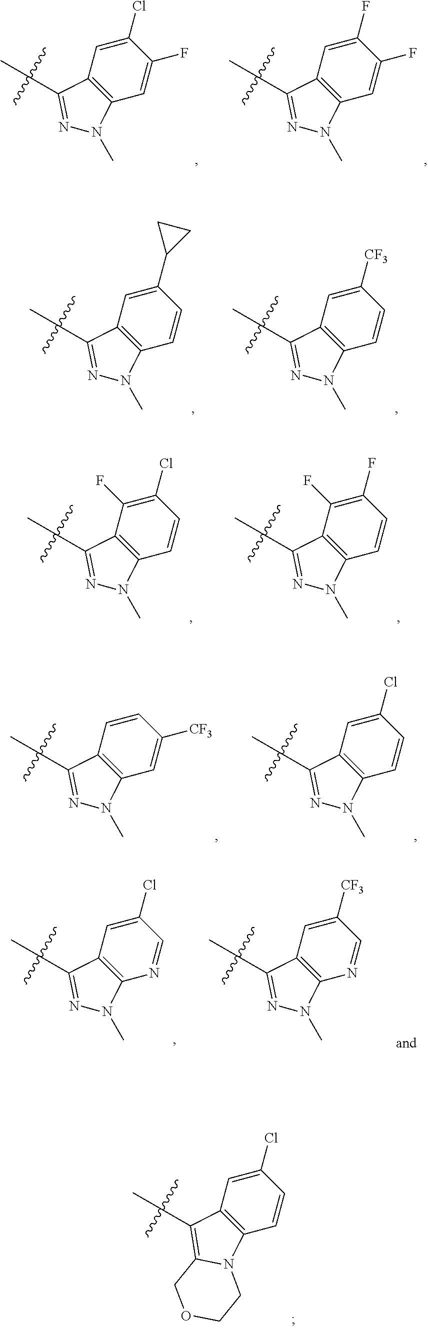

or a pharmaceutically acceptable salt thereof.

In some embodiments, R¹ is 8- to 10-membered bicyclic heterocycle substituted with —Cl, —F, —CF₃, -cyclopropyl or -methyl; or a pharmaceutically acceptable salt thereof.

In some embodiments, both R² together with the carbon atom they are attached to form a C₃-C₆ monocyclic cycloalkyl; or a pharmaceutically acceptable salt thereof.

In some embodiments, both R² together with the carbon atom they are attached to form cyclopropyl; or a pharmaceutically acceptable salt thereof.

In some embodiments, each R² is independently hydrogen, —C₁-C₄ alkyl or cyclopropyl with the proviso that both R² are not hydrogen; or a pharmaceutically acceptable salt thereof.

In some embodiments, one R² is hydrogen and the other R² is —C₁-C₄ alkyl or cyclopropyl; or a pharmaceutically acceptable salt thereof.

In some embodiments, one R² is hydrogen and the other R² is methyl or cyclopropyl; or a pharmaceutically acceptable salt thereof.

In some embodiments, one R² is hydrogen and the other R² is methyl; or a pharmaceutically acceptable salt thereof.

In some embodiments, one R² is hydrogen and the other R² is cyclopropyl; or a pharmaceutically acceptable salt thereof.

In some embodiments, each R² is —C₁-C₄ alkyl; or a pharmaceutically acceptable salt thereof.

In some embodiments, each R² is methyl; or a pharmaceutically acceptable salt thereof.

In some embodiments, one R² is hydrogen and wherein R¹ and the R² which is not hydrogen are cis; or a pharmaceutically acceptable salt thereof.

In some embodiments, one R² is hydrogen and wherein R¹ and the R² which is not hydrogen are trans; or a pharmaceutically acceptable salt thereof.

In some embodiments, Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more —C₁-C₄ alkoxy; or a pharmaceutically acceptable salt thereof.

In some embodiments, Y is pyridinyl which is substituted with one methoxy; or a pharmaceutically acceptable salt thereof.

In some embodiments, Y is

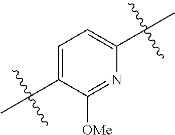 [SMILES: *c1ccc(*)c(OC)n1]

wherein the left most radical is connected to the Z group in Formula (I); or a pharmaceutically acceptable salt thereof.

In some embodiments, Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄ alkoxy and —OCF₃; or a pharmaceutically acceptable salt thereof.

In some embodiments, Z is nitrogen-containing 3- to 7-membered monocyclic heterocycle which is unsubstituted or substituted with one or more —C₁-C₄ alkyl; or a pharmaceutically acceptable salt thereof.

In some embodiments, Z is nitrogen-containing 3- to 7-membered monocyclic heterocycle which is substituted with one methyl; or a pharmaceutically acceptable salt thereof.

In some embodiments, wherein Z is imidazolyl which is unsubstituted or substituted with one methyl; or a pharmaceutically acceptable salt thereof.

In some embodiments, Z is imidazolyl which is substituted with one methyl; or a pharmaceutically acceptable salt thereof.

In some embodiments, Z is

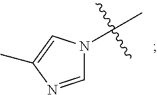 [SMILES: *n1cnc(C)c1]

or a pharmaceutically acceptable salt thereof.

In another aspect, Oxadiazine Compoundss described herein are compounds of Formula (II), below:

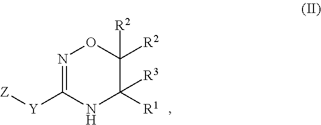

and pharmaceutically acceptable salts thereof,
wherein R¹, R², R³, Y and Z are as defined above for the compounds of Formula (II).

In some embodiments, R¹ is phenyl, 5- to 6-membered aromatic heterocycle, or 8- to 10-membered bicyclic heterocycle, each of which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is phenyl which is unsubstituted or substituted with one or more -halo; or a pharmaceutically acceptable salt thereof.

In some embodiments, R¹ is phenyl, each of which is unsubstituted or substituted with one or more —Cl or —F; or a pharmaceutically acceptable salt thereof.

In some embodiments, R¹ is phenyl substituted with one —Cl; or a pharmaceutically acceptable salt thereof.

In some embodiments, R¹ is 5- to 6-membered aromatic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 5- to 6-membered aromatic heterocycle which is unsubstituted or substituted with one or more -halo; or a pharmaceutically acceptable salt thereof.

In some embodiments, R¹ is 5- to 6-membered aromatic heterocycle which is unsubstituted or substituted with one or more —Cl or —F; or a pharmaceutically acceptable salt thereof.

In some embodiments, R¹ is 5- to 6-membered aromatic heterocycle substituted with one —Cl; or a pharmaceutically acceptable salt thereof.

In some embodiments, R¹ is 8- to 10-membered bicyclic heterocycle which is unsubstituted or substituted with one or more substituents independently selected from the group consisting of -halo, —C₁-C₄ alkyl, —C₃-C₈ monocyclic cycloalkyl and halo-substituted C₁-C₄ alkyl; or a pharmaceutically acceptable salt thereof.

In some embodiments, R¹ is 8- to 10-membered bicyclic heterocycle which is unsubstituted or substituted with one or more -halo or —C₁-C₄ alkyl; or a pharmaceutically acceptable salt thereof.

In some embodiments, R¹ is 8- to 10-membered bicyclic heterocycle which is unsubstituted or substituted with one or more —C₃-C₈ monocyclic cycloalkyl or halo-substituted C₁-C₄ alkyl; or a pharmaceutically acceptable salt thereof.

In some embodiments, R¹ is 11- to 14-membered tricyclic heterocycle which is unsubstituted or substituted with one or more -halo or —C₁-C₄ alkyl; or a pharmaceutically acceptable salt thereof.

In some embodiments, R¹ is 11- to 14-membered tricyclic heterocycle substituted with one -halo; or a pharmaceutically acceptable salt thereof.

In some embodiments, R is

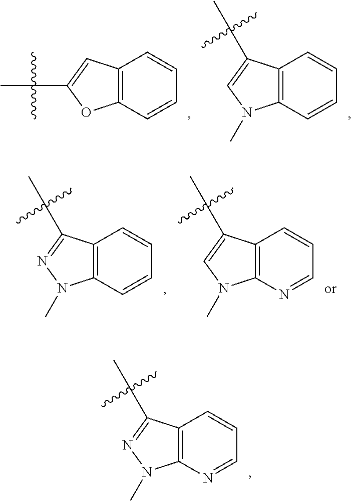 [SMILES: *c1cc2ccccc2o1 ; *c1cn(C)c2ccccc12 ; *c1nn(C)c2ccccc12 ; *c1cn(C)c2ncccc12 ; *c1nn(C)c2ncccc12]

each of which is unsubstituted or substituted with one or more -halo or halo-substituted C₁-C₄ alkyl; or a pharmaceutically acceptable salt thereof.

In some embodiments, R is selected from the group consisting of

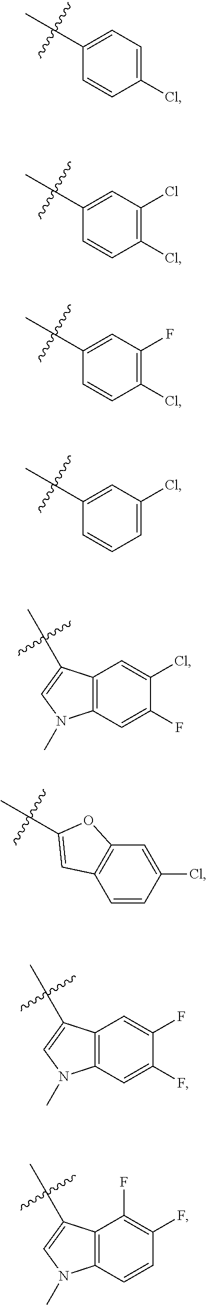

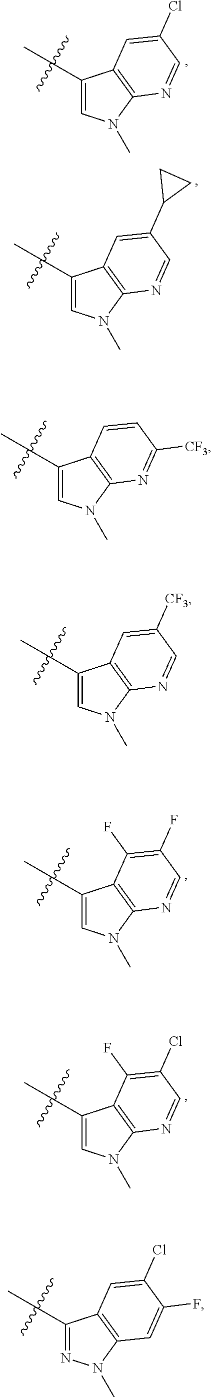

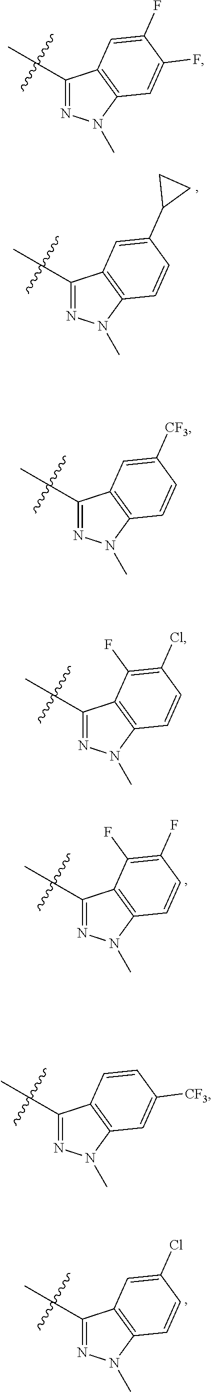

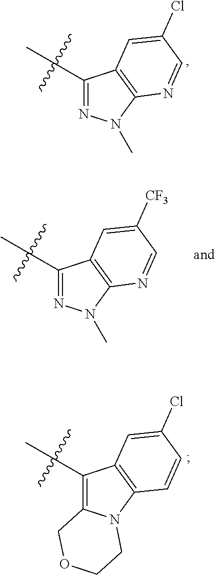

or a pharmaceutically acceptable salt thereof.

In some embodiments, R¹ is 8- to 10-membered bicyclic heterocycle substituted with —Cl, —F, —CF₃, -cyclopropyl or -methyl; or a pharmaceutically acceptable salt thereof.

In some embodiments, both R² together with the carbon atom they are attached to form a C₃-C₆ monocyclic cycloalkyl; or a pharmaceutically acceptable salt thereof.

In some embodiments, both R² together with the carbon atom they are attached to form cyclopropyl; or a pharmaceutically acceptable salt thereof.

In some embodiments, each R² is independently hydrogen, —C₁-C₄ alkyl or cyclopropyl; or a pharmaceutically acceptable salt thereof.

In some embodiments, one R² is hydrogen and the other R² is —C₁-C₄ alkyl or cyclopropyl; or a pharmaceutically acceptable salt thereof.

In some embodiments, one R² is hydrogen and the other R² is methyl or cyclopropyl; or a pharmaceutically acceptable salt thereof.

In some embodiments, one R² is hydrogen and the other R² is methyl; or a pharmaceutically acceptable salt thereof.

In some embodiments, one R² is hydrogen and the other R² is cyclopropyl; or a pharmaceutically acceptable salt thereof.

In some embodiments, each R² is —C₁-C₄ alkyl; or a pharmaceutically acceptable salt thereof.

In some embodiments, each R² is methyl; or a pharmaceutically acceptable salt thereof.

In some embodiments, one R² is hydrogen and wherein R¹ and the R² which is not hydrogen are cis; or a pharmaceutically acceptable salt thereof.

In some embodiments, one R² is hydrogen and wherein R¹ and the R² which is not hydrogen are trans; or a pharmaceutically acceptable salt thereof.

In some embodiments, R³ is —C₁-C₄ alkyl unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄ alkoxy; or a pharmaceutically acceptable salt thereof

In some embodiments, R³ is methyl; or a pharmaceutically acceptable salt thereof.

In some embodiments, R³ is —C₃-C₆ monocyclic cycloalkyl, unsubstituted or substituted with one or more substituents independently selected from the group consisting of -halo, —OH, —C₁-C₄ alkoxy, —O—C₃-C₈ monocyclic cycloalkyl which is unsubstituted or substituted with halo, halo-substituted C₁-C₄ alkyl or halo-substituted C₁-C₄ alkoxy; or a pharmaceutically acceptable salt thereof.

In some embodiments, Y is pyridinyl or phenyl, each of which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more substituents independently selected from the group consisting of -halo, —C₁-C₄ alkoxy, halo-substituted C₁-C₄ alkoxy, —C₁-C₄ alkyl, halo-substituted C₁-C₄ alkyl, —CN and —OH; or a pharmaceutically acceptable salt thereof.

In some embodiments, Y is pyridinyl which is unsubstituted or substituted with one or more —C₁-C₄ alkoxy; or a pharmaceutically acceptable salt thereof.

In some embodiments, Y is pyridinyl which is substituted with one methoxy; or a pharmaceutically acceptable salt thereof.

In some embodiments, Y is

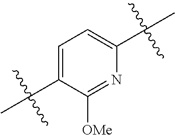 [SMILES: *c1ccc(*)c(OC)n1]

wherein the left most radical is connected to the Z group in Formula (II); or a pharmaceutically acceptable salt thereof.

In some embodiments, Z is nitrogen-containing 3- to 7-membered monocyclic heterocycle which is unsubstituted or substituted with one or more substituents independently selected from the group consisting of -halo, —C₁-C₄ alkyl, halo-substituted C₁-C₄ alkyl, —C₁-C₄ alkoxy and —OCF₃; or a pharmaceutically acceptable salt thereof.

In some embodiments, Z is nitrogen-containing 3- to 7-membered monocyclic heterocycle which is unsubstituted or substituted with one or more —C₁-C₄ alkyl; or a pharmaceutically acceptable salt thereof.

In some embodiments, Z is nitrogen-containing 3- to 7-membered monocyclic heterocycle which is substituted with one methyl; or a pharmaceutically acceptable salt thereof.

In some embodiments, Z is imidazolyl which is unsubstituted or substituted with one methyl; or a pharmaceutically acceptable salt thereof.

In some embodiments, Z is imidazolyl which is substituted with one methyl; or a pharmaceutically acceptable salt thereof.

In some embodiments, Z is

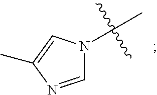 [SMILES: *n1cnc(C)c1]

or a pharmaceutically acceptable salt thereof.

In some embodiments, the invention provides a levorotatory isomer of the compound of Formula (I) or Formula (II); or a pharmaceutically acceptable salt thereof.

In some embodiments, the invention provides a dextrorotatory isomer of the compound of Formula (I) or Formula (II); or a pharmaceutically acceptable salt thereof.

In some embodiments, the compound of Formula (I) and Formula (II) is a compound selected from the compounds in Table I. In some embodiments, the compound of Formula (I) and Formula (II) is a pharmaceutically acceptable salt of a compound selected from the compounds in Table I.

TABLE I
Exemplary Oxadiazine Compounds of Formula (I) and Formula (II)

| Compound | | |
|---|---|---|
| of Example | Structure | Name |
| 18A | 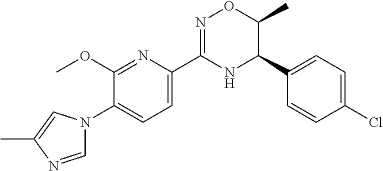 [SMILES: COc1nc(C2=NO[C@@H](C)[C@@H](c3ccc(Cl)cc3)N2)ccc1-n1cnc(C)c1] | (−)-(5R,6S)-5-(4- chlorophenyl)-3-[6-methoxy- 5-(4-methyl-1H-imidazol-1- yl)pyridin-2-yl]-6-methyl-5,6- dihydro-4H-1,2,4-oxadiazine |
| 18B | 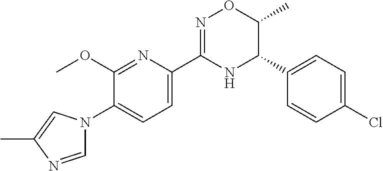 [SMILES: COc1nc(C2=NO[C@H](C)[C@H](c3ccc(Cl)cc3)N2)ccc1-n1cnc(C)c1] | (+)-(5S,6R)-5-(4- chlorophenyl)-3-[6-methoxy- 5-(4-methyl-1H-imidazol-1- yl)pyridin-2-yl]-6-methyl-5,6- dihydro-4H-1,2,4-oxadiazine |
| 19A | 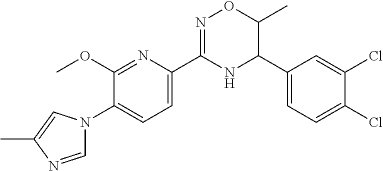 [SMILES: COc1nc(C2=NOC(C)C(c3ccc(Cl)c(Cl)c3)N2)ccc1-n1cnc(C)c1] | (−)-cis-5-(3,4- dichlorophenyl)-3-[6- methoxy-5-(4-methyl-1H- imidazol-1-yl)pyridin-2-yl]-6- methyl-5,6-dihydro-4H-1,2,4- oxadiazine |
| 19B | 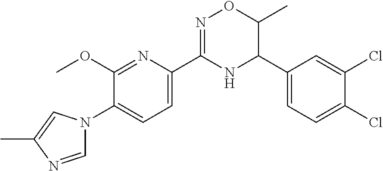 [SMILES: COc1nc(C2=NOC(C)C(c3ccc(Cl)c(Cl)c3)N2)ccc1-n1cnc(C)c1] | (+)-cis-5-(3,4- dichlorophenyl)-3-[6- methoxy-5-(4-methyl-1H- imidazol-1-yl)pyridin-2-yl]-6- methyl-5,6-dihydro-4H-1,2,4- oxadiazine |
| 20A | 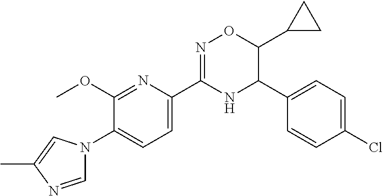 [SMILES: COc1nc(C2=NOC(C3CC3)C(c3ccc(Cl)cc3)N2)ccc1-n1cnc(C)c1] | (−)-cis-5-(4-chlorophenyl)-6- cyclopropyl-3-[6-methoxy-5- (4-methyl-1H-imidazol-1- yl)pyridin-2-yl]-5,6-dihydro- 4H-1,2,4-oxadiazine |
| 20B | 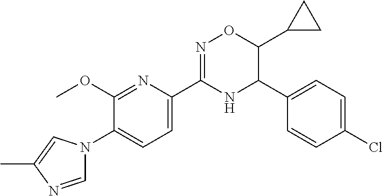 [SMILES: COc1nc(C2=NOC(C3CC3)C(c3ccc(Cl)cc3)N2)ccc1-n1cnc(C)c1] | (+)-cis-5-(4-chlorophenyl)-6- cyclopropyl-3-[6-methoxy-5- (4-methyl-1H-imidazol-1- yl)pyridin-2-yl]-5,6-dihydro- 4H-1,2,4-oxadiazine |
| 21A | 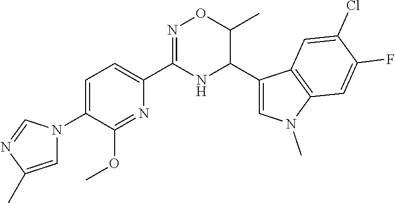 [SMILES: COc1nc(C2=NOC(C)C(c3cn(C)c4cc(F)c(Cl)cc34)N2)ccc1-n1cnc(C)c1] | (−)-5-(5-chloro-6-fluoro-1- methyl-1H-indol-3-yl)-3-(6- methoxy-5-(4-methyl-1H- imidazol-1-yl) pyridin-2-yl)- 6-methyl-5,6-dihydro-4H- 1,2,4-oxadiazine |
| 21B | 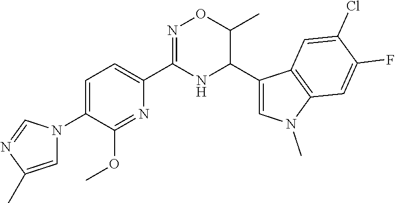 [SMILES: COc1nc(C2=NOC(C)C(c3cn(C)c4cc(F)c(Cl)cc34)N2)ccc1-n1cnc(C)c1] | (+)-5-(5-chloro-6-fluoro-1- methyl-1H-indol-3-yl)-3-(6- methoxy-5-(4-methyl-1H- imidazol-1-yl) pyridin-2-yl)- 6-methyl-5,6-dihydro-4H- 1,2,4-oxadiazine |
| 21C | 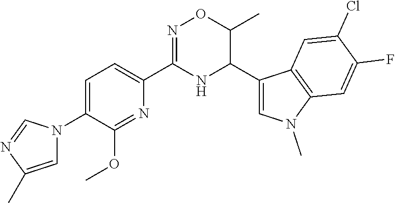 [SMILES: COc1nc(C2=NOC(C)C(c3cn(C)c4cc(F)c(Cl)cc34)N2)ccc1-n1cnc(C)c1] | (−)-5-(5-chloro-6-fluoro-1- methyl-1H-indol-3-yl)-3-(6- methoxy-5-(4-methyl-1H- imidazol-1-yl) pyridin-2-yl)- 6-methyl-5,6-dihydro-4H- 1,2,4-oxadiazine |
| 21D | 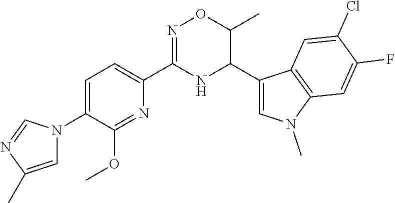 [SMILES: COc1nc(C2=NOC(C)C(c3cn(C)c4cc(F)c(Cl)cc34)N2)ccc1-n1cnc(C)c1] | (+)-5-(5-chloro-6-fluoro-1- methyl-1H-indol-3-yl)-3-(6- methoxy-5-(4-methyl-1H- imidazol-1-yl) pyridin-2-yl)- 6-methyl-5,6-dihydro-4H- 1,2,4-oxadiazine |
| 22A | 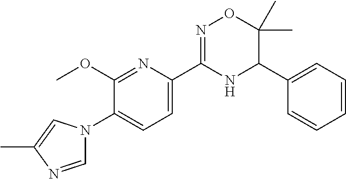 [SMILES: COc1nc(C2=NOC(C)(C)C(c3ccccc3)N2)ccc1-n1cnc(C)c1] | (−)-3-[6-methoxy-5-(4- methyl-1H-imidazol-1- yl)pyridin-2-yl]-6,6-dimethyl- 5-phenyl-5,6-dihydro-4H- 1,2,4-oxadiazine |
| 22B | 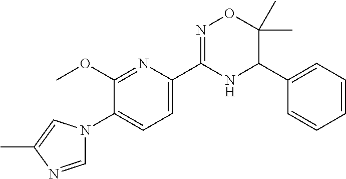 [SMILES: COc1nc(C2=NOC(C)(C)C(c3ccccc3)N2)ccc1-n1cnc(C)c1] | (+)-3-[6-methoxy-5-(4- methyl-1H-imidazol-1- yl)pyridin-2-yl]-6,6-dimethyl- 5-phenyl-5,6-dihydro-4H- 1,2,4-oxadiazine |
| 23A | 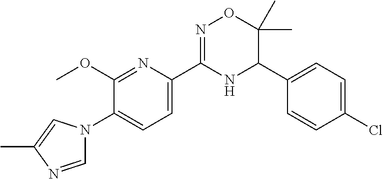 [SMILES: COc1nc(C2=NOC(C)(C)C(c3ccc(Cl)cc3)N2)ccc1-n1cnc(C)c1] | (−)-5-(4-chlorophenyl)-3-[6- methoxy-5-(4-methyl-1H- imidazol-1-yl)pyridin-2-yl]- 6,6-dimethyl-5,6-dihydro- 4H-1,2,4-oxadiazine |
| 23B | 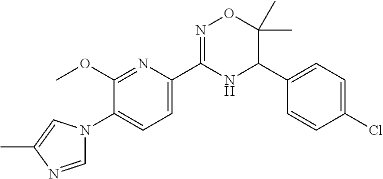 [SMILES: COc1nc(C2=NOC(C)(C)C(c3ccc(Cl)cc3)N2)ccc1-n1cnc(C)c1] | (+)-5-(4-chlorophenyl)-3-[6- methoxy-5-(4-methyl-1H- imidazol-1-yl)pyridin-2-yl]- 6,6-dimethyl-5,6-dihydro-4H- 1,2,4-oxadiazine |
| 24A | 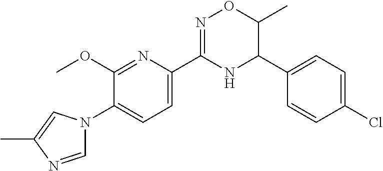 [SMILES: COc1nc(C2=NOC(C)C(c3ccc(Cl)cc3)N2)ccc1-n1cnc(C)c1] | (−)-trans-5-(4-chlorophenyl)- 3-[6-methoxy-5-(4-methyl- 1H-imidazol-1-yl)pyridin-2- yl]-6-methyl-5,6-dihydro-4H- 1,2,4-oxadiazine |
| 24B | 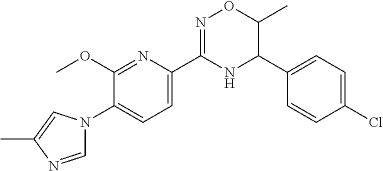 [SMILES: COc1nc(C2=NOC(C)C(c3ccc(Cl)cc3)N2)ccc1-n1cnc(C)c1] | (+)-trans-5-(4-chlorophenyl)- 3-[6-methoxy-5-(4-methyl- 1H-imidazol-1-yl)pyridin-2- yl]-6-methyl-5,6-dihydro-4H- 1,2,4-oxadiazine |
| 25A | 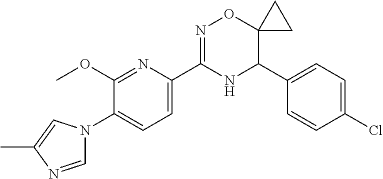 [SMILES: COc1nc(C2=NOC3(CC3)C(c3ccc(Cl)cc3)N2)ccc1-n1cnc(C)c1] | (−)-8-(4-chlorophenyl)-6-[6- methoxy-5-(4-methyl-1H- imidazol-1-yl)pyridin-2-yl]-4- oxa-5,7-diazaspiro[2.5]oct-5- ene |
| 25B | 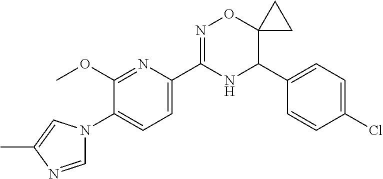 [SMILES: COc1nc(C2=NOC3(CC3)C(c3ccc(Cl)cc3)N2)ccc1-n1cnc(C)c1] | (+)-8-(4-chlorophenyl)-6-[6- methoxy-5-(4-methyl-1H- imidazol-1-yl)pyridin-2-yl]-4- oxa-5,7-diazaspiro[2.5]oct-5- ene |
| 26A | 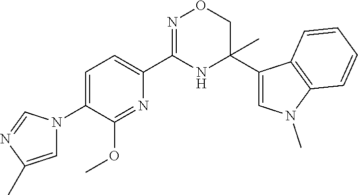 [SMILES: COc1nc(C2=NOCC(C)(c3cn(C)c4ccccc34)N2)ccc1-n1cnc(C)c1] | (+)-3-[3-[6-methoxy-5-(4- methyl-1H-imidazol-1- yl)pyridin-2-yl]-5-methyl-5,6- dihydro-4H-1,2,4-oxadiazin- 5-yl]-1-methyl-1H-indole |
| 26B | 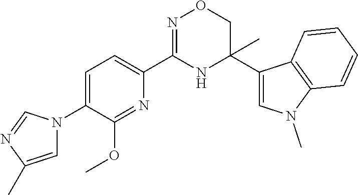 [SMILES: COc1nc(C2=NOCC(C)(c3cn(C)c4ccccc34)N2)ccc1-n1cnc(C)c1] | (−)-3-[3-[6-methoxy-5-(4- methyl-1H-imidazol-1- yl)pyridin-2-yl]-5-methyl-5,6- dihydro-4H-1,2,4-oxadiazin- 5-yl]-1-methyl-1H-indole |
| 28A | 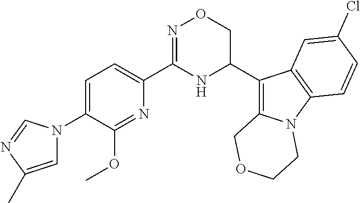 [SMILES: COc1nc(C2=NOCC(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2-yl)- 5,6-dihydro-4H-1,2,4- oxadiazin-5-yl)-3,4-dihydro- 1H-[1,4]oxazino[4,3-a]indole |
| 28B | 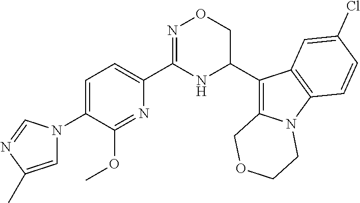 [SMILES: COc1nc(C2=NOCC(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2-yl)- 5,6-dihydro-4H-1,2,4- oxadiazin-5-yl)-3,4-dihydro- 1H-[1,4]oxazino[4,3-a]indole |
| 29A | 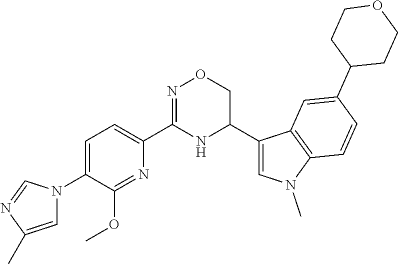 [SMILES: COc1nc(C2=NOCC(c3cn(C)c4ccc(C5CCOCC5)cc34)N2)ccc1-n1cnc(C)c1] | (−)-3-(6-methoxy-5-(4- methyl-1H-imidazol-1- yl)pyridin-2-yl)-5-(1-methyl- 5-(tetrahydro-2H-pyran-4-yl)- 1H-indol-3-yl)-5,6-dihydro- 4H-1,2,4-oxadiazine |
| 29B | 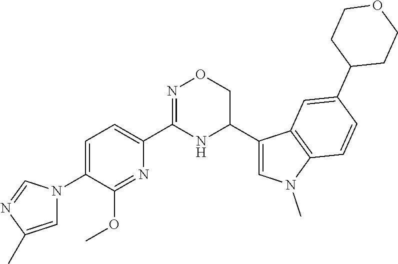 [SMILES: COc1nc(C2=NOCC(c3cn(C)c4ccc(C5CCOCC5)cc34)N2)ccc1-n1cnc(C)c1] | (+)-3-(6-methoxy-5-(4- methyl-1H-imidazol-1- yl)pyridin-2-yl)-5-(1-methyl- 5-(tetrahydro-2H-pyran-4-yl)- 1H-indol-3-yl)-5,6-dihydro- 4H-1,2,4-oxadiazine |
| 30A | 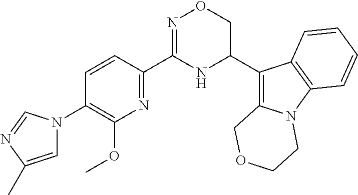 [SMILES: COc1nc(C2=NOCC(c3c4n(c5ccccc35)CCOC4)N2)ccc1-n1cnc(C)c1] | (−)-10-(3-(6-methoxy-5-(4- methyl-1H-imidazol-1- yl)pyridin-2-yl)-5,6-dihydro- 4H-1,2,4-oxadiazin-5-yl)-3 ,4- dihydro-1H-[1,4]oxazino[4,3- a]indole |
| 30B | 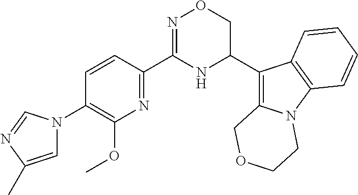 [SMILES: COc1nc(C2=NOCC(c3c4n(c5ccccc35)CCOC4)N2)ccc1-n1cnc(C)c1] | (+)-10-(3-(6-methoxy-5-(4- methyl-1H-imidazol-1- yl)pyridin-2-yl)-5,6-dihydro- 4H-1,2,4-oxadiazin-5-yl)-3,4- dihydro-1H-[1,4]oxazino[4,3- a]indole |
| 37A | 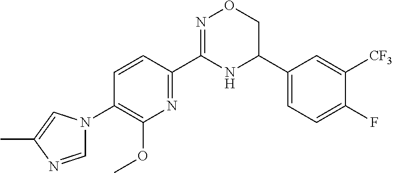 [SMILES: COc1nc(C2=NOCC(c3ccc(F)c(C(F)(F)F)c3)N2)ccc1-n1cnc(C)c1] | (−)-5-(4-fluoro-3- (trifluoromethyl) phenyl)-3- (6-methoxy-5-(4-methyl-1H- imidazol-1-yl) pyridin-2-yl)- 5,6-dihydro-4H-1,2,4- oxadiazine |
| 37B | 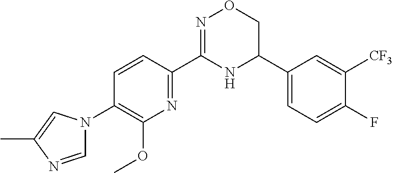 [SMILES: COc1nc(C2=NOCC(c3ccc(F)c(C(F)(F)F)c3)N2)ccc1-n1cnc(C)c1] | (+)-5-(4-fluoro-3- (trifluoromethyl) phenyl)-3- (6-methoxy-5-(4-methyl-1H- imidazol-1-yl) pyridin-2-yl)- 5,6-dihydro-4H-1,2,4- oxadiazine |
| 38A | 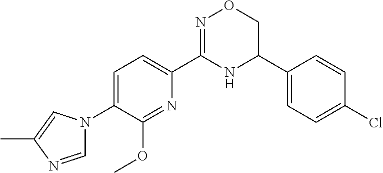 [SMILES: COc1nc(C2=NOCC(c3ccc(Cl)cc3)N2)ccc1-n1cnc(C)c1] | (+)-5-(4-chlorophenyl)-3-(6- methoxy-5-(4-methyl-1H- imidazol-1-yl) pyridin-2-yl)- 5,6-dihydro-4H-1,2,4- oxadiazine |
| 38B | 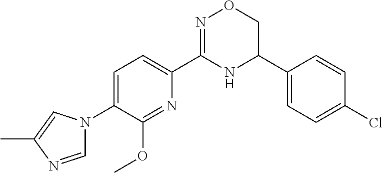 [SMILES: COc1nc(C2=NOCC(c3ccc(Cl)cc3)N2)ccc1-n1cnc(C)c1] | (+)-5-(4-chlorophenyl)-3-(6- methoxy-5-(4-methyl-1H- imidazol-1-yl) pyridin-2-yl)- 5,6-dihydro-4H-1,2,4- oxadiazine |
| 39A | 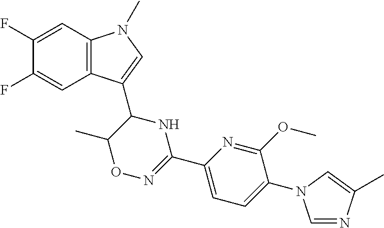 [SMILES: COc1nc(C2=NOC(C)C(c3cn(C)c4cc(F)c(F)cc34)N2)ccc1-n1cnc(C)c1] | (+)-5-(5,6-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 39B | 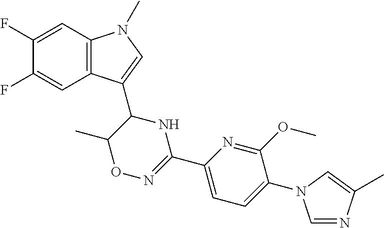 [SMILES: COc1nc(C2=NOC(C)C(c3cn(C)c4cc(F)c(F)cc34)N2)ccc1-n1cnc(C)c1] | (−)-5-(5,6-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 39C | 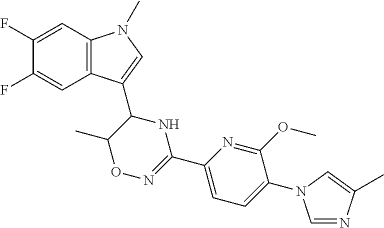 [SMILES: COc1nc(C2=NOC(C)C(c3cn(C)c4cc(F)c(F)cc34)N2)ccc1-n1cnc(C)c1] | (−)-5-(5,6-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 39D | 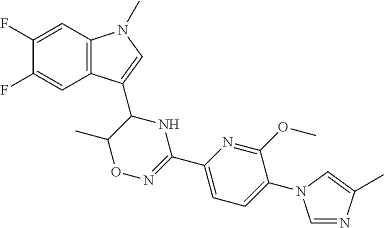 [SMILES: COc1nc(C2=NOC(C)C(c3cn(C)c4cc(F)c(F)cc34)N2)ccc1-n1cnc(C)c1] | (+)-5-(5,6-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 40-IA, 40- IIA | 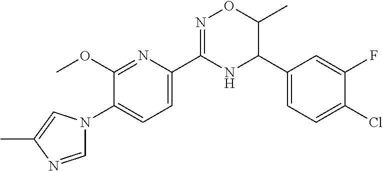 [SMILES: COc1nc(C2=NOC(C)C(c3ccc(Cl)c(F)c3)N2)ccc1-n1cnc(C)c1] | (−)-(cis)-5-(4-chloro-3- fluorophenyl)-3-(6-methoxy- 5-(4-methyl-1H-imidazol-1- yl)pyridin-2-yl)-6-methyl-5,6- dihydro-4H-1,2,4-oxadiazine |
| 40-IB, 40- IIB | 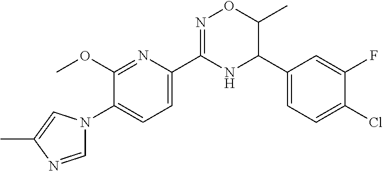 [SMILES: COc1nc(C2=NOC(C)C(c3ccc(Cl)c(F)c3)N2)ccc1-n1cnc(C)c1] | (+)-(cis)-5-(4-chloro-3- fluorophenyl)-3-(6-methoxy- 5-(4-methyl-1H-imidazol-1- yl)pyridin-2-yl)-6-methyl-5,6- dihydro-4H-1,2,4-oxadiazine |
| 41A | 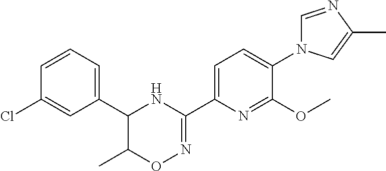 [SMILES: COc1nc(C2=NOC(C)C(c3cccc(Cl)c3)N2)ccc1-n1cnc(C)c1] | (−)-(cis)-5-(3-chlorophenyl)- 3-(6-methoxy-5-(4-methyl- 1H-imidazol-1-yl)pyridin-2- yl)-6-methyl-5,6-dihydro-4H- 1,2,4-oxadiazine |
| 41B | 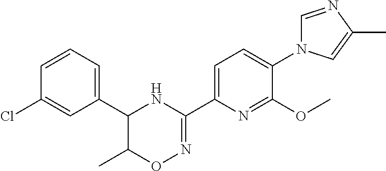 [SMILES: COc1nc(C2=NOC(C)C(c3cccc(Cl)c3)N2)ccc1-n1cnc(C)c1] | (+)-(cis)-5-(3-chlorophenyl)- 3-(6-methoxy-5-(4-methyl- 1H-imidazol-1-yl)pyridin-2- yl)-6-methyl-5,6-dihydro-4H- 1,2,4-oxadiazine |
| 42A | 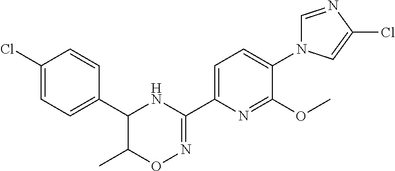 [SMILES: COc1nc(C2=NOC(C)C(c3ccc(Cl)cc3)N2)ccc1-n1cnc(Cl)c1] | (−)-(cis)-3-(5-(4-chloro-1H- imidazol-1-yl)-6- methoxypyridin-2-yl)-5-(4- chlorophenyl)-6-methyl-5,6- dihydro-4H-1,2,4-oxadiazine |
| 42B | 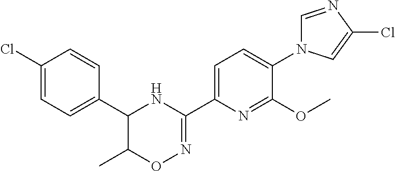 [SMILES: COc1nc(C2=NOC(C)C(c3ccc(Cl)cc3)N2)ccc1-n1cnc(Cl)c1] | (+)-(cis)-3-(5-(4-chloro-1H- methoxypyridin-2-yl)-5-(4- chlorophenyl)-6-methyl-5,6- dihydro-4H-1,2,4-oxadiazine |
| 43A | 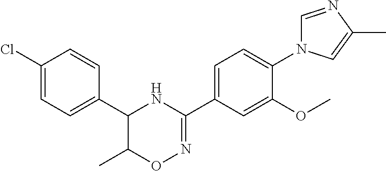 [SMILES: COc1cc(C2=NOC(C)C(c3ccc(Cl)cc3)N2)ccc1-n1cnc(C)c1] | (−)-(cis)-5-(4-chlorophenyl)- 3-(3-methoxy-4-(4-methyl- 1H-imidazol-1-yl)phenyl)-6- methyl-5,6-dihydro-4H-1,2,4- oxadiazine |
| 43B | 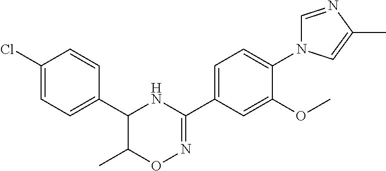 [SMILES: COc1cc(C2=NOC(C)C(c3ccc(Cl)cc3)N2)ccc1-n1cnc(C)c1] | (+)-(cis)-5-(4-chlorophenyl)- 3-(3-methoxy-4-(4-methyl- 1H-imidazol-1-yl)phenyl)-6- methyl-5,6-dihydro-4H-1,2,4- oxadiazine |
| 44A, 45A | 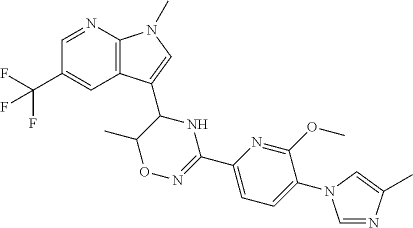 [SMILES: COc1nc(C2=NOC(C)C(c3cn(C)c4ncc(C(F)(F)F)cc34)N2)ccc1-n1cnc(C)c1] | (−)-3-(6-methoxy-5-(4- methyl-1H-imidazol-1- yl)pyridin-2-yl)-6-methyl-5- (1-methyl-5- (trifluoromethyl)-1H- pyrrolo[2,3-b]pyridin-3-yl)- 5,6-dihydro-4H-1,2,4- oxadiazine |
| 44B, 45B | 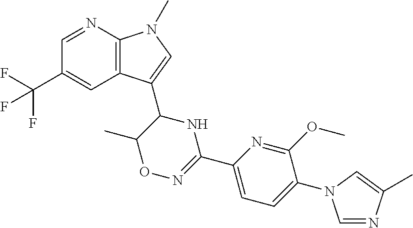 [SMILES: COc1nc(C2=NOC(C)C(c3cn(C)c4ncc(C(F)(F)F)cc34)N2)ccc1-n1cnc(C)c1] | (+)-3-(6-methoxy-5-(4- methyl-1H-imidazol-1- yl)pyridin-2-yl)-6-methyl-5- (1-methyl-5- (trifluoromethyl)-1H- pyrrolo[2,3-b]pyridin-3-yl)- 5,6-dihydro-4H-1,2,4- oxadiazine |
| 44C, 45C | 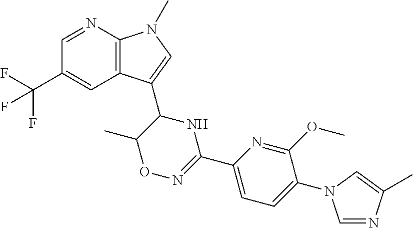 [SMILES: COc1nc(C2=NOC(C)C(c3cn(C)c4ncc(C(F)(F)F)cc34)N2)ccc1-n1cnc(C)c1] | (−)-3-(6-methoxy-5-(4- methyl-1H-imidazol-1- yl)pyridin-2-yl)-6-methyl-5- (1-methyl-5- (trifluoromethyl)-1H- pyrrolo[2,3-b]pyridin-3-yl)- 5,6-dihydro-4H-1,2,4- oxadiazine |
| 44D, 45D | 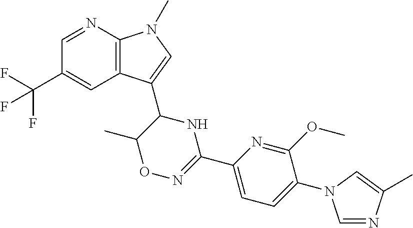 [SMILES: COc1nc(C2=NOC(C)C(c3cn(C)c4ncc(C(F)(F)F)cc34)N2)ccc1-n1cnc(C)c1] | (+)-3-(6-methoxy-5-(4- methyl-1H-imidazol-1- yl)pyridin-2-yl)-6-methyl-5- (1-methyl-5- (trifluoromethyl)-1H- pyrrolo[2,3-b]pyridin-3-yl)- 5,6-dihydro-4H-1,2,4- oxadiazine |
| 46A | 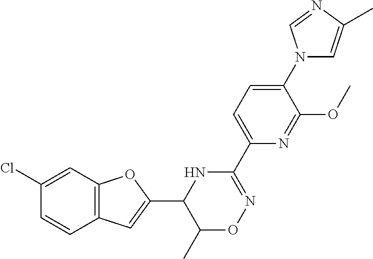 [SMILES: COc1nc(C2=NOC(C)C(c3cc4ccc(Cl)cc4o3)N2)ccc1-n1cnc(C)c1] | (−)-5-(6-chlorobenzofuran-2- yl)-3-(6-methoxy-5-(4- methyl-1H-imidazol-1- yl)pyridin-2-yl)-6-methyl-5,6- dihydro-4H-1,2,4-oxadiazine |
| 46B | 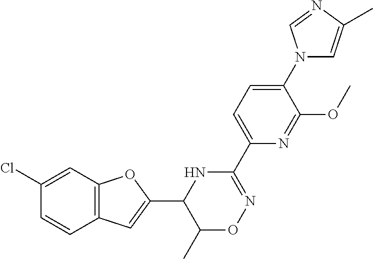 [SMILES: COc1nc(C2=NOC(C)C(c3cc4ccc(Cl)cc4o3)N2)ccc1-n1cnc(C)c1] | (+)-5-(6-chlorobenzofuran-2- yl)-3-(6-methoxy-5-(4- methyl-1H-imidazol-1- yl)pyridin-2-yl)-6-methyl-5,6- dihydro-4H-1,2,4-oxadiazine |
| 46C | 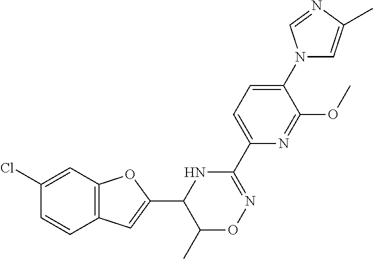 [SMILES: COc1nc(C2=NOC(C)C(c3cc4ccc(Cl)cc4o3)N2)ccc1-n1cnc(C)c1] | (−)-5-(6-chlorobenzofuran-2- yl)-3-(6-methoxy-5-(4- methyl-1H-imidazol-1- yl)pyridin-2-yl)-6-methyl-5,6- dihydro-4H-1,2,4-oxadiazine |
| 46D | 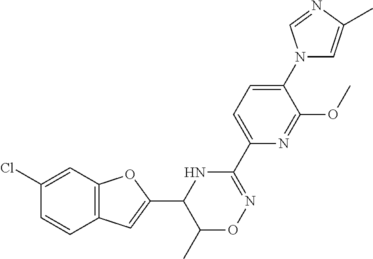 [SMILES: COc1nc(C2=NOC(C)C(c3cc4ccc(Cl)cc4o3)N2)ccc1-n1cnc(C)c1] | (+)-5-(6-chlorobenzofuran-2- yl)-3-(6-methoxy-5-(4- methyl-1H-imidazol-1- yl)pyridin-2-yl)-6-methyl-5,6- dihydro-4H-1,2,4-oxadiazine |
| 47A | 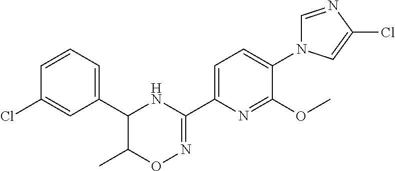 [SMILES: COc1nc(C2=NOC(C)C(c3cccc(Cl)c3)N2)ccc1-n1cnc(Cl)c1] | (−)-(cis)-3-(5-(4-chloro-1H- imidazol-1-yl)-6- methoxypyridin-2-yl)-5-(3- chlorophenyl)-6-methyl-5,6- dihydro-4H-1,2,4-oxadiazine |
| 47B | 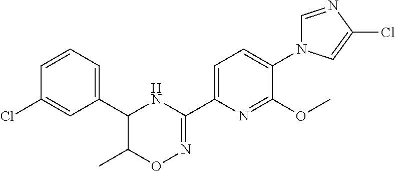 [SMILES: COc1nc(C2=NOC(C)C(c3cccc(Cl)c3)N2)ccc1-n1cnc(Cl)c1] | (+)-(cis)-3-(5-(4-chloro-1H- imidazol-1-yl)-6- methoxypyridin-2-yl)-5-(3- chlorophenyl)-6-methyl-5,6- dihydro-4H-1,2,4-oxadiazine |
| 48A | 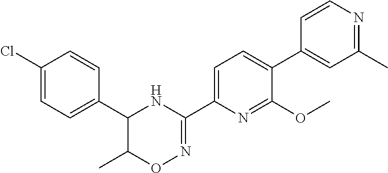 [SMILES: COc1nc(C2=NOC(C)C(c3ccc(Cl)cc3)N2)ccc1-c1ccnc(C)c1] | (−)-(cis)-5-(4-chlorophenyl)- 3-(2-methoxy-2′-methyl-[3,4′- bipyridin]-6-yl)-6-methyl-5,6- dihydro-4H-1,2,4-oxadiazine |
| 48B | 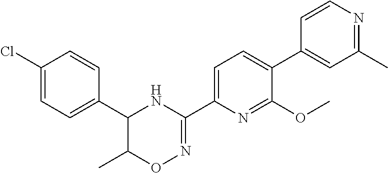 [SMILES: COc1nc(C2=NOC(C)C(c3ccc(Cl)cc3)N2)ccc1-c1ccnc(C)c1] | (+)-(cis)-5-(4-chlorophenyl)- 3-(2-methoxy-2′-methyl-[3,4′- bipyridin]-6-yl)-6-methyl-5,6- dihydro-4H-1,2,4-oxadiazine |
| 49A | 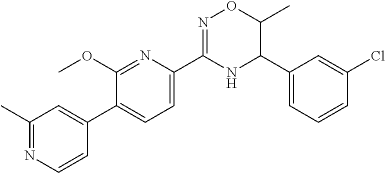 [SMILES: COc1nc(C2=NOC(C)C(c3cccc(Cl)c3)N2)ccc1-c1ccnc(C)c1] | (−)-(cis)-5-(3-chlorophenyl)- 3-(2-methoxy-2′-methyl-[3,4′- bipyridin]-6-yl)-6-methyl-5,6- dihydro-4H-1,2,4-oxadiazine |
| 49B | 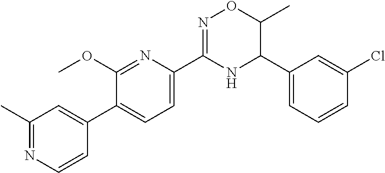 [SMILES: COc1nc(C2=NOC(C)C(c3cccc(Cl)c3)N2)ccc1-c1ccnc(C)c1] | (+)-(cis)-5-(3-chlorophenyl)- 3-(2-methoxy-2′-methyl-[3,4′- bipyridin]-6-yl)-6-methyl-5,6- dihydro-4H-1,2,4-oxadiazine |
| 50A | 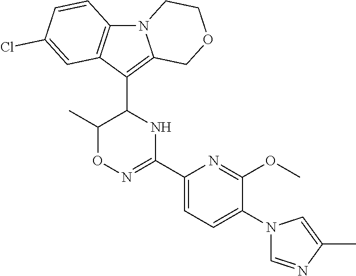 [SMILES: COc1nc(C2=NOC(C)C(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2-yl)-6- methyl-5,6-dihydro-4H-1,2,4 oxadiazin-5-yl)-3,4-dihydro- 1H-[1,4]oxazino [4,3-a]indole |
| 50B | 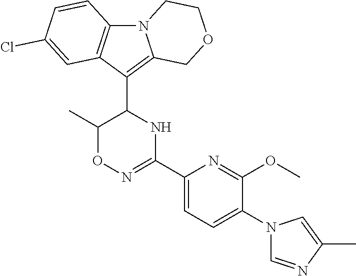 [SMILES: COc1nc(C2=NOC(C)C(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2-yl)-6- methyl-5,6-dihydro-4H-1,2,4- oxadiazin-5-yl)-3,4-dihydro- 1H-[1,4]oxazino[4,3-a]indole |
| 50C | 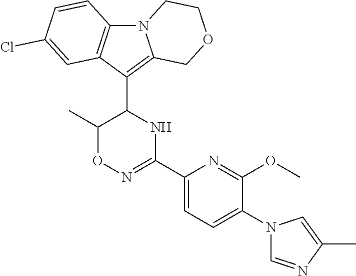 [SMILES: COc1nc(C2=NOC(C)C(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2-yl)-6- methyl-5,6-dihydro-4H-1,2,4- oxadiazin-5-yl)-3,4-dihydro- 1H-[1,4]oxazino[4,3-a]indole |
| 50D | 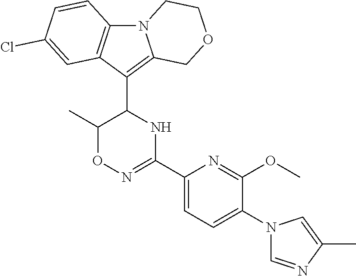 [SMILES: COc1nc(C2=NOC(C)C(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2-yl)-6- methyl-5,6-dihydro-4H-1,2,4- oxadiazin-5-yl)-3,4-dihydro- 1H-[1,4]oxazino[4,3-a]indole |
| 51A | 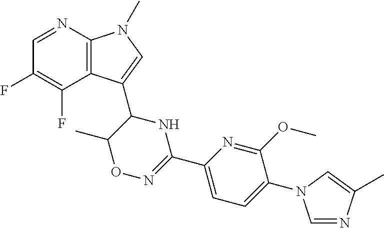 [SMILES: COc1nc(C2=NOC(C)C(c3cn(C)c4ncc(F)c(F)c34)N2)ccc1-n1cnc(C)c1] | (+)-5-(4,5-difluoro-1-methyl- 1H-pyrrolo[2,3-b]pyridin-3- yl)-3-(6-methoxy-5-(4- methyl-1H-imidazol-1- yl)pyridin-2-yl)-6-methyl-5,6- dihydro-4H-1,2,4-oxadiazine |
| 51B | 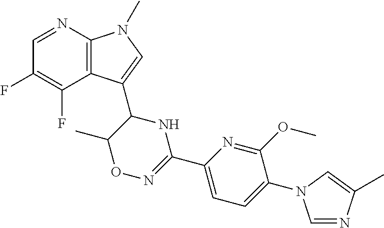 [SMILES: COc1nc(C2=NOC(C)C(c3cn(C)c4ncc(F)c(F)c34)N2)ccc1-n1cnc(C)c1] | (−)-5-(4,5-difluoro-1-methyl- 1H-pyrrolo[2,3-b]pyridin-3- yl)-3-(6-methoxy-5-(4- methyl-1H-imidazol-1- yl)pyridin-2-yl)-6-methyl-5,6- dihydro-4H-1,2,4-oxadiazine |
| 51C | 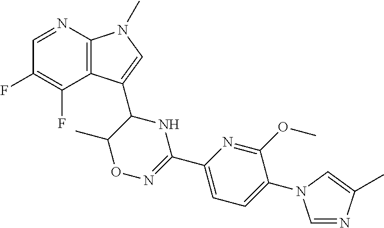 [SMILES: COc1nc(C2=NOC(C)C(c3cn(C)c4ncc(F)c(F)c34)N2)ccc1-n1cnc(C)c1] | (−)-5-(4,5-difluoro-1-methyl- 1H-pyrrolo[2,3-b]pyridin-3- yl)-3-(6-methoxy-5-(4- methyl-1H-imidazol-1- yl)pyridin-2-yl)-6-methyl-5,6- dihydro-4H-1,2,4-oxadiazine |
| 51D | 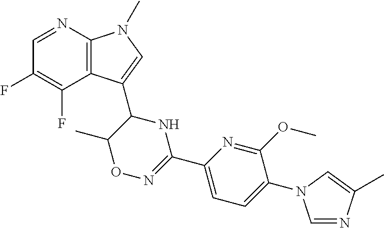 [SMILES: COc1nc(C2=NOC(C)C(c3cn(C)c4ncc(F)c(F)c34)N2)ccc1-n1cnc(C)c1] | (+)-5-(4,5-difluoro-1-methyl- 1H-pyrrolo[2,3-b]pyridin-3- yl)-3-(6-methoxy-5-(4- methyl-1H-imidazol-1- yl)pyridin-2-yl)-6-methyl-5,6- dihydro-4H-1,2,4-oxadiazine |
| 52A | 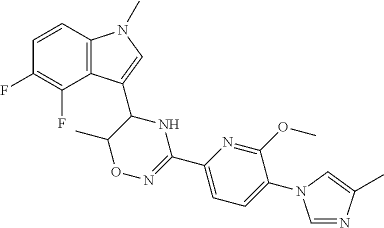 [SMILES: COc1nc(C2=NOC(C)C(c3cn(C)c4ccc(F)c(F)c34)N2)ccc1-n1cnc(C)c1] | (−)-5-(4,5-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 52B | 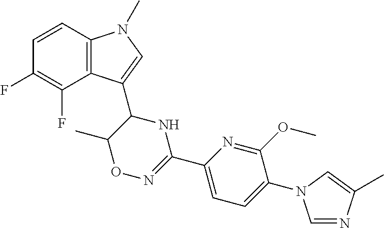 [SMILES: COc1nc(C2=NOC(C)C(c3cn(C)c4ccc(F)c(F)c34)N2)ccc1-n1cnc(C)c1] | (+)-5-(4,5-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 52C | 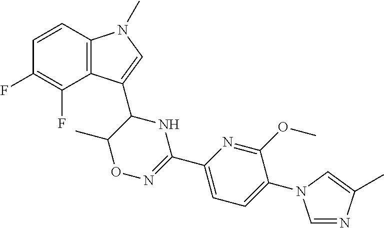 [SMILES: COc1nc(C2=NOC(C)C(c3cn(C)c4ccc(F)c(F)c34)N2)ccc1-n1cnc(C)c1] | (+)-5-(4,5-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 52D | 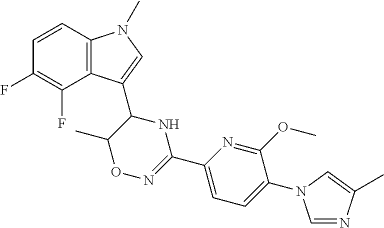 [SMILES: COc1nc(C2=NOC(C)C(c3cn(C)c4ccc(F)c(F)c34)N2)ccc1-n1cnc(C)c1] | (−)-5-(4,5-difluoro-1-methyl- 1H-indol-3-yl)-3-(6-methoxy- 5-(4-methyl-1H-imidazol-1- yl)pyridin-2-yl)-6-methyl-5,6- dihydro-4H-1,2,4-oxadiazine |
| 53A | 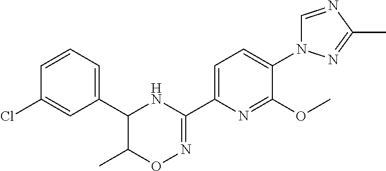 [SMILES: COc1nc(C2=NOC(C)C(c3cccc(Cl)c3)N2)ccc1-n1cnc(C)n1] | (−)-(cis)-5-(3-chlorophenyl)- 3-(6-methoxy-5-(3-methyl- 1H-1,2,4-triazol-1-yl)pyridin- 2-yl)-6-methyl-5,6-dihydro- 4H-1,2,4-oxadiazine |
| 53B | 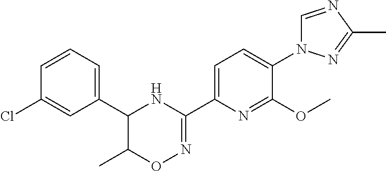 [SMILES: COc1nc(C2=NOC(C)C(c3cccc(Cl)c3)N2)ccc1-n1cnc(C)n1] | (+)-(cis)-5-(3-chlorophenyl)- 3-(6-methoxy-5-(3-methyl- 1H-1,2,4-triazol-1-yl)pyridin- 2-yl)-6-methyl-5,6-dihydro- 4H-1,2,4-oxadiazine |
| 54A | 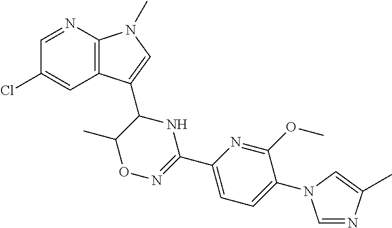 [SMILES: COc1nc(C2=NOC(C)C(c3cn(C)c4ncc(Cl)cc34)N2)ccc1-n1cnc(C)c1] | (−)-5-(5-chloro-1-methyl-1H- pyrrolo[2,3-b]pyridin-3-yl)-3- (6-methoxy-5-(4-methyl-1H- imidazol-1-yl)pyridin-2-yl)-6- methyl-5,6-dihydro-4H-1,2,4- oxadiazine |
| 54B | 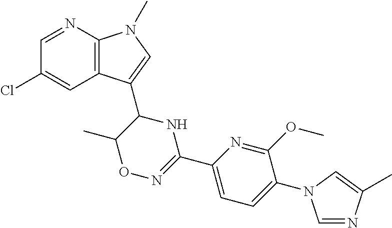 [SMILES: COc1nc(C2=NOC(C)C(c3cn(C)c4ncc(Cl)cc34)N2)ccc1-n1cnc(C)c1] | (+)-5-(5-chloro-1-methyl-1H- pyrrolo[2,3-b]pyridin-3-yl)-3- (6-methoxy-5-(4-methyl-1H- imidazol-1-yl)pyridin-2-yl)-6- methyl-5,6-dihydro-4H-1,2,4- oxadiazine |
| 54C | 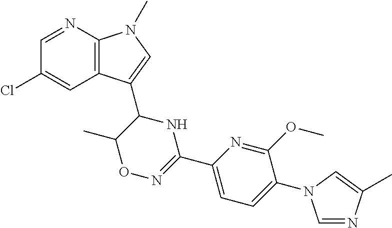 [SMILES: COc1nc(C2=NOC(C)C(c3cn(C)c4ncc(Cl)cc34)N2)ccc1-n1cnc(C)c1] | (−)-5-(5-chloro-1-methyl-1H- pyrrolo[2,3-b]pyridin-3-yl)-3- (6-methoxy-5-(4-methyl-1H- imidazol-1-yl)pyridin-2-yl)-6- methyl-5,6-dihydro-4H-1,2,4- oxadiazine |
| 54D | 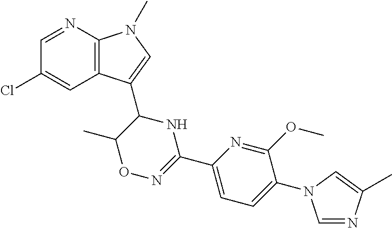 [SMILES: COc1nc(C2=NOC(C)C(c3cn(C)c4ncc(Cl)cc34)N2)ccc1-n1cnc(C)c1] | (+)-5-(5-chloro-1-methyl-1H- pyrrolo[2,3-b]pyridin-3-yl)-3- (6-methoxy-5-(4-methyl-1H- imidazol-1-yl)pyridin-2-yl)-6- methyl-5,6-dihydro-4H-1,2,4- oxadiazine |
| 55A | 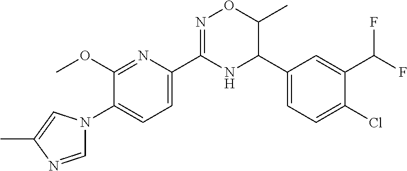 [SMILES: COc1nc(C2=NOC(C)C(c3ccc(Cl)c(C(F)F)c3)N2)ccc1-n1cnc(C)c1] | (+)-(cis)-5-(4-chloro-3- (difluoromethyl)phenyl)-3-(6- methoxy-5-(4-methyl-1H- imidazol-1-yl)pyridin-2-yl)-6- methyl-5,6-dihydro-4H-1,2,4- oxadiazine |
| 55B | 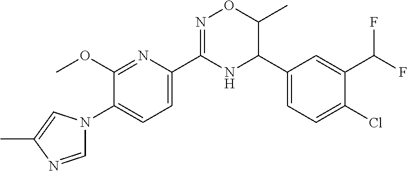 [SMILES: COc1nc(C2=NOC(C)C(c3ccc(Cl)c(C(F)F)c3)N2)ccc1-n1cnc(C)c1] | (−)-(cis)-5-(4-chloro-3- (difluoromethyl)phenyl)-3-(6- methoxy-5-(4-methyl-1H- imidazol-1-yl)pyridin-2-yl)-6- methyl-5,6-dihydro-4H-1,2,4- oxadiazine |
| 56A | 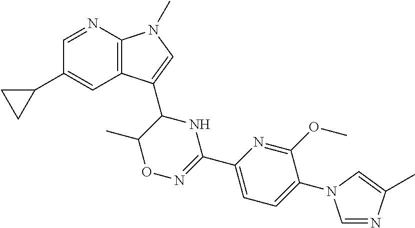 [SMILES: COc1nc(C2=NOC(C)C(c3cn(C)c4ncc(C5CC5)cc34)N2)ccc1-n1cnc(C)c1] | (+)-5-(5-cyclopropyl-1- methyl-1H-pyrrolo[2,3- b]pyridin-3-yl)-3-(6-methoxy- 5-(4-methyl-1H-imidazol-1- yl)pyridin-2-yl)-6-methyl-5,6 dihydro-4H-1,2,4-oxadiazine |
| 56B | 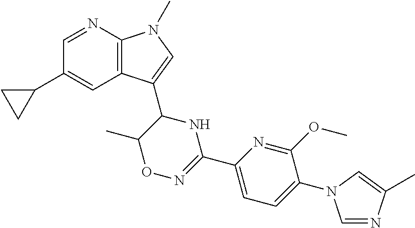 [SMILES: COc1nc(C2=NOC(C)C(c3cn(C)c4ncc(C5CC5)cc34)N2)ccc1-n1cnc(C)c1] | (−)-5-(5-cyclopropyl-1- methyl-1H-pyrrolo[2,3- b]pyridin-3-yl)-3-(6-methoxy- 5-(4-methyl-1H-imidazol-1- yl)pyridin-2-yl)-6-methyl-5,6- dihydro-4H-1,2,4-oxadiazine |
| 56C | 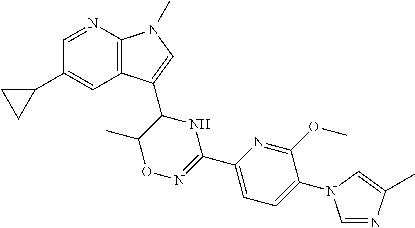 [SMILES: COc1nc(C2=NOC(C)C(c3cn(C)c4ncc(C5CC5)cc34)N2)ccc1-n1cnc(C)c1] | (−)-5-(5-cyclopropyl-1- methyl-1H-pyrrolo[2,3- b]pyridin-3-yl)-3-(6-methoxy- 5-(4-methyl-1H-imidazol-1- yl)pyridin-2-yl)-6-methyl-5,6 dihydro-4H-1,2,4-oxadiazine |
| 56D | 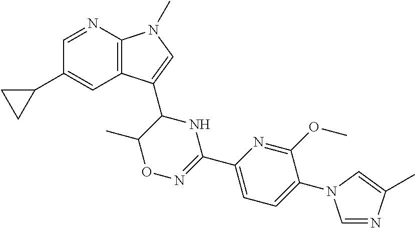 [SMILES: COc1nc(C2=NOC(C)C(c3cn(C)c4ncc(C5CC5)cc34)N2)ccc1-n1cnc(C)c1] | (+)-5-(5-cyclopropyl-1- methyl-1H-pyrrolo[2,3- b]pyridin-3-yl)-3-(6-methoxy- 5-(4-methyl-1H-imidazol-1- yl)pyridin-2-yl)-6-methyl-5,6- dihydro-4H-1,2,4-oxadiazine |
| 57A, 58A | 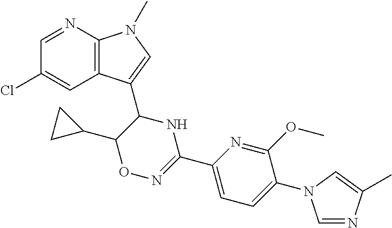 [SMILES: COc1nc(C2=NOC(C3CC3)C(c3cn(C)c4ncc(Cl)cc34)N2)ccc1-n1cnc(C)c1] | (+)-(cis)-5-(5-chloro-1- methyl-1H-pyrrolo[2,3- b]pyridin-3-yl)-6- cyclopropyl-3-(6-methoxy-5- (4-methyl-1H-imidazol-1- yl)pyridin-2-yl)-5,6-dihydro- 4H-1,2,4-oxadiazine |
| 57B, 58B | 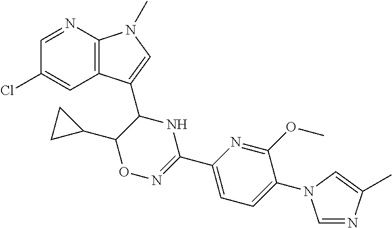 [SMILES: COc1nc(C2=NOC(C3CC3)C(c3cn(C)c4ncc(Cl)cc34)N2)ccc1-n1cnc(C)c1] | (−)-(cis)-5-(5-chloro-1- methyl-1H-pyrrolo[2,3- b]pyridin-3-yl)-6- cyclopropyl-3-(6-methoxy-5- (4-methyl-1H-imidazol-1- yl)pyridin-2-yl)-5,6-dihydro- 4H-1,2,4-oxadiazine |
| 59A | 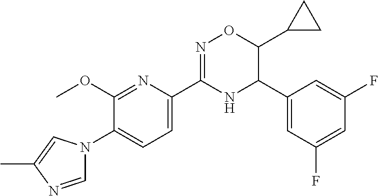 [SMILES: COc1nc(C2=NOC(C3CC3)C(c3cc(F)cc(F)c3)N2)ccc1-n1cnc(C)c1] | (+)-(cis)-6-cyclopropyl-5- (3,5-difluorophenyl)-3-(6- methoxy-5-(4-methyl-1H- imidazol-1-yl)pyridin-2-yl)- 5,6-dihydro-4H-1,2,4- oxadiazine |
| 59B | 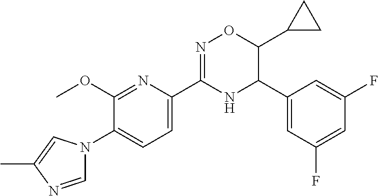 [SMILES: COc1nc(C2=NOC(C3CC3)C(c3cc(F)cc(F)c3)N2)ccc1-n1cnc(C)c1] | (−)-(cis)-6-cyclopropyl-5-(3,5- difluorophenyl)-3-(6- methoxy-5-(4-methyl-1H- imidazol-1-yl)pyridin-2-yl)- 5,6-dihydro-4H-1,2,4- oxadiazine |
| 60A | 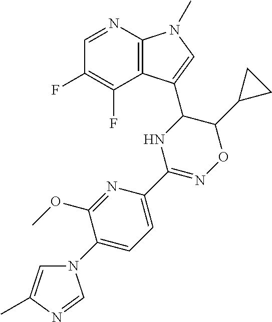 [SMILES: COc1nc(C2=NOC(C3CC3)C(c3cn(C)c4ncc(F)c(F)c34)N2)ccc1-n1cnc(C)c1] | (+)-(cis)-6-cyclopropyl-5- (4,5-difluoro-1-methyl-1H- pyrrolo[2,3-b]pyridin-3-yl)-3- (6-methoxy-5-(4-methyl-1H- imidazol-1-yl)pyridin-2-yl)- 5,6-dihydro-4H-1,2,4- oxadiazine |
| 60B | 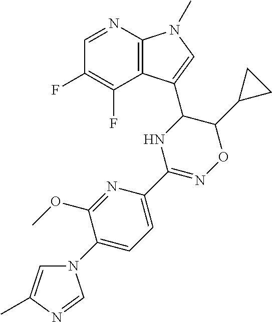 [SMILES: COc1nc(C2=NOC(C3CC3)C(c3cn(C)c4ncc(F)c(F)c34)N2)ccc1-n1cnc(C)c1] | (−)-(cis)-6-cyclopropyl-5-(4,5- difluoro-1-methyl-1H- pyrrolo[2,3-b]pyridin-3-yl)-3- (6-methoxy-5-(4-methyl-1H- imidazol-1-yl)pyridin-2-yl)- 5,6-dihydro-4H-1,2,4- oxadiazine |
| 61A | 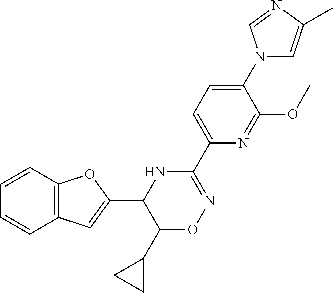 [SMILES: COc1nc(C2=NOC(C3CC3)C(c3cc4ccccc4o3)N2)ccc1-n1cnc(C)c1] | (+)-(cis)-5-(benzofuran-2-yl)- 6-cyclopropyl-3-(6-methoxy- 5-(4-methyl-1H-imidazol-1- yl)pyridin-2-yl)-5,6-dihydro- 4H-1,2,4-oxadiazine |
| 61B | 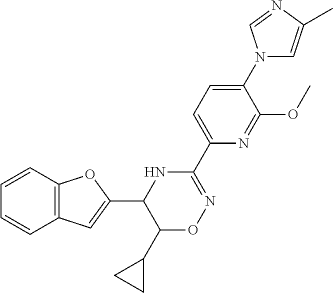 [SMILES: COc1nc(C2=NOC(C3CC3)C(c3cc4ccccc4o3)N2)ccc1-n1cnc(C)c1] | (−)-(cis)-5-(benzofuran-2-yl)- 6-cyclopropyl-3-(6-methoxy- 5-(4-methyl-1H-imidazol-1- yl)pyridin-2-yl)-5,6-dihydro- 4H-1,2,4-oxadiazine |
| 62A | 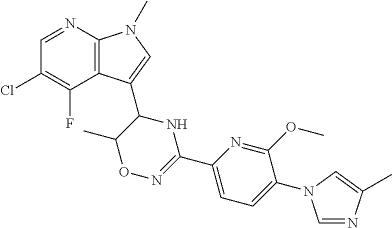 [SMILES: COc1nc(C2=NOC(C)C(c3cn(C)c4ncc(Cl)c(F)c34)N2)ccc1-n1cnc(C)c1] | (+)-(cis)-5-(5-chloro-4-fluoro- 1-methyl-1H-pyrrolo[2,3- b]pyridin-3-yl)-3-(6-methoxy- 5-(4-methyl-1H-imidazol-1- yl)pyridin-2-yl)-6-methyl-5,6- dihydro-4H-1,2,4-oxadiazine |
| 62B | 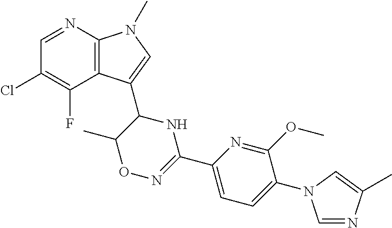 [SMILES: COc1nc(C2=NOC(C)C(c3cn(C)c4ncc(Cl)c(F)c34)N2)ccc1-n1cnc(C)c1] | (−)-(cis)-5-(5-chloro-4-fluoro- 1-methyl-1H-pyrrolo[2,3- b]pyridin-3-yl)-3-(6-methoxy- 5-(4-methyl-1H-imidazol- 1- yl)pyridin-2-yl)-6-methyl-5,6- dihydro-4H-1,2,4-oxadiazine |
| 63A | 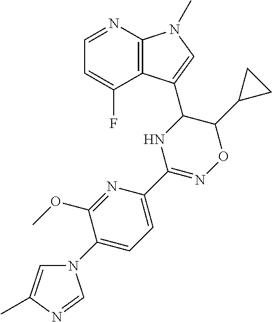 [SMILES: COc1nc(C2=NOC(C3CC3)C(c3cn(C)c4nccc(F)c34)N2)ccc1-n1cnc(C)c1] | (+)-(cis)-6-cyclopropyl-5-(4- fluoro-1-methyl-1H- pyrrolo[2,3-b]pyridin-3-yl)-3- (6-methoxy-5-(4-methyl-1H- imidazol-1-yl)pyridin-2-yl)- 5,6-dihydro-4H-1,2,4- oxadiazine |
| 63B | 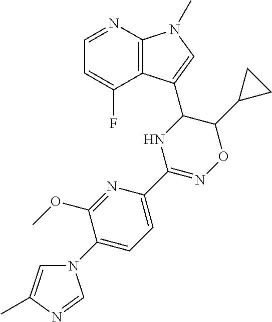 [SMILES: COc1nc(C2=NOC(C3CC3)C(c3cn(C)c4nccc(F)c34)N2)ccc1-n1cnc(C)c1] | (−)-(cis)-6-cyclopropyl-5-(4- fluoro-1-methyl-1H- pyrrolo[2,3-b]pyridin-3-yl)-3- (6-methoxy-5-(4-methyl-1H- imidazol-1-yl)pyridin-2-yl)- 5,6-dihydro-4H-1,2,4- oxadiazine |
| 65A | 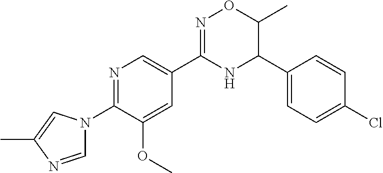 [SMILES: COc1cc(C2=NOC(C)C(c3ccc(Cl)cc3)N2)cnc1-n1cnc(C)c1] | 5-(4-chlorophenyl)-3-(5- methoxy-6-(4-methyl-1H- imidazol-1-yl) pyridin-3-yl)- 6-methyl-5,6-dihydro-4H- 1,2,4-oxadiazine |
| 65B | 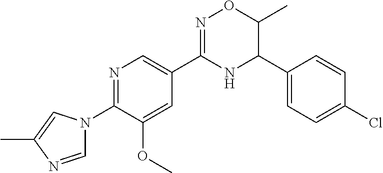 [SMILES: COc1cc(C2=NOC(C)C(c3ccc(Cl)cc3)N2)cnc1-n1cnc(C)c1] | 5-(4-chlorophenyl)-3-(5- methoxy-6-(4-methyl-1H- imidazol-1-yl) pyridin-3-yl)- 6-methyl-5,6-dihydro-4H- 1,2,4-oxadiazine |
| 69A | 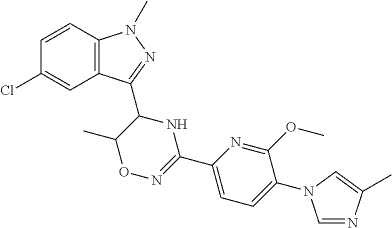 [SMILES: COc1nc(C2=NOC(C)C(c3nn(C)c4ccc(Cl)cc34)N2)ccc1-n1cnc(C)c1] | (+)-(cis)-5-(5-chloro-1- methyl-1H-indazol-3-yl)-3- (6-methoxy-5-(4-methyl-1H- imidazol-1-yl)pyridin-2-yl)-6- methyl-5,6-dihydro-4H-1,2,4- oxadiazine |
| 69B | 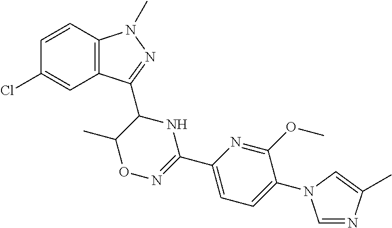 [SMILES: COc1nc(C2=NOC(C)C(c3nn(C)c4ccc(Cl)cc34)N2)ccc1-n1cnc(C)c1] | (−)-(cis)-5-(5-chloro-1- methyl-1H-indazol-3-yl)-3- (6-methoxy-5-(4-methyl-1H- imidazol-1-yl)pyridin-2-yl)-6- methyl-5,6-dihydro-4H-1,2,4- oxadiazine |
| 72A | 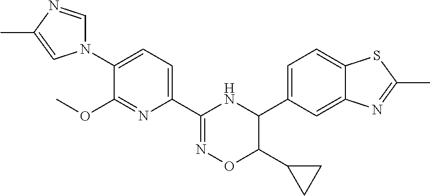 | (−)-(cis)-6-cyclopropyl-3-(6- methoxy-5-(4-methyl-1H- imidazol-1-yl)pyridin-2-yl)-5- (2-methylbenzo[d]thiazol-5- yl)-5,6-dihydro-4H-1,2,4- oxadiazine |
| 72B | 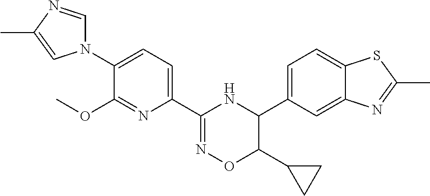 | (+)-(cis)-6-cyclopropyl-3-(6- methoxy-5-(4-methyl-1H- imidazol-1-yl)pyridin-2-yl)-5- (2-methylbenzo[d]thiazol-5- yl)-5,6-dihydro-4H-1,2,4- oxadiazine |

Additional exemplary Oxadizine Compounds are shown in Table I-1.

TABLE I-1
Additional Exemplary Oxadiazine Compounds

| Compound of | | |
|---|---|---|
| Example | Structure | Name |
| 36A | 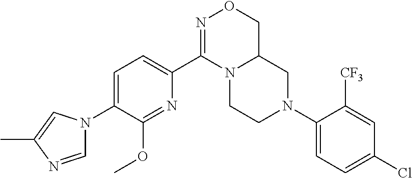 [SMILES: COc1nc(C2=NOCC3CN(c4ccc(Cl)cc4C(F)(F)F)CCN23)ccc1-n1cnc(C)c1] | (+)-8-(4-chloro-2- (trifluoromethyl) phenyl)- 4-(6-methoxy-5-(4- methyl-1H-imidazol-1-yl) pyridin-2-yl)-1, 6, 7, 8, 9, 9a-hexahydropyrazino [1, 2-d] [1,2,4]oxadiazine |
| 36B | 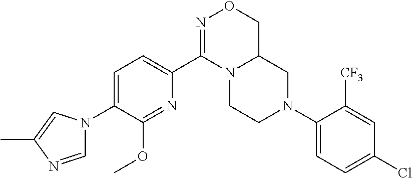 [SMILES: COc1nc(C2=NOCC3CN(c4ccc(Cl)cc4C(F)(F)F)CCN23)ccc1-n1cnc(C)c1] | (−)-8-(4-chloro-2- (trifluoromethyl) phenyl)- 4-(6-methoxy-5-(4- methyl-1H-imidazol-1-yl) pyridin-2-yl)-1,6,7,8,9, 9a-hexahydropyrazino [1, 2-d] [1,2,4]oxadiazine |
| 64A | 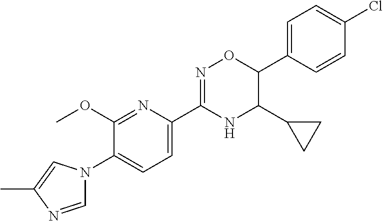 [SMILES: COc1nc(C2=NOC(c3ccc(Cl)cc3)C(C3CC3)N2)ccc1-n1cnc(C)c1] | (−)-(cis)-6-(4- chlorophenyl)-5- cyclopropyl-3-(6- methoxy-5-(4-methyl-1H- imidazol-1-yl)pyridin-2- yl)-5,6-dihydro-4H-1,2,4- oxadiazine |
| 64B | 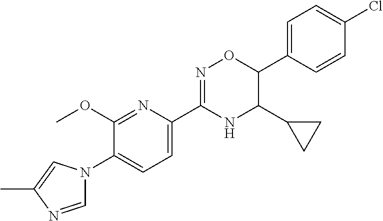 [SMILES: COc1nc(C2=NOC(c3ccc(Cl)cc3)C(C3CC3)N2)ccc1-n1cnc(C)c1] | (+)-(cis)-6-(4- chlorophenyl)-5- cyclopropyl-3-(6- methoxy-5-(4-methyl-1H- imidazol-1-yl)pyridin-2- yl)-5,6-dihydro-4H-1,2,4- oxadiazine |
| 66 | 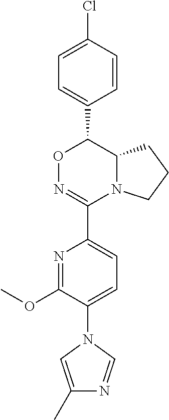 [SMILES: COc1nc(C2=NO[C@H](c3ccc(Cl)cc3)[C@@H]3CCCN23)ccc1-n1cnc(C)c1] | (1R,8αS)-1-(4- chlorophenyl)-4-(6- methoxy-5-(4-methyl-1H- imidazol-1-yl)pyridin-2- yl)-6,7,8,8a-tetrahydro- 1H-pyrrolo[1,2- d][1,2,4]oxadiazine |
| 71A | 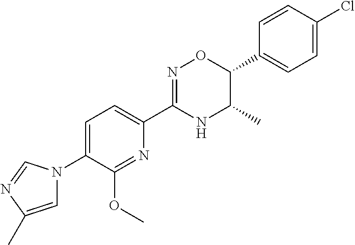 [SMILES: COc1nc(C2=NO[C@H](c3ccc(Cl)cc3)[C@H](C)N2)ccc1-n1cnc(C)c1] | (5S,6R)-6-(4- chlorophenyl)-3-(6- methoxy-5-(4-methyl-1H- imidazol-1-yl)pyridin-2- yl)-5-methyl-5,6-dihydro- 4H-1,2,4-oxadiazine |
| 71B | 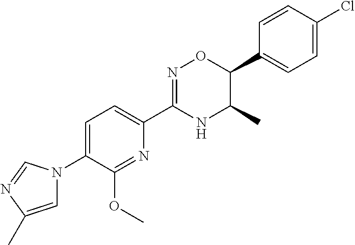 [SMILES: COc1nc(C2=NO[C@@H](c3ccc(Cl)cc3)[C@@H](C)N2)ccc1-n1cnc(C)c1] | (5R,6S)-6-(4- chlorophenyl)-3-(6- methoxy-5-(4-methyl-1H- imidazol-1-yl)pyridin-2- yl)-5-methyl-5,6-dihydro- 4H-1,2,4-oxadiazine |

### 5.3 Methods for Making Oxadiazine Compounds

Methods useful for making the Oxadiazine Compounds are set forth in the Examples below and generalized in Schemes 1-7 for the compounds of Formula (I) and Formula (II).

Schemes 1-7 represent general synthetic schemes for manufacturing Oxadiazine Compounds. These schemes are illustrative and are not meant to limit the possible techniques one skilled in the art may use to manufacture compounds disclosed herein. Different methods will be evident to those skilled in the art. Additionally, the various steps in the synthesis may be performed in an alternate sequence or order to provide the compound(s). Various modifications to these methods may be envisioned by those skilled in the art to achieve similar results to that of the inventors provided below. For example, optional protecting groups can be used as described, for example, in Greene et al., Protective Groups in Organic Synthesis (3^{rd} ed. 1999).

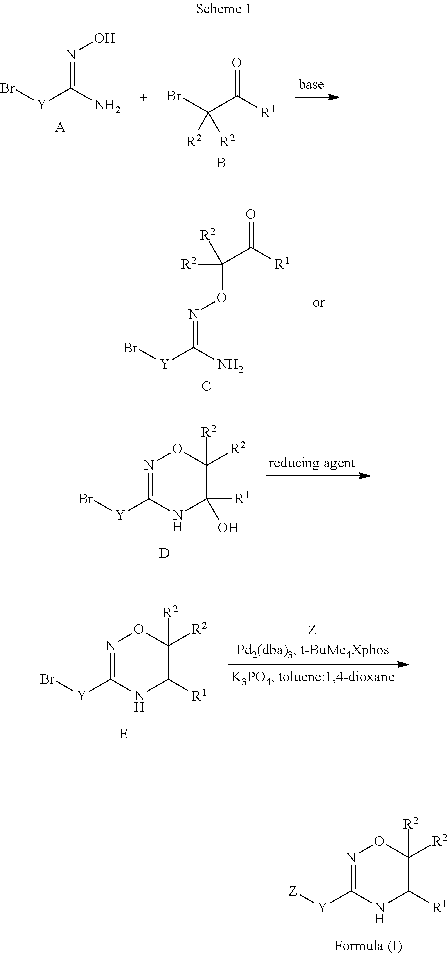

As shown in Scheme 1, a compound of formula A is coupled to the compound of formula B under standard coupling conditions to provide a compound of formula C or formula D. The compound of formula C or formula D is then reduced to provide a compound of formula E. The compound of formula E is coupled to Z under standard conditions to provide an Oxadiazine Compound of Formula (I).

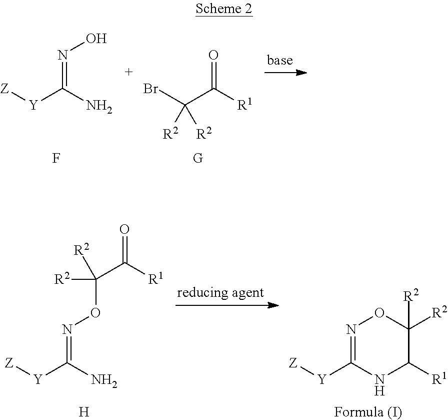

As shown in Scheme 2, a compound of formula F is coupled to the compound of formula G under basic coupling conditions to provide a compound of formula H. The compound of formula H is reduced under standard conditions to provide an Oxadiazine Compound of Formula (I).

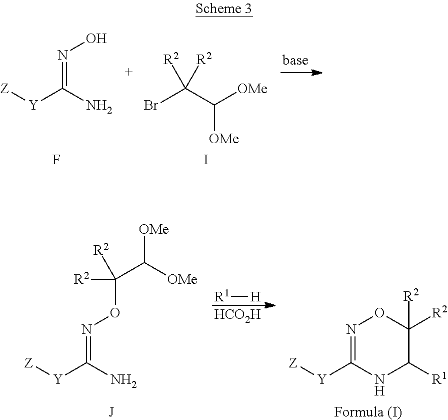

As shown in Scheme 3, a compound of formula F is coupled to the compound of formula I under basic coupling conditions to provide a compound of formula J. The compound of formula J is then cyclized in the presence of R¹-H to provide an Oxadiazine Compound of Formula (I).

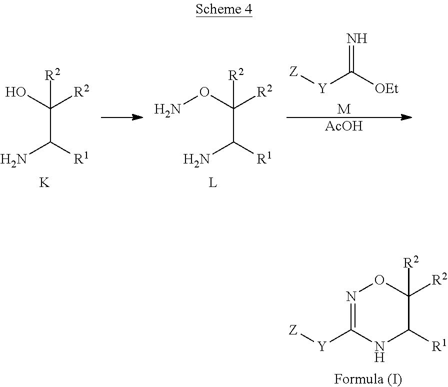

As shown in Scheme 4, a compound of formula K is coupled to amine under standard conditions to provide a compound of formula L. The compound of formula L is reacted with a compound of formula M under acidic conditions to provide an Oxadiazine Compound of Formula (I).

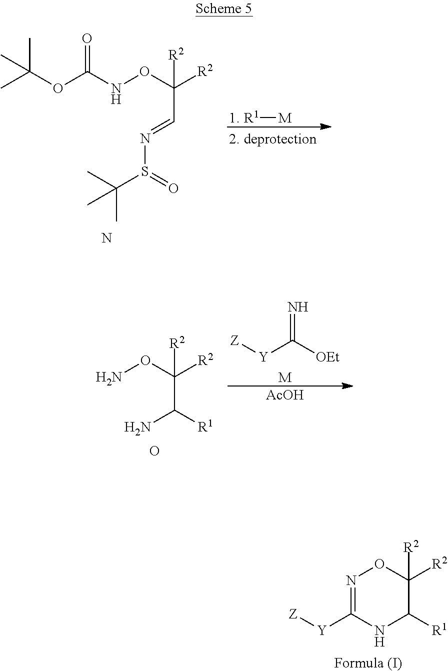

As shown in Scheme 5, a compound of formula N is reacted with R¹-M followed by deprotection to provide a compound of formula O. The compound of formula O is reacted with a compound of formula M under acidic conditions to provide an Oxadiazine Compound of Formula (I).

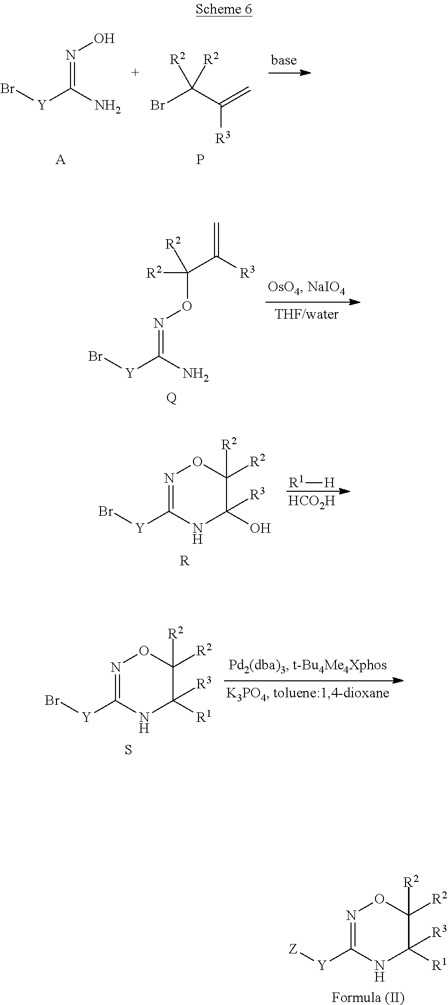

As shown in Scheme 6, a compound of formula A is coupled to the compound of formula P under basic coupling conditions to provide a compound of formula Q. The compound of formula Q is oxidized to provide a compound of formula R. The compound of formula R is then reacted with R¹-H to provide a compound of formula S. The compound of formula S is coupled to Z under standard conditions to provide an Oxadiazine Compound of Formula (II).

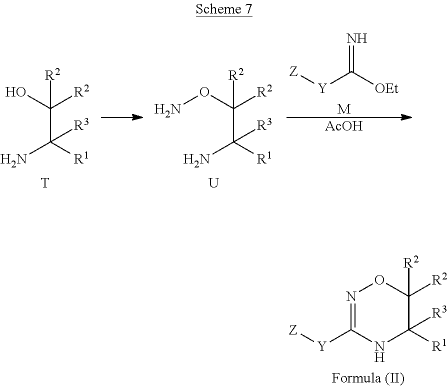

As shown in Scheme 7, a compound of formula T is coupled to amine under standard conditions to provide a compound of formula U. The compound of formula U is reacted with a compound of formula M under acidic conditions to provide an Oxadiazine Compound of Formula (II).

### 5.4 Pharmaceutical Compositions Comprising an Oxadiazine Compound

In another aspect, the present disclosure provides pharmaceutical compositions for treating, preventing, or ameliorating a symptom of a neurodegenerative disease in a subject having a neurodegenerative disease, wherein the pharmaceutical composition comprises a therapeutically effective amount of an Oxadiazine Compound, and a pharmaceutically acceptable carrier or vehicle.

As set out above, in some embodiments, Oxadiazine Compounds are provided in the form of pharmaceutically acceptable salts. These salts can be prepared in situ during the final isolation and purification of the compounds described herein, or by separately reacting a purified compound described herein in its free base or acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, laurylsulphonate, ammonium, amine salts and the like. See, for example, Berge, et al., (1977) “Pharmaceutical Salts”, J. Pharm. Sci. 66:1-19.

The pharmaceutically acceptable salts of Oxadiazine Compounds include the conventional nontoxic salts or acid salts of the compounds, e.g., from non-toxic organic or inorganic acids. For example, such conventional nontoxic salts include those derived from inorganic acids, such as hydrochloride, hydrobromic, sulfuric, sulfamic, phosphoric, nitric, and the like; and the salts prepared from organic acids, such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic and the like.

In general, a suitable dose of an Oxadiazine Compound will be in the range of 0.01 to 100 mg per kilogram body weight of the recipient per day or in the range of 0.2 to 10 mg per kilogram body weight per day. The desired dose can be administered once daily, but may be dosed as two, three, four, five, six or more sub-doses administered at appropriate intervals throughout the day.

The concentration of compounds included in compositions used in the methods described herein can range from about 1 nM to about 100 μM. Effective doses are believed to range from about 10 picomole/kg to about 100 micromole/kg.

An Oxadiazine Compound can be administered as the sole active agent, or in combination with other known therapeutics to be beneficial in the treatment of neurodegenerative diseases. In any event, the administering physician can provide a method of treatment that is prophylactic or therapeutic by adjusting the amount and timing of drug administration on the basis of observations of one or more symptoms (e.g., motor or cognitive function as measured by standard clinical scales or assessments) of the disease being treated.

Details on techniques for formulation and administration are well described in the scientific and patent literature, see, e.g., Remington: The Science and Practice of Pharmacy (2 Volumes), 22nd Edition, 2012), Pharmaceutical Press (“Remington's”). After a pharmaceutical composition has been formulated in an acceptable carrier, it can be placed in an appropriate container and labeled for treatment of an indicated condition. For administration of an Oxadiazine Compound, such labeling would include, e.g., instructions concerning the amount, frequency, and method of administration.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient, which can be combined with a carrier or vehicle material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

The compounds and pharmaceutical compositions described herein can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved.

Methods of preparing these formulations or compositions include the step of bringing into association a compound described herein with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

When an Oxadiazine Compound is administered as pharmaceuticals to humans and animals, it can be given per se or as a pharmaceutical composition containing, for example, 0.1% to 99.5% (more preferably, 0.5% to 90%) of active ingredient in combination with a pharmaceutically acceptable carrier.

The pharmaceutical compositions described herein can be administered in a variety of dosage forms including, but not limited to, a solid dosage form, a liquid dosage form, an oral dosage form, a parenteral dosage form, an intranasal dosage form, a suppository, a lozenge, a troche, a buccal dosage form, a controlled release dosage form, a pulsed release dosage form, an immediate release dosage form, an intravenous solution, a suspension or combinations thereof.

### Oral Formulations and Administration

Pharmaceutical formulations described herein suitable for oral administration can be in the form of capsules, cachets, pills, tablets, caplet, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound described herein as an active ingredient. The dosage can be an oral dosage form that is a controlled release dosage form. An Oxadiazine Compound can also be administered as a bolus, electuary or paste.

In solid dosage forms described herein for oral administration (capsules, tablets, pills, dragees, powders, granules and the like), the active ingredient is mixed with one or more pharmaceutically acceptable carriers and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using a binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

Tablets, and other solid dosage forms of the pharmaceutical compositions described herein, such as dragees, capsules, pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxybutylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and may be of a composition that they release the active ingredient(s) only, or preferentially, in a certain portion of the gastrointestinal tract, optionally, in a delayed manner Examples of embedding compositions which can be used include polymeric substances and waxes. The active ingredient can also be in micro-encapsulated form, if appropriate, with one or more of the above-described excipients.

Capsules for oral use include hard gelatin capsules in which the active ingredient is mixed with a solid diluent, and soft gelatin capsules, wherein the active ingredients is mixed with water or an oil, such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Liquid dosage forms for oral administration of the compounds described herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-13-cyclodextrin, may be used to solubilize compounds.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Besides inert diluents, the oral compositions can also include adjuvants, such as wetting agents, emulsifying and suspending agents, sweetening, flavoring, coloring, perfuming and preservative agents.

Pharmaceutical preparations for oral use can be obtained through combination of an Oxadiazine Compound with a solid excipient, optionally grinding a resultant mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose, such as methyl cellulose, hydroxypropylmethyl-cellulose or sodium carboxymethylcellulose; and gums including arabic and tragacanth; as well as proteins, such as gelatin and collagen. If desired, disintegrating or solubilizing agents can be added, such as the cross-linked polyvinyl pyrrolidone, agar, alginic acid, or a salt thereof, such as sodium alginate.

Pharmaceutical preparations for oral use can be presented as aqueous or liposome formulations. Aqueous suspensions can contain an Oxadiazine Compound in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents, such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives, such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending an Oxadiazine Compound in a vegetable oil, such as arachis oil, olive oil, sesame oil or coconut oil, or in a mineral oil, such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant, such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

### Parenteral Formulations and Administration

In another embodiment, an Oxadiazine Compound can be administered parenterally, such as intravenous (IV) or intramuscular (IM) administration. The formulations for administration will commonly comprise a solution of an Oxadiazine Compound dissolved in a pharmaceutically acceptable carrier. Administration of an Oxadiazine Compound to any of the above mentioned sites can be achieved by direct injection of the pharmaceutical composition comprising the Oxadiazine Compound or by the use of infusion pumps. The pharmaceutical compositions can be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

Pharmaceutical compositions suitable for parenteral administration comprise one or more compounds described herein in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Among the acceptable vehicles and solvents that can be employed for formulation and/or reconstitution are water (e.g., water for injection) and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids, such as oleic acid can likewise be used in the preparation of injectables. These solutions are sterile and generally free of undesirable matter. These formulations may be sterilized by conventional, well known sterilization techniques such as gamma-radiation or electron beam sterilization. The formulations can contain pharmaceutically acceptable auxiliary substances as required to approximate physiological conditions, such as pH adjusting and buffering agents, toxicity adjusting agents, e.g., sodium acetate, sodium chloride, potassium chloride, calcium chloride, sodium lactate and the like. The concentration of an Oxadiazine Compound in these formulations can vary widely, and will be selected primarily based on fluid volumes, viscosities, body weight, and the like, in accordance with the particular mode of administration selected and the subject's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, an Oxadiazine Compound can be administered by introduction into the central nervous system of the subject, e.g., into the cerebrospinal fluid of the subject. The formulations for administration will commonly comprise a solution of the Oxadiazine Compound dissolved in a pharmaceutically acceptable carrier. In certain aspects, the Oxadiazine Compound is introduced intrathecally, e.g., into a cerebral ventricle, the lumbar region, or the cisterna magna.

In some embodiments, the pharmaceutical composition comprising an Oxadiazine Compound is administered into a subject intrathecally. As used herein, the term “intrathecal administration” is intended to include delivering a pharmaceutical composition comprising an Oxadiazine Compound directly into the cerebrospinal fluid of a subject, by techniques including lateral cerebroventricular injection through a borehole or cisternal or lumbar puncture or the like (described in Lazorthes et al., Advances in Drug Delivery Systems and Applications in Neurosurgery, 1991, 18:143-192 and Omaya et al., Cancer Drug Delivery, 1984, 1:169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. In some embodiments, the pharmaceutical composition is administered by injection into the cisterna magna, or lumbar area of a subject.

### Depot Formulations and Administration

An Oxadiazine Compound can be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers, wherein the vehicle is fluid at room temperature and solidifies at body temperature.

Injectable depot forms can be made by forming microencapsule matrices of the subject compounds in biodegradable polymers, such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations can also be prepared by entrapping the drug in liposomes or microemulsions, which are compatible with body tissue.

### Intranasal Formulations and Administration

For administration by inhalation, the compounds are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base, such as lactose or starch.

### Other Formulations and Modes of Administration

For transmucosal administration (e.g., buccal, rectal, nasal, ocular, etc.), penetrants appropriate to the barrier to be permeated are used in the formulation. Such penetrants are generally known in the art.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid and polyanhydrides. Microcapsules of the foregoing polymers containing drugs are described in, for example, U.S. Pat. No. 5,075,109. Delivery systems also include non-polymer systems that are: lipids including sterols, such as cholesterol, cholesterol esters and fatty acids or neutral fats, such as mono-, di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent described herein is contained in a form within a matrix, such as those described in U.S. Pat. Nos. 4,452,775, 4,675,189, and 5,736,152, and (b) diffusional systems in which an active component permeates at a controlled rate from a polymer, such as described in U.S. Pat. Nos. 3,854,480, 5,133,974 and 5,407,686. In addition, pump-based hardware delivery systems can be used, some of which are adapted for implantation.

### 5.5 Treatment, Prevention or Amelioration of Symptoms of a Neurodegenerative Disease

In another aspect, a method for treating a neurodegenerative disease is described, comprising administering to a subject an effective amount a pharmaceutical composition comprising an effective amount of an Oxadiazine Compound.

In some embodiments, the method for treating a neurodegenerative disease is a method for reducing or ameliorating a symptom of the neurodegenerative disease.

In some embodiments, a method for reducing or ameliorating a symptom of a neurological disease is described, comprising administering to a subject in need thereof an effective amount of an Oxadiazine Compound. Ameliorating or reducing the symptoms can be manifested in a variety of ways, for example, by improvement in cognitive function. Such improvement can be assessed relative to the cognitive function of the subject prior to being treated or being administered an Oxadiazine Compound or a pharmaceutical composition comprising an effective amount of an Oxadiazine Compound.

In some embodiments, a method for preventing a neurological disease is described, comprising administering to a subject in need thereof an effective amount of an Oxadiazine Compound.

In some embodiments, a method for stopping progression of a neurological disease is described, comprising administering to a subject in need thereof an effective amount of an Oxadiazine Compound.

Exemplary symptoms of neurological disease that can be reduced or ameliorated by administration of an Oxadiazine Compound include, but are not limited to, progressive loss of memory, progressive loss of cognition, progressive loss of reasoning and/or loss of judgment. The loss of each of memory, cognition, reasoning and/or judgment can be progressive or sudden. Dementia is an exemplary symptom of neurodegenerative disease. Administration of an Oxadiazine Compound can reduce or improve one or more of these symptoms.

Exemplary cognitive functions that can be improved by administration of an Oxadiazine Compound are attention, learning, delayed memory, working memory, visual learning, speed of processing, vigilance, verbal learning, visual motor function, social cognition, long term memory or executive function.

In one embodiment, the neurodegenerative disease is Alzheimer's disease, probable Alzheimer's disease, mild cognitive impairment, age-related cognitive decline, or another neurodegenerative disease with co-existing symptoms of Alzheimer's disease (eg. Lewy body disease or Parkinsons's disease with Alzheimer's disease). In some embodiments, the neurodegenerative disease is early onset Alzheimer's disease. In some embodiments, the early onset Alzheimer's disease is autosomal dominant early onset Alzheimer's disease.

In one embodiment, the neurodegenerative results from injuries which increase amyloid with or without cognitive impairment including post-traumatic encephalopathy and traumatic brain injury.

In some embodiments, the subject is 65 years or older. In some embodiments, the subject is 55 years old or younger, or 50 years old or younger. In some embodiments, the subject is older than 55 years and younger than 65 years. In some embodiments, the subject is older than 55 years.

In some embodiments, the neurodegenerative disease is panic disorder, obsessive compulsive disorder, delusional disorder, drug-induced psychosis, post-traumatic stress disorder, age-related cognitive decline, attention deficit/hyperactivity disorder, personality disorder of the paranoid type, personality disorder of the schizoid type, dyskinesia, choreiform condition, psychosis associated with Parkinson's disease, psychotic symptoms associated with Alzheimer's disease, mood disorder, or dementia.

In some embodiments, the neurodegenerative disease is cognitive impairment, myclonus, seizures, Parkinsonism, extrapyramidal signs (EPS), apraxia, dystonia, dementia with Lewy bodies (DLB), aphasia, visual agnosia, or ataxia.

In some embodiments, the subject has impaired cognitive function including one or more of attention, learning, delayed memory, working memory, visual learning, speed of processing, vigilance, verbal learning, visual motor function, social cognition, long term memory or executive function, aphasias, apraxias or frontal lobe symptoms.

In some embodiments, the subject has a mutation in at least one gene selected from PSEN1, PSEN2 and APP. In some embodiments, the mutation in PSEN1, PSEN2 or APP is a missense mutation.

In some embodiments, the invention provides a method for treating or ameliorating a symptom of neurodegenerative disease (e.g., Alzheimer's disease) in a subject with an increased level of Aβ42 in cerebrospinal fluid, the method comprising administering to a subject in need thereof an effective amount of an Oxadiazine Compound. In such subject, the increased level of Aβ42 in cerebrospinal fluid can be detected relative to the level of Aβ42 in cerebrospinal fluid of a healthy subject. The identification and validation of biomarkers for diagnosing Alzheimer's disease in a subject using ELISA measurements of Aβ42 in cerebrospinal fluid and blood using modern amyloid imaging methods to detect changes in the level of Aβ42 identifies subjects with Alzheimer's disease with high specificity and sensitivity.

In some embodiments, the invention provides a method for lowering Aβ42 concentration in a subject, the method comprising administering to a subject in need thereof an effective amount of an Oxadiazine Compound. In some embodiments, the subject has an elevated Aβ42 concentration relative to a healthy subject.

In some embodiments, the invention provides a method for preventing increase of Aβ42 concentration in a subject, the method comprising administering to a subject in need thereof an effective amount of an Oxadiazine Compound.

### 5.6 Kits

Described herein are kits that can simplify the administration of an Oxadiazine Compound to a subject. The kit can comprise one or more containers filled with one or more of the ingredients of the pharmaceutical compositions described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

A typical kit comprises a unit dosage form of an Oxadiazine Compound. In one embodiment, the unit dosage form is a container, which can be sterile, containing an effective amount of an Oxadiazine Compound and a pharmaceutically acceptable carrier or vehicle. The kit can further comprise a label or printed instructions instructing the use of the Oxadiazine Compound to treat or prevent a neurodegenerative disease. The kit can also further comprise a unit dosage form of another prophylactic or therapeutic agent, for example, a container containing an effective amount of the other prophylactic or therapeutic agent. In one embodiment, the kit comprises a container containing an effective amount of an Oxadiazine Compound and an effective amount of another prophylactic or therapeutic agent. Examples of other prophylactic or therapeutic agents include, but are not limited to, those listed above.

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain additional information, exemplification and guidance which can be adapted to the practice of this invention in its various embodiments and equivalents thereof. The examples do not limit the scope of the invention described in the claims.

### 6. EXAMPLES

### General Experimental Techniques

General experimental details. The ¹H NMR spectra were recorded on a DMX300 spectrometer (300 MHz, Bruker), Avance III 400 spectrometers (400 MHz, Bruker) and an Inova unity 500 spectrometer (500 MHz Varian). Chemical shifts are given in δ units (ppm) relative to internal standard TMS and refer to CDCl₃ or CD₃OD or DMSO-d₆ solutions. LCMS and Mass were performed using an Shimadzu UFLC-LCMS-2010 EV/Agilent 1200 series 6310 ion trap LCMS, an Agilent 1100 system (Bin. Pump: G1312A, DAD: Agilent G1315B, MSD: Agilent LC/MSD G1956B ESI) or an Agilent 1260 system (Bin. Pump: G1312B, DAD: Agilent G1315D, MSD: Agilent LC/MSD G6130B ESI). Analytical High-Performance Liquid Chromatography (HPLC) and UPLC were performed by Shimadzu-SIL HTA/Agilent-1200 series/Waters-Acquity UPLC with ELS/PDA detector respectively. Specific Optical Rotations (SOR) was determined on a JASCO-P2000 Polarimeter and JASCO-P1010 Polarimeter.

Analytical methods: LCMS method A: column: Waters XSelect (C18, 30×2.1 mm, particle size 3.5 μ); flow: 1 mL/min; Column temp: 25° C.; Eluent A: 95% acetonitrile+5% 10 mM ammoniumbicarbonate in water; Eluent B: 10 mM ammoniumbicarbonate in water pH=9.0; Linear Gradient: t=0 min 5% A, t=1.6 min 98% A, t=3 mM 98% A; detection: DAD (220-320 nm). LCMS method B: Column: Phenomenex Gemini NX (C18, 50×2.0 mm, particle size: 3 μ); Flow: 0.8mL/min; Column temp: 25° C.; Eluent A: 95% acetonitrile +5% 10 mM ammoniumbicarbonate in water; Eluent B: 10 mM ammoniumbicarbonate in water pH=9.0; Linear Gradient: t=0 min 5% A, t=3.5 mM 98% A, t=6 mM 98% A; detection: DAD (220-320 nm). LCMS method C: column: Waters XSelect (C18, 30×2.1 mm, particle size 3.5 μ); flow: 1 mL/min; Column temp: 35° C.; eluent A: 0.1% formic acid in acetonitrile; eluent B: 0.1% formic acid in water; Linear gradient: t=0 min 5% A, t=1.6min 98% A, t=3 min 98% A; detection: DAD (220-320 nm). LCMS method D: Column: Waters XSelect (C18, 50×2.1 mm, particle size: 3.5 μ); Flow: 0.8 mL/min; Column temp: 25° C.; Eluent A: 95% acetonitrile+5% 10 mM ammoniumbicarbonate in water; Eluent B: 10 mM ammoniumbicarbonate in water pH=9.0; [0310]Linear Gradient: t=0 min 5% A, t=3.5 min 98% A, t=6 mM 98% A; detection: DAD (220-320 nm). LCMS method E: Column: Waters XSelect (C18, 50×2.1 mm, particle size: 3.5 μ); Flow: 0.8 mL/min; Column temp: 25° C.; Eluent A: 0.1% formic acid in acetonitrile, Eluent B: 0.1% formic acid in water; Linear Gradient: t=0 mM 5% A, t=3.5 mM 98% A, t=6 mM 98% A; detection: DAD (220-320 nm).

GCMS method A: Column: RXi-5MS 20 m, ID 180 μm, df 0.18 μm; Average velocity: 50 cm/s; Injection vol: 1 μl; Injector temp: 250° C.; Split ratio: 20/1; Carrier gas: He; Initial temp: 60° C.; Initial time: 1.0 mM; Solvent delay: 1.3 mM; Rate 50° C/min; Final temp 250° C.; Final time 3.5 mM; detection: FID: Det. temp: 300° C. and MS: 5973 MSD, EI-positive, Det.temp.: 280° C. Mass range: 50-550.

Basic preparative MPLC was performed on a Reveleris Prep system: column: Waters Xselect CSH (C18 145×25 mm, particle size 10 μ); Flow: 40 mL/min; Column temp: room temperature; Eluent A: 10 mM ammoniumbicarbonate in water pH=9.0); Eluent B: 99% acetonitrile +1% 10 mM ammoniumbicarbonate in water; using the indicated gradient and detection wavelength.

Acidic reversed phase MPLC was performed on a Reveleris system: column: Reveleris-C18; Eluent A: 0.1% formic acid in acetonitrile; eluent B: 0.1% formic acid in water using the indicated gradient and detection wavelenght.

### Example 1

### Synthesis of 5-bromo-N′-hydroxy-6-methoxypicolinimidamide

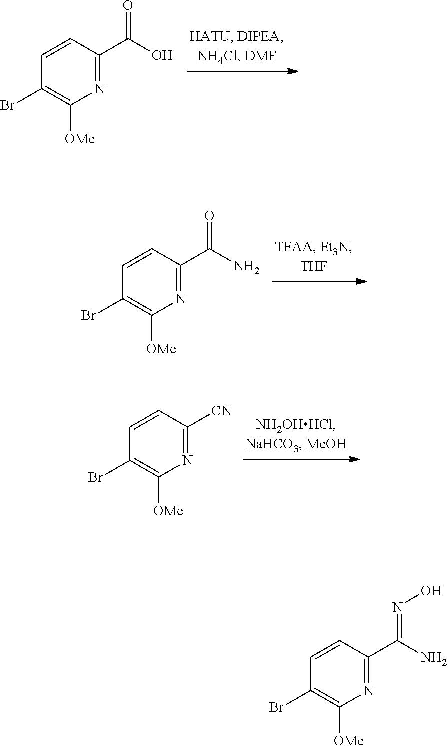 [SMILES: COc1nc(C(=O)O)ccc1Br ; COc1nc(C(N)=O)ccc1Br ; COc1nc(C#N)ccc1Br ; COc1nc(/C(N)=N/O)ccc1Br]

5-bromo-6-methoxypicolinamide: To a stirred solution of 5-bromo-6-methoxypicolinic acid (120 g, 515.06 mmol) in DMF (1200 mL) under an argon atmosphere were added diisopropyl ethylamine (148 mL, 1030.0 mmol), ammonium chloride (41.4 g, 772.5 mmol) and HATU (293.5 g, 772.5 mmol) at 0° C. The reaction mixture was warmed to room temperature and stirred for 16 h. After consumption of the starting material (monitored by TLC), the reaction mixture was diluted with ice cold water (1500 mL), the obtained solid was filtered and dried in vacuo to obtain 5-bromo-6-methoxypicolinamide (96.4 g, 81%) as white solid. ¹H-NMR (DMSO-d₆, 400 MHz): δ 8.17 (d, 1H), 8.04 (br s, 1H), 7.70 (br s, 1H), 7.52 (d, 1H), 4.04 (s, 3H); TLC: 50% EtOAc/hexane (R_{f}:05).

5-bromo-6-methoxypicolinonitrile: To a stirred solution of 5-bromo-6-methoxypicolinamide (100 g, 432.90 mmol) in THF (1000 mL) under an argon atmosphere were added triethyl amine (151 mL, 1082.25 mmol) and trifluoro acetic anhydride (92.1 mL, 649.3 mmol) at −5° C. The reaction mixture was stirred at 0° C. for 2 h. After consumption of the starting material (monitored by TLC), the reaction was diluted with water (1800 mL) and extracted with EtOAc (2×1500 mL). The combined organic extract was dried over sodium sulfate, filtered and concentrated in vacuo to obtain 5-bromo-6-methoxypicolinonitrile (81.5 g, 88%) as an off-white solid. ¹H-NMR (DMSO-d₆, 400 MHz): δ 8.30 (d, 1H), 7.61 (d, 1H), 3.97 (s, 3H); TLC: 20% EtOAc/hexane (R_{f}:06).

5-bromo-N′-hydroxy-6-methoxypicolinimidamide: To a stirred solution of 5-bromo-6-methoxypicolinonitrile (66 g, 308.41 mmol) in MeOH (600 mL) under an argon atmosphere were added hydroxylamine hydrochloride (27.9 g, 400.93 mmol) and sodium bicarbonate (38.8 g, 462.65 mmol) at room temperature. The reaction mixture was stirred at 60° C. for 1 h. After consumption of the starting material (monitored by TLC), the reaction was quenched with saturated ammonium chloride solution (900 mL) and extracted with EtOAc (2×900 mL). The combined organic extract was washed with water (500 mL), brine (450 mL), dried over sodium sulfate, filtered and concentrated in vacuo to obtain 5-bromo-N′-hydroxy-6-methoxypicolinimidamide Example 1 (68 g, 90%) as white solid. ¹H-NMR (CDCl₃, 400 MHz): δ 7.82 (d, 1H), 7.66 (br s, 1H), 7.40 (d, 1H), 5.50 (br s, 2H), 4.06 (s, 3H); LCMS: 99.85%; 247.7 (M+1); (column; X-Select CSH C-18 (4.6*50 mm, 2.5 μm);. Mobile phase:A:0.05% Formic acid in water; B: 0.05% Formic acid in Acetonitrile;Ret. Time:2.357; HPLC (purity): 99.83%; (column; X-Select CSH C-18 (4.6*150 mm, 3.5 μm).

### Example 2

### Synthesis of (Z)-N′-hydroxy-6-methoxy-5-(4-methyl-1H-imidazol-1-yl) picolinimidamide

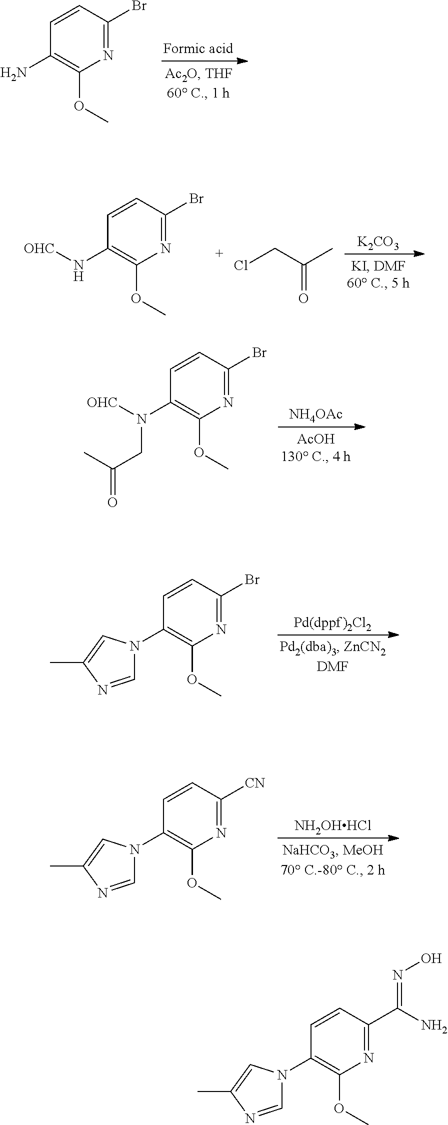

N-(6-bromo-2-methoxypyridin-3-yl) formamide: To the acetic anhydride (8.5 mL) at room temperature under an argon atmosphere was added formic acid (12.5 mL). The reaction mixture was stirred at room temperature for 30 mm Then 6-bromo-2-methoxypyridin-3-amine (5 g, 25 mmol) in THF (22 mL) at room temperature was added to the reaction mixture. The reaction mixture was stirred at 60° C. for 1 h. After consumption of the starting material (monitored by TLC), the reaction mixture was diluted with ice cold water (500 mL) stirred for 30 min to afford the solid. The solid was collected by filtration and dried in vacuo to afford N-(6-bromo-2-methoxypyridin-3-yl) formamide (5.5 g, 98%) as an off-white solid. ¹H NMR (CDCl₃, 500MHz): δ 8.52-8.50 (m, 2H), 7.61 (br s, 1H), 7.09 (d, 1H), 4.05 (s, 3H); LCMS: 99.8%; 232.7 (M+3); (column; Ascentis Express C-18 (50×3.0 mm, 2.7 μm); RT 2.05 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:03).

N-(6-bromo-2-methoxypyridin-3-yl)-N-(2-oxopropyl) formamide: To a stirred solution of N-(6-bromo-2-methoxypyridin-3-yl) formamide (27 g, 117 mmol) in DMF (216 mL) at room temperature under an argon atmosphere were added potassium carbonate (57 mg, 411 mmol), 1-chloropropan-2-one (28.8 g, 293 mmol) and potassium iodide (1.94 g, 12 mmol). The reaction mixture was stirred at 60° C. for 5 h. After consumption of the starting material (monitored by TLC), the reaction mixture was diluted with ice cold water (200 mL) and stirred for 10 min to afford the solid. The solid was collected by filtration and dried in vacuo to afford N-(6-bromo-2-methoxypyridin-3-yl)-N-(2-oxopropyl) formamide (32 g, 94%) as an off-white solid. ¹H NMR (CDCl₃, 500MHz): δ 8.21 (s, 1H), 7.48 (d, 1H), 7.13 (d, 1H), 4.46 (s, 2H), 4.01 (s, 3H), 2.16 (s, 3H); LCMS: 99.4%; 288.7 (M+3); (column; Ascentis Express C-18 (50×3.0 mm, 2.7 μm); RT 2.05 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:02).

6-bromo-2-methoxy-3-(4-methyl-1H-imidazol-1-yl) pyridine: The mixture of ammonium acetate (43 g, 553 mmol) in AcOH (208 mL) at room temperature under an argon atmosphere was stirred for 30 mm Then N-(6-bromo-2-methoxypyridin-3-yl)-N-(2-oxopropyl) formamide (32 g, 111 mmol) was added to the reaction mixture at room temperature. The reaction mixture was stirred at 130° C. for 4 h. After consumption of the starting material (monitored by TLC), the reaction mixture was diluted with ice cold water (200 mL), the aqueous layer was neutralized with 50% sodium hydroxide solution (200 mL) (pH′7) to afford the solid. The solid was collected by filtration, washed with ether (100 mL) and dried in vacuo to afford 6-bromo-2-methoxy-3-(4-methyl-1H-imidazol-1-yl) pyridine (17.5 g, 60%) as an off-white solid. ¹H NMR (CDCl₃, 400 MHz): δ 7.72 (s, 1H), 7.39 (d, 1H), 7.16 (d, 1H), 6.91 (s, 1H), 4.03 (s, 3H), 2.29 (s, 3H); LCMS: 99.3%; 267.9 (M+1); (column; Ascentis Express C-18 (50×3.0 mm, 2.7 μm); RT 1.54 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 40% EtOAc/hexane (R_{f}:02).

6-methoxy-5-(4-methyl-M-imidazol-1-yl) picolinonitrile: To a stirred solution of 6-bromo-2-methoxy-3-(4-methyl-1H-imidazol-1-yl) pyridine (20 g, 74 mmol) in DMF (240 mL) at room temperature under an argon atmosphere were added Pd(dppf)₂Cl₂ (500 mg, 0.9 mmol), Pd₂(dba)₃ (682 mg, 0.7 mmol) and zinc cyanide (5.3 g, 45 mmol). The reaction mixture was stirred at 140° C. for 2 h. After consumption of the starting material (monitored by TLC), the reaction mixture was diluted with 25% NH₄OH solution (240 mL) to afford the solid. The solid was collected by filtration and dried in vacuo to afford 6-methoxy-5-(4-methyl-1H-imidazol-1-yl) picolinonitrile (14 g, 88%) as a pale yellow solid. ¹H NMR (CDCl₃: 400 MHz): δ 7.89 (s, 1H), 7.63 (d, 1H), 7.43 (d, 1H), 7.01 (s, 1H), 4.09 (s, 3H), 2.30 (s, 3H); LCMS: 98.7%; 214.9 (M+1); (column; Ascentis Express C-18 (50×3.0 mm, 2.7 μm); RT 1.19 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: EtOAc (R_{f}: 0.3).

N′-hydroxy-6-methoxy-5-(4-methyl-M-imidazol-1-yl) picolinimidamide: To a stirred solution of 6-methoxy-5-(4-methyl-1H-imidazol-1-yl) picolinonitrile (4 g, 19 mmol) in MeOH (100 mL) at room temperature under an argon atmosphere were added hydroxyl amine hydrochloride (1.7 g, 24 mmol) and sodium bicarbonate (2.35 g, 28 mmol). The reaction mixture was stirred at 70-80° C. for 2 h. After consumption of the starting material (monitored by TLC), the volatiles were evaporated in vacuo. The residue was diluted with ice cold water (100 mL) to afford the solid. The solid was collected by filtration and dried in vacuo to afford N′-hydroxy-6-methoxy-5-(4-methyl-1H-imidazol-1-yl) picolinimidamide Example 2 (4 g, 87%) as a pale yellow solid. LCMS: 99.8%; 248 (M+1); (column; Ascentis Express C-18 (50×3.0 mm, 2.7 μm); RT 0.42 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: EtOAc (R_{f}:02).

### Example 3

### Synthesis of N′-((1,1-dimethoxypropan-2-yl) oxy)-6-methoxy-5-(4-methyl-1H-imidazol-1-yl) picolinimidamide

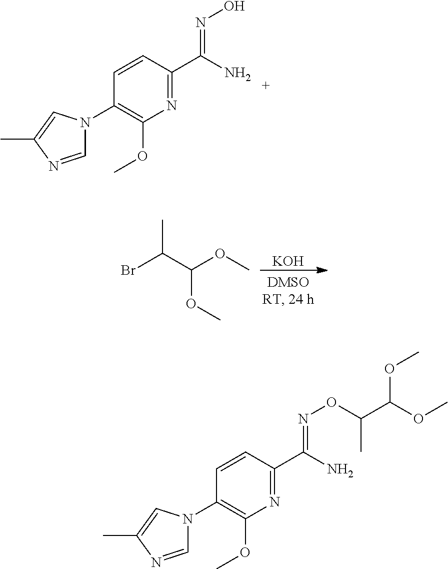

To a stirred solution of N′-hydroxy-6-methoxy-5-(4-methyl-1H-imidazol-1-yl) picolinimidamide (1 g, 4 mmol) in DMSO (5 mL) at 0° C. under an argon atmosphere were added potassium hydroxide (272 mg, 5 mmol) and 2-bromo-1,1-dimethoxypropane (814 mg, 4 mmol). The reaction mixture was warmed to room temperature and stirred for 24 h. After consumption of the starting material (monitored by TLC), the reaction mixture was diluted with ice cold water (30 mL) and extracted with EtOAc (2×30 mL). The combined organic extracts were dried over sodium sulfate, filtered and concentrated in vacuo to obtain N′-((1,1-dimethoxypropan-2-yl) oxy)-6-methoxy-5-(4-methyl-1H-imidazol-1-yl) picolinimidamide Example 3 (300 mg, crude) as an off-white solid used in the next step without further purification. LCMS: 46.2%; 349.9 (M+1); (column; Ascentis Express C-18 (50×3.0 mm, 2.7 μm); RT 1.63 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:06).

### Example 4

### Synthesis of ethyl 6-methoxy-5-(4-methyl-1H-imidazol-1-yl)picolinimidate dihydrochloride

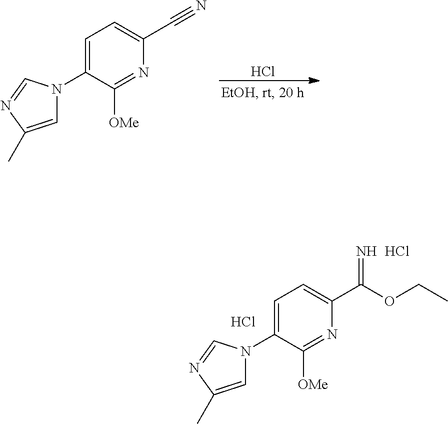 [SMILES: COc1nc(C#N)ccc1-n1cnc(C)c1 ; Cl ; CCOC(=N)c1ccc(-n2cnc(C)c2)c(OC)n1 ; Cl ; Cl]

A solution of 6-methoxy-5-(4-methyl-1H-imidazol-1-yl)picolinonitrile (387 mg, 1.8 mmol) in dry ethanol (15 mL) was saturated with HCl (gas) and stirred at rt for 20 hours. The mixture was concentrated in vacuo and triturated from Et₂O to obtain 6-methoxy-5-(4-methyl-1H-imidazol-1-yl)picolinimidate dihydrochloride Example 4 (550 mg, 91%) as a white solid. LCMS: 85.6%; 261.2 (M+1); RT 1.85 mm (method A).

### Example 5

### Synthesis of 3-(5-bromo-6-methoxypyridin-2-yl)-5-methyl-5,6-dihydro-4H-1,2,4-oxadiazin-5-ol

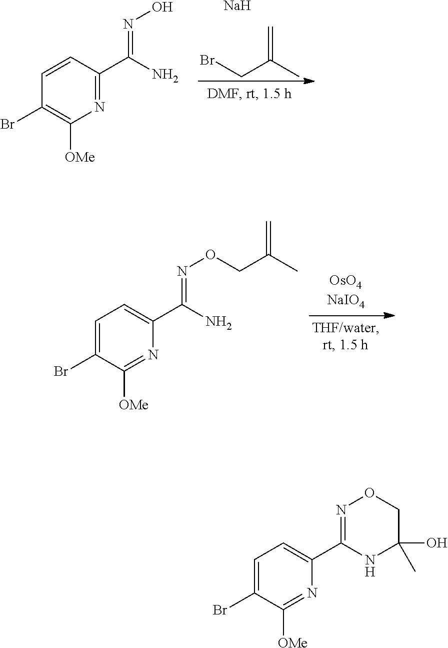

5-bromo-6-methoxy-N′-((2-methylallyl)oxy)picolinimidamide: To a solution of 5-bromo-N′-hydroxy-6-methoxypicolinimidamide (200 mg, 0.8 mmol) in dry N,N-dimethylformamide (8 mL) under argon atmosphere was added sodium hydride (34 mg, 0.9 mmol, 60%). The mixture was stirred for 30 minutes at room temperature. Then a solution of 3-bromo-2-methylpropene (219 mg, 1.6 mmol) in dry N,N-dimethylformamide (2 mL) was added, and the mixture was stirred at room temperature for 1.5 hours. The reaction was quenched with water and extracted twice with ethyl acetate. The combined organic layers were washed with water, brine, dried with sodium sulfate and concentrated in vacuo to afford a yellow oil that was purified by silica column chromatography 110% EtOAc in heptanel to afford 5-bromo-6-methoxy-N′-((2-methylallyl)oxy)picolinimidamide (78 mg, 59%) as a colorless oil. ¹H NMR (CDCl₃, 300 MHz) δ 7.78 (d, J=8.0 Hz, 1H), 7.46 (d, J=8.0 Hz, 1H), 5.41 (s, 2H), 5.06-4.98 (m, 1H), 4.97-4.90 (m, 1H), 4.53 (s, 2H), 4.04 (s, 3H), 1.80 (s, 3H); LCMS: 97.1%; 300.0 (M+1); RT 2.29 min. (method A); TLC: 10% EtOAc/Heptane (R_{f}:031).

3-(5-bromo-6-methoxypyridin-2-yl)-5-methyl-5,6-dihydro-4H-1,2,4-oxadiazin-5-ol: To a solution of 5-bromo-6-methoxy-N′-((2-methylallyl)oxy)picolinimidamide (100 mg, 0.3 mmol) in tetrahydrofuran/water (4/1, 5 mL) were added osmium tetroxide in water (172 mg, 0.03 mmol, 4 wt %) and sodium periodate (178 mg, 0.8 mmol). The resulting mixture was stirred at room temperature for 1.5 hours. The mixture was quenched with a saturated aqueous sodium bicarbonate and extracted twice with ethyl acetate. The combined organic layers were washed with brine, dried with sodium sulfate and concentrated to afford crude 3-(5-bromo-6-methoxypyridin-2-yl)-5-methyl-5,6-dihydro-4H-1,2,4-oxadiazin-5-ol Example 5 (120 mg) as a beige solid. Both LCMS and ¹HNMR showed the material to be a mixture of the aminal and the open form in a 3:1 ratio, the material was used as such in the next step. ¹H NMR (CDCl₃, 300 MHz, major) δ 7.85 (d, J=8.0 Hz, 1H), 7.53 (d, J=8.0 Hz, 1H), 6.83 (s, 1H), 4.11 (dd, J=10.9, 1.4 Hz, 1H), 4.04 (s, 3H), 3.53 (d, J=10.9 Hz, 1H), 3.04 (s, 1H), 1.57 (s, 3H); LCMS: 74.6%; 302.0 (M+1); RT 1.81 min (major), 25.4%; 302.0 (M+1); RT 2.00 min (minor), (method A).

### Example 6

### Synthesis of 2-bromo-1-(4-chlorophenyl) propan-1-one

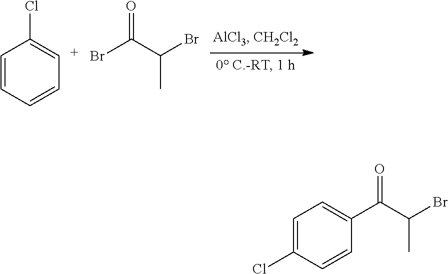

2-bromo-1-(4-chlorophenyl) propan-1-one: To a stirred solution of chlorobenzene (5.7 mL, 56 mmol) in CH₂Cl₂ (100 mL) at 0° C. were added aluminum trichloride (12.3 g, 93 mmol) and 2-bromopropanoyl bromide (10 g, 46 mmol) under an argon atmosphere. The reaction mixture was warmed to room temperature and stirred for 1 h. After consumption of the starting material (monitored by TLC), the reaction mixture was diluted with ice cold water (200 mL) and extracted with CH₂Cl₂ (2×200 mL). The combined organic extract was washed with a sodium bicarbonate solution (200 mL), dried over sodium sulfate, filtered and concentrated in vacuo to obtain 2-bromo-1-(4-chlorophenyl) propan-1-one Example 6 (8.5 g, 74%) as an off-white semi-solid used in the next step without further purification. ¹H NMR (CDCl₃, 500 MHz): δ 7.97 (d, 2H), 7.45 (d, 2H), 5.22-5.19 (m, 1H), 1.90 (d, 3H); LCMS: 98.1%; 249.3 (M+3); (column; X-select CSH C-18 (50×3.0 mm, 2.5 μm); RT 4.36 min; mobile phase: 2.5 mM Aq NH₄OAc: ACN; T/B %: 0.01/10, 0.5/10, 3.5/90, 7/90; flow rate: 0.8 mL/min) (Gradient); TLC: 20% EtOAc/hexane (R_{f}:05).

### Example 7

### Synthesis of 2-bromo-1-(3,4-dichlorophenyl) propan-1-one

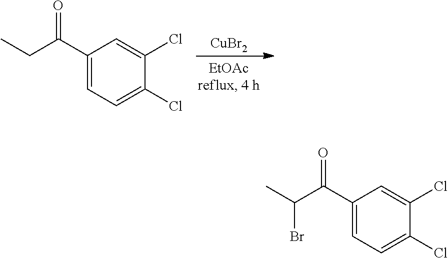

To a stirred solution of 1-(3,4-dichlorophenyl) propan-1-one (4 g, 20 mmol) in EtOAc (100 mL) at room temperature under an argon atmosphere was added copper bromide (9.6 g, 43 mmol). The reaction mixture was refluxed for 4 h. After consumption of the starting material (monitored by TLC), the reaction mixture was filtered. The filtrate was concentrated in vacuo to obtain 2-bromo-1-(3,4-dichlorophenyl) propan-1-one Example 7 (4 g, crude) as colorless liquid used in the next step without further purification. ¹H NMR (CDCl₃, 500 MHz): δ 8.10 (s, 1H), 7.84 (d, 1H), 7.58 (d, 1H), 5.20-5.17 (m, 1H), 1.90 (s, 3H); TLC: 5% CH₂Cl₂/hexane (R_{f}:05).

### Example 8

### Synthesis of 2-bromo-1-(4-chlorophenyl)-2-cyclopropylethanone

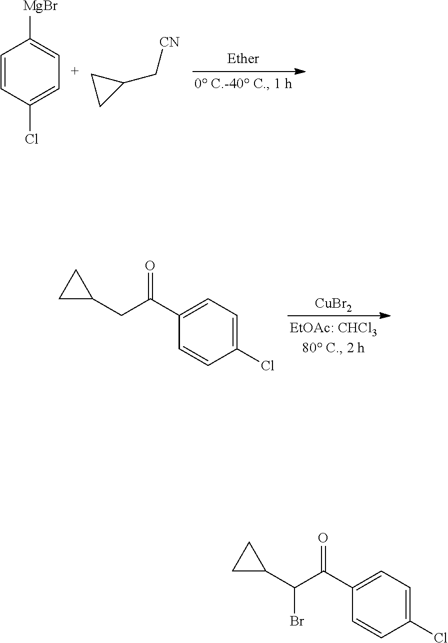

1-(4-chlorophenyl)-2-cyclopropylethan-1-one: To a stirred solution of (4-chlorophenyl) magnesium bromide (1.0 M in ether) (37 mL, 37 mmol) in ether (60 mL) at 0° C. under an argon atmosphere was added 2-cyclopropylacetonitrile (3 g, 37 mmol). The reaction mixture was stirred for 1 h at 40° C. After consumption of the starting material (monitored by TLC), the reaction mixture was diluted with water (100 mL) and extracted with EtOAc (2×100 mL). The combined organic extracts were dried over sodium sulfate, filtered and concentrated in vacuo. The crude material was purified by column chromatography using 2% EtOAc: hexane to afford 1-(4-chlorophenyl)-2-cyclopropylethan-1-one (1.5 g, 21%) as an off-white solid. ¹H NMR (CDCl₃, 500 MHz): δ 7.91 (d, 2H), 7.46 (d, 2H), 2.87 (d, 2H), 1.20-1.12 (m, 1H), 0.65-0.60 (m, 2H), 0.23-0.19 (m, 2H); LCMS: 95.0%; 194.8 (M+1); (column; Ascentis Express C-18 (50×3.0 mm, 2.7 μm); RT 2.88 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% EtOAc/hexane (R_{f}:05).

2-bromo-1-(4-chlorophenyl)-2-cyclopropylethan-1-one: To a stirred solution of 1-(4-chlorophenyl)-2-cyclopropylethan-1-one (1.2 g, 6 mmol) in EtOAc:CHCl₃ (1:1, 36 mL) at room temperature under an argon atmosphere was added copper bromide (3 g, 13 mmol). The reaction mixture was stirred for 2 h at 80° C. After consumption of the starting material (monitored by TLC), the reaction mixture was filtered. The filtrate was concentrated in vacuo. The crude material was purified by column chromatography using 2% EtOAc: hexane to afford 2-bromo-1-(4-chlorophenyl)-2-cyclopropylethan-1-one Example 8 (1.1 g, 65%) as an off-white solid. ¹H NMR (CDCl₃, 500 MHz): δ 7.94 (d, 2H), 7.46 (d, 2H), 4.39 (d, 1H), 1.87-1.79 (m, 1H), 0.96-0.91 (m, 2H), 0.61-0.53 (m, 1H), 0.49-0.41 (m, 1H); TLC: 5% EtOAc/hexane (R_{f}:04).

### Example 9

### Synthesis of 2-bromo-1-(4-chlorophenyl)-2-methylpropan-1-one

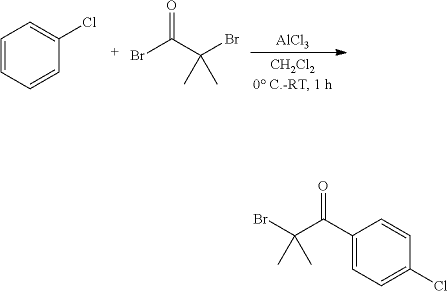

To a stirred solution of chlorobenzene (1 g, 4 mmol) in CH₂Cl₂ (9 mL) at 0° C. under an argon atmosphere were added aluminum trichloride (986 mg, 9 mmol) and 2-bromo-2-methylpropanoyl bromide (586 mg, 5 mmol). The reaction mixture was stirred at 0° C. for 10 mm Then the reaction mixture was warmed to room temperature for 1 h. After consumption of the starting material (monitored by TLC), the reaction mixture was diluted with ice cold water (50 mL) and extracted with CH₂Cl₂ (2×50 mL). The combined organic extracts were washed with a saturated sodium bicarbonate solution (50 mL), dried over sodium sulfate, filtered and concentrated in vacuo to obtain 2-bromo-1-(4-chlorophenyl)-2-methylpropan-1-one Example 9 (750 mg, 66%) as a brown solid used in the next step without further purification. ¹H NMR (CDCl₃, 500 MHz): δ 8.13 (d, 2H), 7.41 (d, 2H), 2.01 (s, 6H); TLC: 5% EtOAc/hexane (R_{f}:07).

### Example 10

### Synthesis of cis-3-(5-Bromo-6-methoxypyridin-2-yl)-5-(4-chlorophenyl)-6-methyl-5,6-dihydro-4H-1,2,4-oxadiazine

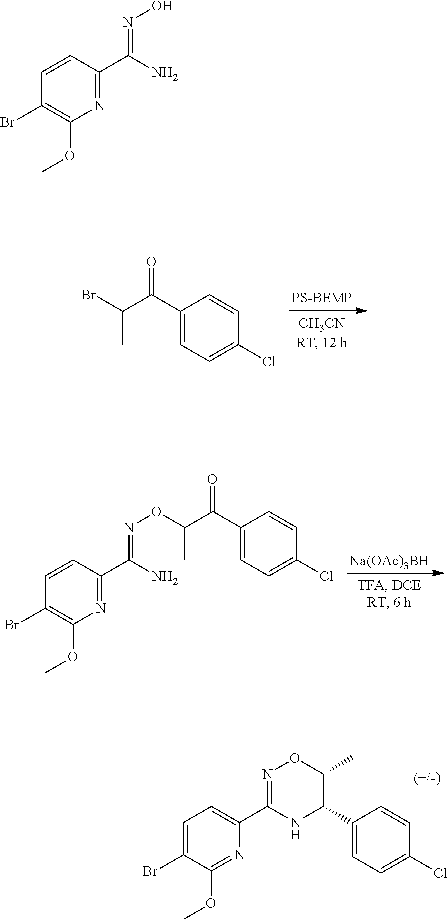 [SMILES: COc1nc(/C(N)=N/O)ccc1Br ; CC(Br)C(=O)c1ccc(Cl)cc1 ; COc1nc(/C(N)=N/OC(C)C(=O)c2ccc(Cl)cc2)ccc1Br ; COc1nc(C2=NO[C@H](C)[C@H](c3ccc(Cl)cc3)N2)ccc1Br ; "Na(OAc)3BH"]

5-Bromo-to-N′-((1-(4-chlorophenyl)-1-oxopropan-2-yl) oxy)-6-methoxypicolinimidamide: To a stirred solution of 5-bromo-N′-hydroxy-6-methoxypicolinimidamide Example 1 (1.5 g, 6 mmol) in CH₃CN (50 mL) at room temperature under an argon atmosphere was added PS-BEMP (2.5 g) and stirred for 10 min Then 2-bromo-1-(4-chlorophenyl) propan-1-one Example 6 (1.81 g, 7.31 mmol) in CH₃CN (25 mL) was added to the reaction mixture at room temperature. The reaction mixture was stirred for 12 h at room temperature. After consumption of the starting material (monitored by TLC), the reaction mixture was filtered. The filtrate was concentrated in vacuo to obtain 5-bromo-N′-((1-(4-chlorophenyl)-1-oxopropan-2-yl) oxy)-6-methoxypicolinimidamide (2.8 g, crude) as brown solid used in the next step without further purification. LCMS: 65.3%; 413.7 (M+3); (column; Ascentis Express C-18 (50×3.0 mm, 2.7 μm); RT 3.37 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:07).

cis-3-(5-Bromo-6-methoxypyridin-2-yl)-5-(4-chlorophenyl)-6-methyl-5,6-dihydro-4H-1,2,4-oxadiazine: To a stirred solution of 5-bromo-N′-((1-(4-chlorophenyl)-1-oxopropan-2-yl) oxy)-6-methoxypicolinimidamide (2.8 g, crude) in 1,2-dichloroethane (56 mL) at room temperature under an argon atmosphere was added trifluoroacetic acid (1.6 mL) and sodium triacetoxyborohydride (2.87 g). The reaction mixture was stirred for 6 h at room temperature. After consumption of starting material (monitored by TLC), the reaction mixture was diluted with a saturated sodium bicarbonate solution (50 mL) and extracted with CH₂Cl₂ (2×50 mL). The combined organic extracts were dried over sodium sulfate, filtered and concentrated in vacuo to obtain cis-3-(5-bromo-6-methoxypyridin-2-yl)-5-(4-chlorophenyl)-6-methyl-5,6-dihydro-4H-1,2,4-oxadiazine Example 10 (1 g, crude) as a brown solid. ¹H NMR (CD₃OD, 400 MHz): δ 7.99 (d, 1H), 7.41 (d, 1H), 7.33 (d, 2H), 7.25 (d, 2H), 4.69 (d, 1H), 4.03-3.99 (m, 4H), 0.98 (d, 3H); LCMS: 94.0%; 397.7 (M+3); (column; Ascentis Express C-18 (50×3.0 mm, 2.7 μm); RT 3.13 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:04).

Racemic compound of Example 10 was separated using a Chiralpak-IC column (250×20 mm, 5 μ) (20 mg loading; 0.1% DEA in n-hexane:CH₂Cl₂:MeOH (50:50) (A:B: 90:10) as mobile phase; flow rate: 20 mL/min) to provide the compounds of Example 10A (Fraction I (−)) and Example 10B (Fraction II (+)).

A sample of Example 10B (Fraction II (+)) was crystallized from MeOH/H₂O, and the structure was determined by X-ray analysis. The configuration of the substituents on the oxadiazine ring was determined to be cis, and the absolute configuration was determined to be 5S, 6R.

Analytical conditions for Example 10A and Example 10B: HPLC: column; zorbax-SB-C-18 150×4.6 mm, 3.5 μm); mobile Phase: ACN+0.5% TFA; 0.5% TFA+5% ACN; flow rate: 1.0 mL/min; Gradient programme: T/B % 0.01/90, 10/10, 25/10: diluent: CH₃CN: Water; Chiral HPLC: (Chiralpak-IC (250×4.6 mm, 5 μm; mobile phase (A) 0.1% DEA in n-hexane (B) CH₂Cl₂:MeOH (50:50) (A:B; 90:10); flow Rate: 1.0 mL/min).

### Example 10A

(−)-(5R,6S)-3-(5-Bromo-6-methoxypyridin-2-yl)-5-(4-chlorophenyl)-6-methyl-5,6-dihydro-4H-1,2,4-oxadiazine, Fraction (I) (−): Mass (ESI): 397.8 [M+3]; HPLC (purity): 99.7%; RT 11.51 min; Chiral HPLC: 99.3% RT=11.75 min: Optical rotation [α]_{D}^{20.03}: −151.76 (c=0.25, CH₂Cl₂).

### Example 10B

(+)-(5S,6R)-3-(5-bromo-6-methoxypyridin-2-yl)-5-(4-chlorophenyl)-6-methyl-5,6-dihydro-4H-1,2,4-oxadiazine, Fraction (II) (+): 1H NMR (CDCl3, 500 MHz): δ 7.88 (d, 1H), 7.62 (d, 1H), 7.33 (d, 2H), 7.23 (d, 2H), 6.74 (br s, 1H), 4.61 (d, 1H), 4.14-4.08 (m, 1H), 4.00 (s, 3H), 1.01 (d, 3H); Mass (ESI): 397.7 1M+31; HPLC (purity): 99.3%; RT 11.52 min; Chiral HPLC: 99.2% RT=13.08 min: Optical rotation [α]_{D}^{20.01}: +157.95 (c=0.25, CH₂Cl₂).

Experimental details for the X-ray crystal structure of Example 10B: Crystals suitable for X-ray diffraction studies were obtained from a methanol/water mixture. After the initial formation of needle-like crystals the compound recrystallized to transparent blocks which were used in this analysis.

For single-crystal X-ray diffraction, a single-crystal was taken out of the mother liquid, immediately coated with low viscosity oil, mounted on a Mitagen Microloop and shock frozen to 150K using liquid nitrogen. Intensity data were collected at 150K. The measurement was performed on a Bruker Kappa ApexII with MoKa radiation, using φ and ω scans.

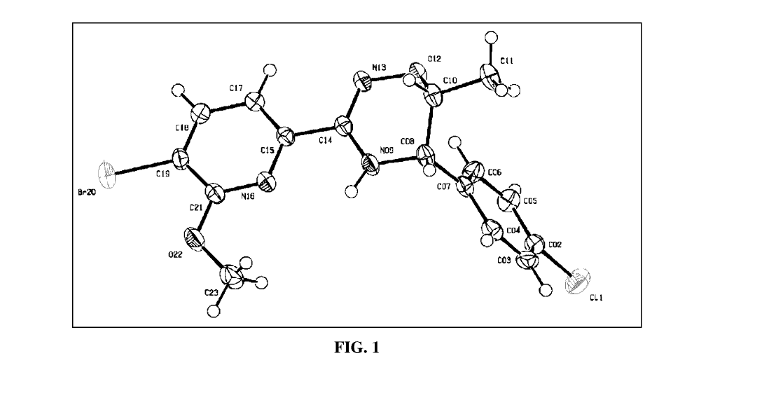

FIG. 1 is a graphic representation of the single crystal structure of the compound of Example 10B. The ORTEP drawing and labelling of the Example 10B molecule is shown with 50% probability anisotropic thermal ellipsoids. The structure was solved using SHELXT. All nonhydrogen atoms were refined with anisotropic temperature factors. The positions of the hydrogen atoms could initially be determined using a difference Fourier map. Hydrogens were subsequently, when possible, replaced by hydrogens at calculated positions and refined riding on the parent atoms. On the completed model Bijvoet analysis was performed to determine the absolute configuration. The crystal quality of the Example 10B crystal was suitable for single-crystal X-ray diffraction. No solvent was incorporated into the crystal structure. No enlarged anisotropic temperature factors or disorder was found. A weak intramolecular hydrogen bond is found in the structure (N09-H09—N16). Due to the excellent quality of the intensity data the absolute configuration could be determined with great certainty.

TABLE II
Crystal Structure and Structure Refinement
General information

| Crystal color | transparent, colorless |
|---|---|
| Crystal dimensions | 0.29 × 0.17 × 0.17/ |
| [mm]/shape | block |
| Crystallization | from methanol/water |
| Empirical formula | C₁₆H₁₅BrClN₃O₂ |
| Formula weight [g/mol] | 396.67 |

Crystal Data

| | |
|---|---|
| Crystal system | orthorhombic |
| Space group | P2₁2₁2₁ (No. 19) |
| Unit cell dimensions | 5.58857(8), 14.2522(2), |
| | 20.8515(4) |
| a, b, c [Å] | 90, 90, 90 |
| α, β, γ [°] | |
| Volume [Å³] | 1660.81(5) |
| Z | 4 |
| Density (calculated) [g/cm³] | 1.586 |
| Absorption coefficient | 2.648 |
| (MoKα) [mm⁻¹] | |
| F(000) | 800 |

Data Collection

| | |
|---|---|
| Temperature during | 150 |
| experiment [K] | |
| Wavelength [Å] | 0.71073 |
| θ Min-Max [°] | 1.7, 27.5 |
| Index range | −7 ≦ h ≦ 7; −18 ≦ k ≦ 18; |
| | −27 ≦ 1 ≦ 27 |
| Tot., Uniq. Data, R(int) | 60844, 3818, 0.018 |
| Observed Data | 3739 |
| [I > 2.0 sigma(I)] | |

Refinement

| | |
|---|---|
| Nref, Npar | 3818, 213 |
| R, wR2, S | 0.0173, 0.0482, 1.034 |
| Min. and Max. | −0.37, 0.36 |
| Resd. Dens. [e/Å³] | |

Bijvoet analysis

| | |
|---|---|
| Number Bijvoet Pairs | 1595(100%) |
| Flack x | 0.005(1) |
| Parsons z | 0.005(1) |
| P2(true) | 1.000 |
| P3(true) | 1.000 |
| P3(rac-twin) | 0.000 |
| Hooft y | 0.000(1) |
| Student-T Nu | 99.38 |

Equipment and Software

| | |
|---|---|
| Single-crystal X-ray Diffraction | |
| Diffractometer | Bruker Kappa ApexII |
| Radiation type | MoKα |
| Source | fine-focus sealed tube |
| Monochomator | graphite |
| Scan type | φ and ω scans |
| Absorption | SADABS2014/4 |
| correction | multi-scan |
| Data Collection | Apex2 |
| Cell refinement | Peakref |
| Data reduction | Eval15 |
| Structure solution | SHELXT |
| Structure refinement | SHELXL-2014 |
| Molecular graphics | PLATON, Shelxle |

TABLE III
Final Coordinates of the non-Hydrogen atoms

| Atom | x | Y | z |
|---|---|---|---|
| Br20 | 0.27342(5) | 0.70639(2) | 0.75853(2) |
| Cl1 | 0.80944(11) | 0.10072(4) | 0.37308(3) |
| O12 | 0.7840(2) | 0.58601(9) | 0.39899(6) |
| O22 | 0.6727(3) | 0.57041(11) | 0.73295(7) |
| N09 | 0.9090(3) | 0.51982(12) | 0.51572(8) |
| N13 | 0.6510(3) | 0.61623(11) | 0.45401(7) |
| N16 | 0.6941(3) | 0.57932(10) | 0.62224(7) |
| C02 | 0.8859(3) | 0.21428(14) | 0.39750(9) |
| C03 | 1.1014(4) | 0.22973(15) | 0.42762(10) |
| C04 | 1.1552(3) | 0.32092(14) | 0.44738(9) |
| C05 | 0.7253(3) | 0.28590(13) | 0.38629(9) |
| C06 | 0.7812(4) | 0.37593(13) | 0.40711(9) |
| C07 | 0.9974(3) | 0.39484(14) | 0.43778(8) |
| C08 | 1.0606(3) | 0.49243(14) | 0.46182(9) |
| C10 | 1.0341(3) | 0.57116(14) | 0.41224(9) |
| C11 | 1.1554(4) | 0.55302(16) | 0.34862(10) |
| C14 | 0.7278(3) | 0.58148(11) | 0.50761(8) |
| C15 | 0.6007(3) | 0.61247(12) | 0.56702(8) |
| C17 | 0.4048(4) | 0.67121(14) | 0.56464(9) |
| C18 | 0.3001(4) | 0.69890(13) | 0.62239(9) |
| C19 | 0.3970(4) | 0.66675(13) | 0.67878(9) |
| C21 | 0.5925(4) | 0.60483(14) | 0.67696(9) |
| C23 | 0.8507(4) | 0.49776(18) | 0.72958(10) |

TABLE IV
Hydrogen Atom Positions

| Atom | x | y | z |
|---|---|---|---|
| H03 | 1.21060 | 0.17970 | 0.43480 |
| H04 | 1.30380 | 0.33270 | 0.46800 |
| H05 | 0.57880 | 0.27390 | 0.36470 |
| H06 | 0.67020 | 0.42540 | 0.40030 |
| H08 | 1.23050 | 0.49150 | 0.47690 |
| H09 | 0.945(5) | 0.5058(18) | 0.5553(13) |
| H10 | 1.10140 | 0.63010 | 0.43090 |
| H11A | 1.12880 | 0.60650 | 0.32000 |
| H11B | 1.08840 | 0.49620 | 0.32920 |
| H11C | 1.32750 | 0.54460 | 0.35550 |
| H17 | 0.34270 | 0.69230 | 0.52470 |
| H18 | 0.16460 | 0.73920 | 0.62270 |
| H23A | 1.00200 | 0.52450 | 0.71440 |
| H23B | 0.79750 | 0.44880 | 0.69980 |
| H23C | 0.87400 | 0.47050 | 0.77230 |

### Example 11

### Synthesis of cis-3-(5-bromo-6-methoxypyridin-2-yl)-5-(4-chlorophenyl)-6-cyclopropyl-5,6-dihydro-4H-1,2,4-oxadiazine

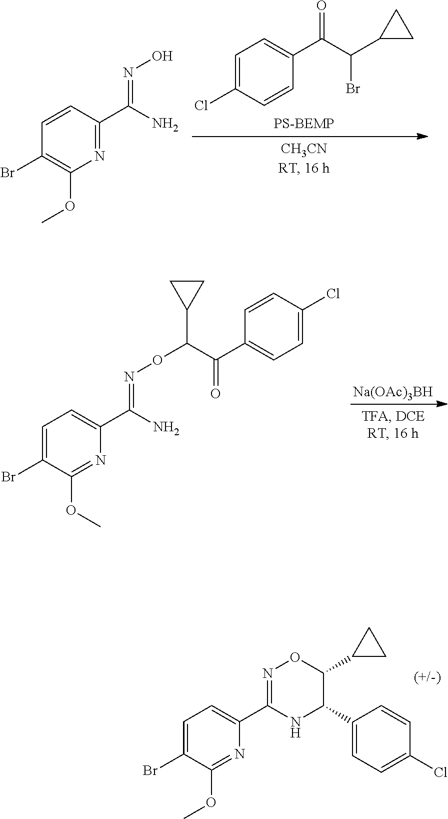 [SMILES: COc1nc(/C(N)=N/O)ccc1Br ; O=C(c1ccc(Cl)cc1)C(Br)C1CC1 ; COc1nc(/C(N)=N/OC(C(=O)c2ccc(Cl)cc2)C2CC2)ccc1Br ; COc1nc(C2=NO[C@H](C3CC3)[C@H](c3ccc(Cl)cc3)N2)ccc1Br ; "Na(OAc)3BH"]

5-bromo-N′-(2-(4-chlorophenyl)-1-cyclopropyl-2-oxoethoxy)-6-methoxypicolinimidamide: To a stirred solution of 5-bromo-N′-hydroxy-6-methoxypicolinimidamide Example 1 (800 mg, 3 mmol) in CH₃CN (20 mL) at room temperature under an argon atmosphere was added PS-BEMP (1.5 g). The reaction mixture was stirred for 5 min at room temperature. Then 2-bromo-1-(4-chlorophenyl)-2-cyclopropylethan-1-one Example 8 (1.32 g, 5 mmol) in CH₃CN (20 mL) was added to the reaction mixture at room temperature. The reaction mixture was stirred for 16 h at room temperature. After consumption of starting material (monitored by TLC), the volatiles were concentrated in vacuo to obtain 5-bromo-N′-(2-(4-chlorophenyl)-1-cyclopropyl-2-oxoethoxy)-6-methoxypicolinimidamide (1.2 g, crude) as brown semi-solid used in the next step without further purification. LCMS: 58.8%; 439.7 (M+3); (column; Ascentis Express C-18 (50×3.0 mm, 2.7 μm); RT 3.19 min; mobile phase: 0.025% Aq TFA+5% ACN: ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:05).

cis-3-(5-bromo-6-methoxypyridin-2-yl)-5-(4-chlorophenyl)-6-cyclopropyl-5,6-dihydro-4H-1,2,4-oxadiazine: To a stirred solution of 5-bromo-N′-(2-(4-chlorophenyl)-1-cyclopropyl-2-oxoethoxy)-6-methoxypicolinimidamide (1.2 g, 3 mmol) in 1,2-dichloroethane (24 mL) at 0° C. under an argon atmosphere were added trifluoroacetic acid (1.56 g, 14 mmol) and sodium triacetoxyborohydride (1.7 g, 8 mmol). The reaction mixture was stirred for 16 h at room temperature. After consumption of starting material (monitored by TLC), the reaction mixture was quenched with a saturated sodium bicarbonate solution (50 mL) and extracted with CH₂Cl₂ (2×50 mL). The combined organic extracts were dried over sodium sulfate, filtered and concentrated in vacuo to obtain cis-3-(5-bromo-6-methoxypyridin-2-yl)-5-(4-chlorophenyl)-6-cyclopropyl-5,6-dihydro-4H-1,2,4-oxadiazine Example 11 (300 mg, 26%) as brown semi solid. The cis relative stereochemistry was assigned based a method of synthesis analogous to that of Example 10. ¹H NMR (DMSO-d6, 500 MHz): δ 8.08 (d, 1H), 7.90 (d, 1H), 7.46 (d, 1H), 7.41 (d, 2H), 7.29 (d, 2H), 4.75-4.73 (m, 1H), 4.00 (s, 3H), 3.02 (dd, 1H), 0.46-0.32 (m, 4H), 0.15-0.09 (m, 1H); LCMS: 86.0%; 423.8 (M+1); (column; Ascentis Express C-18 (50×3.0 mm, 2.7 μm); RT 3.02 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:05).

### Example 12

Synthesis of 3-(5-bromo-6-methoxypyridin-2-yl)-6,6-dimethyl-5-phenyl-5,6-dihydro-4H-1,2,4-oxadiazine

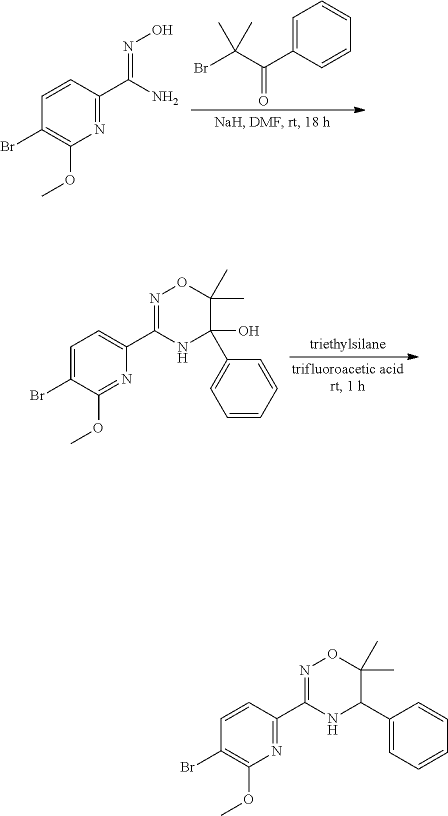 [SMILES: COc1nc(/C(N)=N/O)ccc1Br ; CC(C)(Br)C(=O)c1ccccc1 ; COc1nc(C2=NOC(C)(C)C(O)(c3ccccc3)N2)ccc1Br ; COc1nc(C2=NOC(C)(C)C(c3ccccc3)N2)ccc1Br]

3-(5-bromo-6-methoxypyridin-2-yl)-6,6-dimethyl-5-phenyl-5,6-dihydro-4H-1,2,4-oxadiazin-5-ol: To an ice-bath cooled solution of 5-bromo-N′-hydroxy-6-methoxypicolinimidamide Example 1 (1.0 g, 4.1 mmol) in dry N,N-dimethylformamide (25 mL) under argon atmosphere was added sodium hydride (171 mg, 4.3 mmol, 60%). The mixture was stirred for 20 minutes at 0° C. Then, a solution of 2-bromoisobutyrophenone (969 mg, 4.3 mmol) in dry N,N-dimethylformamide (5 mL) was added, and the mixture was stirred at room temperature for 18 hours. The reaction was quenched with water and extracted three times with ethyl acetate. The combined organic layers were washed with brine, dried with sodium sulfate and concentrated in vacuo to afford a light-yellow oil that was purified by silica column chromatography 125% EtOAc in heptanel to afford 3-(5-bromo-6-methoxypyridin-2-yl)-6,6-dimethyl-5-phenyl-5,6-dihydro-4H-1,2,4-oxadiazin-5-ol (549 mg, 32%) as an off-white solid. ¹H NMR (CDCl₃, 300 MHz) δ 7.88 (d, J=8.0 Hz, 1H), 7.74-7.66 (m, 2H), 7.61 (d, J=8.0 Hz, 1H), 7.43 (m, 3H), 7.08 (s, 1H), 3.99 (s, 3H), 3.29 (s, 1H), 1.35 (s, 3H), 1.12 (s, 3H); LCMS: 93.9%; 392.0 (M+1); RT 2.24 min (method A); TLC: 50% EtOAc/Heptane (R_{f}:066).

3-(5-bromo-6-methoxypyridin-2-yl)-6,6-dimethyl-5-phenyl-5,6-dihydro-4H-1,2,4-oxadiazine: To a solution of 3-(5-bromo-6-methoxypyridin-2-yl)-6,6-dimethyl-5-phenyl-5,6-dihydro-4H-1,2,4-oxadiazin-5-ol (549 mg, 1.4 mmol) in trifluoroacetic acid (8 mL) was added triethylsilane (8 mL). The mixture was stirred at room temperature for 1 hour and then concentrated in vacuo. The residue was purified by SCX-2 column to afford 3-(5-bromo-6-methoxypyridin-2-yl)-6,6-dimethyl-5-phenyl-5,6-dihydro-4H-1,2,4-oxadiazine Example 12 (320 mg, 57%) as a white solid. ¹H NMR (CDCl₃, 300 MHz) δ 7.87 (d, J=8.0 Hz, 1H), 7.64 (d, J=8.0 Hz, 1H), 7.39-7.36 (m, 5H), 6.56 (s, 1H), 4.44 (d, J=1.8 Hz, 1H), 3.98 (s, 3H), 1.37 (s, 3H), 1.02 (s, 3H); LCMS: 93.4%; 376.0 (M+1); RT 2.29 mm (method A).

### Example 13

### Synthesis of 3-(5-Bromo-6-methoxypyridin-2-yl)-5-(4-chlorophenyl)-6,6-dimethyl-5,6-dihydro-4H-1,2,4-oxadiazine

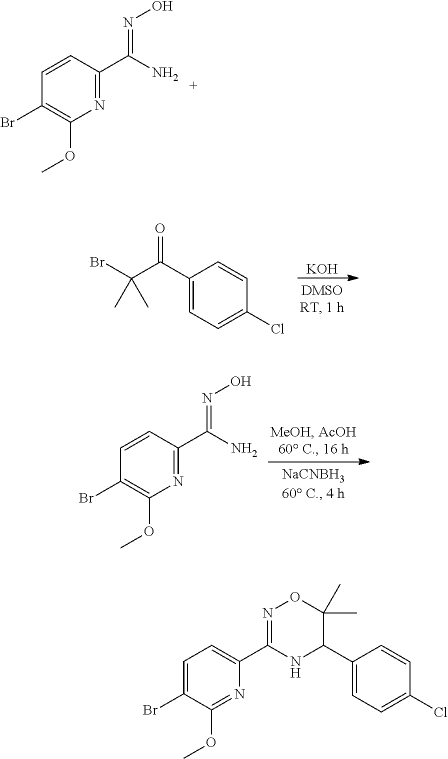 [SMILES: COc1nc(/C(N)=N/O)ccc1Br ; CC(C)(Br)C(=O)c1ccc(Cl)cc1 ; "KOH" ; COc1nc(/C(N)=N/O)ccc1Br ; COc1nc(C2=NOC(C)(C)C(c3ccc(Cl)cc3)N2)ccc1Br]

5-bromo-N′-((1-(4-chlorophenyl)-2-methyl-1-oxopropan-2-yl)oxy)-6-methoxypicolinimidamide: To a stirred solution of N′-hydroxy-6-methoxy-5-(4-methyl-1H-imidazol-1-yl) picolinimidamide Example 1 (500 mg, 2 mmol) in DMSO (3 mL) at 0° C. under an argon atmosphere were added potassium hydroxide (171 mg, 3 mmol) and 2-bromo-1-(4-chlorophenyl)-2-methylpropan-1-one Example 9 (796 mg, 3 mmol). The reaction mixture was warmed to room temperature and stirred for 1 h. After consumption of starting material (monitored by TLC), the reaction mixture was diluted with water (30 mL) and extracted with EtOAc (2×30 mL). The combined organic extracts were dried over sodium sulfate, filtered and concentrated in vacuo to obtain (Z)-5-bromo-N′-((1-(4-chlorophenyl)-2-methyl-1-oxopropan-2-yl) oxy)-6-methoxypicolinimidamide (600 mg, crude) as brown solid used in the next step without further purification. LCMS: 10.7%; 427.7 (M+3); (column; Ascentis Express C-18 (50×3.0 mm, 2.7 μm); RT 2.90 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:05).

3-(5-Bromo-6-methoxypyridin-2-yl)-5-(4-chlorophenyl)-6,6-dimethyl-5,6-dihydro-4H-1,2,4-oxadiazine: To a stirred solution of (Z)-5-bromo-N′-((1-(4-chlorophenyl)-2-methyl-1-oxopropan-2-yl) oxy)-6-methoxypicolinimidamide (600 mg, 1 mmol) in MeOH (20 mL) at room temperature under an argon atmosphere was added acetic acid (5 mL). The reaction mixture was stirred at 60° C. for 16 h. Then sodium cyanoborohydride (177 mg, 3 mmol) was added to the reaction mixture. The reaction mixture was stirred at 60° C. for 4 h. After consumption of starting material (monitored by TLC), the volatiles were evaporated in vacuo. The crude material was purified by column chromatography using 15% EtOAc: hexane to afford 345-bromo-6-methoxypyridin-2-yl)-5-(4-chlorophenyl)-6,6-dimethyl-5,6-dihydro-4H-1,2,4-oxadiazine Example 13 (120 mg, 21%) as an off-white solid. ¹H NMR (CDCl₃, 500 MHz): δ 7.86 (d, 1H), 7.63 (d, 1H), 7.34 (d, 2H), 7.28 (d, 2H), 4.39 (s, 1H), 4.00 (s, 3H), 1.39 (s, 3H), 1.00 (s, 3H); LCMS: 95.1%; 411.8 (M+3); (column; Ascentis Express C-18 (50×3.0 mm, 2.7 μm); RT 2.96 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/hexane (R_{f}:06).

### Example 14

### Synthesis of 3-(5-bromo-6-methoxypyridin-2-yl)-5-methyl-5-(1-methyl-1H-indol-3-yl)-5,6-dihydro-4H-1,2,4-oxadiazine

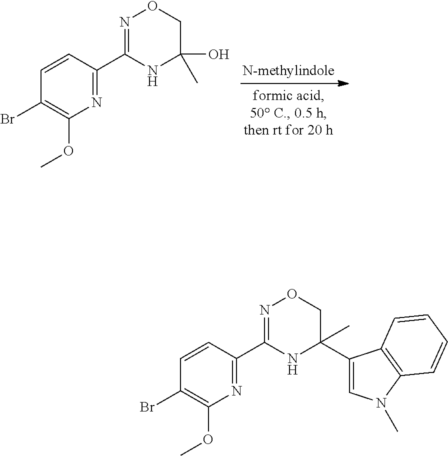 [SMILES: COc1nc(C2=NOCC(C)(O)N2)ccc1Br ; COc1nc(C2=NOCC(C)(c3cn(C)c4ccccc34)N2)ccc1Br]

3-(5-bromo-6-methoxypyridin-2-yl)-5-methyl-5-(1-methyl-1H-indo1-3-yl)-5,6-dihydro-4H-1,2,4-oxadiazine: To a solution of 3-(5-bromo-6-methoxypyridin-2-yl)-5-methyl-5,6-dihydro-4H-1,2,4-oxadiazin-5-ol Example 5 (415 mg, 1.4 mmol) in formic acid (15 mL) was added N-methylindole (360 mg, 2.8 mmol). The mixture was stirred at 50° C. for 0.5 hours and then at room temperature for 20 hours. The mixture was concentrated in vacuo, and the residue was purified by silica column chromatography 1115% EtOAc in heptanel to afford 3-(5-bromo-6-methoxypyridin-2-yl)-5-(1-methyl-1H-indol-3-yl)-5,6-dihydro-4H-1,2,4-oxadiazine Example 14 (409 mg, 72%) as a white solid. ¹H NMR (CDCl₃, 300 MHz) δ 7.88 (d, J=8.0 Hz, 1H), 7.67-7.59 (m, 2H), 7.38-7.31 (m, 1H), 7.29-7.19 (m, 1H), 7.11-7.02 (m, 2H), 6.66 (s, 1H), 4.06 (d, J=10.9 Hz, 1H), 3.98 (dd, J=10.8, 1.0 Hz, 1H), 3.85 (s, 3H), 3.80 (s, 3H), 1.84 (s, 3H); LCMS: 94.1%; 415.0 (M+1); RT 2.29 min (method A); TLC: 15% EtOAc/Heptane (R_{f}:030).

### Example 15

### Synthesis of Synthesis of 5-chloro-6-fluoro-1-methyl-1H-indole

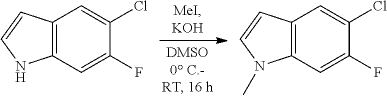

To a stirred solution of 5-chloro-6-fluoro-1H-indole (1 g, 6 mmol) in DMSO (5 mL) at 0° C. under an argon atmosphere was added potassium hydroxide (500 mg, 9 mmol). The reaction mixture was stirred for 15 mm Then methyl iodide (1.25 g, 9 mmol) was added to the reaction mixture at 0° C. The reaction mixture was warmed to room temperature and stirred for 16 h. After consumption of starting material (monitored by TLC), the reaction mixture was diluted with ice cold water (50 mL) and extracted with EtOAc (2×50 mL). The combined organic extracts were dried over sodium sulfate, filtered and concentrated in vacuo to obtain 5-chloro-6-fluoro-1-methyl-1H-indole Example 15 (1 g, crude) as a brown solid used in the next step without further purification. ¹H NMR (CDCl₃, 500 MHz): δ 7.60 (d, 1H), 7.08 (d, 1H), 7.04 (s, 1H), 6.40 (s, 1H), 3.71 (s, 3H); TLC: 30% EtOAc/hexane (R_{f}: 0.6).

### Example 16

### Synthesis of (syn)-2-(aminooxy)-1-(4-chlorophenyl)propan-1-amine

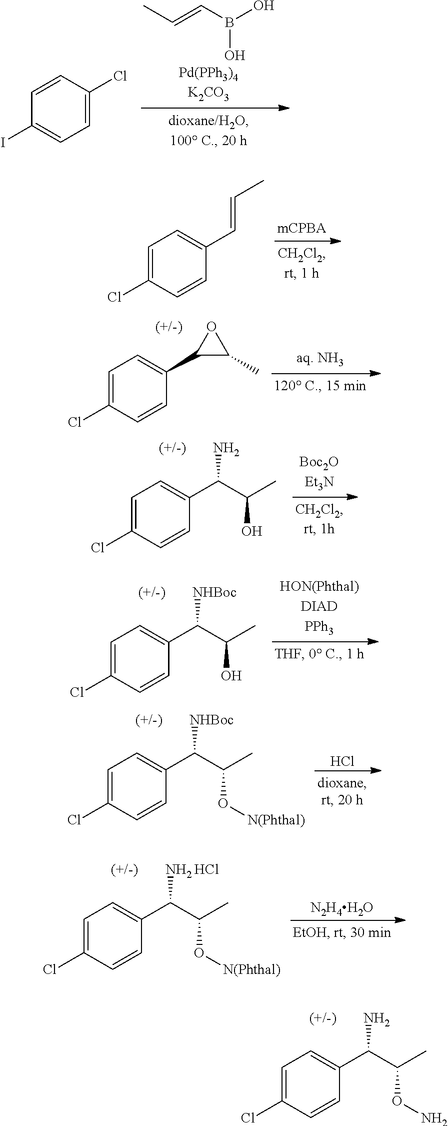

(E)-1-chloro-4-(prop-1-en-1-yl)benzene: Argon was bubbled through a suspension of trans-1-propen-1-ylboronic acid (860 mg, 10.0 mmol), 1-chloro-4-iodobenzene (2.2 g, 9.0 mmol) and potassium carbonate (4.2 g, 30.1 mmol) in 1,4-dioxane/water (4/1, 50 mL) for 5 minutes. Tetrakis(triphenylphosphine)palladium(0) (1.2 g, 1.0 mmol) was added and the mixture was heated at 100° C. for 20 hours. The mixture was diluted with a saturated aqueous ammonium chloride and extracted with diethylether. The organic layer was dried with sodium sulfate, concentrated in vacuo and purified by silica column chromatography [pentane] to afford (E)-1-chloro-4-(prop-1-en-1-yl)benzene (740 mg, 48%) as a colorless oil. ¹H NMR (CDCl₃, 400 MHz) δ 7.24 (s, 4H), 6.35 (dq, J=15.7, 1.5 Hz, 1H), 6.21 (dq, J=15.7, 6.5 Hz, 1H), 1.87 (dd, J=6.5, 1.6 Hz, 3H); TLC: Pentane (R_{f}:068).

trans-2-(4-chlorophenyl)-3-methyloxirane: To a solution of (E)-1-chloro-4-(prop-1-en-1-yl)benzene (740 mg, 4.9 mmol) in dichloromethane (25 mL) were added saturated aqueous sodium hydrogencarbonate (25 mL) and mCPBA (1.3 g, 5.3 mmol, 70 wt %). The mixture was stirred at room temperature for 1 hour. A 10% aqueous solution of sodium metabisulfite (25 mL) was added, and stirring was continued for 10 minutes. The layers were separated, and the aqueous layer was extracted twice with dichloromethane. The combined organic layers were washed with a saturated aqueous NaHCO₃ (2×), water, and brine, dried with sodium sulfate and concentrated in vacuo. The residue was purified by silica column chromatography [0 to 5% diethylether in pentane] to afford trans-2-(4-chlorophenyl)-3-methyloxirane (401 mg, 49%) as a colorless oil. ¹H NMR (CDCl₃, 400 MHz) δ 7.30 (d, J=8.5 Hz, 2H), 7.19 (d, J=8.4 Hz, 2H), 3.55 (d, J=2.0 Hz, 1H), 2.99 (dd, J=5.1, 2.0 Hz, 1H), 1.45 (d, J=5.1 Hz, 3H).

anti-tert-butyl-1-(4-chlorophenyl)-2-hydroxypropyl) carbamate: An emulsion of trans-2-(4-chlorophenyl)-3-methyloxirane (190 mg, 1.1 mmol) in a 32% aqueous ammonia solution (15 mL, 121 mol) was heated by MW irradiation at 120° C. for 15 minutes. The mixture was extracted with EtOAc, and the organic layer was dried with sodium sulfate and concentrated in vacuo to afford a yellow oil. This residue was taken up in dichloromethane (10 mL), and triethylamine (137 mg, 1.4 mmol) and di-tert-butyl dicarbonate (137 mg, 1.2 mmol) were added. The mixture was stirred at room temperature for 1 hour, then washed with water and brine, dried with sodium sulfate and concentrated in vacuo. The residue was purified by silica column chromatography [15 to 30% ethyl acetate in heptane] to afford anti-tert-butyl-1-(4-chlorophenyl)-2-hydroxypropyl) carbamate (116 mg, 36%) as a white solid. 1H NMR (CDCl₃, 400 MHz)) δ 7.33 (d, J=8.5 Hz, 2H), 7.23 (d, J=8.5 Hz, 2H), 5.38 (s, 1H), 4.66-4.45 (m, 1H), 4.18-4.01 (m, 1H), 1.41 (s, 9H), 1.07 (d, J=6.4 Hz, 3H); LCMS: 100%; 230.0 (M+1); RT 2.07 mm (method A); TLC: 25% EtOAc/Heptane (R_{f}:012).

syn-tert-butyl-1-(4-chlorophenyl)-2((1,3-dioxoisoindolin-2-yl)oxy)propyl)carbamate: To a solution of (anti)-tert-butyl-1-(4-chlorophenyl)-2-hydroxypropyl) carbamate (202 mg, 0.7 mmol), N-hydroxyphthalimide (127 mg, 0.8 mmol) and triphenylphosphine (204 mg, 0.8 mmol) in THF (10 mL) at 0° C. was added diisopropyl azodicarboxylate (157 mg, 0.8 mmol). The mixture was stirred at 0° C. for 1 hour, then concentrated in vacuo and purified by silica column chromatography [20 to 40% ethylacetate in heptane] to afford syn-tert-butyl-1-(4-chlorophenyl)-2-((1,3-dioxoisoindolin-2-yl)oxy)propyl)carbamate (210 mg, 69%) as a white solid. 1H NMR (DMSO-d6, 400 MHz) δ 7.89-7.83 (m, 4H), 7.54-7.44 (m, 3H), 7.37 (d, J=8.4 Hz, 2H), 4.96-4.80 (m, 1H), 4.65-4.51 (m, 1H), 1.36 (s, 9H), 1.10 (d, J=6.4 Hz, 3H); LCMS: 98.3%; 331.0 (M-Boc); RT 2.38 mm (method A); TLC: 50% EtOAc/Heptane (R_{f}:033).

syn-1-amino-1-(4-chlorophenyl)propan-2-yl)oxy)isoindoline-1,3-dione hydrochloride: To a solution of syn-tert-butyl-1-(4-chlorophenyl)-2-((1,3-dioxoisoindolin-2-yl)oxy)propyl)carbamate (240 mg, 0.6 mmol in 1,4-dioxane (5 mL) was added hydrochloric acid (4M in dioxane, 3 mL, 12 mmol). The mixture was stirred at room temperature for 20 hours, then concentrated in vacuo and co-evaporated twice with diethylether to afford syn-1-amino-1-(4-chlorophenyl)propan-2-yl)oxy)isoindoline-1,3-dione hydrochloride (195 mg, 95%) as a white solid. 1H NMR (DMSO-d6, 400 MHz) δ 8.72 (s, 3H), 8.00-7.90 (m, 4H), 7.66 (d, J=8.6 Hz, 2H), 7.57 (d, J=8.6 Hz, 2H), 4.81-4.70 (m, 1H), 4.67-4.60 (m, 1H), 1.11 (d, J=6.4 Hz, 3H).

syn-2-(aminooxy)-1-(4-chlorophenyl)propan-1-amine: To a solution of (syn)-1-amino-1-(4-chlorophenyl)propan-2-yl)oxy)isoindoline-1,3-dione hydrochloride (195 mg, 0.5 mmol in ethanol (10 mL) was added hydrazine monohydrate (103 mg, 2.0 mmol). The mixture was stirred at room temperature for 30 minutes, then filtered and concentrated in vacuo. The residue was purified using a SCX column to afford syn-2-(aminooxy)-1-(4-chlorophenyl)propan-1-amine Example 16 (100 mg, 94%) as a sticky oil. 1H NMR (CDCl₃, 400 MHz) δ 7.34-7.27 (m, 4H), 3.90 (d, J=7.9 Hz, 1H), 3.71 (dq, J=7.8, 6.2 Hz, 1H), 0.98 (d, J=6.2 Hz, 3H); LCMS: 95.3%; 201.0 (M+H); RT 2.69 min (method B).

### Example 17

### Synthesis of (1-(aminooxy)cyclopropyl)(4-chlorophenyl)methanamine

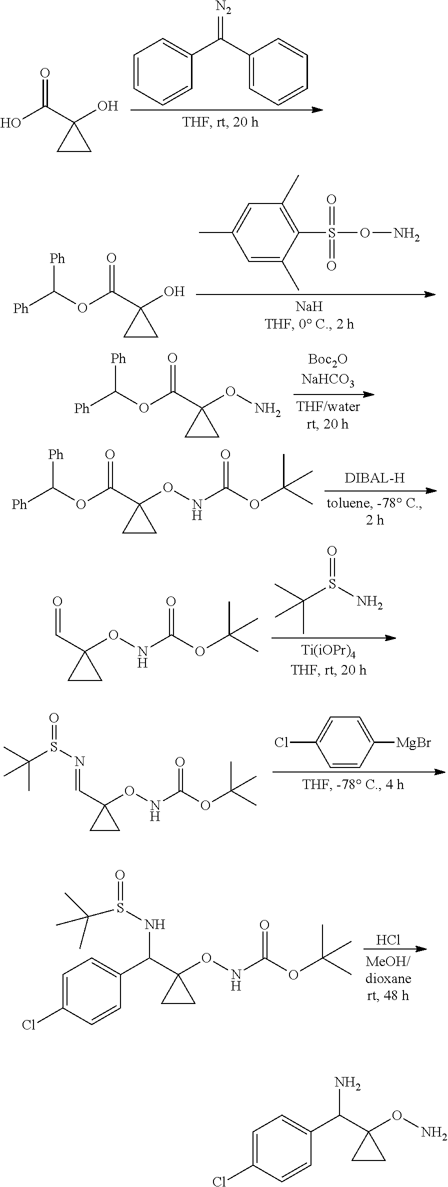

Benzhydryl 1-hydroxycyclopropanecarboxylate: To a solution of 1-hydroxycyclopropane-carboxylic acid (0.4 g, 3.9 mmol) in THF (10 mL) was added a solution of (diazomethylene)dibenzene (0.913 g, 4.7 mmol) in THF (5 mL). The reaction mixture was stirred at room temperature overnight, and then concentrated under reduced pressure. The residue was purified by silica flash chromatography 1120% ethyl acetate in heptanel to afford benzhydryl 1-hydroxycyclopropanecarboxylate (0.93 g, 88%) as a white solid. ¹H NMR (CDCl₃, 400 MHz) δ 7.40-7.29 (m, 10H), 6.94 (s, 1H), 2.99 (s, 1H), 1.45-1.40 (m, 2H), 1.26-1.21 (m, 2H). LCMS: 100%; 291.0 (M+Na); RT 2.14 mm (method A). Benzhydryl 1-(aminooxy)cyclopropanecarboxylate: To a solution of benzhydryl 1-hydroxy-cyclopropanecarboxylate (0.93 g, 3.5 mmol) in dry THF (7 mL) at 0° C. was added sodium hydride (0.17 g, 4.2 mmol, 60% in mineral oil). The mixture was stirred for 20 minutes, and then a solution of O-(mesitylsulfonyl)hydroxylamine (0.97 g, 4.5 mmol) in dry THF (7 mL) was added. The resulting mixture was stirred for 2 hours in an ice bath and then stored in the refrigerator overnight. The mixture was quenched with water and extracted with EtOAc (3×). The combined organic layers were washed with brine, dried over MgSO₄ and concentrated under reduced pressure. The residue was purified by silica flash chromatography [40% ethyl acetate in heptane] to afford benzhydryl 1-(aminooxy)cyclo-propanecarboxylate (0.84 g, 86%) as a white crystalline solid. ¹H NMR (CDCl₃, 400 MHz) δ 7.40-7.29 (m, 10H), 6.97 (s, 1H), 5.54 (s, 2H), 1.52-1.45 (m, 2H), 1.36-1.30 (m, 2H). LCMS: 100%; 306.0 (M+Na); RT 2.14 min (method A).

Benzhydryl 1-(((tert-butoxycarbonyl)amino)oxy) cyclopropanecarboxylate: To a solution of benzhydryl 1-(aminooxy)cyclopropanecarboxylate (0.59 g, 2.1 mmol) in THF/water (40 mL, 1:1 v/v) was added di-tert-butyl dicarbonate (1.82 g, 8.3 mmol) and sodium bicarbonate (0.88 g, 10.4 mmol). The reaction mixture was stirred at room temperature overnight, then diluted with water and extracted with EtOAc (3×). The combined organic layers were washed with brine (1×), dried over Na₂SO₄ and concentrated under reduced pressure. The residue was purified by silica flash chromatography [30% ethyl acetate in heptane) to afford benzhydryl 1-(((tertbutoxycarbonyl)amino)oxy) cyclopropanecarboxylate (0.71 g, 88%) as a white crystalline solid. ¹H NMR (CDCl₃, 400 MHz) δ 7.83 (s, 1H), 7.40-7.29 (m, 10H), 6.95 (s, 1H), 1.65-1.60 (m, 2H), 1.50-1.47 (m, 2H), 1.47 (s, 9H). LCMS: 100%; 406.0 (M+Na); RT 2.21 min (method C). TLC: 25% EtOAc/Heptane (R_{f}:055).

tert-Butyl 1-formylcyclopropoxycarbamate: To a solution of benzhydryl 1-(((tert-butoxycarbonyl)amino)oxy)cyclopropanecarboxylate (0.71 g, 1.8 mmol) in dry toluene (35 mL) at −78° C. under argon was added dropwise a solution of diisobutylaluminium hydride in hexane (1M, 5.5 mL, 5.5 mmol). After 2 hours, an additional equivalent of diisobutylaluminium hydride (1M in hexane, 1.8 mL, 1.8 mmol) was added. After 4 hours, another additional equivalent of diisobutylaluminium hydride (1M in hexane, 1.8 mL, 1.8 mmol) was added. After 6 hours, the mixture was quenched with water (1.5 mL) and warmed to room temperature. Sodium sulfate was added, and the suspension was stirred at room temperature overnight, filtered and concentrated under reduced pressure. The residue was purified by silica flash chromatography (30% to 45% ethyl acetate in heptane) to afford tert-butyl 1-formylcyclopropoxycarbamate (0.11 g, 24%) as a colorless oil. ¹H NMR (CDCl₃, 400 MHz) δ 9.33 (s, 1H), 7.83 (s, 1H), 1.69-1.67 (m, 2H), 1.47 (s, 9H), 1.39-1.37 (m, 2H). LCMS: 82%; 146.0 (M-Boc); RT 1.70 min (method C). TLC: 40% EtOAc/Heptane (R_{f}: 0.51).

tert-Butyl 1-(((tert-butylsulfinyl)imino)methyl)cyclopropoxycarbamate: To a solution of tert-butyl 1-formylcyclopropoxycarbamate (0.11 g, 0.54 mmol) and 2-methyl-2-propanesulfinamide (0.099 g, 0.82 mmol) in dry THF (12 mL) was added titanium(IV) isopropoxide (0.48 mL, 0.47 g, 1.6 mmol). The reaction mixture was stirred at room temperature overnight, then quenched with saturated aqueous NaHCO₃ (10 mL) and stirred for 1 hour at room temperature. The mixture was further diluted with water (15 mL) and extracted with EtOAc (3×). The combined organic layers were washed with brine, dried over Na₂SO₄ and concentrated under reduced pressure. The residue was purified by silica flash chromatography (30% ethyl acetate in heptane) to afford tert-butyl 1-(((tert-butylsulfinyl)imino)methyl) cyclo-propoxycarbamate (0.12 g, 70%) as a colorless oil. ¹H NMR (CDCl₃, 400 MHz) δ 7.78 (s, 1H), 7.74 (s, 1H), 1.74-1.72 (m, 2H), 1.48 (s, 9H), 1.32-1.28 (m, 2H), 1.22 (s, 9H). LCMS: 100%; 305.0 (M+1); RT 1.96 mm (method C). TLC: 40% EtOAc/Heptane (R_{f}:037).

tert-Butyl 1((4-chlorophenyl)(1,1-dimethylethylsulfinamido)methyl) cyclopropoxycarbamate: To a solution of tert-butyl 1-(((tert-butylsulfinyl)imino)methyl) cyclopropoxy-carbamate (0.16 g, 0.53 mmol) in dry THF (12 ml) at −78° C. under argon was added dropwise a solution of 4-chlorophenylmagnesium bromide in THF/toluene (1M, 1.1 mL, 1.1 mmol). The mixture was stirred at −78° C. for 4 hours, then warmed to room temperature, poured into saturated aqueous NH₄Cl (20 mL) and extracted with EtOAc (3×). The combined organic layers were washed with brine, dried over Na₂SO₄ and concentrated under reduced pressure. The residue was purified by silica flash chromatography (50% ethyl acetate in heptane). The racemic diastereoisomers of the title compound were separated on the column to afford diastereoisomer I (0.11 g, 46%, colorless oil, first-eluting diastereoisomer) and diastereoisomer II (0.05 g, 22%, white solid, second-eluting diastereoisomer). Diastereoisomer I, ¹H NMR (CDCl₃, 400 MHz) δ 8.29 (s, 1H), 7.36-7.29 (m, 4H), 4.91 (d, J=6.5 Hz, 1H), 4.51 (d, J=6.5 Hz, 1H), 1.47 (s, 9H), 1.35-1.29 (m, 1H), 1.13 (s, 9H), 1.05 (m, 1H), 0.58 (m, 1H), 0.42 (m, 1H). LCMS: 98.6%; 417.2 (M+1); RT 2.14 min (method C). TLC: 50% EtOAc/Heptane (R_{f}:055). Diastereoisomer II, ¹H NMR (CDCl₃, 400 MHz) δ 8.31 (s, 1H), 7.34-7.21 (m, 4H), 5.05 (d, J=3.2 Hz, 1H), 4.39 (d, J=3.3 Hz, 1H), 1.48 (s, 9H), 1.34 (m, 1H), 1.28 (s, 9H), 1.03 (m, 1H), 0.52 (m, 1H), 0.31 (m, 1H). LCMS: 100%; 417.2 (M+1); RT 2.19 mm (method C). TLC: 50% EtOAc/Heptane (R_{f}:045).

(1-(Aminooxy)cyclopropyl)(4-chlorophenyl)methanamine: To a solution of tert-butyl 1-((4-chlorophenyl)(1,1-di-methylethylsulfinamido)methyl)cyclopropoxycarbamate (0.14 g, 0.35 mmol, mixture of diastereomers recombined from previous step) in MeOH (12 mL) was added hydrochloric acid (4M in dioxane, 0.86 mL, 3.5 mmol). After stirring for 8 hours at room temperature, additional hydrochloric acid (4M in dioxane, 0.86 mL, 3.5 mmol) was added, and the mixture was stirred at room temperature overnight. After 24 hours, additional hydrochloric acid (4M in dioxane, 0.43 mL, 1.7 mmol) was again added, and the mixture was stirred at room temperature overnight. The reaction mixture was purified over an SCX-2 column (column rinsed with MeOH, product eluted with 2M NH₃ in MeOH). The basic product fraction was concentrated under reduced pressure to afford (1-aminooxy)cyclopropyl)(4-chlorophenyl)methanamine Example 17 (0.06 g, 80%) as a colorless oil. ¹H NMR (CDCl₃, 400 MHz) δ 7.41 (d, J=8.2 Hz, 2H), 7.33 (d, J=8.2 Hz, 2H), 4.50 (s, 1H), 4.11 (s, 4H), 0.91 (m, 2H), 0.70 (m, 1H), 0.55 (m, 1H). LCMS: 100% product ; 213.0 (M+1); RT 2.66 min (method B). TLC: Chloroform/7M NH₃ in MeOH=9/1 (R_{f}:071).

### Example 18

### Synthesis of cis-5-(4-chlorophenyl)-3-(6-methoxy-5-(4-methyl-1H-imidazol-1-yl)pyridin-2-yl)-6-methyl-5,6-dihydro-4H-1,2,4-oxadiazine

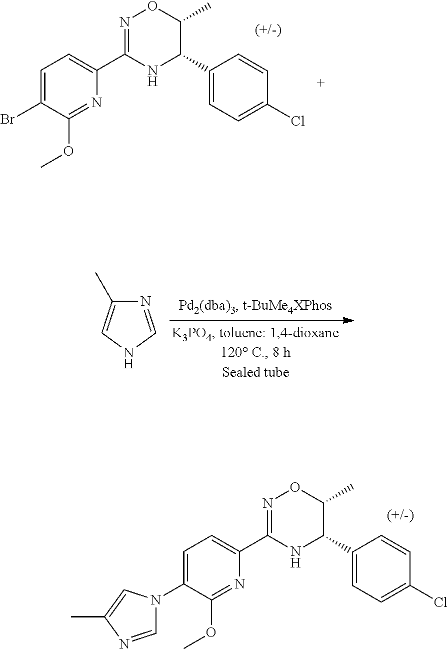

To a dry vial was added a suspension of Pd₂(dba)₃ (120 mg, 0.1 mmol) and tert-butyl tetramethyl Xphos (120 mg, 0.2 mmol) in toluene: 1,4-dioxane (2:1, 7.5 mL) at room temperature. The suspension was degassed with argon, heated to 120° C., and stirred at 120° C. for 3 min. A mixture of racemic cis-3-(5-bromo-6-methoxypyridin-2-yl)-5-(4-chlorophenyl)-6-methyl-5, 6-dihydro-4H-1, 2, 4-oxadiazine Example 10 (1 g, 2 mmol), 4-methyl-1H-imidazole (250 mg, 3 mmol) and potassium phosphate (1.07 g, 5 mmol) in toluene: 1,4-dioxane (2:1, 7.5 mL) was degassed and the catalyst premixture was added. The resulting mixture was stirred at 120° C. for 8 h in a sealed tube. After consumption of starting material (monitored by TLC and LCMS), the reaction mixture was diluted with water (20 mL) and extracted with EtOAc (2×20 mL). The combined organic extracts were washed with water (20 mL), dried over sodium sulfate, filtered and concentrated in vacuo. The crude material was purified by column chromatography using 2% MeOH:CH₂Cl₂ to afford cis-5-(4-chlorophenyl)-3-(6-methoxy-5-(4-methyl-1H-imidazol-1-yl) pyridin-2-yl)-6-methyl-5,6-dihydro-4H-1,2,4-oxadiazine (700 mg, 68%) as a pale yellow solid.

Racemic compound of Example 18 was separated using a Chiralpak-ODH column (250×20 mm, 5 μm) (40 mg loading; 0.1% DEA in n-hexane: EtOH:MeOH (50:50) (A:B: 70:30) as mobile phase; flow rate: 20 mL/min) to provide the compound of Example 18A (Fraction I (−)) and the compound of Example 18B (Fraction II (+)). The absolute configuration of Example 18B ((+)-(5S, 6R)) was determined by preparing a sample of the compound from Example 10B ((+)-(5S,6R)) by the same procedure used to prepare racemic Example 18.

Analytical conditions for Example 18A and Example 18B: HPLC: (column; zorbax-SB-C-18 150×4.6 mm, 3.5 μ); mobile Phase: ACN: 0.05% Aq TFA; flow rate: 1.0 mL/min; Gradient: T/B % 0.01/90, 2/90, 8/10, 15/10: diluent: CH₃CN: Water; Chiral HPLC: (Chiralcel-OD-H (250×4.6 mm, 5 μm; mobile phase (A) 0.1% DEA in n-hexane (B) EtOH:MeOH (50:50) (A:B; 70:30); flow Rate: 1.0 mL/min).

### Example 18A

(−)-(5R,6S)-5-(4-chlorophenyl)-3-(6-methoxy-5-(4-methyl-1H-imidazol-1-yl) pyridin-2-yl)-6-methyl-5,6-dihydro-4H-1,2,4-oxadiazine, Fraction (I) (−):Mass (ESI): 398.3 [M+1]; HPLC (purity): 99.5%; RT 7.65 min; Chiral HPLC: 100% RT=8.84 min: Optical rotation [α]_{D}^{20.02}: −190.00 (c=0.25, CH₂Cl₂).

### Example 18B

(+)-(5S,6R)-5-(4-chlorophenyl)-3-(6-methoxy-5-(4-methyl-1H-imidazol-1-yl) pyridin-2-yl)-6-methyl-5,6-dihydro-4H-1,2,4-oxadiazine, Fraction (II) (+): ¹H NMR (CD₃OD, 400 MHz): δ 7.97 (s, 1H), 7.88 (d, 1H), 7.67 (d, 1H), 7.36 (d, 2H), 7.28 (d, 2H), 7.21 (s, 1H), 4.71 (d, 1H), 4.07 (s, 3H), 4.04 (dd, 1H), 2.25 (s, 3H), 0.99 (d, 3H); Mass (ESI): 398.3 [M+1]; HPLC (purity): 99.5%; RT 7.65 min; Chiral HPLC: 100% RT=13.30 min: Optical rotation [α]_{D}^{19.99}: +196.11 (c=0.25, CH₂Cl₂).

### Example 19

### Synthesis of cis-5-(3,4-dichlorophenyl)-3-(6-methoxy-5-(4-methyl-1H-imidazol-1-yl)pyridin-2-yl)-6-methyl-5,6-dihydro-4H-1,2,4-oxadiazine

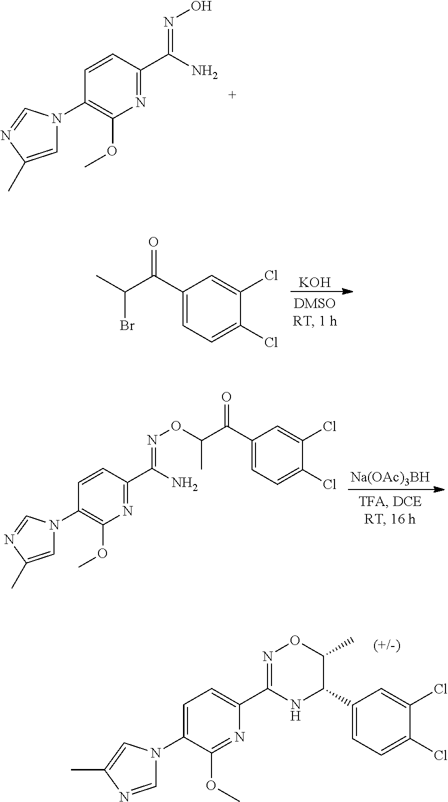

N′-((1-(3,4-Dichlorophenyl)-1-oxopropan-2-yl) oxy)-6-methoxy-5-(4-methyl-1H-imidazol-1-yl) picolinimidamide: To a stirred solution of N-hydroxy-6-methoxy-5-(4-methyl-1H-imidazol-1-yl) picolinimidamide Example 2 (700 mg, 3 mmol) in DMSO (10 mL) at 0° C. under an argon atmosphere was added potassium hydroxide (317 mg, 6 mmol). The reaction mixture was stirred at room temperature for 10 min Then 2-bromo-1-(3,4-dichlorophenyl) propan-1-one Example 7 (1.1 g, 4 mmol) was added to the reaction mixture. The reaction mixture was stirred at room temperature for 1 h. After consumption of starting material (monitored by TLC), the reaction mixture was diluted with ice cold water (30 mL) and extracted with EtOAc (2×30 mL). The combined organic extracts were dried over sodium sulfate, filtered and concentrated in vacuo to obtain N′-((1-(3,4-dichlorophenyl)-1-oxopropan-2-yl) oxy)-6-methoxy-5-(4-methyl-1H-imidazol-1-yl) picolinimidamide (800 mg, crude) as a pale yellow solid used in the next step without further purification. LCMS: 37.1%; 447.9 (M+1); (column; Ascentis Express C-18 (50×3.0 mm, 2.7 μm); RT 2.05 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:03).

cis-5-(3,4-Dichlorophenyl)-3-(6-methoxy-5-(4-methyl-1H-imidazol-1-yl) pyridin-2-yl)-6-methyl-5,6-dihydro-4H-1,2,4-oxadiazine: To a stirred solution of N′-((1-(3,4-dichlorophenyl)-1-oxopropan-2-yl) oxy)-6-methoxy-5-(4-methyl-1H-imidazol-1-yl) picolinimidamide (800 mg, 2 mmol) in 1,2-dichloroethane (10 mL) at room temperature under an argon atmosphere were added trifluoroacetic acid (1 g, 9 mmol) and sodium triacetoxyborohydride (757 mg, 4 mmol). The reaction mixture was stirred for 16 h at room temperature. After consumption of starting material (monitored by TLC), the reaction mixture was diluted with a saturated sodium bicarbonate solution (20 mL) and extracted with EtOAc (2×20 mL). The combined organic extracts were dried over sodium sulfate, filtered and concentrated in vacuo. The crude material was purified by column chromatography using 2% MeOH:CH₂Cl₂ to afford cis-5-(3,4-dichlorophenyl)-3-(6-methoxy-5-(4-methyl-1H-imidazol-1-yl) pyridin-2-yl)-6-methyl-5,6-dihydro-4H-1,2,4-oxadiazine (500 mg, 68%) as an off-white solid. The cis relative stereochemistry was assigned based on the method of synthesis and by comparison of the ¹H NMR spectrum of Example 19 with those of Example 18 and Example 24.

Racemic compound of Example 19 was separated using a Chiralpak-IB column (250×20 mm, 5 μm) (40 mg loading; 0.1% DEA in n-hexane:CH₂Cl₂:MeOH (50:50) (A:B: 80:20) as mobile phase; flow rate: 1 mL/min) to provide the compound of Example 19A (Fraction I (−)) and the compound of Example 19B (Fraction II (+)).

Analytical conditions for Example 19A and Example 19B: (column; zorbax-SB-C-18 150×4.6 mm, 3.5 μm); mobile Phase: ACN: 0.05% TFA; flow rate: 1.0 mL/min; Gradient: T/B % 0.01/90, 10/10, 15/10: diluent: CH₃CN: Water; Chiral HPLC: (Chiralpak-IB (150×4.6 mm, 3μm; mobile phase (A) 0.1% DEA in n-hexane (B) CH₂Cl₂:MeOH (50:50) (A:B; 80:20); flow Rate: 1.0 mL/min).

### Example 19A

(−)-cis-5-(3,4-dichlorophenyl)-3-(6-methoxy-5-(4-methyl-1H-imidazol-1-yl) pyridin-2-yl)-6-methyl-5,6-dihydro-4H-1,2,4-oxadiazine, Fraction (I), (−): Mass (ESI): 431.9 [M+1]; HPLC (purity): 99.8%; RT 7.89 mm; Chiral HPLC: 100% RT=7.98 min: Optical rotation [α]_{D}^{19.98}: −133.93 (c=0.25, CH₂Cl₂).

### Example 19B

(+)-cis-5-(3,4-dichlorophenyl)-3-(6-methoxy-5-(4-methyl-1H-imidazol-1-yl) pyridin-2-yl)-6-methyl-5,6-dihydro-4H-1,2,4-oxadiazine, Fraction (II), (+): ¹H NMR (CD₃OD, 500 MHz): δ 7.99 (s, 1H), 7.89 (d, 1H), 7.69 (d, 1H), 7.52 (d, 1H), 7.45 (d, 1H), 7.27-7.21 (m, 2H), 4.73 (s, 1H), 4.09 (s, 3H), 4.04 (dd, 1H), 2.26 (s, 3H), 1.02 (d, 3H); Mass (ESI): 431.8 [M+1]; HPLC (purity): 99.8%; RT 7.90 mm; Chiral HPLC: 99.3% RT=10.91 min: Optical rotation [α]_{D}^{20.00}: +149.31 (c=0.25, CH₂Cl₂).

### Example 20

### Synthesis of cis-5-(4-chlorophenyl)-6-cyclopropyl-3-(6-methoxy-5-(4-methyl-1H-imidazol-1-yl) pyridin-2-yl)-5,6-dihydro-4H-1,2,4-oxadiazine

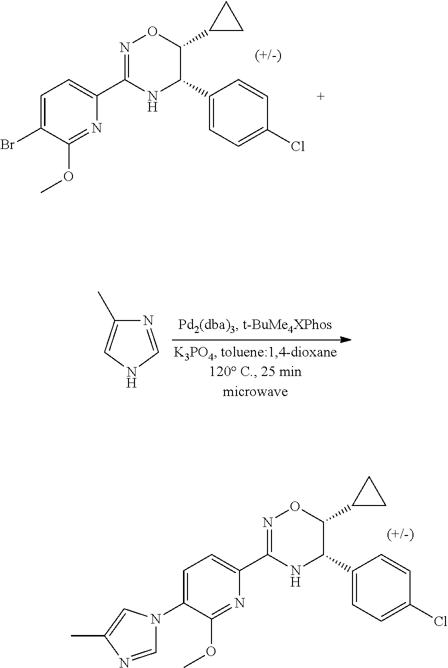

To a dry vial was added a suspension of Pd₂(dba)₃ (21 mg, 0.02 mmol) and tert-butyl tetramethyl XPhos (22 mg, 0.05 mmol) in toluene: 1,4-dioxane (2:1, 3 mL) at room temperature. The suspension was degassed with argon, heated to 120° C., and stirred at 120° C. for 3 min. A mixture of cis-3-(5-bromo-6-methoxypyridin-2-yl)-5-(4-chlorophenyl)-6-cyclopropyl-5,6-dihydro-4H-1,2,4-oxadiazine Example 11 (200 mg, 0.5 mmol), 4-methyl-1H-imidazole (46 mg, 0.5 mmol) and potassium phosphate (130 mg, 1 mmol) in toluene: 1,4-dioxane (2:1, 3 mL) was degassed and the catalyst premixture was added. The resulting mixture was stirred at 120° C. for 25 min in the microwave. After consumption of the starting material (monitored by TLC and LCMS), the reaction mixture was filtered and the filtrate was concentrated in vacuo. The crude material was purified by column chromatography using 3% MeOH:CH₂Cl₂ to afford cis-5-(4-chlorophenyl)-6-cyclopropyl-3-(6-methoxy-5-(4-methyl-1H-imidazol-1-yl) pyridin-2-yl)-5,6-dihydro-4H-1,2,4-oxadiazine (190 mg, 63%) as an off-white solid. The cis relative stereochemistry was assigned based on the method of synthesis and by comparison of the ¹H NMR spectrum of Example 20 with those of Example 18 and Example 24.

Racemic compound of Example 20 was separated using a Chiralpak-OD-H column (250×20 mm, 5 μ) (20 mg loading; 0.1% DEA in n-hexane: EtOH:MeOH (50:50) (A:B: 75:25) as mobile phase; flow rate: 20 mL/min) to provide the compounds of Example 20A (Fraction I (−)) and Example 20B (Fraction II (+)).

Analytical conditions for Example 20A and Example 20B: HPLC: column; zorbax-SB-C-18 150×4.6 mm, 3.5 μm); mobile Phase: ACN+5%0.05% Aq TFA; 0.05% TFA+5% ACN; flow rate: 1.0 mL/min; Gradient: T/B % 0.01/90, 10/10, 15/10: diluent: CH₃CN: Water; Chiral HPLC: (Chiralpak-OD-H (250×4.6 mm, 5 μm; mobile phase (A) 0.1% DEA in n-hexane (B) EtOH:MeOH (50:50) (A:B; 75:25); flow Rate: 1.0 mL/min).

### Example 20A

(−)-cis-5-(4-chlorophenyl)-6-cyclopropyl-3-(6-methoxy-5-(4-methyl-1H-imidazol-1-yl) pyridin-2-yl)-5,6-dihydro-4H-1,2,4-oxadiazine, fraction (I) (−): Mass (ESI): 424 [M+1]; HPLC (purity): 99.6%; RT 7.50 min; Chiral HPLC: 100% RT=9.11 min: Optical rotation [α]_{D}^{20.01}: −112.54 (c=0.25, CH₂Cl₂).

### Example 20B

(+)-cis-5-(4-chlorophenyl)-6-cyclopropyl-3-(6-methoxy-5-(4-methyl-1H-imidazol-1-yl) pyridin-2-yl)-5,6-dihydro-4H-1,2,4-oxadiazine, fraction (II) (+):¹H NMR (DMSO-d₆, 500 MHz): δ 7.95-7.88 (m, 3H), 7.62 (d, 1H), 7.42 (d, 2H), 7.30-7.23 (m, 3H), 4.76-4.73 (m, 1H), 4.00 (s, 3H), 3.02-3.00 (m, 1H), 2.14 (s, 3H), 0.47-0.41 (m, 3H), 0.40-0.37 (m, 1H), 0.17-0.10 (m, 1H); Mass (ESI): 423.9 [M+1]; HPLC (purity): 98.3%; RT 7.49 min; Chiral HPLC: 100% RT=11.64 min: Optical rotation [α]_{D}^{20.00}: +114.76 (c=0.25, CH₂Cl₂).

### Example 21

### Synthesis of 5-(5-chloro-6-fluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine

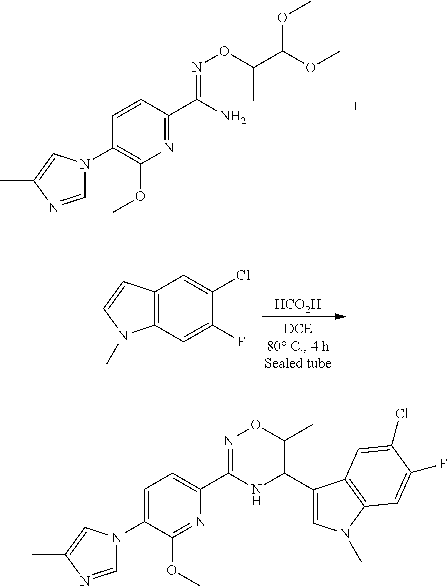

To a stirred solution of N((1,1-dimethoxypropan-2-yl) oxy)-6-methoxy-5-(4-methyl-1H-imidazol-1-yl) picolinimidamide Example 3 (300 mg, 0.8 mmol) in dichloroethane (7 mL) at room temperature under an argon atmosphere were added 5-chloro-6-fluoro-1-methyl-1H-indole Example 15 (188 mg, 1 mmol) and formic acid (7 mL). The reaction mixture was stirred at 80° C. for 4 h. After consumption of starting material (monitored by TLC), the reaction mixture was diluted with a saturated sodium bicarbonate solution (20 mL) and extracted with CH₂Cl₂ (2×20 mL). The combined organic extracts were dried over sodium sulfate, filtered and concentrated in vacuo. The crude material was purified by column chromatography using 2% MeOH:CH₂Cl₂ to afford 5-(5-chloro-6-fluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine (250 mg, 62%) as an off-white solid.

Separation of diastereomers: Racemic compound of Example 21 was separated using an YMC silica column (250×20 mm, 5 μm) (30 mg loading; n-hexane:CH₂Cl₂:MeOH (50:50) (A:B: 85:15) as mobile phase; flow rate: 20 mL/min) to provide the compounds of Example 21X and Example 21Y.

Analytical conditions for Example 21X and Example 21Y: HPLC: column; Kromacil silica (250×4.6 mm, 5 μm); mobile Phase: n-hexane:CH₂Cl₂:MeOH (50:50) (A:B: 85:15); flow rate: 1.0 mL/min.

### Example 21X (major diastereomer): HPLC: RT 21.68 mm; TLC: 5% MeOH/CH₂Cl₂: (Rf: 0.35).

### Example 21Y (minor diastereomer): HPLC: RT 26.84 mm; TLC: 5% MeOH/CH₂Cl₂: (Rf: 0.40).

Separation of enantiomers: Racemic compound of Example 21X was separated using a Chiralpak-IB column (250×20 mm, 5 μ) (40 mg loading; 0.1% DEA in n-hexane: CH₂Cl₂:MeOH (50:50) (A:B: 75:25) as mobile phase; flow rate: 20 mL/min) to provide the compounds of Example 21A (Fraction I (−)) and Example 21B (Fraction II (+)).

Racemic compound of Example 21Y was separated using a Chiralpak-IA column (250×20 mm, 5 μ) (20 mg loading; 0.1% DEA in n-hexane:CH₂Cl₂:MeOH (80:20) (A:B: 80:20) as mobile phase; flow rate: 20 mL/min) to provide the compounds of Example 21C (Fraction III (−)) and Example 21D (Fraction IV (+)).

Analytical conditions for Example 21A and Example 21B: HPLC: column; zorbax-SB-C-18 150×4.6 mm, 3.5 μ); mobile Phase: ACN: 0.05% Aq TFA; flow rate: 1.0 mL/min; Gradient: T/B % 0.01/90, 2/90, 8/10, 15/10: diluent: CH₃CN: Water; Chiral HPLC: (Chiralpak-IB (250×4.6 mm, 5 μm; mobile phase (A) 0.1% DEA in n-hexane (B) CH₂Cl₂:MeOH (50:50) (A:B; 75:25); flow Rate: 1.0 mL/min).

### Example 21A

(−)-5-(5-chloro-6-fluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, Fraction (I), (−): Mass (ESI): 469 [M+1]; HPLC (purity): 99.7%; RT 7.83 mm; Chiral HPLC: 100% RT=7.87 min: Optical rotation [α]_{D}^{20.00}: −122.91 (c=0.25, CH₂Cl₂).

### Example 21B

(+)-5-(5-chloro-6-fluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, Fraction (II), (+): ¹H NMR (CD₃OD, 500 MHz): δ 7.96 (s, 1H), 7.89 (d, 1H), 7.70 (d, 1H), 7.65 (d, 1H), 7.39 (s, 1H), 7.35 (d, 1H), 7.21 (s, 1H), 4.61 (d, 1H), 3.94 (s, 3H), 3.88-3.82 (m, 1H), 3.79 (s, 3H), 2.24 (s, 3H), 1.25 (d, J=6.1 Hz, 3H); Mass (ESI): 469.1 [M+1]; HPLC (purity): 99.4%; RT 7.82 mm; Chiral HPLC: 100% RT=10.69 min: Optical rotation [α]_{D}^{20.00}: +130.12 (c=0.25, CH₂Cl₂).

Analytical conditions for Example 21C and Example 21D: HPLC: column; zorbax-SB-C-18 150×4.6 mm, 3.5 μ); mobile Phase: ACN: 0.05% Aq TFA; flow rate: 1.0 mL/min; Gradient: T/B % 0.01/90, 2/90, 8/10, 15/10: diluent: CH₃CN: Water; Chiral HPLC: (Chiralpak-IA (250×4.6 mm, 5 μm; mobile phase (A) 0.1% DEA in n-hexane (B) CH₂Cl₂:MeOH (80:20) (A:B; 80:20); flow Rate: 1.0 mL/min).

### Example 21C

(−)-5-(5-chloro-6-fluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, Fraction (III), (−): Mass (ESI): 469[M+1]; HPLC (purity): 99.6%; RT 8.03 min; Chiral HPLC: 99.7% RT=12.52 min: Optical rotation [α]_{D}^{20.01}: −29.60 (c=0.25, CH₂Cl₂).

### Example 21D

(+)-5-(5-chloro-6-fluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, Fraction (IV), (+): ¹H NMR (CD₃OD, 500 MHz): δ 7.96 (s, 1H), 7.89 (d, 1H), 7.70 (d, 1H), 7.65 (d, 1H), 7.39 (s, 1H), 7.35 (d, 1H), 7.21 (s, 1H), 4.61 (d, 1H), 3.94 (s, 3H), 3.88-3.82 (m, 1H), 3.79 (s, 3H), 2.24 (s, 3H), 1.25 (d, J=6.1 Hz, 3H); Mass (ESI): 469 [M+1]; HPLC (purity): 99.7%; RT 8.03 mm; Chiral HPLC: 99.0% RT=14.58 mm: Optical rotation [α]_{D}^{20.01}: +41.98 (c=0.25, CH₂Cl₂).

### Example 22

### Synthesis of 3-(6-methoxy-5-(4-methyl-1H-imidazol-1-yl)pyridin-2-yl)-6,6-dimethyl-5-phenyl-5,6-dihydro-4H-1,2,4-oxadiazine

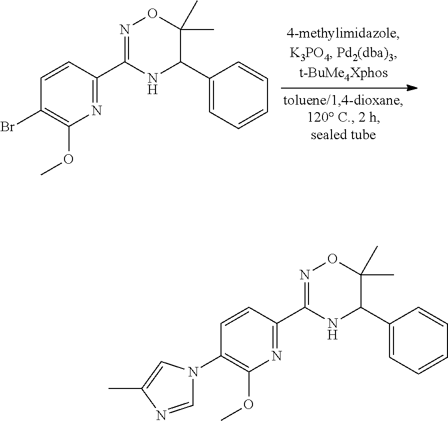 [SMILES: COc1nc(C2=NOC(C)(C)C(c3ccccc3)N2)ccc1Br ; COc1nc(C2=NOC(C)(C)C(c3ccccc3)N2)ccc1-n1cnc(C)c1]

A dry microwave vial was charged with Pd₂(dba)₃ (29 mg, 0.03 mmol) and tert-butyl tetramethyl Xphos (31 mg, 0.06 mmol) and flushed with argon. Next, an argon-degassed solution of toluene/1,4-dioxane (2/1.6 mL) was added at room temperature, and the resultant suspension was thoroughly degassed with argon. The suspension was placed in a pre-heated oil bath at 120° C. and stirred for 3 minutes. A second dry microwave vial was charged with 3-(5-bromo-6-methoxypyridin-2-yl)-6,6-dimethyl-5-phenyl-5,6-dihydro-4H-1,2,4-oxadiazine Example 12 (120 mg, 0.3 mmol), 4-methyl-1H-imidazole (52 mg, 0.6 mmol) and potassium phosphate (135 mg, 0.6 mmol) and flushed with argon. Next, an argon-degassed solution of toluene/1,4-dioxane (2/1, 3 mL) was added at room temperature and the resultant suspension was thoroughly degassed with argon. The catalyst premixture was added, and the vial was capped. The resultant mixture was stirred at 120° C. for 2 hours, then filtered and concentrated in vacuo. The residue was purified by silica column chromatography [0 to 6% methanol in dichloromethane] to afford 3-(6-methoxy-5-(4-methyl-1H-imidazol-1-yl)pyridin-2-yl)-6,6-dimethyl-5-phenyl-5,6-dihydro-4H-1,2,4-oxadiazine (76 mg, 63%) as a white solid.

Racemic compound Example 22 was separated using a Chiralpak-AD-H column (250×20 mm, 5 μm) (10 mg loading; 0.1% DEA in heptane:EtOH (90:10) as mobile phase; flow rate: 18 mL/min) to afford Example 22A (Fraction (I) (−)) and Example 22B (Fraction (II) (+)).

### Example 22A

(−)-3-(6-methoxy-5-(4-methyl-1H-imidazol-1-yl)pyridin-2-yl)-6,6-dimethyl-5-phenyl-5,6-dihydro-4H-1,2,4-oxadiazine, Fraction (I) (−): LCMS: 100%; 378.2 (M+1); RT 3.37 min (method B); Chiral HPLC: 100%; RT=21.6 mm (Chiralpak-AD-H (250×4.6 mm, 5 μm; mobile phase 0.1% DEA in heptane:EtOH (90:10); flow rate: 1.0 mL/min); Optical rotation [α]_{D}^{21.4}: −130.75 (c=0.25, CH₂Cl₂).

### Example 22B

(+)-3-(6-methoxy-5-(4-methyl-1H-imidazol-1-yl)pyridin-2-yl)-6,6-dimethyl-5-phenyl-5,6-dihydro-4H-1,2,4-oxadiazine, Fraction (II) (+): ¹H NMR (CDCl₃, 300 MHz) δ 7.85 (d, J=8.0 Hz, 1H), 7.80 (d, J=1.3 Hz, 1H), 7.62 (d, J=8.0 Hz, 1H), 7.42-7.34 (m, 5H), 6.98 (s, 1H), 6.58 (s, 1H), 4.46 (d, J=1.9 Hz, 1H), 4.00 (s, 3H), 2.30 (d, J=0.9 Hz, 3H), 1.39 (s, 3H), 1.03 (s, 3H); LCMS: 97.9%; 378.2 (M+1); RT 3.37 min (method B); Chiral HPLC: 98.2%; RT=24.1 mm (Chiralpak-AD-H (250×4.6 mm, 5 μm; mobile phase 0.1% DEA in heptane:EtOH (90:10); flow rate: 1.0 mL/min); Optical rotation [α]_{D}^{21.4}: +142.83 (c=0.25, CH₂Cl₂).

### Example 23

### Synthesis of 5-(4-chlorophenyl)-3-(6-methoxy-5-(4-methyl-1H-imidazol-1-yl) pyridin-2-yl)-6,6-dimethyl-5,6-dihydro-4H-1,2,4-oxadiazine

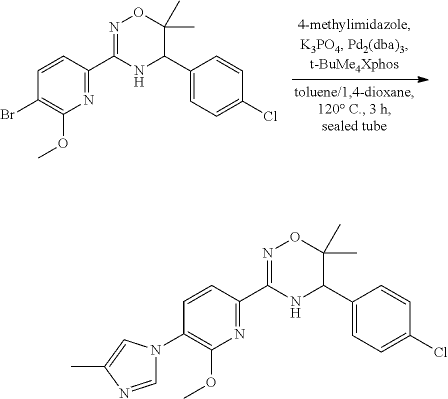 [SMILES: COc1nc(C2=NOC(C)(C)C(c3ccc(Cl)cc3)N2)ccc1Br ; COc1nc(C2=NOC(C)(C)C(c3ccc(Cl)cc3)N2)ccc1-n1cnc(C)c1]

To a dry vial was added a suspension of Pd₂(dba)₃ (11 mg, 0.01 mmol) and tert-butyl tetramethyl XPhos (12 mg, 0.02 mmol) in toluene: 1,4-dioxane (2:1, 1.5 mL) at room temperature. The suspension was degassed with argon, heated to 120° C., and stirred at 120° C. for 3 min. A mixture of 3-(5-bromo-6-methoxypyridin-2-yl)-5-(4-chlorophenyl)-6,6-dimethyl-5,6-dihydro-4H-1,2,4-oxadiazine Example 13 (100 mg, 0.2 mmol), 4-methyl-1H-imidazole (26 mg, 0.3 mmol) and potassium phosphate (103 mg, 0.5 mmol) in toluene: 1,4-dioxane (2:1, 2 mL) was degassed and the catalyst premixture was added. The resulting mixture was stirred at 120° C. for 3 h in a sealed tube. After consumption of the starting material (monitored by TLC and LCMS), the volatiles were evaporated in vacuo. The crude material was purified by column chromatography using 2% MeOH:CH₂Cl₂ to afford 5-(4-chlorophenyl)-3-(6-methoxy-5-(4-methyl-1H-imidazol-1-yl) pyridin-2-yl)-6,6-dimethyl-5,6-dihydro-4H-1,2,4-oxadiazine (400 mg, 40%) as a pale yellow solid.

Racemic compound of Example 23 was separated using a Chiralpak-IB column (250×4.6 mm, 5 μm) (30 mg loading; 0.1% DEA in n-hexane: CH₂Cl₂:MeOH (50:50) (A:B: 80:20) as mobile phase; flow rate: 1 mL/min) to provide the compound of Example 23A (Fraction I (−)) and the compound of Example 23B (Fraction II (+)).

Analytical conditions for Example 23A and Example 23B: (column; zorbax-SB-C-18 150×4.6 mm, 3.5 μm); mobile Phase: ACN+5%0.05%TFA: 0.05%TFA+5% ACN; flow rate: 1.0 mL/min; Gradient: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-hexane (B) CH₂Cl₂:MeOH (50:50) (A:B; 80:20); flow Rate: 1.0 mL/min).

### Example 23A

(−)-5-(4-chlorophenyl)-3-(6-methoxy-5-(4-methyl-1H-imidazol-1-yl) pyridin-2-yl)-6,6-dimethyl-5,6-dihydro-4H-1,2,4-oxadiazine, Fraction (I), (−): Mass (ESI): 412 [M+1]; HPLC (purity): 96.6%; RT 7.26 min; Chiral HPLC: 97.5% RT=10.14 min: Optical rotation [α]_{D}^{19.98}: −87.63 (c=0.25, CH₂Cl₂).

### Example 23B

(+)-5-(4-chlorophenyl)-3-(6-methoxy-5-(4-methyl-1H-imidazol-1-yl) pyridin-2-yl)-6,6-dimethyl-5,6-dihydro-4H-1,2,4-oxadiazine, Fraction (II), (+): ¹H NMR (CD₃OD, 400 MHz): δ 7.97 (s, 1H), 7.89 (d, 1H), 7.69 (d, 1H), 7.36-7.30 (m, 4H), 7.21 (s, 1H), 4.49 (s, 1H), 4.07 (s, 3H), 2.22 (s, 3H), 1.38 (s, 3H), 0.97 (s, 3H); Mass (ESI): 412.1 [M+1]; HPLC (purity): 98.2%; RT 7.25 min; Chiral HPLC: 97.9% RT=16.10 min: Optical rotation [α]_{D}^{20.02} (C=0.25, CH₂Cl₂).

### Example 24

### Synthesis of trans-5-(4-chlorophenyl)-3-(6-methoxy-5-(4-methyl-1H-imidazol-1-yl)pyridin-2-yl)-6-methyl-5,6-dihydro-4H-1,2,4-oxadiazine

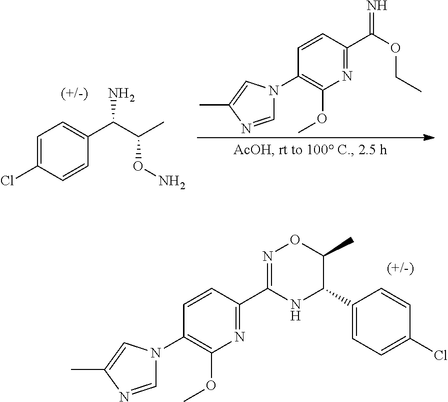

A solution of ethyl 6-methoxy-5-(4-methyl-1H-imidazol-1-yl)picolinimidate dihydrochloride Example 4 (184 mg, 0.5 mmol, 85 wt %) and syn-2-(aminooxy)-1-(4-chlorophenyl)propan-1-amine Example 16 (90 mg, 0.4 mmol) in acetic acid (10 mL) was stirred at room temperature for 1 hour and then at 100° C. for 1.5 hours. The mixture was cooled to room temperature and concentrated in vacuo, desalted using a SCX column and purified by silica column chromatography [2 to 6% methanol in EtOAc] to afford trans-5-(4-chlorophenyl)-3-(6-methoxy-5-(4-methyl-1H-imidazol-1-yl)pyridin-2-yl)-6-methyl-5,6-dihydro-4H-1,2,4-oxadiazine (132 mg, 74%) as a white solid.

Racemic compound Example 24 was separated using a Chiralpak-OD column (250×20 mm, 10 μm) (30 mg loading; heptane:EtOH (50:50) as mobile phase; flow rate: 18 mL/min) to afford the compounds of Example 24A (Fraction (I) (−)) and Example 24B (Fraction (II) (+)).

### Example 24A

(−)-trans-5-(4-chlorophenyl)-3-(6-methoxy-5-(4-methyl-1H-imidazol-1-yl)pyridin-2-yl)-6-methyl-5,6-dihydro-4H-1,2,4-oxadiazine, Fraction (I) (−): LCMS: 100%; 398.0 (M+1); RT 3.56 mm (method B); Chiral HPLC: 100%; RT=7.35 min (Chiralpak-OD (250×4.6 mm, 5 μm); mobile phase heptane:EtOH (50:50); flow Rate: 1.0 mL/min); Optical rotation [α]_{D}^{22.2}: −148.33 (c=0.1, CH₂Cl₂).

### Example 24B

(+)-trans-5-(4-chlorophenyl)-3-(6-methoxy-5-(4-methyl-1H-imidazol-1-yl)pyridin-2-yl)-6-methyl-5,6-dihydro-4H-1,2,4-oxadiazine, fraction (II) (+): 1H NMR (CDCl₃, 400 MHz) δ 7.86-7.79 (m, 2H), 7.64 (d, J=8.0 Hz, 1H), 7.41 (d, J=8.5 Hz, 2H), 7.31 (d, J=8.4 Hz, 2H), 6.99 (s, 1H), 6.35 (s, 1H), 4.30 (d, J=7.5 Hz, 1H), 3.96 (s, 3H), 3.68-3.54 (m, 1H), 2.34-2.28 (m, 3H), 1.25 (d, J=6.2 Hz, 3H); LCMS: 100%; 398.0 (M+1); RT 3.56 min (method B); Chiral HPLC: 100%; RT=14.96 mm (Chiralpak-OD (250×4.6 mm, 5 μm); mobile phase heptane:EtOH (50:50); flow Rate: 1.0 mL/min); Optical rotation [α]_{D}^{22 4}: +135.02 (c=0.1, CH₂Cl₂).

### Example 25

### Synthesis of 8-(4-chlorophenyl)-6-(6-methoxy-5-(4-methyl-1H-imidazol-1-yl)pyridin-2-yl)-4-oxa-5,7-diazaspiro[2.5]oct-5-ene

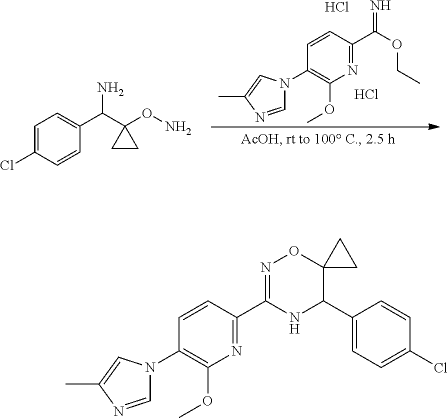 [SMILES: NOC1(C(N)c2ccc(Cl)cc2)CC1 ; CCOC(=N)c1ccc(-n2cnc(C)c2)c(OC)n1 ; Cl ; Cl ; COc1nc(C2=NOC3(CC3)C(c3ccc(Cl)cc3)N2)ccc1-n1cnc(C)c1]

A solution of ethyl 6-methoxy-5-(4-methyl-1H-imidazol-1-yl)-picolin-imidate dihydrochloride Example 4 (96 mg, 0.29 mmol, 85 wt%) and (1-(aminooxy)cyclopropyl)(4-chlorophenyl)methanamine Example 17 (59 mg, 0.28 mmol) in acetic acid (10 mL) was stirred at room temperature for 1 hour and then at 100° C. for 1.5 hours. The mixture was cooled to room temperature and concentrated under reduced pressure, de-salted using an SCX-2 column and purified by silica flash chromatography [5% methanol in EtOAc] to afford 8-(4-chlorophenyl)-6-(6-methoxy-5-(4-methyl-1H-imidazol-1-yl)pyridin-2-yl)-4-oxa-5,7-diaza-spiro [2.5] oct-5-ene (80 mg, 70%) as a beige oil.

Racemic compound Example 25 was separated using a Chiralpak OD-H column (250×20 mm, 10 μm) (30 mg loading; heptane:EtOH (70:30) as mobile phase; flow rate: 18 mL/min) to afford the compounds of Example 25A (Fraction (I) (−)) and Example 25B (Fraction (II) (+)).

### Example 25A

(−)-8-(4-chlorophenyl)-6-(6-methoxy-5-(4-methyl-1H-imidazol-1-yl)pyridin-2-yl)-4-oxa-5,7-diaza-spiro[2.5]oct-5-ene, Fraction (I) (−):LCMS: 100%; 410.0 (M+1); RT 2.98 min (method C); Chiral HPLC: 100%; RT=9.88 min (Chiralcel OD-H (250×4.6 mm, 5 μm); mobile phase heptane:EtOH (70:30); flow Rate: 1.0 mL/min); Optical rotation [α]_{D}^{21.0}: −53.0 (c=0.01, CH₂Cl₂).

### Example 25B

(+)-8-(4-chlorophenyl)-6-(6-methoxy-5-(4-methyl-1H-imidazol-1-yl)pyridin-2-yl)-4-oxa-5,7-diaza-spiro[2.5]oct-5-ene, Fraction (I) (+):¹H NMR (400 MHz, CDCl₃) δ 7.86-7.81 (m, 2H), 7.65 (d, J=8.0 Hz, 1H), 7.39-7.35 (m, 2H), 7.34-7.30 (m, 2H), 7.00 (m, 1H), 6.80 (d, J=2.8 Hz, 1H), 4.57 (d, J=3.1 Hz, 1H), 4.03 (s, 3H), 2.31 (d, J=0.8 Hz, 3H), 1.16 (m, 1H), 1.03 (m, 1H), 0.73 (m, 1H), 0.64 (m, 1H). LCMS: 100%; 410.0 (M+1); RT 2.98 min (method C); Chiral HPLC: 100%; RT=17.06 min (Chiralcel OD-H (250×4.6 mm, 5 μm); mobile phase heptane:EtOH (70:30); flow Rate: 1.0 mL/min); Optical rotation [α]_{D}^{21.0}: +57.3 (c=0.01, CH₂Cl₂).

### Example 26

### Synthesis of 3-(6-methoxy-5-(4-methyl-1H-imidazol-1-yl)pyridin-2-yl)-5-methyl-5-(1-methyl-1H-indol-3-yl)-5,6-dihydro-4H-1,2,4-oxadiazine

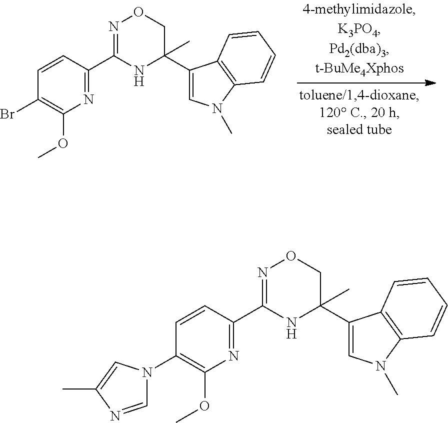 [SMILES: COc1nc(C2=NOCC(C)(c3cn(C)c4ccccc34)N2)ccc1Br ; COc1nc(C2=NOCC(C)(c3cn(C)c4ccccc34)N2)ccc1-n1cnc(C)c1]

A dry microwave vial was charged with Pd₂(dba)₃ (88 mg, 0.1 mmol) and tert-butyl tetramethyl Xphos (93 mg, 0.2 mmol) and flushed with argon. Next, an argon-degassed solution of toluene/1,4-dioxane (2/1, 6 mL) was added at room temperature and the resultant suspension was thoroughly degassed with argon. The suspension was placed in a pre-heated oil bath at 120° C. and stirred for 3 minutes. A second dry microwave vial was charged with 3-(5-bromo-6-methoxypyridin-2-yl)-5-methyl-5-(1-methyl-1H-indo1-3-yl)-5,6-dihydro-4H-1,2,4-oxadiazine Example 14 (400 mg, 1.0 mmol), 4-methyl-1H-imidazole (158 mg, 1.9 mmol) and potassium phosphate (409 mg, 1.9 mmol) and flushed with argon. Next, an argon-degassed solution of toluene/1,4-dioxane (2/1, 12 mL) was added at room temperature, and the resultant suspension was thoroughly degassed with argon. The catalyst premixture was added, and the vial was capped. The resultant mixture was stirred at 120° C. for 20 hours. The reaction mixture was filtered and concentrated in vacuo. The residue was purified by silica column chromatography [10% methanol in EtOAc] to afford 3-(6-methoxy-5-(4-methyl-1H-imidazol-1-yl)pyridin-2-yl)-5-methyl-5-(1-methyl-1H-indol-3-yl)-5,6-dihydro-4H-1,2,4-oxadiazine (185 mg, 46%) as a white solid.

Racemic compound Example 26 was separated using a Chiralpak-AD-H column (250×20 mm, 5 μm) (40 mg loading; heptane:EtOH (60:40) as mobile phase; flow rate: 18 mL/min) to afford Example 26A (Fraction (I) (+)) and Example 26B (Fraction (II) (−)).

### Example 26A

(+)-3-(6-methoxy-5-(4-methyl-1H-imidazol-1-yl)pyridin-2-yl)-5-methyl-5-(1-methyl-1H-indol-3-yl)-5,6-dihydro-4H-1,2,4-oxadiazine, Fraction (I) (+): ¹H NMR (CDCl₃, 300 MHz) δ 7.85 (d, J=8.0 Hz, 1H), 7.82 (d, J=1.3 Hz, 1H), 7.70-7.66 (m, 1H), 7.64 (d, J=8.0 Hz, 1H), 7.35 (d, J=8.2 Hz, 1H), 7.29-7.21 (m, 1H), 7.12-7.03 (m, 2H), 7.01-6.95 (m, 1H), 6.68 (s, 1H), 4.08 (d, J=10.8 Hz, 1H), 4.00 (d, J=10.8 Hz, 1H), 3.87 (s, 3H), 3.81 (s, 3H), 2.30 (d, J=0.9 Hz, 3H), 1.85 (s, 3H); LCMS: 100%; 417.2 (M+1); RT 3.47 mm (method B); Chiral HPLC: 100%; RT =7.65 mm (Chiralpak-AD-H (250×4.6 mm, 5 m; mobile phase heptane:EtOH (60:40); flow Rate: 1.0 mL/min); Optical rotation [α]_{D}^{20.0}: +179.00 (c=0.25, CH₂Cl₂).

### Example 26B

(−)-3-(6-methoxy-5-(4-methyl-1H-imidazol-1-yl)pyridin-2-yl)-5-methyl-5-(1-methyl-1H-indol-3-yl)-5,6-dihydro-4H-1,2,4-oxadiazine, fraction (II) (−): LCMS: 100%; 417.2 (M+1); RT 3.48 mm (method B); Chiral HPLC: 100%; RT=24.52 min (Chiralpak-AD-H (250×4.6 mm, 5 μm; mobile phase heptane:EtOH (60:40); flow Rate: 1.0 mL/min); Optical rotation [α]_{D}^{20.0}: −182.65 (c=0.25, CH₂Cl₂).

### Example 27

### In Vitro Cell Screening Assay and Quantification of Aβ (1-x) and Aβ (1-42) Peptides

Human neuroglioma H4 cells were transfected with a pcDNA3.1 plasmid expressing human wild type APP751 cDNA and a stable cell line was generated using G418 selection. Cells were plated at 15,000 cells/well in Costar 96-well plates and placed at 37° C. and 5% CO₂. Six hours after plating, cells were washed three times with Pro293™ chemically defined medium, followed by addition of compounds (0.003-10 μM, final DMSO concentration of 0.33%). Plates were incubated overnight (16-18 h) and supernatant was removed for quantification of Aβ peptides by sandwich ELISA. Cytotoxicity was evaluated using Cell-Titer 96W AQueous One Solution Cell Proliferation Assay according to the manufacturer's protocol.

### ELISA Measurements of Aβ Peptides

Aβ peptide levels were quantified by sandwich ELISA. 96-well plates were coated with C-terminal specific Aβ antibodies recognizing either Aβ37, Aβ38, Aβ40, Aβ42, Aβ43 or a N-terminal specific Aβ antibody to detect Aβ 1-x. Plates were then blocked overnight at 4° C. with 1% bovine serum albumin (BSA) in PBS-T. Plates were washed and 100 μl of cultured cell supernatant or synthetic Aβ peptide standards and a detection antibody (4G8-HRP) were applied to the blocked plate and incubated overnight at 4° C. The next day, wells were washed before the addition of detection substrate (TMB peroxidase). Plates were then read for absorbance at 450 nm on a Molecular Devices SpectraMax M5e Microplate Reader.

Compound-treated samples were normalized to samples treated with DMSO alone (no inhibition) and to samples treated with DAPT. IC₅₀ values were calculated from values reported as percent of DMSO controls using nonlinear regression, based on a sigmoidal dose—response (variable slope) model. GraphPAD software from Prism used for calculation.

TABLE V
Biological Assay

| | | | Aβ42 H4 |
|---|---|---|---|
| | | | Prot Fre |
| Compound | | | IC50 |
| of Example | Structure | Name | (μM) |
| 18A | 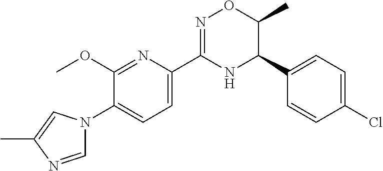 [SMILES: COc1nc(C2=NO[C@@H](C)[C@@H](c3ccc(Cl)cc3)N2)ccc1-n1cnc(C)c1] | (−)-(5R,6S)-5-(4- chlorophenyl)-3-[6- methoxy-5-(4-methyl-1H- imidazol-1-yl)pyridin-2- yl]-6-methyl-5,6-dihydro- 4H-1,2,4-oxadiazine | 2.217 |
| 18B | 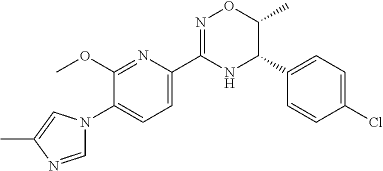 [SMILES: COc1nc(C2=NO[C@H](C)[C@H](c3ccc(Cl)cc3)N2)ccc1-n1cnc(C)c1] | (+)-(5S,6R)-5-(4- chlorophenyl)-3-[6- methoxy-5-(4-methyl-1H- imidazol-1-yl)pyridin-2- yl]-6-methyl-5,6-dihydro- 4H-1,2,4-oxadiazine | 0.019 |
| 19A | 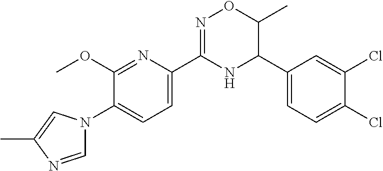 [SMILES: COc1nc(C2=NOC(C)C(c3ccc(Cl)c(Cl)c3)N2)ccc1-n1cnc(C)c1] | (−)-cis-5-(3,4- dichlorophenyl)-3-[6- methoxy-5-(4-methyl-1H- imidazol-1-yl)pyridin-2- yl]-6-methyl-5,6-dihydro- 4H-1,2,4-oxadiazine | 0.91 |
| 19B | 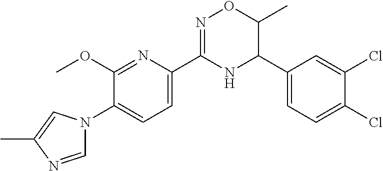 [SMILES: COc1nc(C2=NOC(C)C(c3ccc(Cl)c(Cl)c3)N2)ccc1-n1cnc(C)c1] | (+)-cis-5-(3,4- dichlorophenyl)-3-[6- methoxy-5-(4-methyl-1H- imidazol-1-yl)pyridin-2- yl]-6-methyl-5,6-dihydro- 4H-1,2,4-oxadiazine | 0.006 |
| 20A | 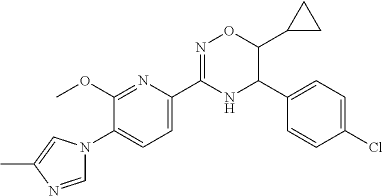 [SMILES: COc1nc(C2=NOC(C3CC3)C(c3ccc(Cl)cc3)N2)ccc1-n1cnc(C)c1] | (−)-cis-5-(4-chlorophenyl)- 6-cyclopropyl-3-[6- methoxy-5-(4-methyl-1H- imidazol-1-yl)pyridin-2- yl]-5,6-dihydro-4H-1,2,4- oxadiazine | 1.779 |
| 20B | 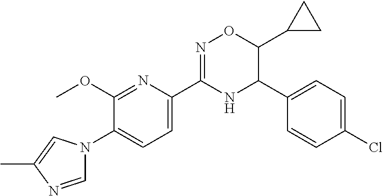 [SMILES: COc1nc(C2=NOC(C3CC3)C(c3ccc(Cl)cc3)N2)ccc1-n1cnc(C)c1] | (+)-cis-5-(4- chlorophenyl)-6- cyclopropyl-3-[6- methoxy-5-(4-methyl-1H- imidazol-1-yl)pyridin-2- yl]-5,6-dihydro-4H-1,2,4- oxadiazine | 0.009 |
| 21A | 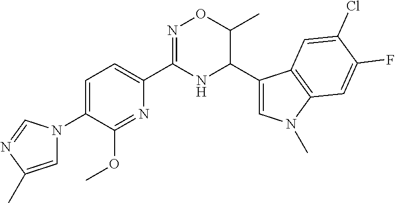 [SMILES: COc1nc(C2=NOC(C)C(c3cn(C)c4cc(F)c(Cl)cc34)N2)ccc1-n1cnc(C)c1] | (−)-5-(5-chloro-6-fluoro-1- methyl-1H-indol-3-yl)-3- (6-methoxy-5-(4-methyl- 1H-imidazol-1-yl) pyridin-2-yl)-6-methyl- 5,6-dihydro-4H-1,2,4- oxadiazine | 0.065 |
| 21B | 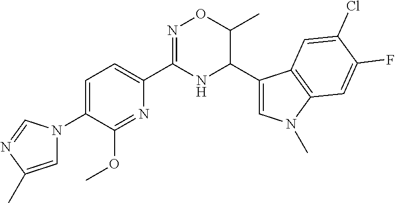 [SMILES: COc1nc(C2=NOC(C)C(c3cn(C)c4cc(F)c(Cl)cc34)N2)ccc1-n1cnc(C)c1] | (+)-5-(5-chloro-6-fluoro- 1-methyl-1H-indol-3-yl)- 3-(6-methoxy-5-(4- methyl-1H-imidazol-1-yl) pyridin-2-yl)-6-methyl- 5,6-dihydro-4H-1,2,4- oxadiazine | 0.003 |
| 21C | 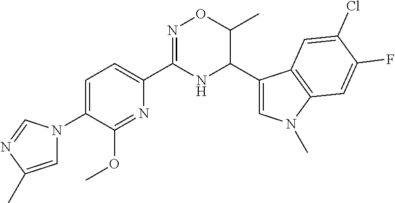 [SMILES: COc1nc(C2=NOC(C)C(c3cn(C)c4cc(F)c(Cl)cc34)N2)ccc1-n1cnc(C)c1] | (−)-5-(5-chloro-6-fluoro- 1-methyl-1H-indol-3-yl)-3- (6-methoxy-5-(4-methyl- 1H-imidazol-1-yl) pyridin-2-yl)-6-methyl- 5,6-dihydro-4H-1,2,4- oxadiazine | 0.37 |
| 21D | 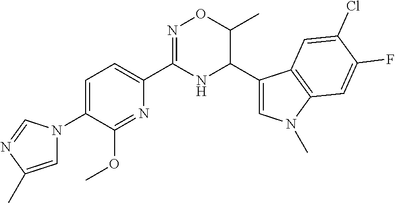 [SMILES: COc1nc(C2=NOC(C)C(c3cn(C)c4cc(F)c(Cl)cc34)N2)ccc1-n1cnc(C)c1] | (+)-5-(5-chloro-6-fluoro- 1-methyl-1H-indol-3-yl)- 3-(6-methoxy-5-(4- methyl-1H-imidazol-1-yl) pyridin-2-yl)-6-methyl- 5,6-dihydro-4H-1,2,4- oxadiazine | 0.002 |
| 22A | 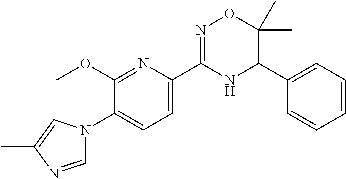 [SMILES: COc1nc(C2=NOC(C)(C)C(c3ccccc3)N2)ccc1-n1cnc(C)c1] | (−)-3-[6-methoxy-5-(4- methyl-1H-imidazol-1- yl)pyridin-2-yl]-6,6- dimethyl-5-phenyl-5,6- dihydro-4H-1,2,4- oxadiazine | >3 |
| 22B | 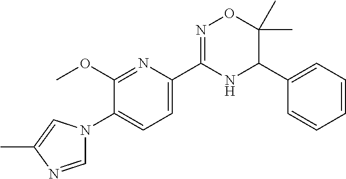 [SMILES: COc1nc(C2=NOC(C)(C)C(c3ccccc3)N2)ccc1-n1cnc(C)c1] | (+)-3-[6-methoxy-5-(4- methyl-1H-imidazol-1- yl)pyridin-2-yl]-6,6- dimethyl-5-phenyl-5,6- dihydro-4H-1,2,4- oxadiazine | 0.075 |
| 23A | 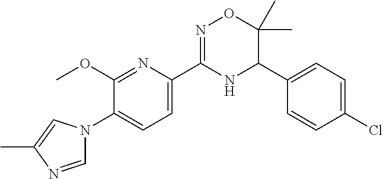 [SMILES: COc1nc(C2=NOC(C)(C)C(c3ccc(Cl)cc3)N2)ccc1-n1cnc(C)c1] | (−)-5-(4-chlorophenyl)-3- [6-methoxy-5-(4-methyl- 1H-imidazol-1-yl)pyridin- 2-yl]-6,6-dimethyl-5,6- dihydro-4H-1,2,4- oxadiazine | 1.142 |
| 23B | 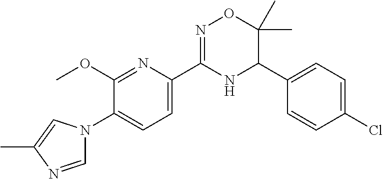 [SMILES: COc1nc(C2=NOC(C)(C)C(c3ccc(Cl)cc3)N2)ccc1-n1cnc(C)c1] | (+)-5-(4-chlorophenyl)-3- [6-methoxy-5-(4-methyl- 1H-imidazol-1-yl)pyridin- 2-yl]-6,6-dimethyl-5,6- dihydro-4H-1,2,4- oxadiazine | 0.023 |
| 24A | 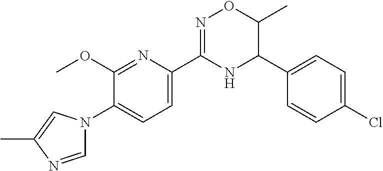 [SMILES: COc1nc(C2=NOC(C)C(c3ccc(Cl)cc3)N2)ccc1-n1cnc(C)c1] | (−)-trans-5-(4- chlorophenyl)-3-[6- methoxy-5-(4-methyl-1H- imidazol-1-yl)pyridin-2- yl]-6-methyl-5,6-dihydro- 4H-1,2,4-oxadiazine | 1.203 |
| 24B | 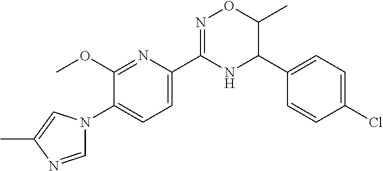 [SMILES: COc1nc(C2=NOC(C)C(c3ccc(Cl)cc3)N2)ccc1-n1cnc(C)c1] | (+)-trans-5-(4- chlorophenyl)-3-[6- methoxy-5-(4-methyl-1H- imidazol-1-yl)pyridin-2- yl]-6-methyl-5,6-dihydro- 4H-1,2,4-oxadiazine | 0.015 |
| 25A | 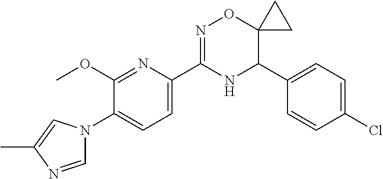 [SMILES: COc1nc(C2=NOC3(CC3)C(c3ccc(Cl)cc3)N2)ccc1-n1cnc(C)c1] | (−)-8-(4-chlorophenyl)-6- [6-methoxy-5-(4-methyl- 1H-imidazol-1-yl)pyridin- 2-yl]-4-oxa-5,7- diazaspiro[2.5]oct-5-ene | 2.605 |
| 25B | 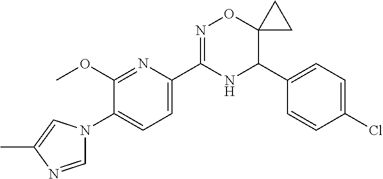 [SMILES: COc1nc(C2=NOC3(CC3)C(c3ccc(Cl)cc3)N2)ccc1-n1cnc(C)c1] | (+)-8-(4-chlorophenyl)-6- [6-methoxy-5-(4-methyl- 1H-imidazol-1-yl)pyridin- 2-yl]-4-oxa-5,7- diazaspiro[2.5]oct-5-ene | 0.026 |
| 26A | 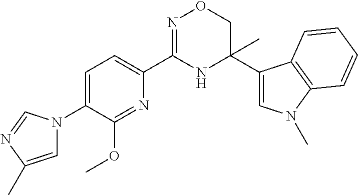 [SMILES: COc1nc(C2=NOCC(C)(c3cn(C)c4ccccc34)N2)ccc1-n1cnc(C)c1] | (+)-3-[3-[6-methoxy-5-(4- methyl-1H-imidazol-1- yl)pyridin-2-yl]-5-methyl- 5,6-dihydro-4H-1,2,4- oxadiazin-5-yl]-1-methyl- 1H-indole | 0.048 |
| 26B | 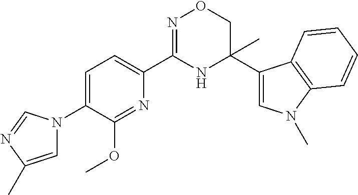 [SMILES: COc1nc(C2=NOCC(C)(c3cn(C)c4ccccc34)N2)ccc1-n1cnc(C)c1] | (−)-3-[3-[6-methoxy-5-(4- methyl-1H-imidazol-1- yl)pyridin-2-yl]-5-methyl- 5,6-dihydro-4H-1,2,4- oxadiazin-5-yl]-1-methyl- 1H-indole | 0.31 |
| 28A | 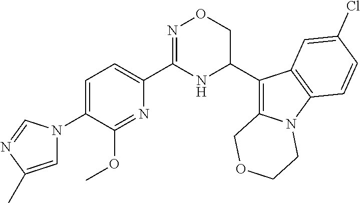 [SMILES: COc1nc(C2=NOCC(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2- yl)-5,6-dihydro-4H-1,2,4- oxadiazin-5-yl)-3,4- dihydro-1H- [1,4]oxazino[4,3-a]indole | 0.0053 |
| 28B | 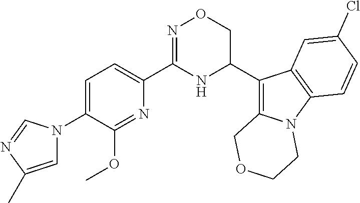 [SMILES: COc1nc(C2=NOCC(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2- yl)-5,6-dihydro-4H-1,2,4- oxadiazin-5-yl)-3,4- dihydro-1H- [1,4]oxazino[4,3-a]indole | 0.0618 |
| 29A | 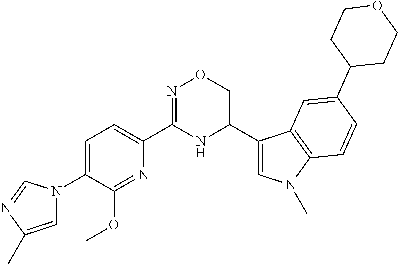 [SMILES: COc1nc(C2=NOCC(c3cn(C)c4ccc(C5CCOCC5)cc34)N2)ccc1-n1cnc(C)c1] | (−)-3-(6-methoxy-5-(4- methyl-1H-imidazol-1- yl)pyridin-2-yl)-5-(1- methyl-5-(tetrahydro-2H- pyran-4-yl)-1H-indol-3- yl)-5,6-dihydro-4H-1,2,4- oxadiazine | 0.6627 |
| 29B | 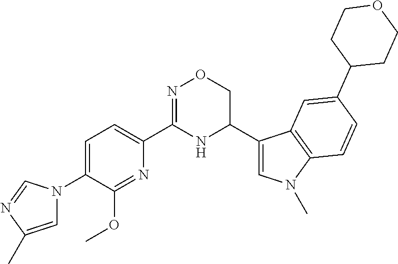 [SMILES: COc1nc(C2=NOCC(c3cn(C)c4ccc(C5CCOCC5)cc34)N2)ccc1-n1cnc(C)c1] | (+)-3-(6-methoxy-5-(4- methyl-1H-imidazol-1- yl)pyridin-2-yl)-5-(1- methyl-5-(tetrahydro-2H- pyran-4-yl)-1H-indol-3- yl)-5,6-dihydro-4H-1,2,4- oxadiazine | 0.0205 |
| 30A | 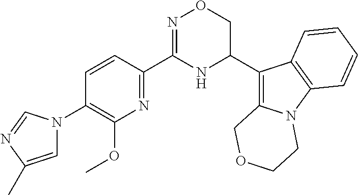 [SMILES: COc1nc(C2=NOCC(c3c4n(c5ccccc35)CCOC4)N2)ccc1-n1cnc(C)c1] | (−)-10-(3-(6-methoxy-5- (4-methyl-1H-imidazol-1- yl)pyridin-2-yl)-5,6- dihydro-4H-1,2,4- oxadiazin-5-yl)-3,4- dihydro-1H- [1,4]oxazino[4,3-a]indole | 0.3226 |
| 30B | 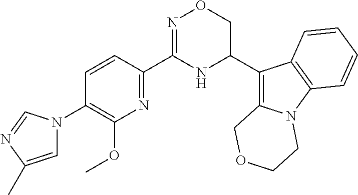 [SMILES: COc1nc(C2=NOCC(c3c4n(c5ccccc35)CCOC4)N2)ccc1-n1cnc(C)c1] | (+)-10-(3-(6-methoxy-5- (4-methyl-1H-imidazol-1- yl)pyridin-2-yl)-5,6- dihydro-4H-1,2,4- oxadiazin-5-yl)-3,4- dihydro-1H- [1,4]oxazino[4,3-a]indole | 0.0169 |
| 36A | 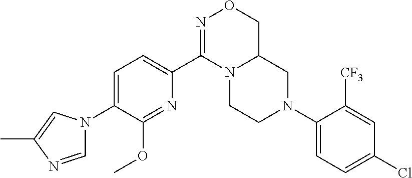 [SMILES: COc1nc(C2=NOCC3CN(c4ccc(Cl)cc4C(F)(F)F)CCN23)ccc1-n1cnc(C)c1] | (+)-8-(4-chloro-2- (trifluoromethyl) phenyl)- 4-(6-methoxy-5-(4- methyl-1H-imidazol-1-yl) pyridin-2-yl)-1, 6, 7, 8, 9, 9a-hexahydropyrazino [1, 2-d] [1, 2, 4]oxadiazine | 0.064 |
| 36B | 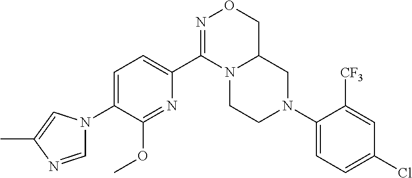 [SMILES: COc1nc(C2=NOCC3CN(c4ccc(Cl)cc4C(F)(F)F)CCN23)ccc1-n1cnc(C)c1] | (−)-8-(4-chloro-2- (trifluoromethyl) phenyl)- 4-(6-methoxy-5-(4- methyl-1H-imidazol-1-yl) pyridin-2-yl)-1, 6, 7, 8, 9, 9 a-hexahydropyrazino [1, 2-d] [1,2, 4]oxadiazine | 0.3074 |
| 39A | 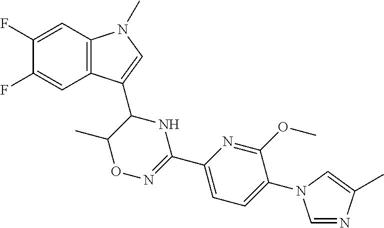 [SMILES: COc1nc(C2=NOC(C)C(c3cn(C)c4cc(F)c(F)cc34)N2)ccc1-n1cnc(C)c1] | (+)-5-(5,6-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.0054 |
| 39B | 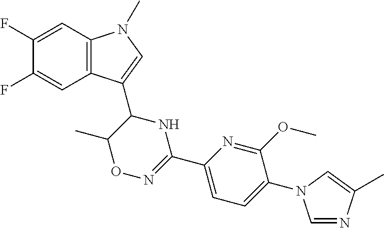 [SMILES: COc1nc(C2=NOC(C)C(c3cn(C)c4cc(F)c(F)cc34)N2)ccc1-n1cnc(C)c1] | (−)-5-(5,6-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.0704 |
| 39C | 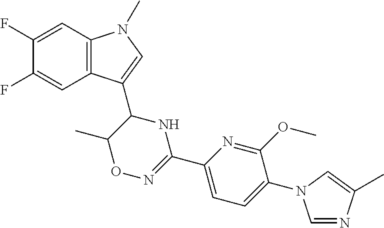 [SMILES: COc1nc(C2=NOC(C)C(c3cn(C)c4cc(F)c(F)cc34)N2)ccc1-n1cnc(C)c1] | (−)-5-(5,6-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.7294 |
| 39D | 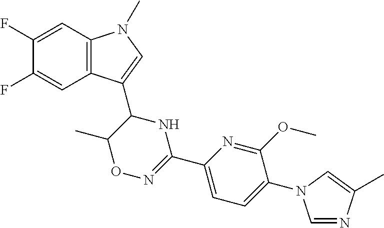 [SMILES: COc1nc(C2=NOC(C)C(c3cn(C)c4cc(F)c(F)cc34)N2)ccc1-n1cnc(C)c1] | (+)-5-(5,6-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.0044 |
| 40-IA or 40- IIA | 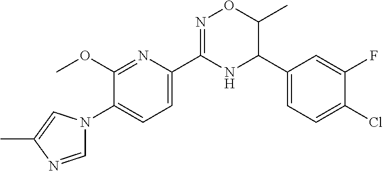 [SMILES: COc1nc(C2=NOC(C)C(c3ccc(Cl)c(F)c3)N2)ccc1-n1cnc(C)c1] | (−)-(cis)-5-(4-chloro-3- fluorophenyl)-3-(6- methoxy-5-(4-methyl-1H- imidazol-1-yl)pyridin-2- yl)-6-methyl-5,6-dihydro- 4H-1,2,4-oxadiazine | 1.8398 |
| 40-IB or 40- IIB | 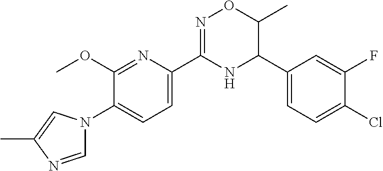 [SMILES: COc1nc(C2=NOC(C)C(c3ccc(Cl)c(F)c3)N2)ccc1-n1cnc(C)c1] | (+)-(cis)-5-(4-chloro-3- fluorophenyl)-3-(6- methoxy-5-(4-methyl-1H- imidazol-1-yl)pyridin-2- yl)-6-methyl-5,6-dihydro- 4H-1,2,4-oxadiazine | 0.0075 |
| 41A | 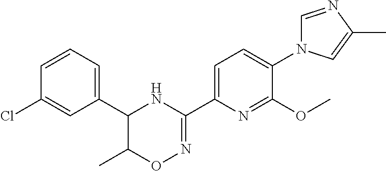 [SMILES: COc1nc(C2=NOC(C)C(c3cccc(Cl)c3)N2)ccc1-n1cnc(C)c1] | (−)-(cis)-5-(3- chlorophenyl)-3-(6- methoxy-5-(4-methyl-1H- imidazol-1-yl)pyridin-2- yl)-6-methyl-5,6-dihydro- 4H-1,2,4-oxadiazine | 1.4279 |
| 41B | 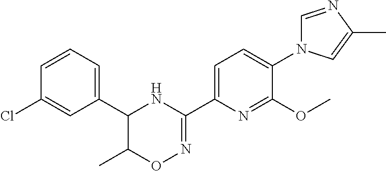 [SMILES: COc1nc(C2=NOC(C)C(c3cccc(Cl)c3)N2)ccc1-n1cnc(C)c1] | (+)-(cis)-5-(3- chlorophenyl)-3-(6- methoxy-5-(4-methyl-1H- imidazol-1-yl)pyridin-2- yl)-6-methyl-5,6-dihydro- 4H-1,2,4-oxadiazine | 0.0142 |
| 42A | 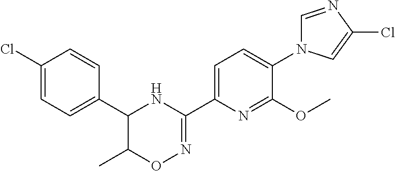 [SMILES: COc1nc(C2=NOC(C)C(c3ccc(Cl)cc3)N2)ccc1-n1cnc(Cl)c1] | (−)-(cis)-3-(5-(4-ch1oro- 1H-imidazol-1-yl)-6- methoxypyridin-2-yl)-5- (4-chlorophenyl)-6- methyl-5,6-dihydro-4H- 1,2,4-oxadiazine | >3 |
| 42B | 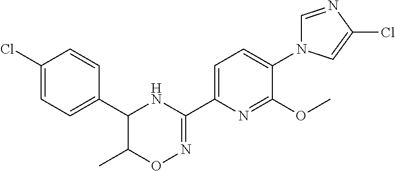 [SMILES: COc1nc(C2=NOC(C)C(c3ccc(Cl)cc3)N2)ccc1-n1cnc(Cl)c1] | (+)-(cis)-3-(5-(4-chloro- 1H-imidazol-1-yl)-6- methoxypyridin-2-yl)-5- (4-chlorophenyl)-6- methyl-5,6-dihydro-4H- 1,2,4-oxadiazine | 0.0184 |
| 43A | 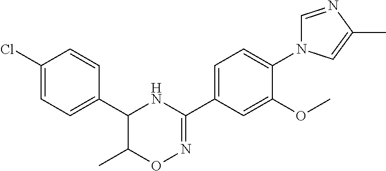 [SMILES: COc1cc(C2=NOC(C)C(c3ccc(Cl)cc3)N2)ccc1-n1cnc(C)c1] | (−)-(cis)-5-(4- chlorophenyl)-3-(3- methoxy-4-(4-methyl-1H- imidazol-1-yl)phenyl)-6- methyl-5,6-dihydro-4H- 1,2,4-oxadiazine | >3 |
| 43B | 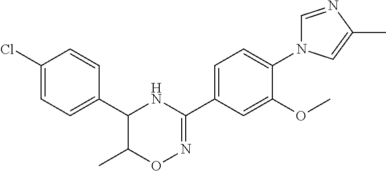 [SMILES: COc1cc(C2=NOC(C)C(c3ccc(Cl)cc3)N2)ccc1-n1cnc(C)c1] | (+)-(cis)-5-(4- chlorophenyl)-3-(3- methoxy-4-(4-methyl-1H- imidazol-1-yl)phenyl)-6- methyl-5,6-dihydro-4H- 1,2,4-oxadiazine | 0.0805 |
| 44A or 45A | 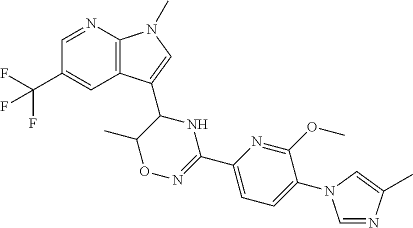 [SMILES: COc1nc(C2=NOC(C)C(c3cn(C)c4ncc(C(F)(F)F)cc34)N2)ccc1-n1cnc(C)c1] | (−)-3-(6-methoxy-5-(4- methyl-1H-imidazol-1- yl)pyridin-2-yl)-6-methyl- 5-(1-methyl-5- (trifluoromethyl)-1H- pyrrolo[2,3-b]pyridin-3- yl)-5,6-dihydro-4H- 1,2,4-oxadiazine | 0.1726 |
| 44B or 45B | 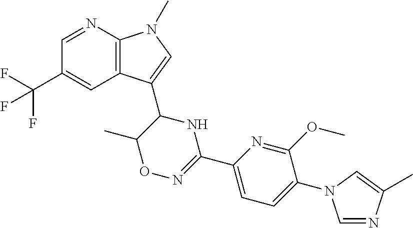 [SMILES: COc1nc(C2=NOC(C)C(c3cn(C)c4ncc(C(F)(F)F)cc34)N2)ccc1-n1cnc(C)c1] | (+)-3-(6-methoxy-5-(4- methyl-1H-imidazol-1- yl)pyridin-2-yl)-6-methyl- 5-(1-methyl-5- (trifluoromethyl)-1H- pyrrolo[2,3-b]pyridin-3-yl)- 5,6-dihydro-4H-1,2,4- oxadiazine | 0.0082 |
| 44C or 45C | 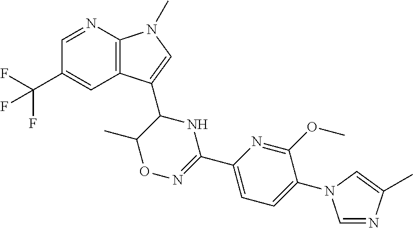 [SMILES: COc1nc(C2=NOC(C)C(c3cn(C)c4ncc(C(F)(F)F)cc34)N2)ccc1-n1cnc(C)c1] | (−)-3-(6-methoxy-5-(4- methyl-1H-imidazol-1- yl)pyridin-2-yl)-6-methyl- 5-(1-methyl-5- (trifluoromethyl)-1H- pyrrolo[2,3-b]pyridin-3- yl)-5,6-dihydro-4H-1,2,4- oxadiazine | ~1.3808 |
| 44D or 45D | 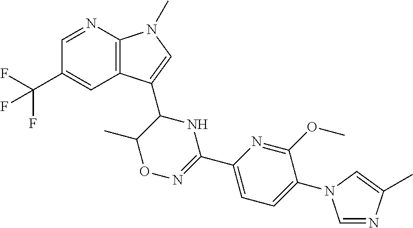 [SMILES: COc1nc(C2=NOC(C)C(c3cn(C)c4ncc(C(F)(F)F)cc34)N2)ccc1-n1cnc(C)c1] | (+)-3-(6-methoxy-5-(4- methyl-1H-imidazol-1- yl)pyridin-2-yl)-6-methyl- 5-(1-methyl-5- (trifluoromethyl)-1H- pyrrolo[2,3-b]pyridin-3- yl)-5,6-dihydro-4H-1,2,4- oxadiazine | 0.0043 |
| 46A | 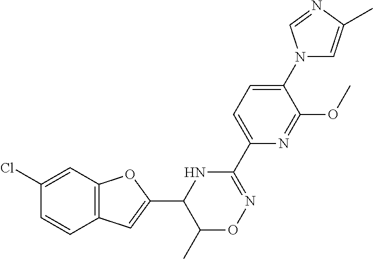 [SMILES: COc1nc(C2=NOC(C)C(c3cc4ccc(Cl)cc4o3)N2)ccc1-n1cnc(C)c1] | (−)-5-(6-chlorobenzofuran- 2-yl)-3-(6-methoxy-5-(4- methyl-1H-imidazol-1- yl)pyridin-2-yl)-6-methyl- 5,6-dihydro-4H-1,2,4- oxadiazine | 0.2248 |
| 46B | 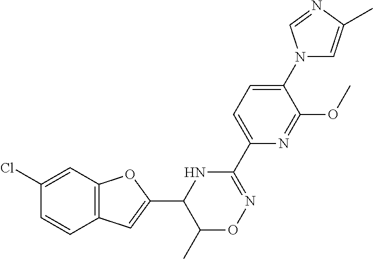 [SMILES: COc1nc(C2=NOC(C)C(c3cc4ccc(Cl)cc4o3)N2)ccc1-n1cnc(C)c1] | (+)-5-(6- chlorobenzofuran-2-yl)-3- (6-methoxy-5-(4-methyl- 1H-imidazol-1-yl)pyridin- 2-yl)-6-methyl-5,6- dihydro-4H-1,2,4- oxadiazine | 0.0051 |
| 46C | 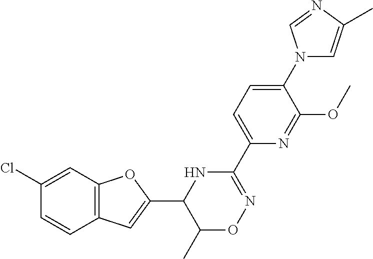 [SMILES: COc1nc(C2=NOC(C)C(c3cc4ccc(Cl)cc4o3)N2)ccc1-n1cnc(C)c1] | (−)-5-(6-chlorobenzofuran- 2-yl)-3-(6-methoxy-5-(4- methyl-1H-imidazol-1- yl)pyridin-2-yl)-6-methyl- 5,6-dihydro-4H-1,2,4- oxadiazine | 0.2907 |
| 46D | 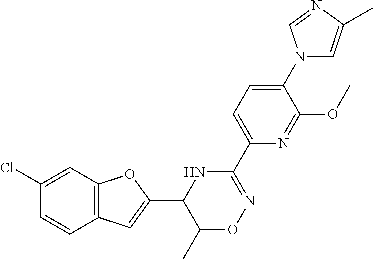 [SMILES: COc1nc(C2=NOC(C)C(c3cc4ccc(Cl)cc4o3)N2)ccc1-n1cnc(C)c1] | (+)-5-(6- chlorobenzofuran--2-yl)-3- (6-methoxy-5-(4-methyl- 1H-imidazol-1-yl)pyridin- 2-yl)-6-methyl-5,6- dihydro-4H-1,2,4- oxadiazine | 0.0028 |
| 47A | 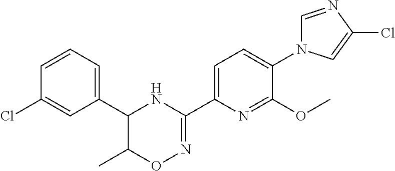 [SMILES: COc1nc(C2=NOC(C)C(c3cccc(Cl)c3)N2)ccc1-n1cnc(Cl)c1] | (−)-(cis)-3-(5-(4-chloro- 1H-imidazol-1-yl)-6- methoxypyridin-2-yl)-5- (3-chlorophenyl)-6- methyl-5,6-dihydro-4H- 1,2,4-oxadiazine | >3 |
| 47B | 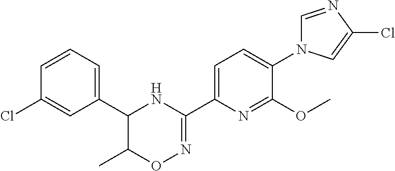 [SMILES: COc1nc(C2=NOC(C)C(c3cccc(Cl)c3)N2)ccc1-n1cnc(Cl)c1] | (+)-(cis)-3-(5-(4-chloro- 1H-imidazol-1-yl)-6- methoxypyridin-2-yl)-5- (3-chlorophenyl)-6- methyl-5,6-dihydro-4H- 1,2,4-oxadiazine | 0.0362 |
| 48A | 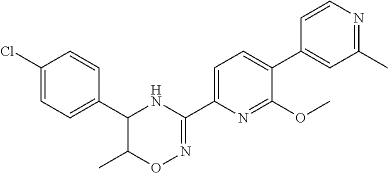 [SMILES: COc1nc(C2=NOC(C)C(c3ccc(Cl)cc3)N2)ccc1-c1ccnc(C)c1] | (−)-(cis)-5-(4- chlorophenyl)-3-(2- methoxy-2′-methyl-[3,4′- bipyridin]-6-yl)-6-methyl- 5,6-dihydro-4H-1,2,4- oxadiazine | 0.4381 |
| 48B | 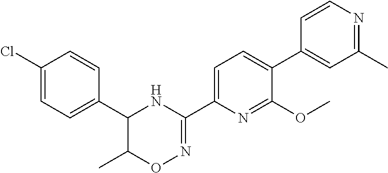 [SMILES: COc1nc(C2=NOC(C)C(c3ccc(Cl)cc3)N2)ccc1-c1ccnc(C)c1] | (+)-(cis)-5-(4- chlorophenyl)-3-(2- methoxy-2′-methyl-[3,4′- bipyridin]-6-yl)-6-methyl- 5,6-dihydro-4H-1,2,4- oxadiazine | 0.0182 |
| 49A | 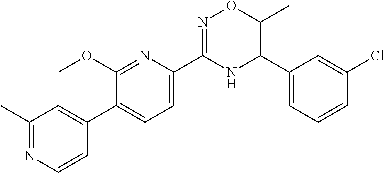 [SMILES: COc1nc(C2=NOC(C)C(c3cccc(Cl)c3)N2)ccc1-c1ccnc(C)c1] | (−)-(cis)-5-(3- chlorophenyl)-3-(2- methoxy-2′-methyl-[3,4′- bipyridin]-6-yl)-6-methyl- 5,6-dihydro-4H-1,2,4- oxadiazine | 0.5475 |
| 49B | 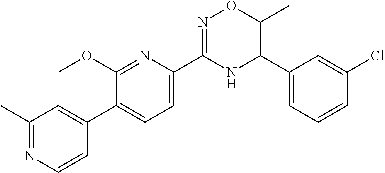 [SMILES: COc1nc(C2=NOC(C)C(c3cccc(Cl)c3)N2)ccc1-c1ccnc(C)c1] | (+)-(cis)-5-(3- chlorophenyl)-3-(2- methoxy-2′-methyl-[3,4′- bipyridin]-6-yl)-6-methyl- 5,6-dihydro-4H-1,2,4- oxadiazine | 0.0158 |
| 50A | 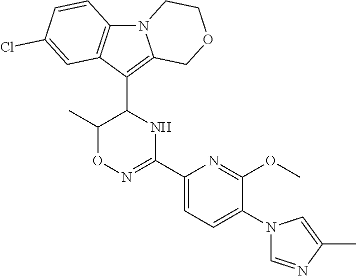 [SMILES: COc1nc(C2=NOC(C)C(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2- yl)-6-methyl-5,6-dihydro- 4H-1,2,4-oxadiazin-5-yl)- 3,4-dihydro-1H- [1,4]oxazino[4,3-a]indole | 0.0032 |
| 50B | 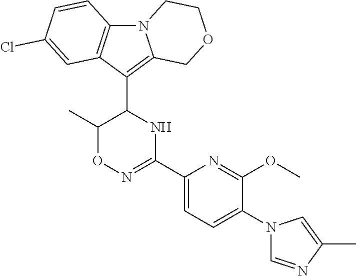 [SMILES: COc1nc(C2=NOC(C)C(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2- yl)-6-methyl-5,6-dihydro- 4H-1,2,4-oxadiazin-5-yl)- 3,4-dihydro-1H- [1,4]oxazino[4,3-a]indole | 0.027 |
| 50C | 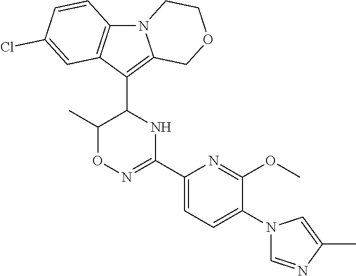 [SMILES: COc1nc(C2=NOC(C)C(c3c4n(c5ccc(Cl)cc35)CCOC4)N2)ccc1-n1cnc(C)c1] | (−)-8-chloro-10-(3-(6- methoxy-5-(4-methyl-1H- imidazol-1-yl)pyridin-2- yl)-6-methyl-5,6-dihydro- 4H-1,2,4-oxadiazin-5-yl)- 3,4-dihydro-1H- [1,4]oxazino[4,3-a]indole | 0.3523 |
| 50D | 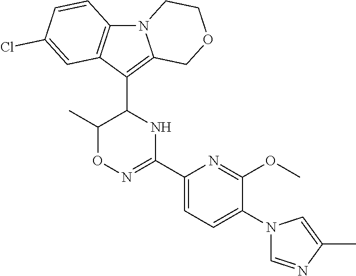 [SMILES: COc1nc(C2=NOC(C)C(c3c4n(c5ccc(Cl)cc35)CCOC4)N2)ccc1-n1cnc(C)c1] | (+)-8-chloro-10-(3-(6- methoxy-5-(4-methyl-1H- imidazol-1-yl)pyridin-2- yl)-6-methyl-5,6-dihydro- 4H-1,2,4-oxadiazin-5-yl)- 3,4-dihydro-1H- [1,4]oxazino[4,3-a]indole | 0.0063 |
| 51A | 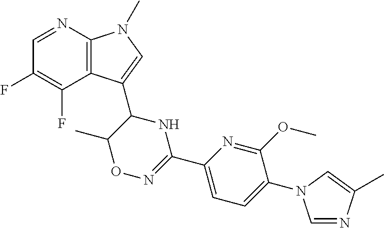 [SMILES: COc1nc(C2=NOC(C)C(c3cn(C)c4ncc(F)c(F)c34)N2)ccc1-n1cnc(C)c1] | (+)-5-(4,5-difluoro-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.0117 |
| 51B | 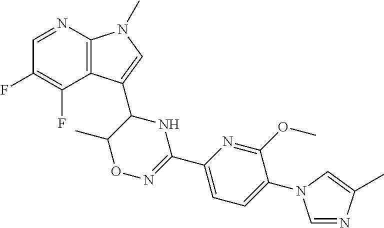 [SMILES: COc1nc(C2=NOC(C)C(c3cn(C)c4ncc(F)c(F)c34)N2)ccc1-n1cnc(C)c1] | (−)-5-(4,5-difluoro-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.2178 |
| 51C | 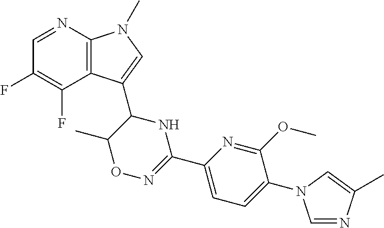 [SMILES: COc1nc(C2=NOC(C)C(c3cn(C)c4ncc(F)c(F)c34)N2)ccc1-n1cnc(C)c1] | (−)-5-(4,5-difluoro-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.9749 |
| 51D | 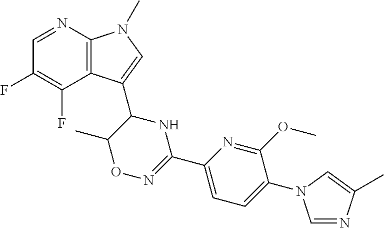 [SMILES: COc1nc(C2=NOC(C)C(c3cn(C)c4ncc(F)c(F)c34)N2)ccc1-n1cnc(C)c1] | (+)-5-(4,5-difluoro-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.0689 |
| 52A | 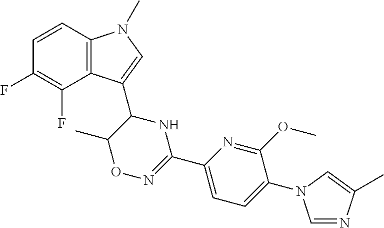 [SMILES: COc1nc(C2=NOC(C)C(c3cn(C)c4ccc(F)c(F)c34)N2)ccc1-n1cnc(C)c1] | (−)-5-(4,5-difluoro- 1-methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.084 |
| 52B | 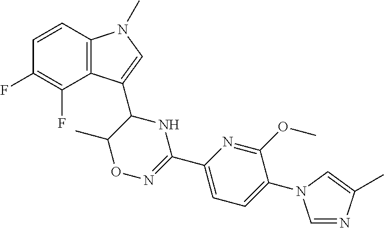 [SMILES: COc1nc(C2=NOC(C)C(c3cn(C)c4ccc(F)c(F)c34)N2)ccc1-n1cnc(C)c1] | (+)-5-(4,5-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.0029 |
| 52C | 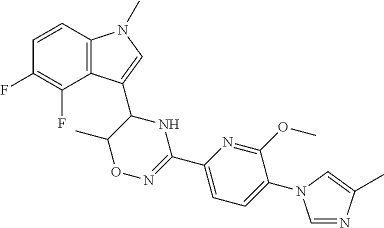 [SMILES: COc1nc(C2=NOC(C)C(c3cn(C)c4ccc(F)c(F)c34)N2)ccc1-n1cnc(C)c1] | (+)-5-(4,5-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.0058 |
| 52D | 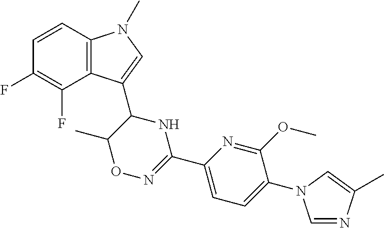 [SMILES: COc1nc(C2=NOC(C)C(c3cn(C)c4ccc(F)c(F)c34)N2)ccc1-n1cnc(C)c1] | (−)-5-(4,5-difluoro-1- methyl-1H-indol-3-yl)-3- (6-methoxy-5-(4-methyl- 1H-imidazol-1-yl)pyridin- 2-yl)-6-methyl-5,6- dihydro-4H-1,2,4- oxadiazine | 0.4014 |
| 53A | 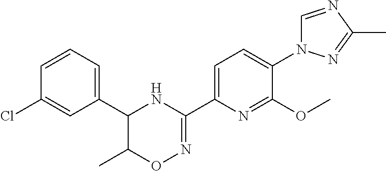 [SMILES: COc1nc(C2=NOC(C)C(c3cccc(Cl)c3)N2)ccc1-n1cnc(C)n1] | (−)-(cis)-5-(3- chlorophenyl)-3-(6- methoxy-5-(3-methyl-1H- 1,2,4-triazol-1-yl)pyridin- 2-yl)-6-methyl-5,6- dihydro-4H-1,2,4- oxadiazine | >3 |
| 53B | 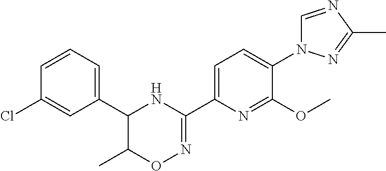 [SMILES: COc1nc(C2=NOC(C)C(c3cccc(Cl)c3)N2)ccc1-n1cnc(C)n1] | (+)-(cis)-5-(3- chlorophenyl)-3-(6- methoxy-5-(3-methyl-1H- 1,2,4-triazol-1-yl)pyridin- 2-yl)-6-methyl-5,6- dihydro-4H-1,2,4- oxadiazine | 0.1715 |
| 54A | 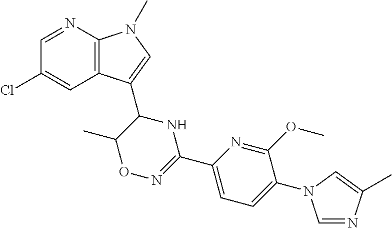 [SMILES: COc1nc(C2=NOC(C)C(c3cn(C)c4ncc(Cl)cc34)N2)ccc1-n1cnc(C)c1] | (−)-5-(5-chloro-1-methyl- 1H-pyrrolo[2,3-b]pyridin- 3-yl)-3-(6-methoxy-5-(4- methyl-1H-imidazol-1- yl)pyridin-2-yl)-6-methyl- 5,6-dihydro-4H-1,2,4- oxadiazine | 0.1903 |
| 54B | 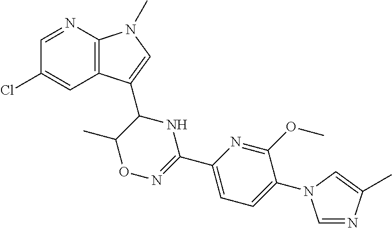 [SMILES: COc1nc(C2=NOC(C)C(c3cn(C)c4ncc(Cl)cc34)N2)ccc1-n1cnc(C)c1] | (+)-5-(5-chloro-1-methyl- 1H-pyrrolo[2,3-b]pyridin- 3-yl)-3-(6-methoxy-5-(4- methyl-1H-imidazol-1- yl)pyridin-2-yl)-6-methyl- 5,6-dihydro-4H-1,2,4- oxadiazine | 0.0063 |
| 54C | 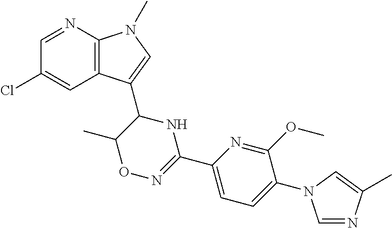 [SMILES: COc1nc(C2=NOC(C)C(c3cn(C)c4ncc(Cl)cc34)N2)ccc1-n1cnc(C)c1] | (−)-5-(5-chloro-1-methyl- 1H-pyrrolo[2,3-b]pyridin- 3-yl)-3-(6-methoxy-5-(4- methyl-1H-imidazol-1- yl)pyridin-2-yl)-6-methyl- 5,6-dihydro-4H-1,2,4- oxadiazine | 0.4637 |
| 54D | 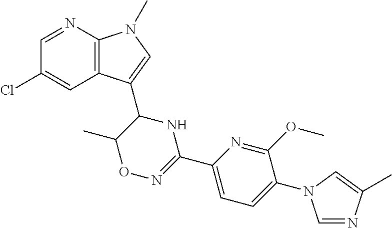 [SMILES: COc1nc(C2=NOC(C)C(c3cn(C)c4ncc(Cl)cc34)N2)ccc1-n1cnc(C)c1] | (+)-5-(5-chloro-1-methyl- 1H-pyrrolo [2,3-1Apyridin- 3-yl)-3-(6-methoxy-5-(4- methyl-1H-imidazol-1- yl)pyridin-2-yl)-6-methyl- 5,6-dihydro-4H-1,2,4- oxadiazine | 0.0051 |
| 55A | 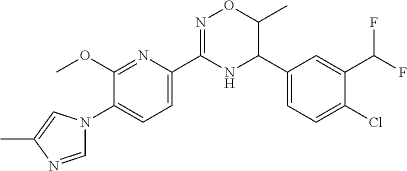 [SMILES: COc1nc(C2=NOC(C)C(c3ccc(Cl)c(C(F)F)c3)N2)ccc1-n1cnc(C)c1] | (+)-(cis)-5-(4-chloro-3- (difluoromethyl)phenyl)- 3-(6-methoxy-5-(4- methyl-1H-imidazol-1- yl)pyridin-2-yl)-6-methyl- 5,6-dihydro-4H-1,2,4- oxadiazine | 0.0116 |
| 55B | 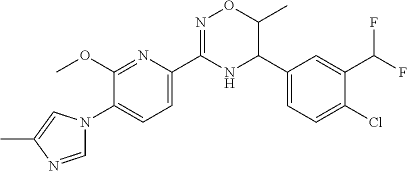 [SMILES: COc1nc(C2=NOC(C)C(c3ccc(Cl)c(C(F)F)c3)N2)ccc1-n1cnc(C)c1] | (−)-(cis)-5-(4-chloro-3- (difluoromethyl)phenyl)- 3-(6-methoxy-5-(4- methyl-1H-imidazol-1- yl)pyridin-2-yl)-6-methyl- 5,6-dihydro-4H-1,2,4- oxadiazine | 0.3516 |
| 56A | 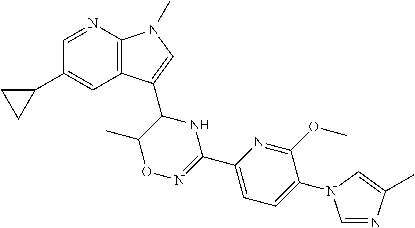 [SMILES: COc1nc(C2=NOC(C)C(c3cn(C)c4ncc(C5CC5)cc34)N2)ccc1-n1cnc(C)c1] | (+)-5-(5-cyclopropyl-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.7465 |
| 56B | 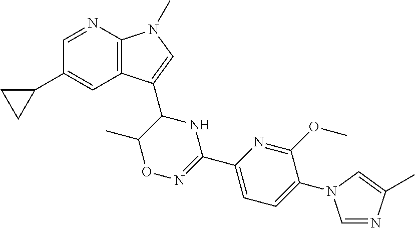 [SMILES: COc1nc(C2=NOC(C)C(c3cn(C)c4ncc(C5CC5)cc34)N2)ccc1-n1cnc(C)c1] | (−)-5-(5-cyclopropyl-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.013 |
| 56C | 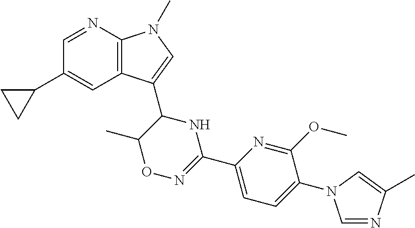 [SMILES: COc1nc(C2=NOC(C)C(c3cn(C)c4ncc(C5CC5)cc34)N2)ccc1-n1cnc(C)c1] | (−)-5-(5-cyclopropyl-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.2208 |
| 56D | 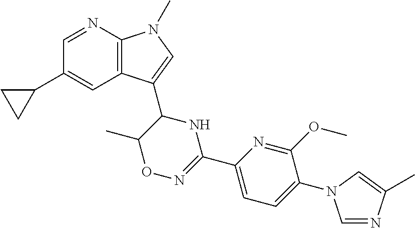 [SMILES: COc1nc(C2=NOC(C)C(c3cn(C)c4ncc(C5CC5)cc34)N2)ccc1-n1cnc(C)c1] | (+)-5-(5-cyclopropyl-1- methyl-1H-pyrrolo[2,3- b]pyridin-3-yl)-3-(6- methoxy-5-(4-methyl-1H- imidazol-1-yl)pyridin-2- yl)-6-methyl-5,6-dihydro- 4H-1,2,4-oxadiazine | 0.0125 |
| 57A or 58A | 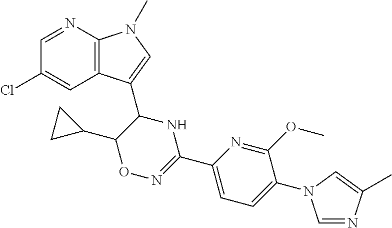 [SMILES: COc1nc(C2=NOC(C3CC3)C(c3cn(C)c4ncc(Cl)cc34)N2)ccc1-n1cnc(C)c1] | (+)-(cis)-5-(5-chloro-1- methyl-1H-pyrrolo[2,3- b]pyridin-3-yl)-6- cyclopropyl-3-(6- methoxy-5-(4-methyl-1H- imidazol-1-yl)pyridin-2- yl)-5,6-dihydro-4H-1,2,4- oxadiazine | 1.2437 |
| 57B or 58B | 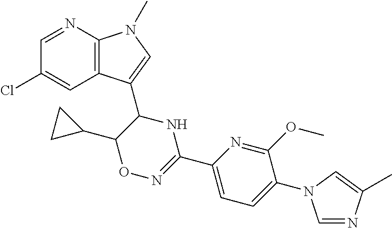 [SMILES: COc1nc(C2=NOC(C3CC3)C(c3cn(C)c4ncc(Cl)cc34)N2)ccc1-n1cnc(C)c1] | (−)-(cis)-5-(5-chloro-1- methyl-1H-pyrrolo[2,3- b]pyridin-3-yl)-6- cyclopropyl-3-(6- methoxy-5-(4-methyl-1H- imidazol-1-yl)pyridin-2- yl)-5,6-dihydro-4H-1,2,4- oxadiazine | 0.0067 |
| 59A | 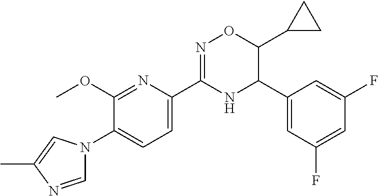 [SMILES: COc1nc(C2=NOC(C3CC3)C(c3cc(F)cc(F)c3)N2)ccc1-n1cnc(C)c1] | (+)-(cis)-6-cyclopropyl-5- (3,5-difluorophenyl)-3-(6- methoxy-5-(4-methyl-1H- imidazol-1-yl)pyridin-2- yl)-5,6-dihydro-4H-1,2,4- oxadiazine | 0.0207 |
| 59B | 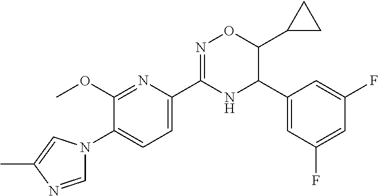 [SMILES: COc1nc(C2=NOC(C3CC3)C(c3cc(F)cc(F)c3)N2)ccc1-n1cnc(C)c1] | (−)-(cis)-6-cyclopropyl-5- (3,5-difluorophenyl)-3-(6- methoxy-5-(4-methyl-1H- imidazol-1-yl)pyridin-2- yl)-5,6-dihydro-4H-1,2,4- oxadiazine | 0.8541 |
| 60A | 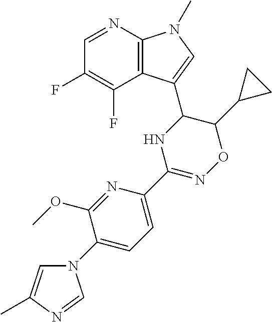 [SMILES: COc1nc(C2=NOC(C3CC3)C(c3cn(C)c4ncc(F)c(F)c34)N2)ccc1-n1cnc(C)c1] | (+)-(cis)-6-cyclopropyl-5- (4,5-difluoro-1-methyl- 1H-pyrrolo[2,3-b]pyridin- 3-yl)-3-(6-methoxy-5-(4- methyl-1H-imidazol-1- yl)pyridin-2-yl)-5,6- dihydro-4H-1,2,4- oxadiazine | 0.3672 |
| 60B | 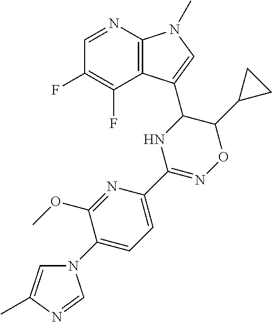 [SMILES: COc1nc(C2=NOC(C3CC3)C(c3cn(C)c4ncc(F)c(F)c34)N2)ccc1-n1cnc(C)c1] | (−)-(cis)-6-cyclopropyl-5- (4,5-difluoro-1-methyl- 1H-pyrrolo[2,3-b]pyridin- 3-yl)-3-(6-methoxy-5-(4- methyl-1H-imidazol-1- yl)pyridin-2-yl)-5,6- dihydro-4H-1,2,4- oxadiazine | 0.0427 |
| 61A | 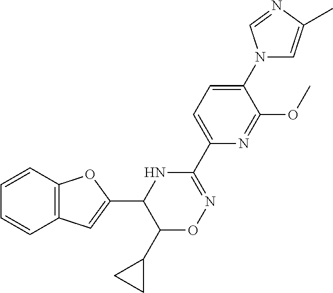 [SMILES: COc1nc(C2=NOC(C3CC3)C(c3cc4ccccc4o3)N2)ccc1-n1cnc(C)c1] | (+)-(cis)-5-(benzofuran-2- yl)-6-cyclopropyl-3-(6- methoxy-5-(4-methyl-1H- imidazol-1-yl)pyridin-2- yl)-5,6-dihydro-4H-1,2,4- oxadiazine | 0.0168 |
| 61B | 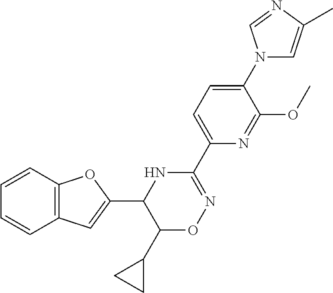 [SMILES: COc1nc(C2=NOC(C3CC3)C(c3cc4ccccc4o3)N2)ccc1-n1cnc(C)c1] | (−)-(cis)-5-(benzofuran-2- yl)-6-cyclopropyl-3-(6- methoxy-5-(4-methyl-1H- imidazol-1-yl)pyridin-2- yl)-5,6-dihydro-4H-1,2,4- oxadiazine | 0.5981 |
| 62A | 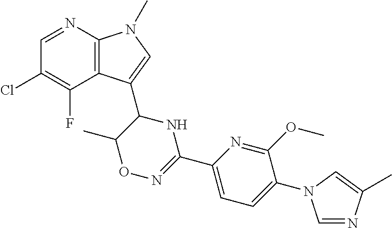 [SMILES: COc1nc(C2=NOC(C)C(c3cn(C)c4ncc(Cl)c(F)c34)N2)ccc1-n1cnc(C)c1] | (+)-(cis)-5-(5-chloro-4- fluoro-1-methyl-1H- pyrrolo[2,3-b]pyridin-3- yl)-3-(6-methoxy-5-(4- methyl-1H-imidazol-1- yl)pyridin-2-yl)-6-methyl- 5,6-dihydro-4H-1,2,4- oxadiazine | 0.0317 |
| 62B | 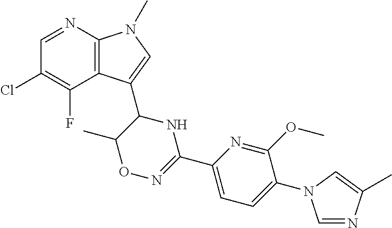 [SMILES: COc1nc(C2=NOC(C)C(c3cn(C)c4ncc(Cl)c(F)c34)N2)ccc1-n1cnc(C)c1] | (−)-(cis)-5-(5-chloro-4- fluoro-1-methyl-1H- pyrrolo[2,3-b]pyridin-3- yl)-3-(6-methoxy-5-(4- methyl-1H-imidazol-1- yl)pyridin-2-yl)-6-methyl- 5,6-dihydro-4H-1,2,4- oxadiazine | 0.4838 |
| 63A | 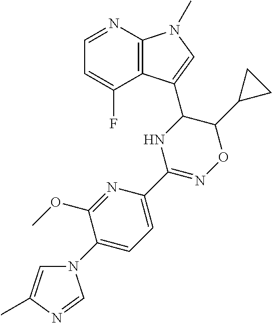 [SMILES: COc1nc(C2=NOC(C3CC3)C(c3cn(C)c4nccc(F)c34)N2)ccc1-n1cnc(C)c1] | (+)-(cis)-6-cyclopropyl-5- (4-fluoro-1-methyl-1H- pyrrolo[2,3-b]pyridin-3- yl)-3-(6-methoxy-5-(4- methyl-1H-imidazol-1- yl)pyridin-2-yl)-5,6- dihydro-4H-1,2,4- oxadiazine | 1.0587 |
| 63B | 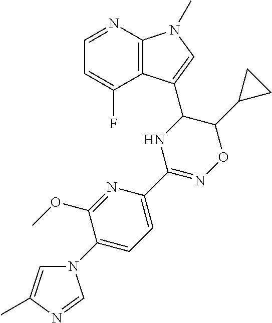 [SMILES: COc1nc(C2=NOC(C3CC3)C(c3cn(C)c4nccc(F)c34)N2)ccc1-n1cnc(C)c1] | (−)-(cis)-6-cyclopropyl-5- (4-fluoro-1-methyl-1H- pyrrolo[2,3-b]pyridin-3- yl)-3-(6-methoxy-5-(4- methyl-1H-imidazol-1- yl)pyridin-2-yl)-5,6- dihydro-4H-1,2,4- oxadiazine | 0.1048 |
| 66 | 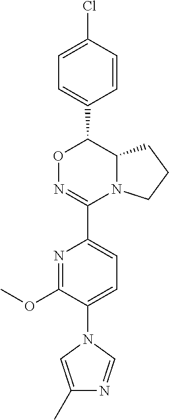 [SMILES: COc1nc(C2=NO[C@H](c3ccc(Cl)cc3)[C@@H]3CCCN23)ccc1-n1cnc(C)c1] | (1R,8αS)-1-(4- chlorophenyl)-4-(6- methoxy-5-(4-methyl-1H- imidazol-1-yl)pyridin-2- yl)-6,7,8,8 a-tetrahydro- 1H-pyrrolo[1,2- d][1,2,4]oxadiazine | 0.0057 |
| 69A | 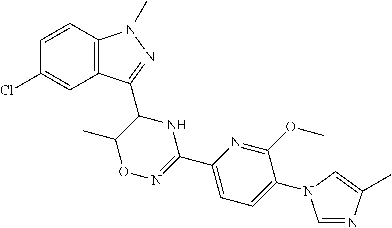 [SMILES: COc1nc(C2=NOC(C)C(c3nn(C)c4ccc(Cl)cc34)N2)ccc1-n1cnc(C)c1] | (+)-(cis)-5-(5-chloro-1- methyl-1H-indazol-3-yl)- 3-(6-methoxy-5-(4- methyl-1H-imidazol-1- yl)pyridin-2-yl)-6-methyl- 5,6-dihydro-4H-1,2,4- oxadiazine | 0.0059 |
| 69B | 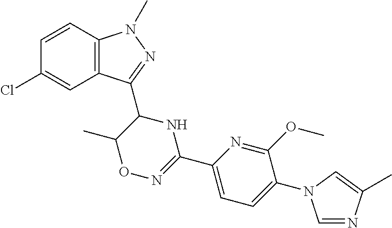 [SMILES: COc1nc(C2=NOC(C)C(c3nn(C)c4ccc(Cl)cc34)N2)ccc1-n1cnc(C)c1] | (−)-(cis)-5-(5-chloro-1- methyl-1H-indazol-3-yl)- 3-(6-methoxy-5-(4- methyl-1H-imidazol-1- yl)pyridin-2-yl)-6-methyl- 5,6-dihydro-4H-1,2,4- oxadiazine | 0.7057 |
| 71A | 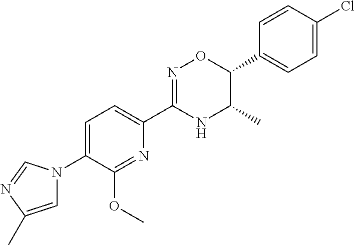 [SMILES: COc1nc(C2=NO[C@H](c3ccc(Cl)cc3)[C@H](C)N2)ccc1-n1cnc(C)c1] | (5S,6R)-6-(4- chlorophenyl)-3-(6- methoxy-5-(4-methyl-1H- imidazol-1-yl)pyridin-2- yl)-5-methyl-5,6-dihydro- 4H-1,2,4-oxadiazine | 0.0127 |

### Examples 28-30

### Synthesis of Additional Oxadiazine Compounds

Following the synthetic schemes described above and the procedures described in Examples 1-27, the following compounds were prepared and characterized:

TABLE VI
Characterization Data for Additional Oxadiazine Compounds

| | | | | ESI Mass | Optical |
|---|---|---|---|---|---|
| | | | | Observed | Rotation |
| | | | | And Mass | And |
| Compound | | | | Ion | Optical |
| of | | HPLC Retention | Molecular | (M + H or | Rotation |
| Example | Structure and Name | Time and Condition | Weight | other) | Condition |
| 28A | 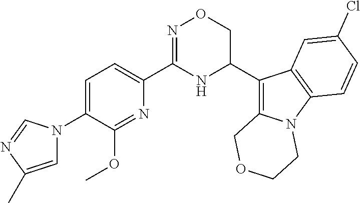 [SMILES: COc1nc(C2=NOCC(c3c4n(c5ccc(Cl)cc35)CCOC4)N2)ccc1-n1cnc(C)c1] | 7.51 min (column; zorbax-SB-C- 18 150 × 4.6 mm, 3.5 μm); mobile Phase: ACN + 0.5% TFA; 0.5% TFA + 5% ACN; flow rate: 1.0 mL/min; Gradient programme: T/B% 0.01/90, 10/10, 15/10: diluent: CH₃CN: Water | 478.93 | 479 [M + 1] | 137.29 C = 0.25, CH₂Cl₂ |
| | (+)-8-chloro-10-(3-(6-methoxy-5- | | | | |
| | (4-methyl-1H-imidazol-1- | | | | |
| | yl)pyridin-2-yl)-5,6-dihydro-4H- | | | | |
| | 1,2,4-oxadiazin-5-yl)-3,4-dihydro- | | | | |
| | 1H-[1,4]oxazino[4,3-a]indole | | | | |
| 28B | 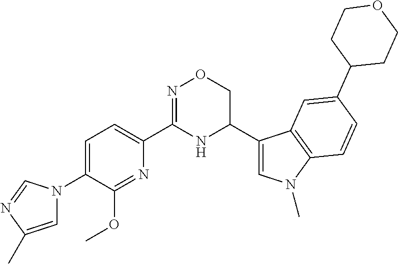 | 7.51 min (column; zorbax-SB-C- 18 150 × 4.6 mm, 3.5 μm); mobile Phase: ACN + 0.5% TFA; 0.5% TFA + 5% ACN; flow rate: 1.0 mL/min; Gradient programme: T/B% 0.01/90, 10/10, 15/10: diluent: CH₃CN: Water | 478.93 | 479 [M + 1] | −129.79 C = 0.25, CH₂Cl₂ |
| | (−)-8-chloro-10-(3-(6-methoxy-5- | | | | |
| | (4-methyl-1H-imidazol-1- | | | | |
| | yl)pyridin-2-yl)-5,6-dihydro-4H- | | | | |
| | 1,2,4-oxadiazin-5-yl)-3,4-dihydro- | | | | |
| | 1H-[1,4]oxazino[4,3-a]indole | | | | |
| 29A | 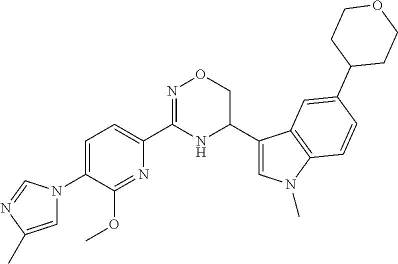 [SMILES: COc1nc(C2=NOCC(c3cn(C)c4ccc(C5CCOCC5)cc34)N2)ccc1-n1cnc(C)c1] | 6.67 min (column; X-select CSH- C18 150 × 4.6 mm, 3.5 μ); mobile Phase: ACN: 0.5% TFA; flow rate: 1.0 mL/min; Gradient programme: T/B% 0.01/90, 10/10, 15/10: diluent: CH₃CN: Water | 486.57 | 487.1 [M + 1] | −68.25 C = 0.25, CH₂Cl₂ |
| | (−)-3-(6-methoxy-5-(4-methyl-1H- | | | | |
| | imidazol-1-yl)pyridin-2-yl)-5-(1- | | | | |
| | methyl-5-(tetrahydro-2H-pyran-4- | | | | |
| | yl)-1H-indol-3-yl)-5,6-dihydro- | | | | |
| | 4H-1,2,4-oxadiazine | | | | |
| 29B | 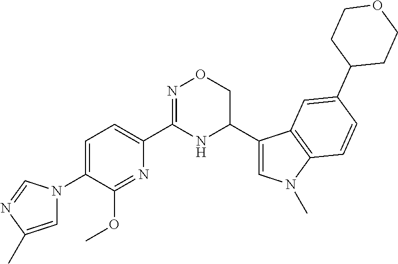 [SMILES: COc1nc(C2=NOCC(c3cn(C)c4ccc(C5CCOCC5)cc34)N2)ccc1-n1cnc(C)c1] | 6.68 min (column; X-select CSH- C18 150 × 4.6 mm, 3.5 μ); mobile Phase: ACN: 0.5% TFA; flow rate: 1.0 mL/min; Gradient programme: T/B% 0.01/90, 10/10, 15/10: diluent: CH₃CN: Water | 486.57 | 487.1 [M + 1] | 65.1 C = 0.25, CH₂Cl₂ |
| | (+)-3-(6-methoxy-5-(4-methyl-1H- | | | | |
| | imidazol-1-yl)pyridin 2 yl)-5-(1- | | | | |
| | methyl-5-(tetrahydro-2H-pyran-4- | | | | |
| | yl)-1H-indol-3-yl)-5,6-dihydro- | | | | |
| | 4H-1,2,4-oxadiazine | | | | |
| 30A | 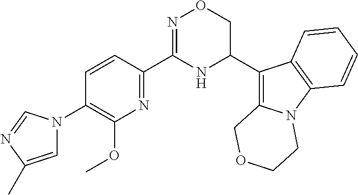 [SMILES: COc1nc(C2=NOCC(c3c4n(c5ccccc35)CCOC4)N2)ccc1-n1cnc(C)c1] | 6.72 min (column; zorbax-SB-C- 18 150 × 4.6 mm, 3.5 μ); mobile Phase: ACN: 0.05% TFA; flow rate: 1.0 mL/min; Gradient programme: T/B% 0.01/90, 10/10, 15/10: diluent: CH₃CN: Water | 444.49 | 445 [M + 1] | −57.65 C = 0.25, CH₂Cl₂ |
| | (−)-10-(3-(6-methoxy-5-(4-methyl- | | | | |
| | 1H-imidazol-1-yl)pyridin-2-yl)- | | | | |
| | 5,6-dihydro-4H-1,2,4-oxadiazin-5- | | | | |
| | yl)-3,4-dihydro-1H- | | | | |
| | [1,4]oxazino[4,3-a]indole | | | | |
| 30B | 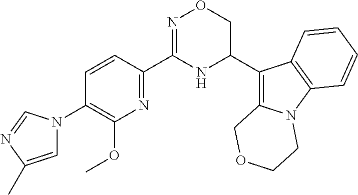 [SMILES: COc1nc(C2=NOCC(c3c4n(c5ccccc35)CCOC4)N2)ccc1-n1cnc(C)c1] | 6.70 min (column; zorbax-SB-C- 18 150 × 4.6 mm, 3.5 μ); mobile Phase: ACN: 0.05% TFA; flow rate: 1.0 mL/min; Gradient programme: T/B% 0.01/90, 10/10, 15/10: diluent: CH₃CN: Water | 444.49 | 445 [M + 1] | 47.1 C = 0.25, CH₂Cl₂ |
| | (+)-10-(3-(6-methoxy-5-(4- | | | | |
| | methyl-1H-imidazol-1-yl)pyridin- | | | | |
| | 2-yl)-5,6-dihydro-4H-1,2,4- | | | | |
| | oxadiazin-5-yl)-3,4-dihydro-1H- | | | | |
| | [1,4]oxazino[4,3-a]indole | | | | |

### Example 31

### Synthesis of (Z)-5-bromo-N′-hydroxy-6-methoxypicolinimidamide

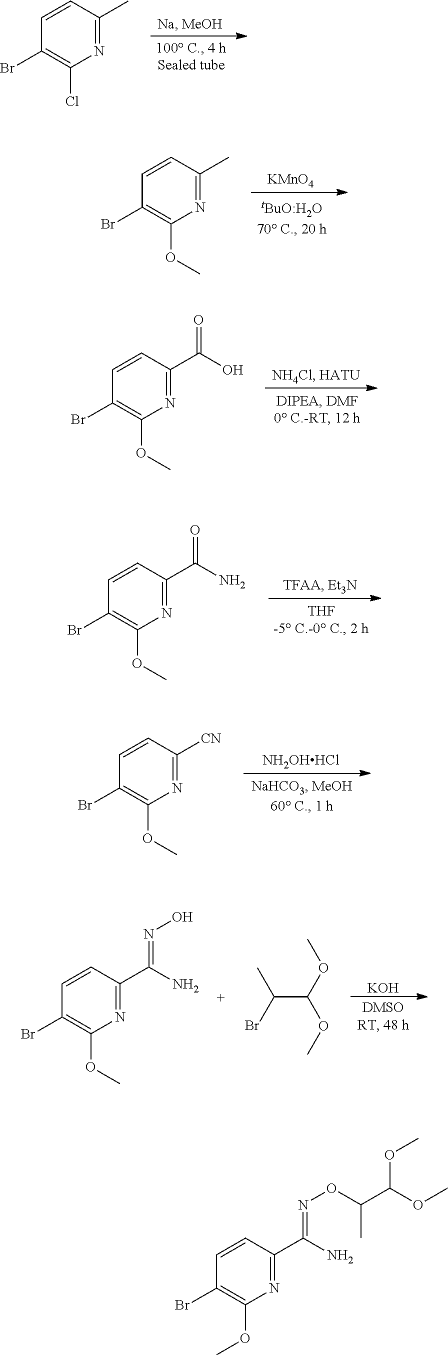

### Synthesis of 3-bromo-2-methoxy-6-methylpyridine

To a stirred solution of sodium metal (4.46 g, 194 mmol) in MeOH (200 mL) under an argon atmosphere was added 3-bromo-2-chloro-6-methylpyridine (20 g, 97 mmol) at 0° C. The reaction mixture was stirred at 100° C. for 4 h in a sealed tube. After consumption of starting material (by TLC), volatiles were evaporated in vacuo. The residue was quenched with water (200 mL) and extracted with EtOAc (2×250 mL). The combined organic extract was dried over sodium sulfate, filtered and concentrated in vacuo to obtain 3-bromo-2-methoxy-6-methylpyridine (18 g, 92%) as colorless liquid. ¹H-NMR (CDCl₃, 400 MHz): δ 7.61 (d, 1H), 6.60 (d, 1H), 3.99 (s, 3H), 2.39 (s, 3H); LCMS: 99.6%; 201.8 (M+1); (column; Ascentis Express C-18 (50×3.0 mm, 2.7 μm); RT 2.67 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 10% EtOAc/Hexane (R_{f}:0.7).

### Synthesis of 5-bromo-6-methoxypicolinic acid

To a stirred solution of 3-bromo-2-methoxy-6-methylpyridine (11 g, 54 mmol) in ^{t}BuOH:water (1:2, 900 mL) was added KMnO₄ (8.6 g, 54 mmol) at room temperature. The reaction mixture was stirred at 70° C. for 2 h. Then another 1 eq of KMnO₄ (8.6 g, 54 mmol) was added and stirred at 70° C. for 2 h. Again 1 eq of KMnO₄ (8.6 g, 54 mmol) was added and stirred at 70° C. for 16 h. After consumption of starting material (by TLC), the reaction was diluted with 1 M HCl solution (150 mL) and stirred for 30 min, filtered and washed with EtOAc (2×200 mL). The filtrate was extracted with EtOAc (2×100 mL) and the combined organic extracts were basified with 0.5 N sodium hydroxide solution (2×200 mL). The organic layer was separated, aqeous layer was acidified with concentrated hydrochloric acid to pH 2 and extracted with CH₂Cl₂(2×200 mL) The combined organic extracts were washed with brine (200 mL), dried over sodium sulfate, filtered and concentrated in vacuo to obtain 5-bromo-6-methoxypicolinic acid (6.4 g, 51%) as a white solid.

¹H-NMR (CDCl₃, 400 MHz): δ 8.03 (d, 1H), 7.71 (d, 1H), 4.10 (s, 3H); Mass (ESI): 234.1 [M+2]; LCMS: 97.0%; 233.7 (M+2); (column; Ascentis Express C-18 (50×3.0 mm, 2.7 μm); RT 1.94 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); HPLC (purity): 96.5%; (column; Eclipse XDB-C-18, 150×4.6 mm, 5.0 μm); RT 7.75 min; mobile phase: ACN: 0.05% Aq TFA; flow rate: 1.0 mL/min; Gradient program: T/B % 0.01/80, 3/80, 10/10, 20/10; diluent: CH₃CN: Water; TLC: 80% EtOAc/Hexane (R_{f}:0.1).

### Synthesis of 5-bromo-6-methoxypicolinamide

To a stirred solution of 5-bromo-6-methoxypicolinic acid (5 g, 21 mmol) in DMF (50 mL) under an argon atmosphere were added diisopropylethylamine (7.5 mL, 43 mmol), ammonium chloride (1.72 g, 32 mmol) and HATU (12.28 g, 32 mmol) at 0° C. The reaction mixture was warmed to room temperature and stirred for 12 h. After consumption of starting material (by TLC), the reaction was diluted with ice cold water (100 mL), the obtained solid was filtered and dried in vacuo to obtain 5-bromo-6-methoxypicolinamide (4.5 g, 90%) as a white solid used in the next step without further purification. ¹1-1-NMR (DMSO-d₆, 400 MHz): δ 8.18 (d, 1H), 8.05 (br s, 1H), 7.81 (br s, 1H), 7.51 (d, 1H), 4.02 (s, 3H); TLC: 50% EtOAc/Hexane (R_{f}:0.5).

### Synthesis of 5-bromo-6-methoxypicolinonitrile

To a stirred solution of 5-bromo-6-methoxypicolinamide (4.5 g, 19 mmol) in THF (45 mL) under an argon atmosphere were added triethylamine (6.8 mL, 48 mmol) and trifluoroacetic anhydride (3.3 mL, 23 mmol) at -5° C. The reaction mixture was stirred at 0° C. for 2 h. After consumption of starting material (by TLC), the reaction was diluted with water (100 mL) and extracted with EtOAc (2×100 mL). The combined organic extract was dried over sodium sulfate, filtered and concentrated in vacuo to obtain 5-bromo-6-methoxypicolinonitrile (3.6 g, 87%) as an off-white solid used in the next step without further purification. ¹1-1-NMR (DMSO-d₆, 400 MHz): δ 8.30 (d, 1H), 7.60 (d, 1H), 3.94 (s, 3H); LCMS: 97.6%; 214.8 (M+2); (column; Ascentis Express C-18 (50×3.0 mm, 2.7 μm); RT 2.51 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:0.6).

### Synthesis of (Z)-5-bromo-N′-hydroxy-6-methoxypicolinimidamide

To a stirred solution of 5-bromo-6-methoxypicolinonitrile (3.6 g, 17 mmol) in MeOH (85 mL) under an argon atmosphere were added hydroxylamine hydrochloride (1.5 g, 22 mmol) and sodium bicarbonate (2.12 g, 25 mmol) at room temperature. The reaction mixture was stirred at 60° C. for 1 h. After consumption of starting material (by TLC), the reaction was quenched with saturated ammonium chloride solution (100 mL) and extracted with EtOAc (2×100 mL). The combined organic extract was washed with water (100 mL), brine (150 mL), dried over sodium sulfate, filtered and concentrated in vacuo to obtain (Z)-5-bromo-N′-hydroxy-6-methoxypicolinimidamide (3.2 g, 77%) as a white solid used in the next step without further purification. ¹H-NMR (CDCl₃, 400 MHz): δ 7.80 (d, 1H), 7.40 (d, 1H), 6.67 (br s, 1H), 5.45 (br s, 2H), 4.06 (s, 3H); LCMS: 95.9%; 245.8 (M+); (column; Ascentis Express C-18 (50×3.0 mm, 2.7 μm); RT 1.51 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); UPLC (purity): 95.2%; (column; Acquity UPLC BEH C-18, 50×2.1 mm, 1.7 μ); RT 1.38 mm; mobile phase: ACN: 0.025% Aq TFA; flow rate: 0.5 mL/min; Gradient program: T/B % 0.01/90, 0.5/90, 3/10, 6/10: diluent: CH₃CN: Water; TLC: 20% EtOAc/Hexane (R_{f}:0.2).

### Synthesis of (Z)-5-bromo-N′-((1,1-dimethoxypropan-2-yl)oxy)-6-methoxypicolinimidamide

To a stirred solution of (Z)-5-bromo-N′-hydroxy-6-methoxypicolinimidamide (5 g, 20 mmol) in DMSO (50 mL) at 0° C. under an argon atmosphere were added potassium hydroxide (1.7 g, 31 mmol) and 2-bromo-1,1-dimethoxypropane (4.46 g, 24 mmol). The reaction mixture was warmed to room temperature and stirred for 48 h. After consumption of starting material (monitored by TLC), the reaction mixture was diluted with ice cold water (200 mL) and extracted with EtOAc (2×200 mL). The combined organic extracts were dried over sodium sulfate, filtered and concentrated in vacuo. The crude material was purified by column chromatography using 20% EtOAc: Hexane to afford (Z)-5-bromo-N′-((1,1-dimethoxypropan-2-yl)oxy)-6-methoxypicolinimidamide (930 mg, 13%) as pale yellow solid. ¹H-NMR (CDCl₃, 400 MHz): δ 7.80 (d, 1H), 7.50 (d, 1H), 5.49 (br s, 2H), 4.50 (d, 1H), 4.31-4.25 (m, 1H), 4.01 (s, 3H), 3.45 (s, 6H), 1.31 (d, 3H); TLC: 30% EtOAc/Hexane (R_{f}:0.6)

### Example 32

### Synthesis of N′-((1,1-dimethoxypropan-2-yl)oxy)-6-methoxy-5-(4-methyl-1H-imidazol-1-yl)picolinimidamide

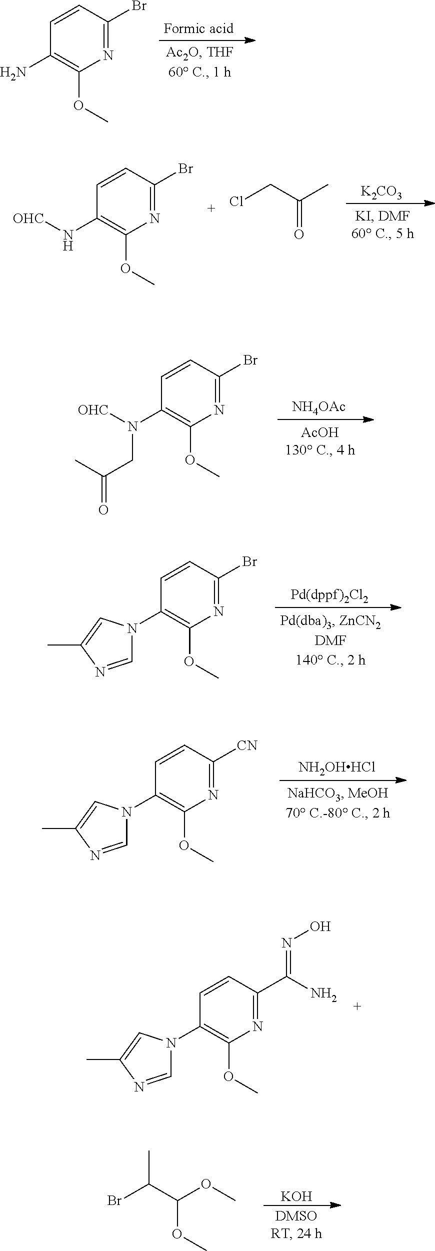

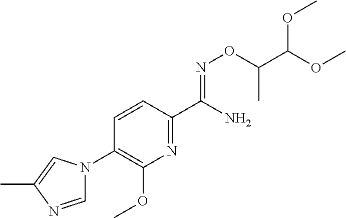

### Synthesis of N-(6-bromo-2-methoxypyridin-3-yl) formamide

To the acetic anhydride (8.5 mL) at room temperature under an argon atmosphere was added formic acid (12.5 mL). The reaction mixture was stirred at room temperature for 30 min. Then 6-bromo-2-methoxypyridin-3-amine (5 g, 25 mmol) in THF (22 mL) at room temperature was added to the reaction mixture. The reaction mixture was stirred at 60° C. for 1 h. After consumption of starting material (by TLC), the reaction mixture was diluted with ice cold water (500 mL) stirred for 30 mm to obtain the solid. The solid was collected by filtration and dried in vacuo to obtain N′-(6-bromo-2-methoxypyridin-3-yl) formamide (5.5 g, 98%) as an off-white solid. ¹H NMR (CDCl₃, 500MHz): δ 8.52-8.50 (m, 2H), 7.61 (br s, 1H), 7.09 (d, 1H), 4.05 (s, 3H); LCMS: 99.8%; 232.7 (M+3); (column; Ascentis Express C-18 (50×3.0 mm, 2.7 μm); RT 2.05 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:0.3).

### Synthesis of N-(6-bromo-2-methoxypyridin-3-yl)-N-(2-oxopropyl) formamide

To a stirred solution of N-(6-bromo-2-methoxypyridin-3-yl) formamide (27 g, 117 mmol) in DMF (216 mL) at room temperature under an argon atmosphere were added potassium carbonate (57 mg, 411 mmol), 1-chloropropan-2-one (28.8 g, 293 mmol) and potassium iodide (1.94 g, 12 mmol). The reaction mixture was stirred at 60° C. for 5 h. After consumption of starting material (by TLC), the reaction mixture was diluted with ice cold water (200 mL) and stirred for 10 mm to obtain the solid. The solid was collected by filtration and dried in vacuo to obtain N-(6-bromo-2-methoxypyridin-3-yl)-N-(2-oxopropyl) formamide (32 g, 94%) as an off-white solid. ¹H NMR (CDCl₃, 500MHz): δ 8.21 (s, 1H), 7.48 (d, 1H), 7.13 (d, 1H), 4.46 (s, 2H), 4.01 (s, 3H), 2.16 (s, 3H); LCMS:99.4%; 288.7 (M+3); (column; Ascentis Express C-18 (50×3.0 mm, 2.7 μm); RT 2.05 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:0.2).

### Synthesis of 6-bromo-2-methoxy-3-(4-methyl-M-imidazol-1-yl) pyridine

The mixture of ammonium acetate (43 g, 553 mmol) in AcOH (208 mL) at room temperature under an argon atmosphere was stirred for 30 mm Then N-(6-bromo-2-methoxypyridin-3-yl)-N-(2-oxopropyl) formamide (32 g, 111 mmol) was added to the reaction mixture at room temperature. The reaction mixture was stirred at 130° C. for 4 h. After consumption of starting material (by TLC), the reaction mixture was diluted with ice cold water (200 mL), the aqeous layer was neutralised with 50% sodium hydroxide solution (200 mL) (pH′7) to obtain the solid. The solid was collected by filtration, washed with ether (100 mL) and dried in vacuo to obtain 6-bromo-2-methoxy-3-(4-methyl-1H-imidazol-1-yl) pyridine (17.5 g, 60%) as an off-white solid.

¹H NMR (CDCl₃, 400 MHz): δ 7.72 (s, 1H), 7.39 (d, 1H), 7.16 (d, 1H), 6.91 (s, 1H), 4.03 (s, 3H), 2.29 (s, 3H); LCMS: 99.3%; 267.9 (M+1); (column; Ascentis Express C-18 (50×3.0 mm, 2.7 μm); RT 1.54 mm; mobile phase: 0.025% Aq TFA+5% ACN:ACN+5% 0.025% Aq TFA; T/B %:0.01/5, 0.5/5, 3/100, 5/100; flow rate: 1.2 mL/min) (Gradient); TLC:40% EtOAc/Hexane (R_{f}:0.2).

### Synthesis of 6-methoxy-5-(4-methyl-1H-imidazol-1-yl) picolinonitrile

To a stirred solution of 6-bromo-2-methoxy-3-(4-methyl-1H-imidazol-1-yl) pyridine (20 g, 74 mmol) in DMF (240 mL) at room temperature under an argon atmosphere were added Pd(dppf)₂Cl₂ (500 mg, 0.9 mmol), Pd₂(dba)₃ (682 mg, 0.7 mmol) and zinc cyanide (5.3 g, 45 mmol). The reaction mixture was stirred at 140° C. for 2 h. After consumption of starting material (by TLC), the reaction mixture was diluted with 25% NH₄OH solution (240 mL) to obtain the solid. The solid was collected by filtration and dried in vacuo to obtain 6-methoxy-5-(4-methyl-1H-imidazol-1-yl) picolinonitrile (14 g, 88%) as a pale yellow solid. ¹H NMR (CDCl₃: 400 MHz): δ 7.89 (s, 1H), 7.63 (d, 1H), 7.43 (d, 1H), 7.01 (s, 1H), 4.09 (s, 3H), 2.30 (s, 3H); LCMS: 98.7%; 214.9 (M+1); (column; Ascentis Express C-18 (50×3.0 mm, 2.7 μm); RT 1.19 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC:EtOAc (R_{f}:0.3).

### Synthesis of (Z)-N′-hydroxy-6-methoxy-5-(4-methyl-1H-imidazol-1-yl) picolinimidamide

To a stirred solution of 6-methoxy-5-(4-methyl-1H-imidazol-1-yl) picolinonitrile (4 g, 19 mmol) in MeOH (100 mL) at room temperature under an argon atmosphere were added hydroxyl amine hydrochloride (1.7 g, 24 mmol) and sodium bicarbonate (2.35 g, 28 mmol). The reaction mixture was stirred at 70-80° C. for 2 h. After consumption of starting material (by TLC), the volatiles were evaporated in vacuo. The residue was diluted with ice cold water (100 mL) to obtain the solid. The solid was collected by filtration and dried in vacuo to obtain (Z)-N′-hydroxy-6-methoxy-5-(4-methyl-1H-imidazol-1-yl) picolinimidamide (4 g, 87%) as a pale yellow solid. LCMS: 99.8%; 248 (M+1); (column; Ascentis Express C-18 (50×3.0 mm, 2.7 μm); RT 0.42 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC:EtOAc (R_{f}:0.2).

### Synthesis of (Z)-N′-((1,1-dimethoxypropan-2-yl) oxy)-6-methoxy-5-(4-methyl-1H-imidazol-1-yl) picolinimidamide

(Z)-N′-hydroxy-6-methoxy-5-(4-methyl-1H-imidazol-1-yl) picolinimidamide was prepared as described in Example 3.

### Example 33

### Synthesis of (Z)-N′-hydroxy-3-methoxy-4-(4-methyl-1H-imidazol-1-yl) benzimidamide

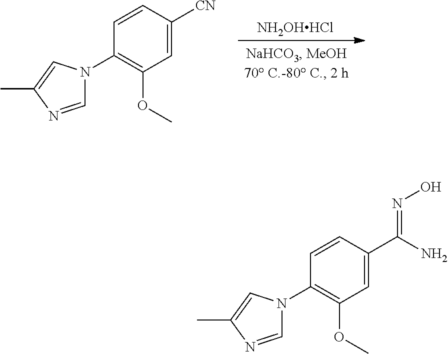 [SMILES: COc1cc(C#N)ccc1-n1cnc(C)c1 ; "NH2OH·HCl" ; COc1cc(/C(N)=N/O)ccc1-n1cnc(C)c1]

### Synthesis of (Z)-N′-hydroxy-3-methoxy-4-(4-methyl-1H-imidazol-1-yl) benzimidamide

To a stirred solution of 3-methoxy-4-(4-methyl-1H-imidazol-1-yl) benzonitrile (450 mg, 2 mmol) in MeOH (23 mL) at room temperature under an argon atmosphere were added hydroxyl amine hydrochloride (230 mg, 2 mmol) and sodium bicarbonate (230 mg, 3 mmol). The reaction mixture was stirred at 85° C. for 3 h. After consumption of starting material (by TLC), the volatiles were evaporated in vacuo. The residue was diluted with ice cold water (50 mL), stirred for 10 mm, to obtain the solid. The solid was collected by filtration and dried in vacuo to obtain (Z)-N′-hydroxy-3-methoxy-4-(4-methyl-1H-imidazol-1-yl) benzimidamide (450 mg, 88%) as an off-white solid.

¹H NMR (DMSO-d₆, 500 MHz): δ 9.73 (s, 1H), 7.79 (s, 1H), 7.47 (s, 1H), 7.37 (s, 2H), 5.92 (s, 2H), 3.82 (s, 3H), 2.15 (s, 3H); LCMS: 92.4%; 247 (M+1); (column; X-select CSH C-18 (50×3.0 mm, 2 7 μm); RT 2.06 mm; mobile phase: 2.5 mM NH₄OOCH in water+5% ACN: ACN+5% 2.5mM NH₄OOCH in water; T/B %: 0.01/5, 0.5/5, 3/100, 5/100; flow rate: 1.2 mL/min) (Gradient); TLC: 3% MeOH/CH₂Cl₂ (R_{f}:0.3).

### Example 34

### Synthesis of 3-(6-methoxy-5-(4-methyl-1H-imidazol-1-yl) pyridin-2-yl)-6-methyl-5,6-dihydro-4H-1,2,4-oxadiazin-5-ol

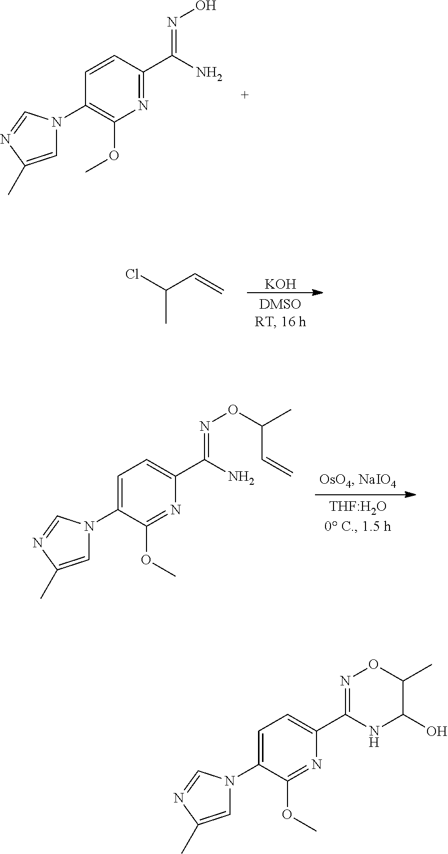

### Synthesis of (Z)-N′-(but-3-en-2-yloxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (2 g, 8 mmol) in DMSO (20 mL) at room temperature under an argon atmosphere were added potassium hydroxide (910 mg, 16 mmol) and 3-chlorobut-1-ene (1.21 mL, 12 mmol). The reaction mixture was warmed to room temperature and stirred for 16 h. After consumption of starting material (by TLC), the reaction mixture was diluted with ice cold water (100 mL) and extracted with EtOAc (2×100 mL). The combined organic extracts were dried over sodium sulfate, filtered and concentrated in vacuo to obtain (Z)-N′-(but-3-en-2-yloxy)-6-methoxy-5-(4-methyl-1H-imidazol-1-yl) picolinimidamide (2 g, 82%) as brown syrup used in the next step without further purification. ¹H NMR (CD₃OD, 400 MHz): δ 7.90 (s, 1H), 7.74 (dd, 1H), 7.57 (dd, 1H), 7.19 (s, 1H), 6.00-5.91 (m, 1H), 5.22 (d, 1H), 5.10 (d, 1H), 4.66-4.60 (m, 1H), 4.07 (s, 3H), 2.23 (s, 3H), 1.38 (dd, 3H); TLC: 5% MeOH/CH₂Cl₂ (R_{f}:0.4).

### Synthesis of 3-(6-methoxy-5-(4-methyl-1H-imidazol-1-yl) pyridin-2-yl)-6-methyl-5,6-dihydro-4H-1,2,4-oxadiazin-5-ol

To a stirred solution of (Z)-N′-(but-3-en-2-yloxy)-6-methoxy-5-(4-methyl-1H-imidazol-1-yl) picolinimidamide (2 g, 6 mmol) in THF:water (4:1, 120 mL) at 0° C. under an argon atmosphere was added osmium tetraoxide in tertiary butanol (169 mg, 0.6 mmol). The reaction mixture was stirred at 0° C. for 5 mm Then sodium periodate (4.26 g, 20 mmol) was added to the reaction mixture at 0° C. The reaction mixture was stirred at 0° C. for 1.5 h. After consumption of starting material (by TLC), the reaction mixture was diluted with saturated sodium bicarbonate solution (100 mL) and extracted with EtOAc (2×100 mL). The combined organic extracts were dried over sodium sulfate, filtered and concentrated in vacuo. The crude washed with dichloro ethane: n-pentane (1:9, 2×10 mL) to afford 3-(6-methoxy-5-(4-methyl-1H-imidazol-1-yl) pyridin-2-yl)-6-methyl-5,6-dihydro-4H-1,2,4-oxadiazin-5-ol (2 g, 99%) as brown solid used in the next step without further purification. ¹H NMR (CD₃OD, 400 MHz): δ 7.98-7.90 (m, 1H), 7.84 (d, 1H), 7.60 (d, 1H), 7.10 (s, 1H), 4.76-4.74 (m, 1H), 4.13 (s, 3H), 4.12-4.09 (m, 1H), 3.70-3.60 (m, 1H), 2.22 (s, 3H), 1.30 (d, 3H); 5% MeOH/CH₂Cl₂ (R_{f}:0.2).

### Example 35

### Synthesis of 3-(5-bromo-6-methoxypyridin-2-yl)-5-(3-chlorophenyl)-6-methyl-5,6-dihydro-4H-1,2,4-oxadiazine

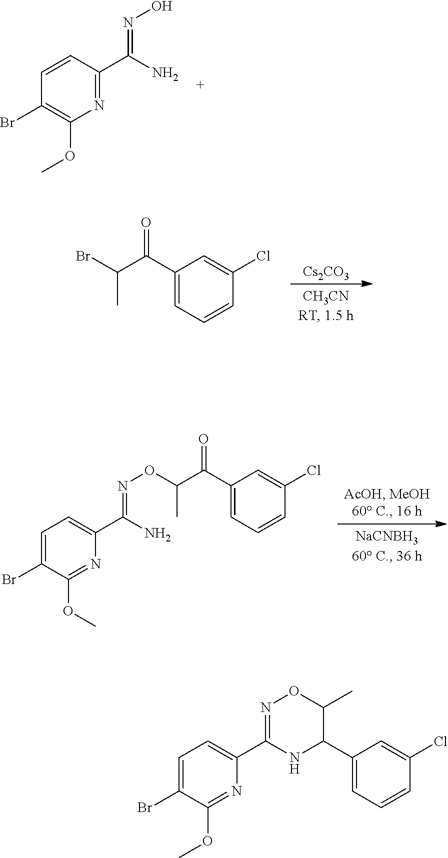

### Synthesis of (Z)-5-bromo-N′-((1-(3-chlorophenyl)-1-oxopropan-2-yl) oxy)-6-methoxypicolinimidamide

To a stirred solution of (Z)-5-bromo-N′-hydroxy-6-methoxypicolinimidamide (2.5 g, 10 mmol) in CH₃CN (125 mL) at room temperature under an argon atmosphere were added cesium carbonate (6.6 g, 20.32 mmol) and 2-bromo-1-(3-chlorophenyl) propan-1-one (3.75 g, 15 mmol). The reaction mixture was stirred at room temperature for 1.5 h. After consumption of starting material (by TLC), the reaction mixture was filtered. The filtrate was concentrated in vacuo to obtain (Z)-5-bromo-N′-((1-(3-chlorophenyl)-1-oxopropan-2-yl) oxy)-6-methoxypicolinimidamide (3.44 g, crude) as brown solid used in the next step without further purification. LCMS: 71.1%; 413.7 (M+3); (column; Ascentis Express C-18 (50×3.0 mm, 2.7 μm); RT 3.06 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:0.5).

### Synthesis of 3-(5-bromo-6-methoxypyridin-2-yl)-5-(3-chlorophenyl)-6-methyl-5,6-dihydro-4H-1,2,4-oxadiazine

To a stirred solution of (Z)-5-bromo-N′-((1-(3-chlorophenyl)-1-oxopropan-2-yl) oxy)-6-methoxypicolinimidamide (2.8 g, 7 mmol) in MeOH (80 mL) at room temperature under an argon atmosphere was added acetic acid (14 mL). The reaction mixture was stirred for 16 h at 60° C. Then sodium cyanoborohydride (512 mg, 8 mmol) was added to the reaction mixture at room temperature. The reaction mixture was stirred for 3 h at 60° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo to obtain 3-(5-bromo-6-methoxypyridin-2-yl)-5-(3-chlorophenyl)-6-methyl-5,6-dihydro-4H-1,2,4-oxadiazine (650 mg, 25%) as colorless solid. LCMS: 30.5%; 397.7 (M+3); (column; Ascentis Express C-18 (50×3.0 mm, 2.7 μm); RT 2.84 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:0.5).

### Example 36

### Synthesis of 8-(4-chloro-2-(trifluoromethyl) phenyl)-4-(6-methoxy-5-(4-methyl-1H-imidazol-1-yl) pyridin-2-yl)-1,6,7,8,9,9a-hexahydropyrazino [1,2-d] [1,2,4] oxadiazine

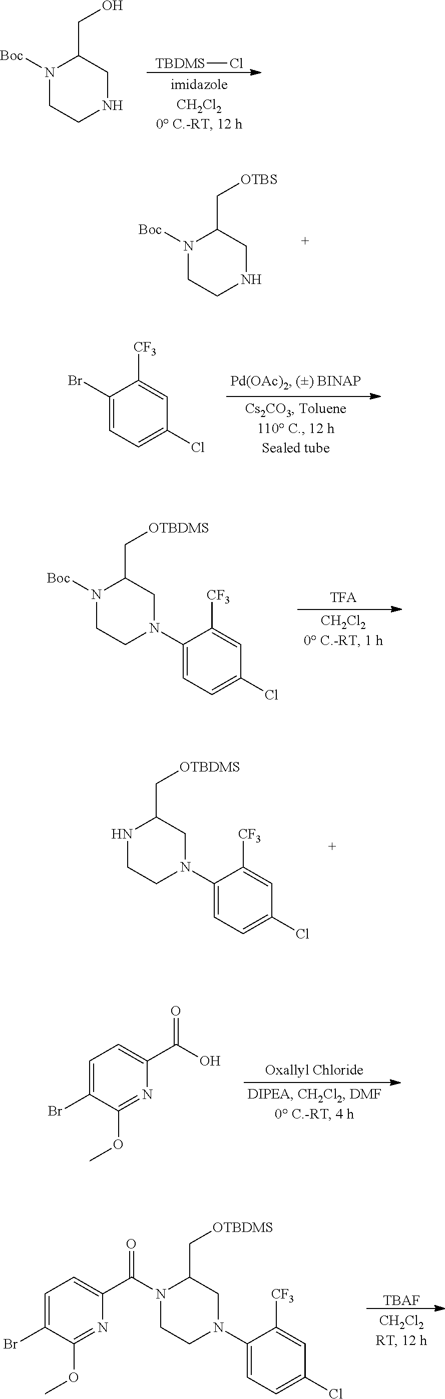

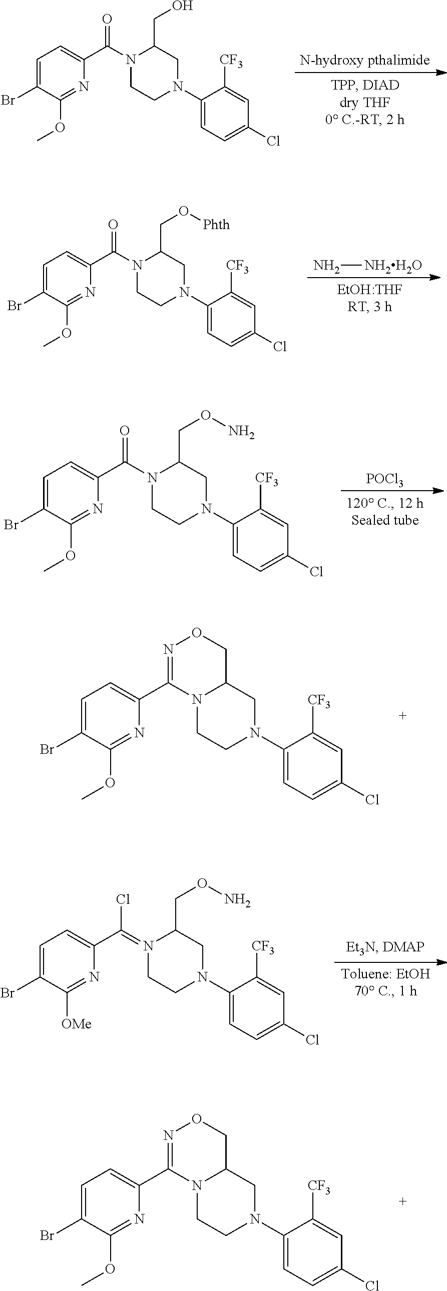

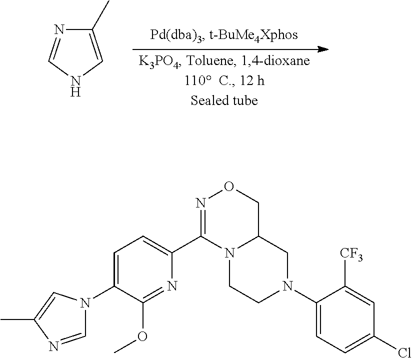

### Synthesis of tert-butyl 2-(((tert-butyldimethylsilyl) oxy) methyl) piperazine-1-carboxylate

To a stirred solution of tert-butyl 2-(hydroxymethyl) piperazine-1-carboxylate (3 g, 14 mmol) in CH₂Cl₂ (30 mL) under an argon atmosphere were added imidazole (2.1 g, 29 mmol) and TBDMS-chloride (4.05 g, 28 mmol) at 0° C. The reaction mixture was stirred at room temperature for 12 h. After consumption of starting material (by TLC), the reaction mixture was diluted with water (30 mL) and extracted with CH₂Cl₂ (2×50 mL). The combined organic extracts were dried over sodium sulfate, filtered and concentrated in vacuo to obtain tert-butyl 2-(((tert-butyldimethylsilyl) oxy) methyl) piperazine-1-carboxylate (4 g, 87%) as a colorless syrup. TLC: 50% EtOAc/Hexane (R_{f}:0.1).

### Synthesis of tert-butyl 2-(((tert-butyldimethylsilyl) oxy) methyl)-4-(4-chloro-2-(trifluoromethyl) phenyl) piperazine-1-carboxylate

To a stirred solution of tert-butyl 2-(((tert-butyldimethylsilyl) oxy) methyl) piperazine-1-carboxylate (3 g, 9 mmol) in toluene (30 mL) under an argon atmosphere were added 1-bromo-4-chloro-2-(trifluoromethyl) benzene (4.7 g, 18 mmol), (±) BINAP (560 mg, 1 mmol), Pd(OAc)₂ (203 mg, 1 mmol) and cesium carbonate (8.8 g, 3 mmol) at room temperature and purged under an argon atmosphere for 10 mm. The reaction mixture was stirred at 110° C. for 12 h in a sealed tube. After consumption of starting material (by TLC), the reaction mixture was filtered and the filtrate was concentrated in vacuo. The crude material was purified by column chromatography using 10% EtOAc/Hexane to afford tert-butyl 2-(((tert-butyldimethylsilyl) oxy) methyl)-4-(4-chloro-2-(trifluoromethyl) phenyl) piperazine-1-carboxylate (3.7 g, 80%) as a brown syrup. ¹H NMR (DMSO-d₆, 400 MHz): δ 7.76-7.69 (m, 2H), 7.64 (d, 1H), 4.08 (br s, 1H), 3.91-3.71 (m, 3H), 3.00-2.93 (m, 3H), 2.84 (d, 1H), 2.74-2.66 (m, 1H), 1.42 (s, 9H), 0.85 (s, 9H), 0.05-0.04 (m, 6H); LCMS: 93.0%; 409 (M-Boc); (column; Ascentis Express C-18 (50×3.0 mm, 2.7 μm); RT 4.42 mm; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexane (R_{f}:0.7).

### Synthesis of 3-(((tert-butyldimethylsilyl) oxy) methyl)-1-(4-chloro-2-(trifluoromethyl) phenyl) piperazine

To a stirred solution of tert-butyl 2-(((tert-butyldimethylsilyl) oxy) methyl)-4-(4-chloro-2-(trifluoromethyl) phenyl) piperazine-1-carboxylate (700 mg, 1 mmol) in CH₂Cl₂ (7 mL) under an argon atmosphere was added trifluoroacetic acid (3.5 mL) at 0° C. The reaction mixture was stirred at room temperature for 1 h. After consumption of starting material (by TLC), the reaction mixture was diluted with a saturated sodium bicarbonate solution (100 mL) and extracted with EtOAc (2×50 mL). The combined organic extracts were dried over sodium sulfate, filtered and concentrated in vacuo to afford 3-(((tert-butyldimethylsilyl) oxy) methyl)-1-(4-chloro-2-(trifluoromethyl) phenyl) piperazine (560 mg, 99%) as a colorless syrup which was used in the next step without further purification. LCMS: 62.5%; 409 (M+); (column; Ascentis Express C-18 (50×3.0 mm, 2.7 μm); RT 2.46 mm; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 40% EtOAc/Hexane (R_{f}:0.4).

### Synthesis of (5-bromo-6-methoxypyridin-2-yl) (2-(((tert-butyldimethylsilyl) oxy) methyl)-4-(4-chloro-2-(trifluoromethyl) phenyl) piperazin-1-yl) methanone

To a stirred solution of 5-bromo-6-methoxypicolinic acid (350 mg, 1 mmol) in CH₂Cl₂ (2 mL) under an argon atmosphere were added oxalyl chloride (347 mg, 2 mmol) and DMF (catalytic amount) at 0° C. The reaction mixture was stirred at room temperature for 2 h. After consumption of acid (by TLC), the volatiles were evaporated in vacuo to give 5-bromo-6-methoxypicolinoyl chloride.

To a stirred solution of 3-(((tert-butyldimethylsilyl) oxy) methyl)-1-(4-chloro-2-(trifluoromethyl) phenyl) piperazine (560 mg, 1 mmol) in CH₂Cl₂ (2 mL) under an argon atmosphere were added diisopropylethylamine (0.73 mL, 4 mmol) and the above acid chloride in CH₂Cl₂ (1.6 mL) at 0° C. The reaction mixture was stirred at room temperature for 2 h. After consumption of acid (by TLC), the reaction mixture was quenched with water (20 mL) and extracted with CH₂Cl₂ (2×20 mL). The combined organic extract was washed with water (20 mL), dried over sodium sulfate, filtered and concentrated in vacuo to obtain (5-bromo-6-methoxypyridin-2-yl) (2-(((tert-butyldimethylsilyl) oxy) methyl)-4-(4-chloro-2-(trifluoromethyl) phenyl) piperazin-1-yl) methanone (600 mg, crude) as a pale yellow liquid used in the next step without further purification. LCMS: 53.7%; 623.9 (M+); (column; Ascentis Express C-18 (50×3.0 mm, 2.7 μm); RT 3.98 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexanes (R_{f}:0.7).

### Synthesis of (5-bromo-6-methoxypyridin-2-yl) (4-(4-chloro-2-(trifluoromethyl) phenyl)-2-(hydroxymethyl) piperazin-1-yl) methanone

To a stirred solution of (5-bromo-6-methoxypyridin-2-yl) (2-(((tert-butyldimethylsilyl) oxy) methyl)-4-(4-chloro-2-(trifluoromethyl) phenyl) piperazin-1-yl) methanone (600 mg, 1 mmol) in CH₂Cl₂ (6 mL) under an argon atmosphere was added tetrabutylammonium fluoride (2 mL, 1 M in THF solution) at room temperature. The reaction mixture was stirred at room temperature for 12 h. After consumption of starting material (by TLC), the reaction mixture was diluted with saturated sodium bicarbonate solution (20 mL) and extracted with CH₂Cl₂ (2×20 mL). The combined organic extracts were dried over sodium sulfate, filtered and concentrated in vacuo. The crude material was purified by column chromatography using 20% EtOAc/Hexane to afford (5-bromo-6-methoxypyridin-2-yl) (4-(4-chloro-2-(trifluoromethyl) phenyl)-2-(hydroxymethyl) piperazin-1-yl) methanone (400 mg, 81%) as a white solid. LCMS: 96.9%; 509.8 (M+); (column; Ascentis Express C-18 (50×3.0 mm, 2.7 μm); RT 2.84 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 20% EtOAc/Hexane (R_{f}:0.4).

### Synthesis of 2-((1-(5-bromo-6-methoxypicolinoyl)-4-(4-chloro-2-(trifluoromethyl) phenyl) piperazin-2-yl) methoxy) isoindoline-1, 3-dione

To a stirred solution of (5-bromo-6-methoxypyridin-2-yl) (2-(((tert-butyldimethylsilyl) oxy) methyl)-4-(4-chloro-2-(trifluoromethyl) phenyl) piperazin-1-yl) methanone (700 mg, 1 mmol) in dry THF (7 mL) under an argon atmosphere were added molecular sieves (1 g), diisopropylazodicarboxylate (415 mg, 2 mmol), triphenylphosphine (541 mg, 2 mml) and N-hydroxyphthalimide (269 mg, 1 mmol) at 0° C. The reaction mixture was warmed to room temperature and stirred for 2 h. After consumption of starting material (by TLC), the reaction mixture was diluted with water (20 mL) and extracted with EtOAc (2×5 mL). The combined organic extracts were dried over sodium sulfate, filtered and concentrated in vacuo. The crude material was purified by preparative HPLC (YMC Actus Triart C-18 (250×20 mm, 5t (155 mg loading; CH₃CN: 0.05% TFA (0.1/90, 2/80, 10/60, 20/30, 25/10, 35/10)) to afford 2-((1-(5-bromo-6-methoxypicolinoyl)-4-(4-chloro-2-(trifluoromethyl) phenyl) piperazin-2-yl) methoxy) isoindoline-1, 3-dione (350 mg, 39%) as a white solid. LCMS: 98.1%; 654.6 (M+); (column; Ascentis Express C-18 (50×3.0 mm, 2.7 μm); RT 3.21 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexane (R_{f}:0.7).

### Synthesis of (2-((aminooxy) methyl)-4-(4-chloro-2-(trifluoromethyl) phenyl) piperazin-1-yl) (5-bromo-6-methoxypyridin-2-yl) methanone

To a stirred solution of 2-((1-(5-bromo-6-methoxypicolinoyl)-4-(4-chloro-2-(trifluoromethyl) phenyl) piperazin-2-yl) methoxy) isoindoline-1, 3-dione (320 mg, 0.49 mmol) in EtOH: THF (2:1, 7.36 mL) under an argon atmosphere was added hydrazine hydrate (0.48 mL) at room temperature. The reaction mixture was stirred at room temperature for 3 h. After consumption of starting material (by TLC), the volatiles were evaporated in vacuo. The residue was dissolved in ether and the obtained solid was filtered. The filtrate was washed with water (30 mL) and extracted with EtOAc (2×20 mL). The combined organic extracts were dried over sodium sulphate, filtered and concentrated in vacuo to obtain (2-((aminooxy) methyl)-4-(4-chloro-2-(trifluoromethyl) phenyl) piperazin-1-yl) (5-bromo-6-methoxypyridin-2-yl) methanone (300 mg, crude) as a white solid which was used in the next step without further purification. LCMS: 78.0%; 524.7 (M+); (column; Ascentis Express C-18 (50×3.0 mm, 2.7 μm); RT 2.20 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexane (R_{f}:0.7).

### Synthesis of 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine

To a stirred solution of (2-((aminooxy) methyl)-4-(4-chloro-2-(trifluoromethyl) phenyl) piperazin-1-yl) (5-bromo-6-methoxypyridin-2-yl) methanone (300 mg, 0.56 mmol) in POCl₃ (3 mL) under an argon atmosphere was stirred at 120° C. for 12 h in a sealed tube. After consumption of starting material (by TLC), the reaction mixture was diluted with saturated sodium bicarbonate solution (20 mL) and extracted with EtOAc (2×5 mL). The combined organic extracts were dried over sodium sulfate, filtered and concentrated in vacuo to obtain 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine (350 mg, crude) as a white solid. LCMS: 54.8%; 506.8 (M+2); (column; Ascentis Express C-18 (50×3.0 mm, 2.7 μm); RT 3.15 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexane (R_{f}:07).

### Synthesis of 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine

To a stirred solution of 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine (350 mg, 0.64 mmol) in toluene:EtOH (2:1, 3 mL) under an argon atmosphere were added triethylamine (0.88 mL, 6.40 mmol) and dimethylaminopyridine (78 mg, 0.64 mmol) at 0° C. The reaction mixture was stirred at 70° C. for 1 h. After consumption of starting material (by TLC), the reaction mixture was diluted with saturated sodium bicarbonate solution (20 mL) and extracted with EtOAc (2×5 mL). The combined organic extracts were dried over sodium sulfate, filtered and concentrated in vacuo to afford 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine (90 mg, 28%) as a white solid. LCMS: 89.4%; 506.8 (M+2); (column; Ascentis Express C-18 (50×3.0 mm, 2.7 μm); RT 3.06 min; mobile phase: 0.025% Aq TFA+5% ACN:ACN+5% 0.025% Aq TFA; T/B % 0.01/5, 0.5/5, 3/100, 5/100: flow rate: 1.2 mL/min) (Gradient); TLC: 50% EtOAc/Hexane (R_{f}:07).

### Synthesis of 8-(4-chloro-2-(trifluoromethyl) phenyl)-4-(6-methoxy-5-(4-methyl-1H-imidazol-1-yl) pyridin-2-yl)-1,6,7,8,9,9a-hexahydropyrazino [1,2-d] [1,2,4] oxadiazine

To a dry vial was added a suspension of Pd₂(dba)₃ (8 mg, 0.008 mmol) and tert-butyl tetra methyl X-phos (8.5 mg, 0.02 mmol) in toluene: 1,4-dioxane (2:1, 0.67 mL) at room temperature. The suspension was degassed with argon, heated to 120° C., and stirred at 120° C. for 3 min. A mixture of 4-(5-bromo-6-methoxypyridin-2-yl)-8-(4-chloro-2-(trifluoromethyl) phenyl)-1,6,7,8,9,9a-hexahydropyrazino [1,2-d] [1,2,4] oxadiazine (90 mg, 0.17 mmol), 4-methyl-1H-imidazole (17 mg, 0.21 mmol) and potassium phosphate (76 mg, 0.35 mmol) in toluene: 1, 4-dioxane (2:1, 0.67 mL) was degassed and the catalyst premix was added. The resulting mixture was stirred at 110° C. for 12 h in a sealed tube. After consumption of the starting material (monitored by TLC and LCMS), the reaction mixture was filtered and the filtrate was concentrated in vacuo. The crude material was purified by column chromatography using 5%MeOH/CH₂Cl₂ to afford 8-(4-chloro-2-(trifluoromethyl) phenyl)-4-(6-methoxy-5-(4-methyl-1H-imidazol-1-yl) pyridin-2-yl)-1,6,7,8,9,9a-hexahydropyrazino [1,2-d] [1,2,4] oxadiazine (50 mg, 55%) as a pale yellow thick syrup. Racemic compound of Example 36 was separated using a Chiralpak-ADH column (250×20 mm, 5 μm) (20 mg loading; 0.1% DEA in n-Hexane:EtOH (A:B: 85:15) as mobile phase) to afford the compounds of Example 36A (Fraction (I) (+)) and Example 36B (Fraction II (−)). Analytical conditions for Example 36A and Example 36B. HPLC (purity): (column; Eclipse XDB C-18, 150×4.6 mm, 5.0 μm); mobile phase: ACN: 0.05% Aq TFA; flow rate: 1.0 mL/min; Gradient program: T/B % 0.01/90, 2/90, 8/10, 15/10: Diluent: CH₃CN: Water: Chiral HPLC: (Chiralpak-ADH (250×4.6 mm, 5 μm; mobile phase (A) 0.1% DEA in n-Hexane (B) EtOH (A::B; 85:15); flow Rate: 1.0 mL/min).

### Example 36A

8-(4-chloro-2-(trifluoromethyl) phenyl)-4-(6-methoxy-5-(4-methyl-1H-imidazol-1-yl) pyridin-2-yl)-1, 6, 7, 8, 9, 9a-hexahydropyrazino [1, 2-d] [1, 2, 4] oxadiazine, fraction (I): ¹H NMR (CD₃OD, 400 MHz): δ 7.99 (s, 1H), 7.89 (d, 1H), 7.68-7.60 (m, 2H), 7.57 (d, 1H), 7.32 (d, 1H), 7.21 (s, 1H), 4.19-4.13 (m, 1H), 4.07 (s, 3H), 4.95-4.80 (m, 2H), 4.75-4.71 (m, 1H), 3.10-2.90 (m, 5H), 2.23 (s, 3H); Mass (ESI): 507.6 [M+1]; HPLC (purity): 99.1%; RT 8.34 mm; Chiral HPLC: 99.2% RT =15.47 mm.

### Example 36B

8-(4-chloro-2-(trifluoromethyl) phenyl)-4-(6-methoxy-5-(4-methyl-1H-imidazol-1-yl) pyridin-2-yl)-1,6,7,8,9,9a-hexahydropyrazino [1,2-d] [1,2,4] oxadiazine, fraction (II): Mass (ESI): 507.7 [M+1]; HPLC (purity): 99.1%; RT 8.34 mm; Chiral HPLC: 96.5% RT =23.98 min

### Example 37

### Synthesis of 5-(4-fluoro-3-(trifluoromethyl) phenyl)-3-(6-methoxy-5-(4-methyl-1H-imidazol-1-yl) pyridin-2-yl)-5,6-dihydro-4H-1,2,4-oxadiazine

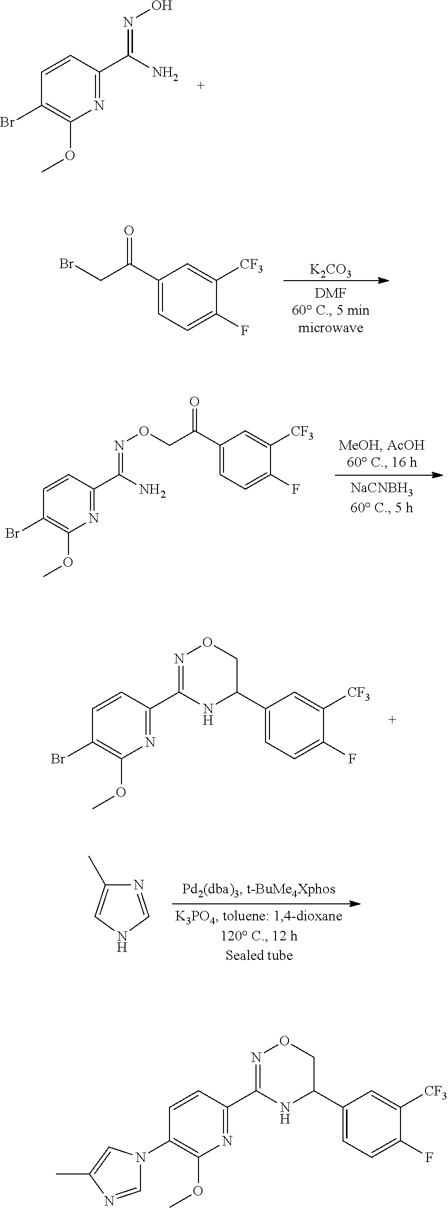

### Synthesis of (Z)-5-bromo-N′-(2-(4-fluoro-3-(trifluoromethyl) phenyl)-2-oxoethoxy)-6-methoxypicolinimidamide

To a stirred solution of (Z)-5-bromo-N′-hydroxy-6-methoxypicolinimidamide (1 g, 4 mmol) in DMF (10 mL) at room temperature under an argon atmosphere were added potassium carbonate (840 mg, 6 mmol) and 2-bromo-1-(4-fluoro-3-(trifluoromethyl) phenyl) ethan-1-one (1.4 g, 5 mmol). The reaction mixture was stirred at 60° C. for 5 mm After consumption of starting material (by TLC), the reaction mixture was diluted with water (50 mL) and extracted with EtOAc (2×50 mL). The combined organic extracts were dried over sodium sulfate, filtered and concentrated in vacuo to obtain (Z)-5-bromo-N′-(2-(4-fluoro-3-(trifluoromethyl) phenyl)-2-oxoethoxy)-6-methoxypicolinimidamide (1.6 g, crude) as brown semi solid used in the next step without further purification. LCMS: 25.5%; 449.9 (M+1); (column; Ascentis Express C-18 (50×3.0 mm, 2.7 μm); RT 2.73 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 40% EtOAc/Hexane (R_{f}:06).

### Synthesis of 3-(5-bromo-6-methoxypyridin-2-yl)-5-(4-fluoro-3-(trifluoromethyl) phenyl)-5,6-dihydro-4H-1,2,4-oxadiazine

To a stirred solution of (Z)-5-bromo-N′-(2-(4-fluoro-3-(trifluoromethyl) phenyl)-2-oxoethoxy)-6-methoxypicolinimidamide (1.6 g, 3.5 mmol) in MeOH (20 mL) at room temperature under an argon atmosphere was added acetic acid (4 mL). The reaction mixture was stirred for 16 h at 60° C. Then sodium cyanoborohydride (671 mg, 10 mmol) was added to the reaction mixture at room temperature. The reaction mixture was stirred for 5 h at 60° C. After consumption of starting material (by TLC), the reaction mixture was basified with saturated sodium bicarbonate solution (10 mL) and extracted with CH₂Cl₂ (2×10 mL). The combined organic extracts were dried over sodium sulfate, filtered and concentrated in vacuo to obtain 3-(5-bromo-6-methoxypyridin-2-yl)-5-(4-fluoro-3-(trifluoromethyl) phenyl)-5,6-dihydro-4H-1,2,4-oxadiazine (620 mg, crude) as brown semi solid. LCMS: 61.6%; 433.9 (M+1); (column; Ascentis Express C-18 (50×3.0 mm, 2.7 μm); RT 2.84 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% EtOAc/Hexane (R_{f}:02).

### Synthesis of 5-(4-fluoro-3-(trifluoromethyl) phenyl)-3-(6-methoxy-5-(4-methyl-1H-imidazol-1-yl) pyridin-2-yl)-5,6-dihydro-4H-1,2,4-oxadiazine

To a dry vial was added a suspension of Pd₂(dba)₃ (10 mg, 0.05 mmol) and tert-butyl tetramethyl Xphos (10 mg, 0.01 mmol) in toluene: 1,4-dioxane (2:1, 0.75 mL) at room temperature. The suspension was degassed with argon, heated to 120° C., and stirred at 120° C. for 3 min. A mixture of 3-(5-bromo-6-methoxypyridin-2-yl)-5-(4-fluoro-3-(trifluoromethyl) phenyl)-5,6-dihydro-4H-1,2,4-oxadiazine (50 mg, 0.1 mmol), 4-methyl-1H-imidazole (11 mg, 0.1 mmol) and potassium phosphate (49 mg, 0.2 mmol) in toluene: 1,4-dioxane (2:1, 0.75 mL) was degassed and the catalyst premix was added. The resulting mixture was stirred at 120° C. for 12 h in a sealed tube. After consumption of the starting material (monitored by TLC and LCMS), the volatiles were concentrated in vacuo. The crude material was purified by column chromatography using 20%EtOAc: Hexane to afford 5-(4-fluoro-3-(trifluoromethyl) phenyl)-3-(6-methoxy-5-(4-methyl-1H-imidazol-1-yl) pyridin-2-yl)-5,6-dihydro-4H-1,2,4-oxadiazine (35 mg, 70%) as an off-white solid.

Racemic compound of Example 37 was separated using a Chiralpak-ODH column (250×20 mm, 5 μm) (40 mg loading; 0.1% DEA in n-Hexane:EtOH:MeOH (50:50) (A:B: 70:30) as mobile phase; flow rate: 20 mL/min) to provide the compound of Example 37A (Fraction I (−)) and the compound of Example 37B (Fraction II (+)); TLC: 40%EtOAc/Hexane (R_{f}:03).

Analytical conditions for Example 37A and Example 37B. HPLC (purity): (column; zorbax-SB-C-18 150×4.6 mm, 3.5 μm); mobile Phase: ACN: 0.05% Aq TFA; flow rate: 1.0 mL/min; Gradient program: T/B % 0.01/90, 2/90, 8/10, 15/10: diluent: CH₃CN: Water; Chiral HPLC: (Chiralcel-ODH (250×4.6 mm, 5 μm; mobile phase (A) 0.1% DEA in n-Hexane (B) EtOH:MeOH (50:50) (A::B; 70:30); flow Rate: 1.0 mL/min).

### Example 37A

(−)-5-(4-fluoro-3-(trifluoromethyl) phenyl)-3-(6-methoxy-5-(4-methyl-1H-imidazol-1-yl) pyridin-2-yl)-5,6-dihydro-4H-1,2,4-oxadiazine, fraction (I), (−): Mass (ESI): 436.1 [M+1]; HPLC (purity): 98.1%; RT 7.70 min; Chiral HPLC: 99.8% RT=8.97 min; Optical rotation [α]_{D}^{19.99}: −167.16 (c=0.25, CH₂Cl₂).

### Example 37B

(+)-5-(4-fluoro-3-(trifluoromethyl) phenyl)-3-(6-methoxy-5-(4-methyl-1H-imidazol-1-yl) pyridin-2-yl)-5,6-dihydro-4H-1,2,4-oxadiazine, fraction (II), (+): ¹H NMR (CD₃OD, 500 MHz): δ 8.13 (s, 1H), 8.04 (d, 1H), 7.88-7.83 (m, 2H), 7.80 (d, 1H), 7.51 (t, 1H), 7.37 (s, 1H), 5.12 (t, 1H), 4.24 (s, 3H), 4.23-4.21 (m, 1H), 4.17-4.14 (m, 1H), 2.40 (s, 3H); Mass (ESI): 436 [M+1]; HPLC (purity): 96.9%; RT 7.70 min; Chiral HPLC: 99.7% RT=15.19 min; Optical rotation [α]_{D}^{19.97}: +145.56 (c=0.25, CH₂Cl₂).

### Example 38

### Synthesis of 5-(4-chlorophenyl)-3-(6-methoxy-5-(4-methyl-1H-imidazol-1-yl) pyridin-2-yl)-5,6-dihydro-4H-1,2,4-oxadiazine

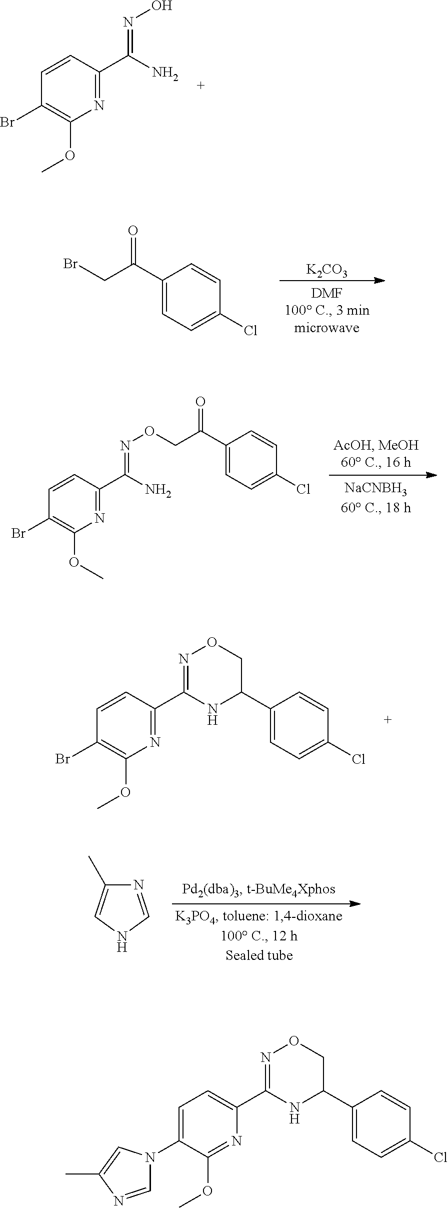

### Synthesis of (Z)-5-bromo-N′-(2-(4-chlorophenyl)-2-oxoethoxy)-6-methoxypicolinimidamide

To a stirred solution of (Z)-5-bromo-N′-hydroxy-6-methoxypicolinimidamide (1 g, 4 mmol) in DMF (10 mL) at room temperature under an argon atmosphere were added potassium carbonate (1.1 g, 8 mmol) and 2-bromo-1-(4-chlorophenyl) ethan-1-one (1.4 g, 6 mmol). The reaction mixture was stirred at 100° C. for 3 min in microwave. After consumption of starting material (by TLC), the reaction mixture was diluted with water (50 mL) and extracted with EtOAc (2×50 mL). The combined organic extracts were dried over sodium sulfate, filtered and concentrated in vacuo to obtain (Z)-5-bromo-N′-(2-(4-chlorophenyl)-2-oxoethoxy)-6-methoxypicolinimidamide (1.2 g, crude) as brown solid used in the next step without further purification. LCMS: 33.9%; 399.7 (M+1); (column; Ascentis Express C-18 (50×3.0 mm, 2.7 μm); RT 2.96 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:05).

### Synthesis of 3-(5-bromo-6-methoxypyridin-2-yl)-5-(4-chlorophenyl)-5,6-dihydro-4H-1,2,4-oxadiazine

To a stirred solution of (Z)-5-bromo-6-methoxy-N′-(2-(4-(methylsulfonyl) phenyl)-2-oxoethoxy) picolinimidamide (4.6 g, 11 mmol) in MeOH (90 mL) at room temperature under an argon atmosphere was added acetic acid (18.4 mL). The reaction mixture was stirred for 16 h at 60° C. Then sodium cyanoborohydride (1.09 g, 17 mmol) was added to the reaction mixture at room temperature. The reaction mixture was stirred at 60° C. for 18 h. After consumption of starting material (by TLC), the reaction mixture was diluted with saturated sodium bicarbonate solution (100 mL) and extracted with CH₂Cl₂ (2×100 mL). The combined organic extracts were dried over sodium sulfate, filtered and concentrated in vacuo to obtain 3-(5-bromo-6-methoxypyridin-2-yl)-5-(4-chlorophenyl)-5,6-dihydro-4H-1,2,4-oxadiazine (1.6 g, 36%) as brown semi solid. LCMS: 89.8%; 383.4 (M+1); (column; Ascentis Express C-18 (50×3.0 mm, 2.7 μm); RT 2.81 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:05).

### Synthesis of 5-(4-chlorophenyl)-3-(6-methoxy-5-(4-methyl-1H-imidazol-1-yl) pyridin-2-yl)-5,6-dihydro-4H-1,2,4-oxadiazine

To a dry vial was added a suspension of Pd₂(dba)₃ (35 mg, 0.04 mmol) and tert-butyl tetramethyl Xphos (37 mg, 0.08 mmol) in toluene: 1,4-dioxane (2:1, 6 mL) at room temperature. The suspension was degassed with argon, heated to 120° C., and stirred at 120° C. for 3 min. A mixture of 3-(5-bromo-6-methoxypyridin-2-yl)-5-(4-chlorophenyl)-5,6-dihydro-4H-1,2,4-oxadiazine (300 mg, 0.8 mmol), 4-methyl-1H-imidazole (128 mg, 1.8 mmol) and potassium phosphate (332 mg, 1.6 mmol) in toluene: 1,4-dioxane (2:1, 6 mL) was degassed and the catalyst premix was added. The resulting mixture was stirred at 100° C. for 2 h in a sealed tube. After consumption of the starting material (monitored by TLC and LCMS), the reaction mixture was diluted with water (30 mL) and extracted with EtOAc (2×30 mL). The combined organic extracts were dried over sodium sulfate, filtered and concentrated in vacuo. The crude material was purified by column chromatography using 3%MeOH:CH₂Cl₂to afford 5-(4-chlorophenyl)-3-(6-methoxy-5-(4-methyl-1H-imidazol-1-yl) pyridin-2-yl)-5,6-dihydro-4H-1,2,4-oxadiazine (350 mg, crude) as an off-white solid.

Racemic compound of Example 38 was separated using a Chiralpak-ADH column (250×20 mm, 5 μm) (50 mg loading; 0.1% DEA in n-Hexane:EtOH:MeOH (50:50) (A:B: 70:30) as mobile phase; flow rate: 20 mL/min) to provide the compound of Example 38A (Fraction I (−)) and the compound of Example 38B (Fraction II (+));TLC: 5%MeOH/CH₂Cl₂ (R_{f}:03).

Analytical conditions for Example 38A and Example 38B: HPLC (purity): (column; zorbax-SB-C-18 150×4.6 mm, 5 μm); mobile Phase: ACN: 0.05% Aq TFA; flow rate: 1.0 mL/min; Gradient program: T/B % 0.01/90, 2/90, 8/10, 15/10: diluent: CH₃CN: Water; Chiral HPLC: (Chiralpak-ADH (250×4.6 mm, 5 μm; mobile phase (A) 0.1% DEA in n-Hexane (B) EtOH:MeOH (50:50) (A::B; 70:30); flow Rate: 1.0 mL/min).

### Example 38A

(−)-5-(4-chlorophenyl)-3-(6-methoxy-5-(4-methyl-1H-imidazol-1-yl) pyridin-2-yl)-5,6-dihydro-4H-1,2,4-oxadiazine, fraction (I), (−). Mass (ESI): 383.9 [M+1]; HPLC (purity): 95.8%; RT 7.37 mm; Chiral HPLC: 97.8% RT=8.40 min: Optical rotation [α]_{D}^{20.00}: −205.15 (c=0.25, CH₂Cl₂).

### Example 38B

(+)-5-(4-chlorophenyl)-3-(6-methoxy-5-(4-methyl-1H-imidazol-1-yl) pyridin-2-yl)-5,6-dihydro-4H-1,2,4-oxadiazine, fraction (II), (+). ¹H NMR (CD₃OD, 500 MHz): δ 7.97 (s, 1H), 7.88 (d, 1H), 7.64 (d, 1H), 7.38 (s, 4H), 7.22 (s, 1H), 4.86 (t, 1H), 4.10 (d, 1H), 4.08 (s, 3H), 3.92 (dd, 1H), 2.25 (s, 3H); Mass (ESI): 383.9 [M+1]; HPLC (purity): 98.4%; RT 7.38 mm; Chiral HPLC: 99.3% RT=13.48 mm; Optical rotation [α]_{D}^{20.01}: +209.95 (c=0.25, CH₂Cl₂).

### Example 39

### Synthesis of 5-(5,6-difluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine

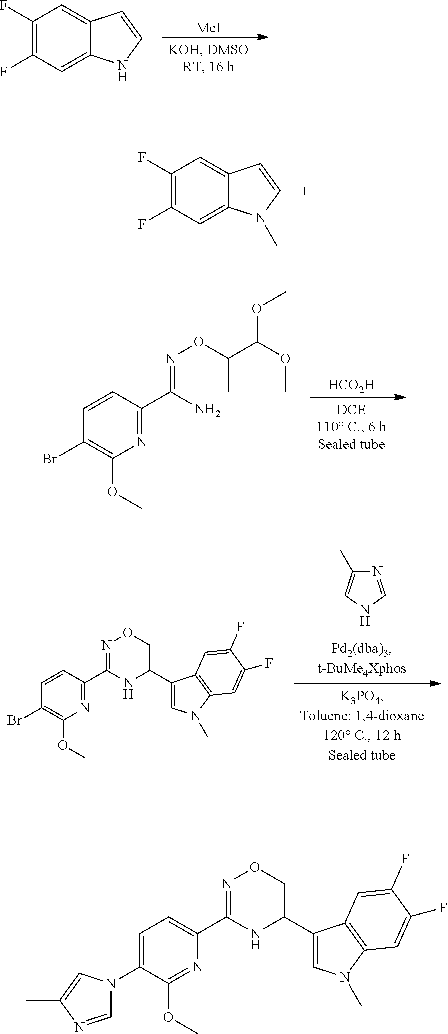

### Synthesis of 5,6-difluoro-1-methyl-1H-indole

To a stirred solution of 5,6-difluoro-1H-indole (1 g, 7 mol) in DMSO (5 mL) at 0° C. under an argon atmosphere were added potassium hydroxide (402 mg, 7 mmol) and methyl iodide (1.14 g, 9 mmol). The reaction mixture was warmed to room temperature and stirred for 16 h. After consumption of starting material (by TLC), the reaction mixture was diluted with water (50 mL) and extracted with EtOAc (2×50 mL). The combined organic extracts were dried over sodium sulfate, filtered and concentrated in vacuo to obtain 5,6-difluoro-1-methyl-1H-indole (1 g, 91%) as brown solid used in the next step without further purification. ¹H NMR (DMSO-d₆, 500 MHz): δ 7.55-7.50 (m, 2H), 7.38 (s, 1H), 6.42 (s, 1H), 3.76 (s, 3H); TLC: 30% EtOAc/Hexane (R_{f}:06).

### Synthesis of 3-(5-bromo-6-methoxypyridin-2-yl)-5-(5,6-difluoro-1-methyl-1H-indo1-3-yl)-6-methyl-5,6-dihydro-4H-1,2,4-oxadiazine

To a stirred solution of (Z)-5-bromo-N((1,1-dimethoxypropan-2-yl) oxy)-6-methoxypicolinimidamide (600 mg, 2 mmol) in 1,2-dichloro ethane (12 mL) at room temperature under an argon atmosphere was added 5,6-difluoro-1-methyl-1H-indole (572 mg, 3 mmol) and formic acid (12 mL). The reaction mixture was stirred at 110° C. for 6 h in a sealed tube. After consumption of starting material (by TLC), the reaction mixture was diluted with sodium bicarbonate solution (20 mL) and extracted with EtOAc (2×20 mL). The combined organic extracts were dried over sodium sulfate, filtered and concentrated in vacuo. The crude material was purified by column chromatography using 2% MeOH:CH₂Cl₂ to afford 3-(5-bromo-6-methoxypyridin-2-yl)-5-(5,6-difluoro-1-methyl-1H-indo1-3-yl)-6-methyl-5,6-dihydro-4H-1,2,4-oxadiazine (600 mg, 66%) as an off-white solid.

¹H NMR (CD₃OD, 400 MHz): δ 8.01 (d, 1H), 7.50-7.41 (m, 2H), 7.38-7.31 (m, 2H), 4.58 (d, 1H), 3.90 (s, 3H), 3.84-3.80 (m, 1H), 3.79 (s, 3H), 1.25 (d, 3H); TLC: 5% MeOH/CH₂Cl₂ (R_{f}:02).

### Synthesis of 5-(5,6-difluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine

To a dry vial was added a suspension of Pd₂(dba)₃ (40 mg, 0.04 mmol) and tert-butyl tetramethyl Xphos (42 mg, 0.08 mmol) in toluene: 1,4-dioxane (2:1, 6 mL) at room temperature. The suspension was degassed with argon, heated to 120° C., and stirred at 120° C. for 3 min. A mixture of methyl 3-(5-bromo-6-methoxypyridin-2-yl)-5-(5,6-difluoro-1-methyl-1H-indol-3-yl)-6-methyl-5,6-dihydro-4H-1,2,4-oxadiazine (400 mg, 1 mmol), 4-methyl-1H-imidazole (145 mg, 2 mmol) and potassium phosphate (376 mg, 2 mmol) in toluene: 1,4-dioxane (2:1, 6 mL) was degassed and the catalyst premix was added. The resulting mixture was stirred at 120° C. for 6 h in a sealed tube. After consumption of the starting material (monitored by TLC and LCMS), the reaction mixture was filtered and the filtrate was concentrated in vacuo. The crude material was purified by column chromatography using 3%MeOH:CH₂Cl₂ to afford 5-(5,6-difluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine (250 mg, 62%) as an off-white solid.

### Separation of Diastereomers:

Racemic compound of Example 39 was separated using a YMC silica column (250×20 mm, (50 mg loading; n-Hexane: CH₂Cl₂:MeOH (50:50) (A:B: 80:20) as mobile phase; flow rate: 20 mL/min) to provide the compounds of Example 39X and Example 39Y.

Analytical conditions for Example 39X and Example 39Y: HPLC: column; X-Select CSH-C-18 (150×4.6 mm, 3.5 μm); mobile Phase: ACN: 0.5% TFA; flow rate: 1.0 mL/min; Gradient program: T/B % 0.01/90, 10/10, 15/10: diluent: CH₃CN: Water.

### Example 39X

5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 98.5%; RT 7.08 min

### Example 39Y

5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 84.6%; RT 7.54 min

### Separation of Enantiomers:

Racemic compound of Example 39X was separated using a Chiralpak-IA column (250×20 mm, 5 μm) (20 mg loading; 0.1% DEA in n-Hexane: CH₂Cl₂:MeOH (90:10) (A:B: 75:25) as mobile phase; flow rate: 20 mL/min) to afford the compounds of Example 39A (Fraction I (+)) and Example 39B (Fraction II (−)).

Analytical conditions for Example 39A and Example 39B. HPLC (purity): (column; zorbax-SB-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralcel-ADH (250×4.6 mm, 5 μm; mobile phase (A) 0.1% DEA in n-Hexane (B) EtOH:MeOH (50:50) (A::B; 75:25); flow Rate: 1.0 mL/min):

### Example 39A

(+)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (I), (+): ¹H NMR (CD₃OD, 400 MHz): δ 7.96 (s, 1H), 7.89 (d, 1H), 7.65 (d, 1H), 7.47 (dd, 1H), 7.39 (s, 1H), 7.35 (dd, 1H), 7.21 (s, 1H), 4.60 (d, 1H), 3.94 (s, 3H), 3.80-3.79 (m, 1H), 3.79 (s, 3H), 2.25 (s, 3H), 1.25 (d, 3H); Mass (ESI): 453 1M+11; HPLC (purity): 98.3%; RT 7.33 mm; Chiral HPLC: 100% RT=12.23 mm; Optical rotation [α]_{D}^{20.01}: +106.28 (c=0.25, CH₂Cl₂).

### Example 39B

(−)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (II), (−): Mass (ESI): 453 [M+1]; HPLC (purity): 99.5%; RT 7.34 min; Chiral HPLC: 99.2% RT=20.40 min; Optical rotation [α]_{D}^{20.01}: −100.06 (c=0.25, CH₂Cl₂).

Racemic compound of Example 39Y was separated using a Chiralpak-IA column (250×20 mm, 5 μm) (20 mg loading; 0.1% DEA in n-Hexane: CH₂Cl₂:MeOH (90:10) (A:B: 75:25) as mobile phase; flow rate: 20 mL/min) to afford the compounds of Example 39C (Fraction III (−)) and Example 39D (Fraction IV (+)).

Analytical conditions for Example 39C and Example 39D. HPLC (purity): (column; X-select-CSH-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralcel-ADH (250×4.6 mm, 5 μm; mobile phase (A) 0.1% DEA in n-Hexane (B) EtOH:MeOH (50:50) (A::B; 75:25); flow Rate: 1.0 mL/min):

### Example 39C

(−)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (III), (−): Mass (ESI): 453.1 [M+1]; HPLC (purity): 98.4%; RT 7.04 mm; Chiral HPLC: 100% RT=8.07 min; Optical rotation [α]_{D}^{20.01}: −52.88 (c=0.25, CH₂Cl₂).

### Example 39D

(+)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (IV), (+): ¹H NMR (CD₃OD, 400 MHz): δ 7.96 (s, 1H), 7.89 (d, 1H), 7.65 (d, 1H), 7.47 (dd, 1H), 7.39 (s, 1H), 7.35 (dd, 1H), 7.21 (s, 1H), 4.60 (d, 1H), 3.94 (s, 3H), 3.86-3.82 (m, 1H), 3.79 (s, 3H), 2.25 (s, 3H), 1.25 (d, 3H); Mass (ESI): 453 1M+11; HPLC (purity): 98.4%; RT 7.04 mm; Chiral HPLC: 98.6% RT=10.51 mm; Optical rotation [α]_{D}^{20.01}: +53.77 (c=0.25, CH₂Cl₂).

### Example 40-1

### Synthesis of 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine

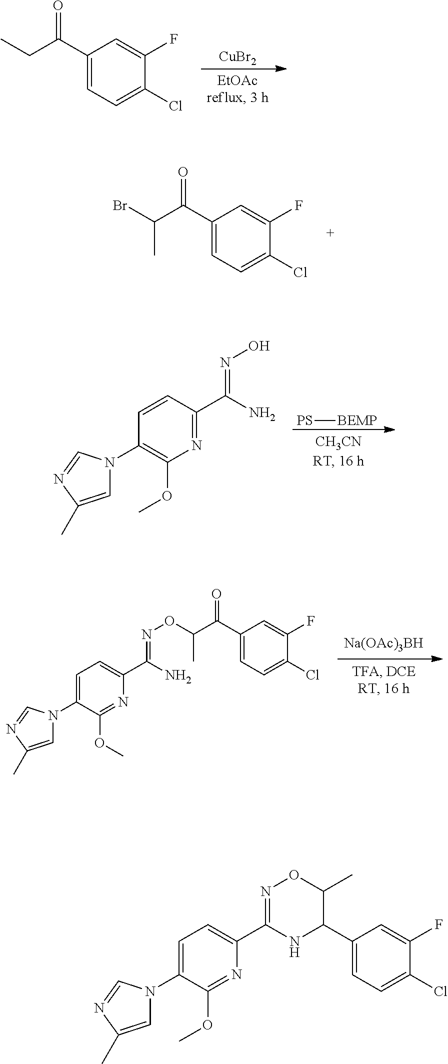

### Synthesis of 2-bromo-1-(4-chloro-3-fluorophenyl) propan-1-one

To a stirred solution of 1-(4-chloro-3-cyclopropylphenyl) ethan-1-one (500 mg, 3 mmol) in EtOAc (25 mL) at room temperature under an argon atmosphere was added copper bromide (1.14 g, 5 mmol). The reaction mixture was stirred for 4 h at 80° C. After consumption of starting material (by TLC), the reaction mixture was filtered. The filtrate was concentrated in vacuo to obtain 2-bromo-1-(4-chloro-3-cyclopropylphenyl) ethan-1-one (460 mg, crude) as brown syrup used in the next step without further purification. ¹H NMR (DMSO-d₆, 400 MHz): δ 8.00 (d, 1H), 7.90-7.70 (m, 2H), 5.83-5.80 (m, 1H), 1.79 (d, 3H); TLC: 10% EtOAc/Hexane (R_{f}:06).

### Synthesis of (Z)-N′-((1-(4-chloro-3-fluorophenyl)-1-oxopropan-2-yl) oxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (300 mg, 1.2 mmol) in CH₃CN (3 mL) at room temperature under an argon atmosphere was added PS-BEMP (600 mg). The reaction mixture was stirred for 5 mm at room temperature. Then 2-bromo-1-(4-chloro-3-cyclopropylphenyl) ethan-1-one (480 mg, 2 mmol) in CH₃CN (3 mL) was added to the reaction mixture at room temperature. The reaction mixture was stirred for 16 h at room temperature. After consumption of starting material (by TLC), the volatiles were concentrated in vacuo to obtain (Z)-N′-((1-(4-chloro-3-fluorophenyl)-1-oxopropan-2-yl) oxy)-6-methoxy-5-(4-methyl-1H-imidazol-1-yl) picolinimidamide (500 mg, crude) as brown semi solid used in the next step without further purification. LCMS: 43.6%; 431.9 (M+1); (column; Ascentis Express C-18 (50×3.0 mm, 2.7 μm); RT 2.21 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 70% EtOAc/Hexane (R_{f}:05).

### Synthesis of 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine

To a stirred solution of (Z)-N′-((1-(4-chloro-3-fluorophenyl)-1-oxopropan-2-yl) oxy)-6-methoxy-5-(4-methyl-1H-imidazol-1-yl) picolinimidamide (500 g, 1 mmol) in 1, 2-dichloro ethane (10 mL) at room temperature under an argon atmosphere were added trifluoroacetic acid (2 mL) and sodium triacetoxyborohydride (491 mg, 2 mmol). The reaction mixture was stirred for 16 h at room temperature. After consumption of starting material (by TLC), the reaction mixture was diluted with saturated sodium bicarbonate solution (20 mL) and extracted with CH₂Cl₂ (2×20 mL). The combined organic extracts were dried over sodium sulfate, filtered and concentrated in vacuo. The crude material was purified by column chromatography using 2% MeOH:CH₂Cl₂ to afford 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine (210 mg, 44%) as an off-white solid.

Racemic compound of Example 40-I was separated using a Chiralpak-ODH column (250×20 mm, 5 μ) (30 mg loading; 0.1% DEA in n-Hexane:EtOH:MeOH (50:50) (A:B: 70:30) as mobile phase; flow rate: 20 mL/min) to afford the compounds of Example 40-IA (Fraction I (−)) and Example 40-IB (Fraction II (+)).

Analytical conditions for Example 40-IA and Example 40-IB. HPLC (purity): (column; Zorbax SBC-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-ODH (250×4.6 mm, 5 μm; mobile phase (A) 0.1% DEA in n-Hexane (B) EtOH:MeOH (50:50) (A::B; 70:30); flow Rate: 1.0 mL/min).

### Example 40-IA

(+)-5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine, fraction (I) (−): Mass (ESI): 416 [M+1]; HPLC (purity): 99.3%; RT 7.29 mm; Chiral HPLC: 99.7% RT=9.54 mm; Optical rotation [α]_{D}^{20.01}: −200.59 (c=0.25, CH₂Cl₂).

### Example 40-IB

(+)-5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine, fraction (II) (+): ¹H NMR (CD₃OD, 400 MHz): δ 7.99 (s, 1H), 7.89 (d, 1H), 7.69 (d, 1H), 7.47 (t, 1H), 7.23 (s, 1H), 7.19-7.10 (m, 2H), 4.74 (d, 1H), 4.09 (s, 3H), 4.04 (dd, 1H), 2.26 (s, 3H), 1.02 (d, 3H); Mass (ESI): 416 [M+1]; HPLC (purity): 99.4%; RT 7.29 min; Chiral HPLC: 100% RT=13.06 min; Optical rotation [α]_{D}^{20.03}: +201.04 (c=0.25, CH₂Cl₂).

### Example 40-II

### Synthesis of 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine

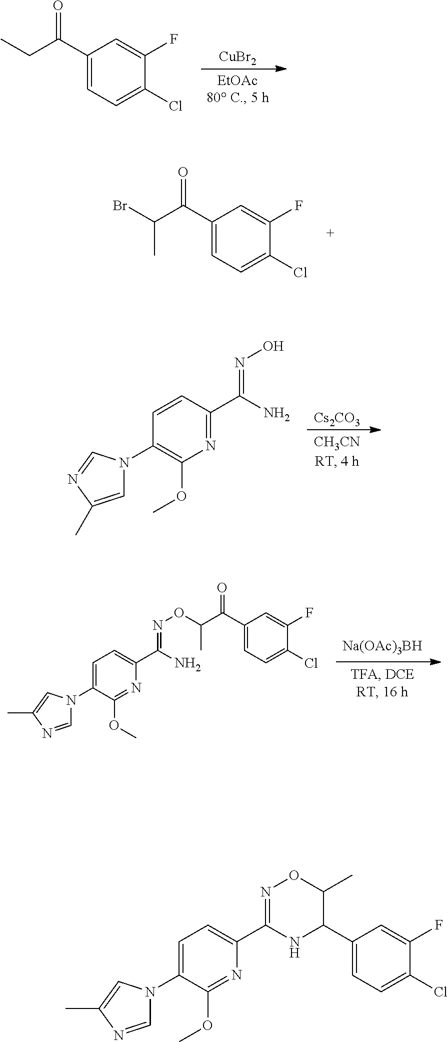

### Synthesis of 2-bromo-1-(4-chloro-3-fluorophenyl) propan-1-one

To a stirred solution of 1-(4-chloro-3-cyclopropylphenyl) ethan-1-one (20 g, 107 mmol) in EtOAc (200 mL) at room temperature under an argon atmosphere was added copper bromide (47.8 g, 214 mmol). The reaction mixture was stirred at 80° C. for 5 h. After consumption of starting material (by TLC), the reaction mixture was filtered. The filtrate was concentrated in vacuo. The crude material was purified by column chromatography using 2-3% Acetone: Hexane to afford 2-bromo-1-(4-chloro-3-fluorophenyl) propan-1-one (20.5 g, 70%) as brown syrup. ¹H NMR (DMSO-d₆, 400 MHz): δ 8.00 (d, 1H), 7.90-7.70 (m, 2H), 5.83-5.80 (m, 1H), 1.79 (d, 3H); TLC: 5% Acetone/Hexane (R_{f}:03).

### Synthesis of (Z)-N′-((1-(4-chloro-3-fluorophenyl)-1-oxopropan-2-yl) oxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (10 g, 40 mmol) in CH₃CN (500 mL) at room temperature under an argon atmosphere was added cesium carbonate (26.31 g, 80 mmol). The reaction mixture was stirred for 5 min at room temperature. Then 2-bromo-1-(4-chloro-3-cyclopropylphenyl) ethan-1-one (16.09 g, 61 mmol) was added to the reaction mixture at room temperature. The reaction mixture was stirred for 4 h at room temperature. After consumption of starting material (by TLC), the reaction mixture was filtered. The filtrate was concentrated in vacuo to obtain (Z)-N′-((1-(4-chloro-3-fluorophenyl)-1-oxopropan-2-yl) oxy)-6-methoxy-5-(4-methyl-1H-imidazol-1-yl) picolinimidamide (13 g, crude) as brown semi solid used in the next step without further purification. LCMS: 43.6%; 431.9 (M+1); (column; Ascentis Express C-18 (50×3.0 mm, 2.7 μm); RT 2.21 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:05).

### Synthesis of 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine

To a stirred solution of (Z)-N′-((1-(4-chloro-3-fluorophenyl)-1-oxopropan-2-yl) oxy)-6-methoxy-5-(4-methyl-1H-imidazol-1-yl) picolinimidamide (13 g, 30 mmol) in 1,2-dichloro ethane (390 mL) at room temperature under an argon atmosphere were added trifluoroacetic acid (7.19 g, 151 mmol) and sodium triacetoxyborohydride (19.09 g, 90 mmol). The reaction mixture was stirred for 16 h at room temperature. After consumption of starting material (by TLC), the reaction mixture was diluted with saturated sodium bicarbonate solution (800 mL) and extracted with 5% MeOH:CH₂Cl₂ (2×500 mL). The combined organic extracts were dried over sodium sulfate, filtered and concentrated in vacuo. The crude material was purified by column chromatography using 3% MeOH:CH₂Cl₂ to afford 5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine (3.7 g, 68%) as an off-white solid.

Racemic compound of Example 40-II was separated using a Chiralpak-ODH column (250×20 mm, 5 μ) (30 mg loading; 0.1% DEA in n-Hexane:EtOH:MeOH (50:50) (A:B: 70:30) as mobile phase; flow rate: 20 mL/min) to afford the compounds of Example 40-IIA (Fraction I (−)) and Example 40-IIB (Fraction II (+)).

Analytical conditions for Example 40-IIA and Example 40-IIB. HPLC (purity): (column; Zorbax SBC-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-ODH (250×4.6 mm, 5 μm; mobile phase (A) 0.1% DEA in n-Hexane (B) EtOH:MeOH (50:50) (A::B; 70:30); flow Rate: 1.0 mL/min).

### Example 40-IIA

(−)-5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine, fraction (I) (−): Mass (ESI): 416 [M+1]; HPLC (purity): 99.3%; RT 7.29 min; Chiral HPLC: 99.7% RT=9.54 min; Optical rotation [α]_{D}^{20.01}: −200.59 (c=0.25, CH₂Cl₂).

### Example 40-IIB

(+)-5-(4-chloro-3-fluorophenyl)-3-(6-methoxy-5-(4-methyl-1H-imidazol-1-yl) pyridin-2-yl)-6-methyl-5,6-dihydro-4H-1,2,4-oxadiazine, fraction (II) (+): ¹H NMR (CD₃OD, 400 MHz): δ 7.99 (s, 1H), 7.89 (d, 1H), 7.69 (d, 1H), 7.47 (t, 1H), 7.23 (s, 1H), 7.19-7.10 (m, 2H), 4.74 (d, 1H), 4.09 (s, 3H), 4.04 (dd, 1H), 2.26 (s, 3H), 1.02 (d, 3H); Mass (ESI): 416 [M+1]; HPLC (purity): 99.4%; RT 7.29 min; Chiral HPLC: 100% RT=13.06 min; Optical rotation [α]_{D}^{20.03}: +201.04 (c=0.25, CH₂Cl₂).

### Example 41

### Synthesis of 5-(3-chlorophenyl)-3-(6-methoxy-5-(4-methyl-1H-imidazol-1-yl) pyridin-2-yl)-6-methyl-5,6-dihydro-4H-1,2,4-oxadiazine

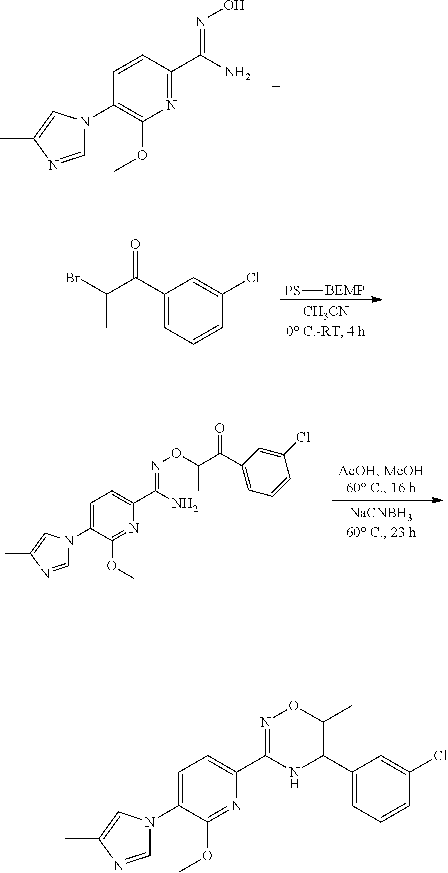

### Synthesis of (Z)-N′-((1-(3-chlorophenyl)-1-oxopropan-2-yl) oxy)-6-methoxy-5-(4-methyl-1H-imidazol-1-yl) picolinimidamide

To a stirred solution of (Z)-5-bromo-N′-hydroxy-6-methoxypicolinimidamide (800 mg, 3 mmol) in CH₃CN (20 mL) at room temperature under an argon atmosphere was added PS-BEMP (1.76 g). The reaction mixture was stirred for 5 mm at room temperature. Then 2-bromo-1-(3-chlorophenyl) propan-1-one (1.2 g, 5 mmol) in CH₃CN (20 mL) was added to the reaction mixture at room temperature. The reaction mixture was stirred at room temperature for 4 h. After consumption of starting material (by TLC), the reaction mixture was filtered and the filtrate was concentrated in vacuo to obtain (Z)-N′-((1-(3-chlorophenyl)-1-oxopropan-2-yl) oxy)-6-methoxy-5-(4-methyl-1H-imidazol-1-yl) picolinimidamide (1.5 g, crude) as brown semi solid used in the next step without further purification. LCMS: 67.1%; 414 (M+1); (column; Ascentis Express C-18 (50×3.0 mm, 2.7 μm); RT 2.19 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:05).

### Synthesis of 5-(3-chlorophenyl)-3-(6-methoxy-5-(4-methyl-1H-imidazol-1-yl) pyridin-2-yl)-6-methyl-5,6-dihydro-4H-1,2,4-oxadiazine

To a stirred solution of (Z)-N′-((1-(3-chlorophenyl)-1-oxopropan-2-yl) oxy)-6-methoxy-5-(4-methyl-1H-imidazol-1-yl) picolinimidamide (1.5 g, 4 mmol) in MeOH (40 mL) at room temperature under an argon atmosphere was added acetic acid (5 mL). The reaction mixture was stirred for 16 h at 60° C. Then sodium cyanoborohydride (275 mg, 4 mmol) was added to the reaction mixture at room temperature. The reaction mixture was stirred for 23 h at 60° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 2% MeOH:CH₂Cl₂ to afford 5-(3-chlorophenyl)-3-(6-methoxy-5-(4-methyl-1H-imidazol-1-yl)pyridin-2-yl)-6-methyl-5,6-dihydro-4H-1,2,4-oxadiazine (700 mg, 50%) as an off-white solid.

Racemic compound of Example 41 was separated using a Chiralpak-IB column (250×20 mm, 5 μm) (15 mg loading; 0.1% DEA in n-Hexane: CH₂Cl₂:MeOH (50:50) (A:B: 90:10) as mobile phase; flow rate: 20 mL/min) to afford the compounds of Example 41A (Fraction I (−)) and Example 41B (Fraction II (+)).

Analytical conditions for Example 41A and Example 41B. HPLC (purity): (column; Zorbox SB-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 85:15); flow Rate: 1.0 mL/min).

### Example 41A

5-(3-chlorophenyl)-3-(6-methoxy-5-(4-methyl-1H-imidazol-1-yl) pyridin-2-yl)-6-methyl-5,6-dihydro-4H-1,2,4-oxadiazine, fraction (I) (−): Mass (ESI): 398 [M+1]; HPLC (purity): 96.4%; RT 7.11 mm; Chiral HPLC: 96.4% RT=11.96 mm; Optical rotation [α]_{D}^{19.97}: −119.42 (c=0.25, CH₂Cl₂).

### Example 41B

5-(3-chlorophenyl)-3-(6-methoxy-5-(4-methyl-1H-imidazol-1-yl) pyridin-2-yl)-6-methyl-5,6-dihydro-4H-1,2,4-oxadiazine, fraction (II) (+): ¹H NMR (DMSO-d₆, 400 MHz): δ 7.99 (d, 1H), 7.95-7.90 (m, 2H), 7.66 (d, 1H), 7.41-7.34 (m, 2H), 7.28-7.20 (m, 3H), 4.71 (dd, 1H), 4.02 (s, 3H), 3.90 (dd, 1H), 2.16 (s, 3H), 0.91 (d, 3H); Mass (ESI): 398 [M+1]; HPLC (purity): 99.8%; RT 7.11 mm; Chiral HPLC: 100% RT=14.56 mm; Optical rotation [α]_{D}^{19.99}: +132.72 (c=0.25, CH₂Cl₂).

### Example 42

### Synthesis of 3-(5-(4-chloro-1H-imidazol-1-yl)-6-methoxypyridin-2-yl)-5-(4-chlorophenyl)-6-methyl-5,6-dihydro-4H-1,2,4-oxadiazine

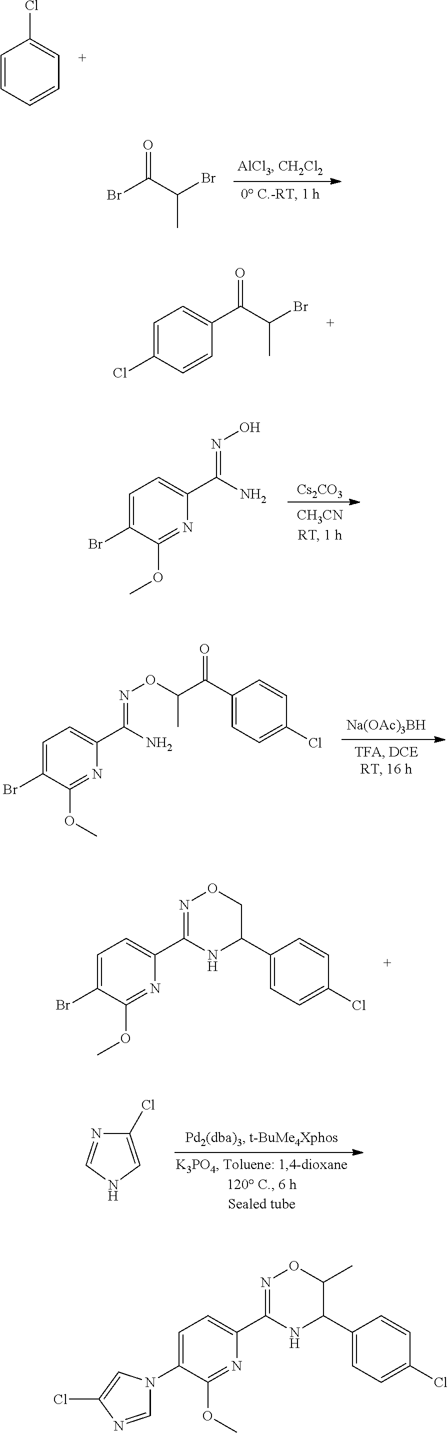

### Synthesis of 3-(5-(4-chloro-M-imidazol-1-yl)-6-methoxypyridin-2-yl)-5-(4-chlorophenyl)-6-methyl-5,6-dihydro-4H-1,2,4-oxadiazine

To a dry vial was added a suspension of Pd₂(dba)₃ (46 mg, 0.05 mmol) and tert-butyl tetramethyl Xphos (49 mg, 0.10 mmol) in toluene: 1,4-dioxane (2:1, 0.3 mL) at room temperature. The suspension was degassed with argon, heated to 120° C., and stirred at 120° C. for 3 min. A mixture of 3-(5-bromo-6-methoxypyridin-2-yl)-5-(4-chlorophenyl)-6-methyl-5,6-dihydro-4H-1,2,4-oxadiazine (250 mg, 1 mmol), 4-chloro-1H-imidazole (123 mg, 1 mmol) and potassium phosphate (430 mg, 2 mmol) in toluene: 1,4-dioxane (2:1, 0.3 mL) was degassed and the catalyst premix was added. The resulting mixture was stirred at 120° C. for 6 h in a sealed tube. After consumption of the starting material (monitored by TLC and LCMS), the reaction mixture was filtered and the filtrate was concentrated in vacuo. The crude material was purified by column chromatography using 3%MeOH:CH₂Cl₂ to afford 3-(5-(4-chloro-1H-imidazol-1-yl)-6-methoxypyridin-2-yl)-5-(4-chlorophenyl)-6-methyl-5,6-dihydro-4H-1,2,4-oxadiazine (160 mg, 37%) as an off-white solid.

Racemic compound of Example 42 was separated using a Chiralpak-IB column (250×20 mm, 5 μm) (25 mg loading; 0.1% DEA in n-Hexane: CH₂Cl₂:MeOH (50:50) (A:B: 75:25) as mobile phase; flow rate: 20 mL/min) to afford the compounds of Example 42A (Fraction I (−)) and Example 42B (Fraction II (+)).

Analytical conditions for Example 42A and Example 42B. HPLC (purity): (column; X-select CSH-C-18 150×4.6 mm, 3.5 μ); mobile Phase: ACN: 0.5% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 70:30); flow Rate: 1.0 mL/min).

### Example 42A

(−)-3-(5-(4-chloro-1H-imidazol-1-yl)-6-methoxypyridin-2-yl)-5-(4-chlorophenyl)-6-methyl-5,6-dihydro-4H-1,2,4-oxadiazine, fraction (I) (−): Mass (ESI): 417.9 [M+1]; HPLC (purity): 98.6%; RT 10.16 min; Chiral HPLC: 100% RT=6.18 min; Optical rotation [α]_{D}^{20.03}: −91.18 (c=0.25, CH₂Cl₂).

### Example 42B

(+)-3-(5-(4-chloro-1H-imidazol-1-yl)-6-methoxypyridin-2-yl)-5-(4-chlorophenyl)-6-methyl-5,6-dihydro-4H-1,2,4-oxadiazine, fraction (II) (+): ¹H NMR (CD₃OD, 400 MHz): δ 8.01 (s, 1H), 7.94 (d, 1H), 7.70 (d, 1H), 7.53 (s, 1H), 7.37 (d, 2H), 7.29 (d, 2H), 4.72 (d, 1H), 4.09 (s, 3H), 4.05 (dd, 1H), 1.00 (d, 3H); Mass (ESI): 417.9 [M+1]; HPLC (purity): 98.5%; RT 10.15 min; Chiral HPLC: 100% RT=7.64 min; Optical rotation [α]_{D}^{20.03}: +102.94 (c=0.25, CH₂Cl₂).

### Example 43

### Synthesis of 5-(4-chlorophenyl)-3-(3-methoxy-4-(4-methyl-1H-imidazol-1-yl) phenyl)-6-methyl-5,6-dihydro-4H-1,2,4-oxadiazine

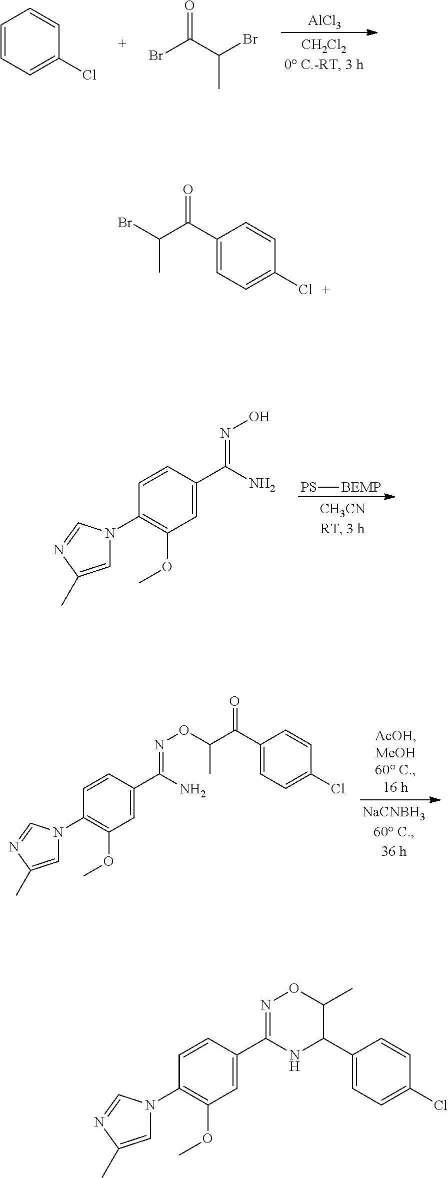

### Synthesis of 2-bromo-1-(4-chlorophenyl) propan-1-one

To a stirred solution of chlorobenzene (1.71 mL, 17 mmol) in CH₂Cl₂ (30 mL) at 0° C. under an argon atmosphere was added AlCl₃ (3.7 g, 28 mmol) and 2-bromopropanoyl bromide (3 g, 14 mmol). The reaction mixture was stirred for 3 h at room temperature. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo to obtain 2-bromo-1-(4-chlorophenyl) propan-1-one (2.1 g, 62%) as an off-white solid. ¹H NMR (CDCl₃, 500 MHz): δ 7.97 (d, 2H), 7.46 (d, 2H), 5.24-5.20 (m, 1H), 1.90 (d, 3H); TLC: 10% EtOAc/Hexane (R_{f}:05).

### Synthesis of (Z)-N′-((1-(4-chlorophenyl)-1-oxopropan-2-yl) oxy)-3-methoxy-4-(4-methyl-1H-imidazol-1-yl) benzimidamide

To a stirred solution of 2-bromo-1-(4-chlorophenyl) propan-1-one (900 mg, 3 mmol) in CH₃CN (45 mL) at room temperature under an argon atmosphere was added PS-BEMP (2 g, 5 mmol). The reaction mixture was stirred for 5 mm at room temperature. Then 2-bromo-1-(4-chlorophenyl) propan-1-one (1.35 g, 5 mmol) in CH₃CN (25 mL) was added to the reaction mixture at room temperature. The reaction mixture was stirred for 3 h at room temperature. After consumption of starting material (by TLC), the reaction mixture was filtered and the filtrate was concentrated in vacuo to obtain (Z)-N′-((1-(3-chlorophenyl)-1-oxopropan-2-yl) oxy)-6-methoxy-5-(4-methyl-1H-imidazol-1-yl) picolinimidamide (1.35 g, crude) as brown semi solid used in the next step without further purification. LCMS: 63.6%; 413 (M+3); (column; Ascentis Express C-18 (50×3.0 mm, 2.7 μm); RT 2.08 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 40% EtOAc/Hexane (R_{f}:05).

### Synthesis of 5-(4-chlorophenyl)-3-(3-methoxy-4-(4-methyl-1H-imidazol-1-yl) phenyl)-6-methyl-5,6-dihydro-4H-1,2,4-oxadiazine

To a stirred solution of (Z)-N′-((1-(3-chlorophenyl)-1-oxopropan-2-yl) oxy)-6-methoxy-5-(4-methyl-1H-imidazol-1-yl) picolinimidamide (1.35 g, 3 mmol) in MeOH (40 mL) at room temperature under an argon atmosphere was added acetic acid (5 mL). The reaction mixture was stirred for 16 h at 60° C. Then sodium cyanoborohydride (155 mg, 4 mmol) was added to the reaction mixture at room temperature. The reaction mixture was stirred for 36 h at 60° C. After consumption of starting material (by TLC), the reaction mixture was diluted with water (50 mL), saturated sodium bicarbonate solution (50 mL) and extracted with EtOAc (2×50 mL). The combined organic extracts were dried over sodium sulfate, filtered and concentrated in vacuo. The crude material was purified by column chromatography using 2% MeOH:CH₂Cl₂ to afford 5-(4-chlorophenyl)-3-(3-methoxy-4-(4-methyl-1H-imidazol-1-yl) phenyl)-6-methyl-5,6-dihydro-4H-1,2,4-oxadiazine (675 mg, 80%) as an off-white solid.

Racemic compound of Example 43 was separated using a Chiralpak-IC column (250×20 mm, 5 μm) (45 mg loading; 0.1% DEA in n-Hexane: CH₂Cl₂:MeOH (80:20) (A:B: 65:35) as mobile phase; flow rate: 20 mL/min) to afford the compounds of Example 43A (Fraction I (−)) and Example 43B (Fraction II (+)).

Analytical conditions for Example 43A and Example 43B. HPLC (purity): (column; X-Select CSH-C-18 150×4.6 mm, 3.5 μ); mobile Phase: ACN: 0.5% TFA; flow rate: 1.0 mL/min; Gradient program: T/B % 0.01/90, 10/10, 15/10: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (80:20) (A::B; 65:35); flow Rate: 1.0 mL/min).

### Example 43A

(−)-5-(4-chlorophenyl)-3-(3-methoxy-4-(4-methyl-1H-imidazol-1-yl) phenyl)-6-methyl-5,6-dihydro-4H-1,2,4-oxadiazine, fraction (I) (−): Mass (ESI): 397.1 [M+1]; HPLC (purity): 99.4%; RT 6.34 min; Chiral HPLC: 100% RT=12.49 min; Optical rotation [α]_{D}^{19.98}: −54.64 (c=0.25, CH₂Cl₂).

### Example 43B

(+)-5-(4-chlorophenyl)-3-(3-methoxy-4-(4-methyl-1H-imidazol-1-yl) phenyl)-6-methyl-5,6-dihydro-4H-1,2,4-oxadiazine, fraction (II) (+): ¹H NMR (DMSO-d₆, 400 MHz): δ 7.94 (d, 1H), 7.81 (s, 1H), 7.51 (s, 1H), 7.45-7.38 (m, 4H), 7.29 (d, 2H), 7.17 (s, 1H), 4.64 (dd, 1H), 3.92 (dd, 1H), 3.87 (s, 3H), 2.15 (s, 3H), 0.87 (d, 3H); Mass (ESI): 397 [M+1]; HPLC (purity): 99.6%; RT 6.36 min; Chiral HPLC: 100% RT=16.30 min; Optical rotation [α]_{D}^{19.99}: +60.09 (c=0.25, CH₂Cl₂).

### Example 44

### Synthesis of 3-(6-methoxy-5-(4-methyl-1H-imidazol-1-yl) pyridin-2-yl)-6-methyl-5-(1-methyl-5-(trifluoromethyl)-1H-pyrrolo [2,3-b]pyridin-3-yl)-5,6-dihydro-4H-1,2,4-oxadiazine

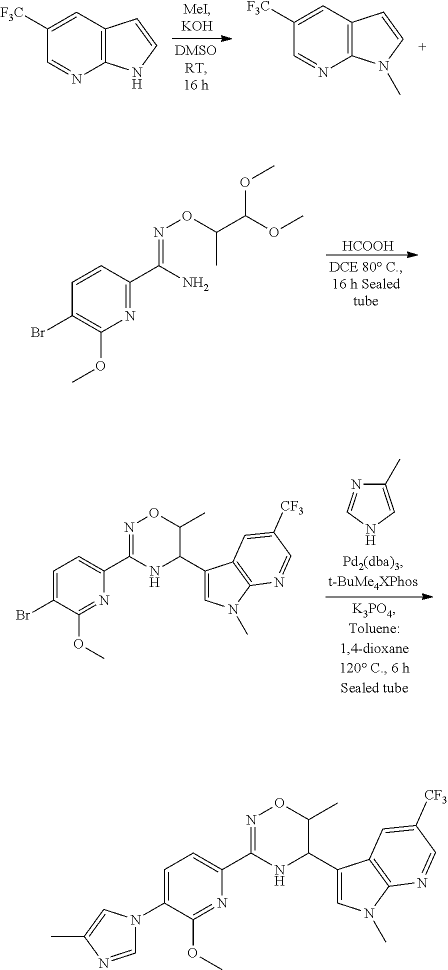

### Synthesis of 1-methyl-5-(trifluoromethyl)-1H-pyrrolo [2,3-b] pyridine

To a stirred solution of 5-(trifluoromethyl)-1H-pyrrolo [2,3-b] pyridine (500 mg, 3 mmol) in DMSO (2 mL) at 0° C. under an argon atmosphere were added potassium hydroxide (302 mg, 5 mmol) and methyl iodide (422 Mg, 3 mmol). The reaction mixture was warmed to room temperature and stirred for 16 h. After consumption of starting material (by TLC), the reaction mixture was quenched with water (50 mL) and extracted with EtOAc (2×50 mL). The combined organic extracts were dried over sodium sulfate, filtered and concentrated in vacuo. The crude material was purified by column chromatography using 7% EtOAc: Hexane to afford 1-methyl-5-(trifluoromethyl)-1H-pyrrolo [2,3-b] pyridine (450 mg, 83%) as a pale yellow solid. ¹H NMR (CDCl₃, 500 MHz): δ 8.61 (s, 1H), 8.20 (s, 1H), 7.34 (d, 1H), 6.60 (d, 1H), 4.00 (s, 3H); TLC: 30% EtOAc/Hexane (R_{f}:06).

### Synthesis of 3-(5-bromo-6-methoxypyridin-2-yl)-6-methyl-5-(1-methyl-5-(trifluoromethyl)-1H-pyrrolo [2,3-b] pyridin-3-yl)-5,6-dihydro-4H-1,2,4-oxadiazine

To a stirred solution of (Z)-5-bromo-N((1,1-dimethoxypropan-2-yl) oxy)-6-methoxypicolinimidamide (250 mg, 0.7 mmol) in 1, 2-dichloro ethane (2.5 mL) at room temperature under an argon atmosphere were added 1-methyl-5-(trifluoromethyl)-1H-pyrrolo [2,3-b] pyridine (288 mg, 1 mmol) and formic acid (2.5 mL). The reaction mixture was stirred at 80° C. for 16 h in a sealed tube. After consumption of starting material (by TLC), the reaction mixture was diluted with sodium bicarbonate solution (20 mL) and extracted with EtOAc (2×20 mL). The combined organic extracts were dried over sodium sulfate, filtered and concentrated in vacuo. The crude material was purified by column chromatography using 20-30% EtOAc: Hexane to afford 3-(5-bromo-6-methoxypyridin-2-yl)-6-methyl-5-(1-methyl-5-(trifluoromethyl)-1H-pyrrolo [2,3-b] pyridin-3-yl)-5,6-dihydro-4H-1,2,4-oxadiazine (348 mg, 59%) as an off-white solid. ¹H NMR (DMSO-d₆, 500 MHz): δ 8.63 (s, 1H), 8.49 (s, 1H), 8.07 (d, 1H), 7.80 (s, 1H), 7.43 (d, 1H), 5.02-5.00 (m, 1H), 4.70 (d, 1H), 3.90-3.83 (m, 7H), 1.19 (d, 3H); TLC: 50% EtOAc/Hexane (R_{f}:03).

### Synthesis of 3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine

To a dry vial was added a suspension of Pd₂(dba)₃ (41 mg, 0.04 mmol) and tert-butyl tetramethyl Xphos (43 mg, 0.09 mmol) in toluene: 1,4-dioxane (2:1, 4 mL) at room temperature. The suspension was degassed with argon, heated to 120° C., and stirred at 120° C. for 3 min. A mixture of 3-(5-bromo-6-methoxypyridin-2-yl)-6-methyl-5-(1-methyl-5-(trifluoromethyl)-1H-pyrrolo [2,3-b] pyridin-3-yl)-5,6-dihydro-4H-1,2,4-oxadiazine (375 mg, 1 mmol), 4-methyl-1H-imidazole (147 mg, 2 mmol) and potassium phosphate (381 mg, 2 mmol) in toluene: 1,4-dioxane (2:1, 4 mL) was degassed and the catalyst premix was added. The resulting mixture was stirred at 120° C. for 6 h in a sealed tube. After consumption of the starting material (monitored by TLC and LCMS), the reaction mixture was filtered and the filtrate was concentrated in vacuo. The crude material was purified by column chromatography using 2%MeOH:CH₂Cl₂to afford 3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine (250 mg, 66%) as an off-white solid.

### Separation of Diastereomers:

Racemic compound of Example 44 was separated using an Inertsil Diol column (250×20 mm, 5 μ) (50 mg loading; n-Hexane: CH₂Cl₂:MeOH (80:20) (A:B: 80:20) as mobile phase; flow rate: 20 mL/min) to provide the compounds of Example 44X and Example 44Y.

Analytical conditions for Example 44X and Example 44Y: HPLC: column; Zorbax SB-C-18 (150×4.6 mm, 3.5 μm); mobile Phase: Acetonitrile: 0.05% TFA; flow rate: 1.0 mL/min; Gradient program: T/B % 0.01/90, 10/10, 15/10: diluent: CH₃CN: Water.

### Example 44X

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine: HPLC (purity): 99.6%; RT 7.07 min

### Example 44Y

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine: HPLC (purity): 98.1%; RT 7.02 min

### Separation of Enantiomers:

Racemic compound of Example 44X was separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 44A (Fraction I (−)) and Example 44B (Fraction II (+)).

Analytical conditions for Example 44A and Example 44B. HPLC (purity): (column; Zorbax SBC-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 44A

(−)-3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (I), (−): Mass (ESI): 486.1 [M+1]; HPLC (purity): 99.8%; RT 7.07 min; Chiral HPLC: 100% RT=8.81 mm; Optical rotation [α]_{D}^{19.99}: −98.43 (c=0.25, CH₂Cl₂).

### Example 44B

(+)-3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (II), (+): ¹H NMR (CD₃OD, 400 MHz): δ 8.59 (s, 1H), 8.36 (s, 1H), 7.97 (s, 1H), 7.90 (d, 1H), 7.72 (s, 1H), 7.67 (d, 1H), 7.21 (s, 1H), 4.73 (d, 1H), 3.95-3.92 (m, 7H), 2.25 (s, 3H), 1.29 (d, 3H); Mass (ESI): 486.1 [M+1]; HPLC (purity): 99.7%; RT 7.08 mm; Chiral HPLC: 99.8% RT=13.45 mm; Optical rotation [α]_{D}^{20.01}: +99.13 (c=0.25, CH₂Cl₂).

Racemic compound of Example 44Y separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 44C (Fraction III (−)) and Example 44D (Fraction IV (+)).

Analytical conditions for Example 44C and Example 44D. HPLC (purity): (column; Zorbax SBC-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 44C

(−)3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (I), (−): Mass (ESI): 486.1 [M+1]; HPLC (purity): 97.7%; RT 7.02 min; Chiral HPLC: 99.3% RT=8.12 min; Optical rotation [α]_{D}^{19.98}: −34.43 (c=0.25, CH₂Cl₂).

### Example 44D

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (II), (+): ¹H NMR (CD₃OD, 400 MHz): δ 8.59 (s, 1H), 8.36 (s, 1H), 7.97 (s, 1H), 7.90 (d, 1H), 7.72 (s, 1H), 7.67 (d, 1H), 7.21 (s, 1H), 4.73 (d, 1H), 3.95-3.92 (m, 7H), 2.25 (s, 3H), 1.29 (d, 3H); Mass (ESI): 486.1 [M+1]; HPLC (purity): 99.6%; RT 7.03 mm; Chiral HPLC: 99.6% RT=9.35 mm; Optical rotation [α]_{D}^{19.98}: +28.43 (c=0.25, CH₂Cl₂).

### Example 45

### Synthesis of 3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine

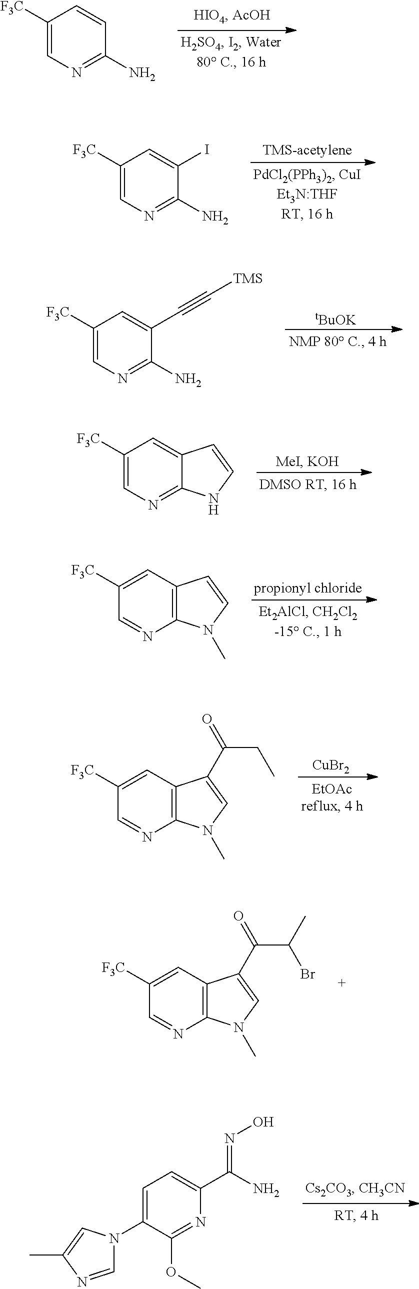

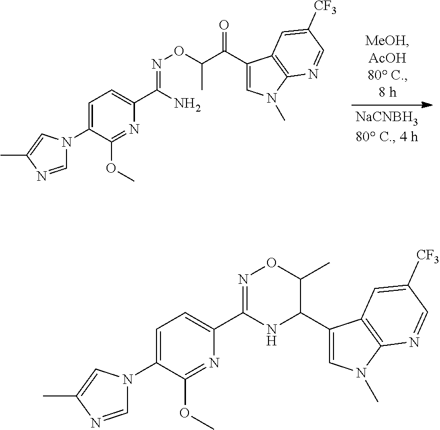 [SMILES: COc1nc(/C(N)=N/OC(C)C(=O)c2cn(C)c3ncc(C(F)(F)F)cc23)ccc1-n1cnc(C)c1 ; COc1nc(C2=NOC(C)C(c3cn(C)c4ncc(C(F)(F)F)cc34)N2)ccc1-n1cnc(C)c1]

### Synthesis of 3-iodo-5-(trifluoromethyl) pyridin-2-amine

To a stirred solution of 5-(trifluoromethyl) pyridin-2-amine (100 g, 617 mmol) in acetic acid (1 Lit) at room temperature under an argon atmosphere were added concentrated sulfuric acid (5 mL), periodic acid (26 g, 123 mmol) and iodine (62 g, 246 mmol). The reaction mixture was stirred at 80° C. for 16 h. After consumption of starting material (by TLC), the reaction mixture was basified with sodium hydroxide solution (200 mL) at 0° C., to obtain the solid. The solid was filtered and concentrated in vacuo to obtain 3-iodo-5-(trifluoromethyl) pyridin-2-amine (120 g, 67%) as an off-white solid. ¹H NMR (DMSO-d₆, 500 MHz): δ 8.27 (s, 1H), 8.16 (s, 1H), 6.87 (brs, 2H); TLC: 10% EtOAc/Hexane (R_{f}:04).

### Synthesis of 5-(trifluoromethyl)-3-((trimethylsilyl) ethynyl) pyridin-2-amine

To a stirred solution of 3-iodo-5-(trifluoromethyl) pyridin-2-amine (150 g, 520 mmol) in THF (500 mL) at room temperature under an argon atmosphere were added triethyl amine (1 Lit), copper iodide (980 mg, 5.2 mmol) Pd(PPh₃)₂Cl₂ (3.6 g, 5 mmol) and TMS-acetylene (160 mL, 130 mmol). The reaction mixture was stirred at room temperature for 16 h. After consumption of starting material (by TLC), the reaction mixture was filtered and the filtrate was concentrated in vacuo to obtain 5-(trifluoromethyl)-3-((trimethylsilyl) ethynyl) pyridin-2-amine (150 g, crude) as block solid. TLC: 10% EtOAc/Hexane (R_{f}:05).

### Synthesis of 5-(trifluoromethyl)-1H-pyrrolo [2,3-b] pyridine

To a stirred solution of 5-(trifluoromethyl)-3-((trimethylsilyl) ethynyl) pyridin-2-amine (100 g, 329 mmol) in NMP (600 mL) at room temperature under an argon atmosphere was added potassium tertiary butoxide (92 g, 778 mmol). The reaction mixture was stirred at 80° C. for 4 h. After consumption of starting material (by TLC), the reaction mixture was diluted with water (200 mL) and extracted with EtOAc (2×200 mL). The combined organic extracts were dried over sodium sulfate, filtered and concentrated in vacuo to obtain 5-(trifluoromethyl)-1H-pyrrolo [2,3-b] pyridine (35 g, 48%) as a pale brown solid. ¹H NMR (DMSO-d₆, 400 MHz): δ 12.18 (s, 1H), 8.54 (s, 1H), 8.38 (s, 1H), 7.69 (d, 1H), 6.60 (s, 1H); TLC: 30% EtOAc/Hexane (R_{f}:03).

### Synthesis of 1-methyl-5-(trifluoromethyl)-1H-pyrrolo [2,3-b] pyridine

To a stirred solution of 5-(trifluoromethyl)-1H-pyrrolo [2,3-b] pyridine (5.3 g, 28 mmol) in DMSO (30 mL) at 0° C. under an argon atmosphere were added potassium hydroxide (2.3 g, 42 mmol) and methyl iodide (6.06 g, 42 mmol). The reaction mixture was warmed to room temperature and stirred for 16 h. After consumption of starting material (by TLC), the reaction mixture was quenched with water (100 mL) and extracted with EtOAc (2×100 mL). The combined organic extracts were dried over sodium sulfate, filtered and concentrated in vacuo. The crude material was purified by column chromatography using 10% EtOAc: Hexane to afford 1-methyl-5-(trifluoromethyl)-1H-pyrrolo [2,3-b] pyridine (4.3 g, 76%) as a pale yellow solid. ¹H NMR (CDCl₃, 500 MHz): δ 8.59 (s, 1H), 8.15 (s, 1H), 7.30 (d, 1H), 6.56 (d, 1H), 3.93 (s, 3H); LCMS: 98.9%; 201 (M+1); (column; Ascentis Express C-18 (50×3.0 mm, 2.7 μm); RT 2.44 mm; mobile phase: 0.025% Aq TFA++5% ACN:ACN+5% 0.05% Aq TFA; T/B %: 0.01/5, 0.5/5, 3/100, 5/100; flow rate: 1.2 mL/min) (Gradient); TLC: 30% EtOAc/Hexane (R_{f}:06).

### Synthesis of 1-(1-methyl-5-(trifluoromethyl)-1H-pyrrolo [2,3-b] pyridin-3-yl) propan-1-one

To a stirred solution of 1-methyl-5-(trifluoromethyl)-1H-pyrrolo [2,3-b] pyridine (30 g, 150 mmol) in CH₂Cl₂ (1.2 Lit) at -15° C. under an argon atmosphere were added diethyl aluminum chloride (180 mL, 180 mmol) and propionyl chloride (20 g, 225 mmol). The reaction mixture was stirred at −15° C. for 1 h. After consumption of starting material (by TLC), the reaction mixture was quenched with saturated ammonium chloride solution (300 mL) and extracted with CH₂Cl₂ (2×200 mL). The combined organic extracts were dried over sodium sulfate, filtered and concentrated in vacuo. The crude material was purified by column chromatography using 20-30% EtOAc: Hexane to afford 1-(1-methyl-5-(trifluoromethyl)-1H-pyrrolo [2,3-b] pyridin-3-yl) propan-1-one (10 g, 34%) as a pale yellow solid. ¹H NMR (CDCl₃, 500 MHz): δ 8.93 (s, 1H), 8.67 (s, 1H), 7.96 (s, 1H), 4.00 (s, 3H), 2.93-2.89 (m, 2H), 1.28 (t, 3H); LCMS: 93.1%; 256.8 (M+1); (column; Ascentis Express C-18 (50×3.0 mm, 2.7 μm); RT 2.48 min; mobile phase: 0.025% Aq TFA++5% ACN: ACN+5% 0.05% Aq TFA; T/B %: 0.01/5, 0.5/5, 3/100, 5/100; flow rate: 1.2 mL/min) (Gradient); TLC: 50% EtOAc/Hexane (R_{f}:05).

### Synthesis of 2-bromo-1-(1-methyl-5-(trifluoromethyl)-1H-pyrrolo [2,3-b] pyridin-3-yl) propan-1-one

To a stirred solution of 1-(1-methyl-5-(trifluoromethyl)-1H-pyrrolo [2,3-b] pyridin-3-yl) propan-1-on (25 g, 97 mmol) in EtOAc (500 mL) at room temperature under an argon atmosphere was added copper bromide (60 g, 195 mmol). The reaction mixture was stirred at reflux for 4 h. After consumption of starting material (by TLC), the reaction mixture was filtered. The filtrate was concentrated in vacuo. The crude material was purified by column chromatography using 20% EtOAc: Hexane to afford 2-bromo-1-(1-methyl-5-(trifluoromethyl)-1H-pyrrolo [2,3-b] pyridin-3-yl) propan-1-one (16 g, 50%) as colorless liquid. ¹H NMR (CDCl₃, 500 MHz): δ 8.93 (s, 1H), 8.69 (s, 1H), 8.13 (s, 1H), 5.11-5.07 (m, 1H), 4.06-3.99 (m, 3H), 1.95 (d, 3H); LCMS: 74.5%; 336.8 (M+1); (column; Ascentis Express C-18 (50×3.0 mm, 2.7 μm); RT 2.72 min; mobile phase: 0.025% Aq TFA++5% ACN:ACN+5% 0.05% Aq TFA; T/B %: 0.01/5, 0.5/5, 3/100, 5/100; flow rate: 1.2 mL/min) (Gradient); TLC: 30% EtOAc/Hexane (R_{f}:05).

### Synthesis of (Z)-6-methoxy-5-(4-methyl-1H-imidazol-1-yl)-N′-((1-(1-methyl-5-(trifluoromethyl)-1H-pyrrolo [2,3-b] pyridin-3-yl)-1-oxopropan-2-yl) oxy) picolinimidamide

To a stirred solution of (Z)-N′-hydroxy-6-methoxy-5-(4-methyl-1H-imidazol-1-yl) picolinimidamide (7 g, 28 mmol) in CH₃CN (200 mL) at room temperature under an argon atmosphere were added cesium carbonate (18.3 g, 56 mmol) and 2-bromo-1-(1-methyl-5-(trifluoromethyl)-1H-pyrrolo [2,3-b] pyridin-3-yl) propan-1-one (14.2 g, 42 mmol) in CH₃CN (200 mL). The reaction mixture was stirred at room temperature for 4 h. After consumption of starting material (by TLC), the reaction mixture was filtered. The filtrate was concentrated in vacuo to (Z)-6-methoxy-5-(4-methyl-1H-imidazol-1-yl)-N′-((1-(1-methyl-5-(trifluoromethyl)-1H-pyrrolo [2,3-b] pyridin-3-yl)-1-oxopropan-2-yl) oxy) picolinimidamide (18 g, crude) as colorless liquid used in the next step without further purification. LCMS: 87.7%; 502.1 (M+1); (column; Ascentis Express C-18 (50×3.0 mm, 2.7 μm); RT 2.06 min; mobile phase: 0.025% Aq TFA++5% ACN:ACN+5% 0.05% Aq TFA; T/B %: 0.01/5, 0.5/5, 3/100, 5/100; flow rate: 1.2 mL/min) (Gradient); TLC: 5% MeOH/CH₂Cl₂ (R_{f}: 0.5).

### Synthesis of 3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine

To a stirred solution of (Z)-6-methoxy-5-(4-methyl-1H-imidazol-1-yl)-N′-((1-(1-methyl-5-(trifluoromethyl)-1H-pyrrolo [2,3-b] pyridin-3-yl)-1-oxopropan-2-yl) oxy) picolinimidamide (13 g, 26 mmol) in MeOH (400 mL) at room temperature under an argon atmosphere was added acetic acid (100 mL). The reaction mixture was stirred for 8 h at 60° C. Then sodium cyanoborohydride (3.26 g, 52 mmol) was added to the reaction mixture at room temperature. The reaction mixture was stirred for 4 h at 80° C. After consumption of starting material (by TLC), the volatiles were evaporated in vacuo. The residue was poured in to cold saturated sodium bicarbonate solution (250 mL) and extracted with EtOAc (2×150 mL). The combined organic extracts were washed with brine (300 m), dried over sodium sulfate, filtered and concentrated in vacuo. The crude material was purified by column chromatography using 2% MeOH:CH₂Cl₂to afford 3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine (7 g, 56%, over two steps) as a pale yellow solid.

### Separation of Diastereomers:

Racemic compound of Example 45 was separated using an Inertsil Diol column (250×20 mm, 5 μ) (50 mg loading; n-Hexane: CH₂Cl₂:MeOH (80:20) (A:B: 80:20) as mobile phase; flow rate: 20 mL/min) to provide the compounds of Example 45X and Example 45Y. Analytical conditions for Example 45X and Example 45Y: HPLC: column; Zorbax SB-C-18 (150×4.6 mm, 3.5 μm); mobile Phase: Acetonitrile: 0.05% TFA; flow rate: 1.0 mL/min; Gradient program: T/B % 0.01/90, 10/10, 15/10: diluent: CH₃CN: Water.

### Example 45X

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine: HPLC (purity): 99.6%; RT 7.07 min

### Example 45Y

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine: HPLC (purity): 98.1%; RT 7.02 min

### Separation of Enantiomers:

Racemic compound of Example 45X was separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 45A (Fraction I (−)) and Example 45B (Fraction II (+)).

Analytical conditions for Example 45A and Example 45B. HPLC (purity): (column; Zorbax SBC-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 45A

(−)-3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (I), (−): Mass (ESI): 486.1 [M+1]; HPLC (purity): 99.8%; RT 7.07 min; Chiral HPLC: 100% RT=8.81 min; Optical rotation [α]_{D}^{19.99}: −98.43 (c=0.25, CH₂Cl₂).

### Example 45B

(+)-3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (II), (+): ¹H NMR (CD₃OD, 400 MHz): δ 8.59 (s, 1H), 8.36 (s, 1H), 7.97 (s, 1H), 7.90 (d, 1H), 7.72 (s, 1H), 7.67 (d, 1H), 7.21 (s, 1H), 4.73 (d, 1H), 3.95-3.92 (m, 7H), 2.25 (s, 3H), 1.29 (d, 3H); Mass (ESI): 486.1 [M+1]; HPLC (purity): 99.7%; RT 7.08 min; Chiral HPLC: 99.8% RT=13.45 min; Optical rotation [α]_{D}^{20.01}: +99.13(c=0.25, CH₂Cl₂).

Racemic compound of Example 45Y separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 45C (Fraction III (−)) and Example 45D (Fraction IV (+)).

Analytical conditions for Example 45C and Example 45D. HPLC (purity): (column; Zorbax SBC-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 45C

(−)3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (I), (−): Mass (ESI): 486.1 [M+1]; HPLC (purity): 97.7%; RT 7.02 min; Chiral HPLC: 99.3% RT=8.12 min; Optical rotation [α]_{D}^{19.98}: −34.43 (c=0.25, CH₂Cl₂).

### Example 45D

3-(6-methoxy-5-(4-methyl-1H-imidazol-1-yl) pyridin-2-yl)-6-methyl-5-(1-methyl-5-(trifluoromethyl)-1H-pyrrolo [2,3-b] pyridin-3-yl)-5,6-dihydro-4H-1,2,4-oxadiazine, fraction (II), (+): ¹H NMR (CD₃OD, 400 MHz): δ 8.59 (s, 1H), 8.36 (s, 1H), 7.97 (s, 1H), 7.90 (d, 1H), 7.72 (s, 1H), 7.67 (d, 1H), 7.21 (s, 1H), 4.73 (d, 1H), 3.95-3.92 (m, 7H), 2.25 (s, 3H), 1.29 (d, 3H); Mass (ESI): 486.1 [M+1]; HPLC (purity): 99.6%; RT 7.03 mm; Chiral HPLC: 99.6% RT=9.35 mm; Optical rotation [α]_{D}^{19.98}: +28.43 (c=0.25, CH₂Cl₂).

### Example 46

### Synthesis of 5-(6-chlorobenzofuran-2-yl)-3-(6-methoxy-5-(4-methyl-1H-imidazol-1-yl) pyridin-2-yl)-6-methyl-5,6-dihydro-4H-1,2,4-oxadiazine

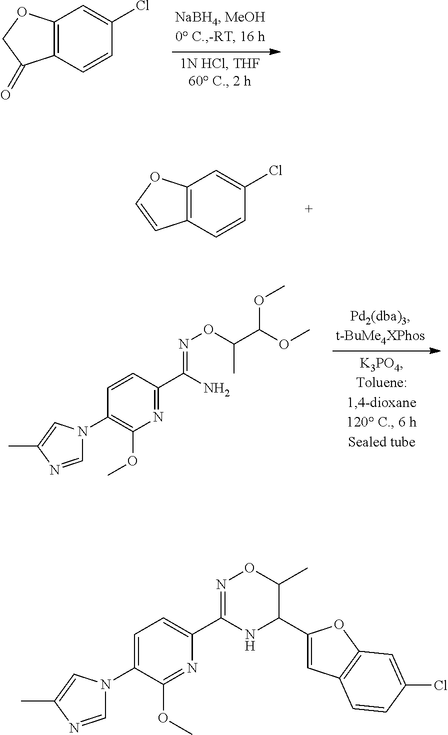

### Synthesis of 6-chlorobenzofuran

To a stirred solution of 6-chlorobenzofuran-3(2H)-one (2 g, 12 mmol) in MeOH (20 mL) at 0° C. under an argon atmosphere was added sodium borohydride (540 mg, 14 mmol). The reaction mixture was warmed to room temperature and stirred for 16 h. After consumption of starting material (by TLC), the reaction mixture was diluted with saturated sodium bicarbonate solution (100 mL) and extracted with CH₂Cl₂ (2×50 mL). The combined organic extracts were dried over sodium sulfate, filtered and concentrated in vacuo.

To the above residue in THF (20 mL) was added 1 N HCl (5 mL) was added at room temperature. The reaction mixture was stirred at 60° C. for 2 h. After consumption of starting material (by TLC), the reaction mixture was diluted with water (100 mL) and extracted with Hexane (2×100 mL). The combined organic extracts were dried over sodium sulfate, filtered and concentrated in vacuo to obtain 6-chlorobenzofuran (1.2 g, 70%) as an off-white solid used for next step without further purification. ¹H NMR (CDCl₃, 500 MHz): δ 7.33 (d, 1H), 6.93 (d, 1H), 6.89 (s, 1H), 5.34-5.30 (m, 1H), 4.60-4.55 (m, 1H), 4.47-4.43 (m, 1H); TLC: Hexane (R_{f}:06).

### Synthesis of 5-(6-chlorobenzofuran-2-yl)-3-(6-methoxy-5-(4-methyl-1H-imidazol-1-yl) pyridin-2-yl)-6-methyl-5,6-dihydro-4H-1,2,4-oxadiazine

To a dry vial was added a suspension of Pd₂(dba)₃ (31 mg, 0.03 mmol) and tert-butyl tetramethyl XPhos (33 mg, 0.07 mmol) in toluene: 1,4-dioxane (2:1, 4.5 mL) at room temperature. The suspension was degassed with argon, heated to 120° C., and stirred at 120° C. for 3 min. A mixture of (Z)-N′-((1,1-dimethoxypropan-2-yl) oxy)-6-methoxy-5-(4-methyl-1H-imidazol-1-yl) picolinimidamide (300 mg, 0.7 mmol), 4-methyl-1H-imidazole (68 mg, 0.8 mmol) and potassium phosphate (376 mg, 2 mmol) in toluene: 1,4-dioxane (2:1, 6 mL) was degassed and the catalyst premix was added. The resulting mixture was stirred at 120° C. for 6 h in a sealed tube. After consumption of the starting material (monitored by TLC and LCMS), the reaction mixture was filtered and the filtrate was concentrated in vacuo. The crude material was purified by column chromatography using 3%MeOH:CH₂Cl₂ to afford 5-(6-chlorobenzofuran-2-yl)-3-(6-methoxy-5-(4-methyl-1H-imidazol-1-yl) pyridin-2-yl)-6-methyl-5,6-dihydro-4H-1,2,4-oxadiazine (250 mg, 62%) as an off-white solid.

### Separation of Diastereomers:

Racemic compound of Example 46 was separated using a Inertsil Diol column (250×20 mm, 5 μ) (50 mg loading; n-Hexane: CH₂Cl₂:MeOH (80:20) (A:B: 80:20) as mobile phase; flow rate: 20 mL/min) to provide the compounds of Example 46X and Example 46Y.

Analytical conditions for Example 46X and Example 46Y: HPLC: column; Zorbax SB-C-18 (150×4.6 mm, 3.5 μm); mobile Phase: Acetonitrile: 0.05% TFA; flow rate: 1.0 mL/min; Gradient program: T/B % 0.01/90, 10/10, 15/10: diluent: CH₃CN: Water. Example 46X, 5-(6-chlorobenzofuran-2-yl)-3-(6-methoxy-5-(4-methyl-1H-imidazol-1-yl) pyridin-2-yl)-6-methyl-5,6-dihydro-4H-1,2,4-oxadiazine: HPLC (purity): 99.1%; RT 7.11 min.

### Example 46Y

5-(6-chlorobenzofuran-2-yl)-3-(6-methoxy-5-(4-methyl-1H-imidazol-1-yl) pyridin-2-yl)-6-methyl-5,6-dihydro-4H-1,2,4-oxadiazine: HPLC (purity): 99.6%; RT 7.29 min

### Separation of Enantiomers:

Racemic compound of Example 46X was separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 46A (Fraction I (−)) and Example 46B (Fraction II (+)).

Analytical conditions for Example 46A and Example 46B. HPLC (purity): (column; zorbax-SB-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 46A

(−)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (I) (−): Mass (ESI): 438.1 [M+1]; HPLC (purity): 97.8%; RT 7.88 min; Chiral HPLC: 100% RT=8.64 min; Optical rotation [α]_{D}^{19.98}: −82.88 (c=0.25, CH₂Cl₂).

### Example 46B

(+)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (II) (+): ¹H NMR (CD₃OD, 400 MHz): δ 7.98 (s, 1H), 7.88 (d, 1H), 7.65 (d, 1H), 7.58-7.54 (m, 2H), 7.25 (dd, 1H), 7.23-7.21 (m, 1H), 6.88 (s, 1H), 4.66 (d, 1H), 4.12-4.06 (m, 4H), 2.25 (s, 3H), 1.37 (d, 3H); Mass (ESI): 438.1 [M+1]; HPLC (purity): 99.1%; RT 7.85 min; Chiral HPLC: 99.8% RT=12.75 min; Optical rotation [α]_{D}^{19.99}: +68.40 (c=0.25, CH₂Cl₂).

Racemic compound of Example 46Y was separated using a Chiralpak-IB column (250×20 mm, 5 μm) (10 mg loading; 0.1% DEA in n-Hexane: CH₂Cl₂:MeOH (50:50) (A:B: 80:20) as mobile phase; flow rate: 20 mL/min) to afford the compounds of Example 46C (Fraction III (−)) and Example 46D (Fraction IV (+)).

Analytical conditions for Example 46C and Example 46D. HPLC (purity): (column; zorbax-SB-C-18 150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 46C

(−)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (III) (−): Mass (ESI): 438.1 [M+1]; HPLC (purity): 96.3%; RT 7.66 min; Chiral HPLC: 99.3% RT=8.36 min; Optical rotation [α]_{D}^{20.00}: −85.16 (c=0.25, CH₂Cl₂).

### Example 46D

(+)-5-(5,6-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (IV) (+): ¹H NMR (CD₃OD, 400 MHz): δ 7.98 (s, 1H), 7.88 (d, 1H), 7.65 (d, 1H), 7.58-7.54 (m, 2H), 7.25 (dd, 1H), 7.23-7.21 (m, 1H), 6.88 (s, 1H), 4.66 (d, 1H), 4.12-4.06 (m, 4H), 2.25 (s, 3H), 1.37 (d, 3H); Mass (ESI): 438.1 [M+1]; HPLC (purity): 97.3%; RT 7.69 min; Chiral HPLC: 100% RT=10.10 min; Optical rotation [α]_{D}^{19.99}: +96.24 (c=0.25, CH₂Cl₂).

### Example 47

### Synthesis of 3-(5-(4-chloro-1H-imidazol-1-yl)-6-methoxypyridin-2-yl)-5-(3-chlorophenyl)-6-methyl-5,6-dihydro-4H-1,2,4-oxadiazine

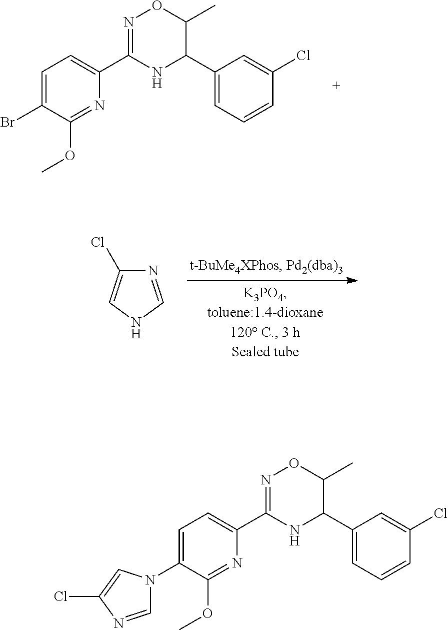 [SMILES: COc1nc(C2=NOC(C)C(c3cccc(Cl)c3)N2)ccc1Br ; Clc1c[nH]cn1 ; COc1nc(C2=NOC(C)C(c3cccc(Cl)c3)N2)ccc1-n1cnc(Cl)c1]

### Synthesis of 3-(5-(4-chloro-M-imidazol-1-yl)-6-methoxypyridin-2-yl)-5-(3-chlorophenyl)-6-methyl-5,6-dihydro-4H-1,2,4-oxadiazine

To a dry vial was added a suspension of Pd₂(dba)₃ (12 mg, 0.01 mmol) and tert-butyl tetramethyl XPhos (12 mg, 0.02 mmol) in toluene: 1,4-dioxane (2:1, 1.5 mL) at room temperature. The suspension was degassed with argon, heated to 120° C., and stirred at 120° C. for 3 min. A mixture of 3-(5-bromo-6-methoxypyridin-2-yl)-5-(3-chlorophenyl)-6-methyl-5,6-dihydro-4H-1,2,4-oxadiazine (100 mg, 0.25 mmol), 4-chloro-1H-imidazole (25 mg, 0.2 mmol) and potassium phosphate (107 mg, 0.5 mmol) in toluene: 1,4-dioxane (2:1, 1.5 mL) was degassed and the catalyst premix was added. The resulting mixture was stirred at 120° C. for 3 h in a sealed tube. After consumption of the starting material (monitored by TLC and LCMS), the reaction mixture was diluted with water (20 mL) and extracted with EtOAc (2×20 mL). The combined organic extracts were dried over sodium sulfate, filtered and concentrated in vacuo. The crude material was purified by column chromatography using 2-3%MeOH:CH₂Cl₂ to afford 3-(5-(4-chloro-1H-imidazol-1-yl)-6-methoxypyridin-2-yl)-5-(3-chlorophenyl)-6-methyl-5,6-dihydro-4H-1,2,4-oxadiazine (25 mg, 26%) as an off-white solid. Racemic compound of Example 47 was separated using a Chiralpak-IA column (250×20 mm, 5 μm) (20 mg loading; 0.1% DEA in n-Hexane: CH₂Cl₂:MeOH (80:20) (A:B: 80:20) as mobile phase; flow rate: 20 mL/min) to afford the compounds of Example 47A (Fraction I (−)) and Example 47B (Fraction II (+)).

Analytical conditions for Example 47A and Example 47B. HPLC (purity): (column; X-select CSH C-18 150×4.6 mm, 3.5 μm); mobile Phase: ACN: 0.5% TFA; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (80:20) (A::B; 80:20); flow Rate: 1.0 mL/min).

### Example 47A

(−)-3-(5-(4-chloro-1H-imidazol-1-yl)-6-methoxypyridin-2-yl)-5-(3-chlorophenyl)-6-methyl-5,6-dihydro-4H-1,2,4-oxadiazine, fraction (I), (−): Mass (ESI): 418 [M+1]; HPLC (purity): 99.7%; RT 10.10 mm; Chiral HPLC: 100% RT=10.60 min; Optical rotation [α]_{D}^{20.00}: −186.57 (c=0.25, CH₂Cl₂).

### Example 47B

(+)-3-(5-(4-chloro-1H-imidazol-1-yl)-6-methoxypyridin-2-yl)-5-(3-chlorophenyl)-6-methyl-5,6-dihydro-4H-1,2,4-oxadiazine, fraction (II), (+): ¹H NMR (DMSO-d₆, 400 MHz): δ 8.04-7.97 (m, 3H), 7.71-7.67 (m, 2H), 7.42-7.33 (m, 2H), 7.27-7.19 (m, 2H), 4.72-4.70 (m, 1H), 4.03 (s, 3H), 3.92-3.90 (m, 1H), 0.91 (d, 3H); Mass (ESI): 418 [M+1]; HPLC (purity): 99.4%; RT 10.12 min; Chiral HPLC: 100% RT=12.04 mm; Optical rotation [α]_{D}^{20.01}: +199.93 (c=0.25, CH₂Cl₂).

### Example 48

### Synthesis of 3-(5-(4-chloro-1H-imidazol-1-yl)-6-methoxypyridin-2-yl)-5-(3-chlorophenyl)-6-methyl-5,6-dihydro-4H-1,2,4-oxadiazine

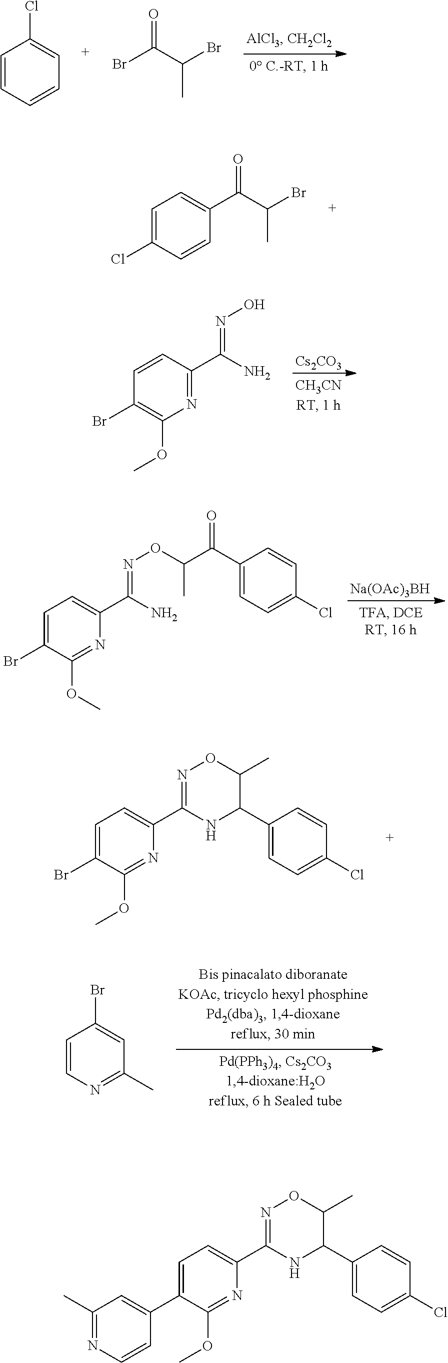

### Synthesis of 2-bromo-1-(4-chlorophenyl) propan-1-one

To a stirred solution of chlorobenzene (5.7 mL, 56 mmol) in CH₂Cl₂ (100 mL) at 0° C. were added aluminum trichloride (12.3 g, 93 mmol) and 2-bromopropanoyl bromide (10 g, 46 mmol) under an argon atmosphere. The reaction mixture was warmed to room temperature and stirred for 1 h. After consumption of the starting material (by TLC), the reaction mixture was diluted with ice cold water (200 mL) and extracted with CH₂Cl₂ (2×200 mL). The combined organic extract was washed with sodium bicarbonate solution (200 mL), dried over sodium sulfate, filtered and concentrated in vacuo to obtain 2-bromo-1-(4-chlorophenyl)propan-1-one (8.5 g, 74%) as an off-white semi-solid used in the next step without further purification. ¹H NMR (CDCl₃, 500 MHz): δ 7.97 (d, 2H), 7.45 (d, 2H), 5.22-5.19 (m, 1H), 1.90 (d, 3H); LCMS: 98.1%; 249.3 (M+3); (column; X-select CSH C-18 (50×3.0 mm, 2.5 μm); RT 4.36 min; mobile phase: 2.5 mM Aq NH₄OAc: ACN; T/B %: 0.01/10, 0.5/10, 3.5/90, 7/90; flow rate: 0.8 mL/min) (Gradient); TLC: 20% EtOAc/Hexane (R_{f}:05). Synthesis of (Z)-5-bromo-N′-((1-(4-chlorophenyl)-1-oxopropan-2-yl) oxy)-6-methoxypicolinimidamide

To a stirred solution of (Z)-5-bromo-N′-hydroxy-6-methoxypicolinimidamide (1.6 g, 6 mmol) in CH₃CN (30 mL) at room temperature under an argon atmosphere were added cesium carbonate (4.21 g, 3 mmol). The reaction mixture was stirred at room temperature for 10 min Then 2-bromo-1-(4-chlorophenyl) propan-1-one (2.39 g, 10 mmol) in CH₃CN (10 mL) was added to the reaction mixture at room temperature. The reaction mixture was stirred at room temperature for 1 h. After consumption of starting material (by TLC), the reaction mixture was filtered. The filtrate was concentrated in vacuo to obtain (Z)-5-bromo-N′4(1-(4-chlorophenyl)-1-oxopropan-2-yl) oxy)-6-methoxypicolinimidamide (2 g, crude) as brown solid used in the next step without further purification. LCMS: 30.7%; 413.8 (M+3); (column; Ascentis Express C-18 (50×3.0 mm, 2.7 μm); RT 2.83 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:05).

### Synthesis of 3-(5-bromo-6-methoxypyridin-2-yl)-5-(4-chlorophenyl)-6-methyl-5,6-dihydro-4H-1,2,4-oxadiazine

To a stirred solution of (Z)-5-bromo-N4(1-(4-chlorophenyl)-1-oxopropan-2-yl) oxy)-6-methoxypicolinimidamide (2 g, 5 mmol) in 1,2-dichloro ethane (40 mL) at room temperature under an argon atmosphere were added trifluoro acetic acid (1.1 mL, 14 mmol) and sodium triacetoxyborohydride (5.1 g, 24 mmol). The reaction mixture was stirred for 16 h at room temperature. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 20-30%EtOAc: Hexane to afford 3-(5-bromo-6-methoxypyridin-2-yl)-5-(4-chlorophenyl)-6-methyl-5,6-dihydro-4H-1,2,4-oxadiazine (1.2 g, 63%) as an off-white solid. LCMS: 89.8%; 397.7 (M+3); (column; Ascentis Express C-18 (50×3.0 mm, 2.7 μm); RT 2.93 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:03).

### Synthesis of 5-(4-chlorophenyl)-3-(2-methoxy-2′-methyl-[3,4′-bipyridin]-6-yl)-6-methyl-5,6-dihydro-4H-1,2,4-oxadiazine

To a stirred solution of 4-bromo-6-methylpyridin-3-ylium (750 mg, 4 mmol) in 1, 4-dioxane (10 mL) at room temperature under an argon atmosphere were added Bis pinacolato diboron (1.1 g, 4 mmol), potassium acetate (855 mg, 9 mmol), tricyclo hexylphosphine (1.8 g, 6 mmol) and purged under argon for 30 mm. Then Pd₂(dba)₃(2 g, 2 mmol) was added to the reaction mixture at room temperature. The reaction mixture was heated to reflux for 4 h.

To a stirred solution of above reaction mixture were added in 1, 4-dioxane: water (1:2, 30 mL) at room temperature under an argon atmosphere were added Pd(PPh₃)₄ (501 mg, 4 mmol), cesium carbonate (5.5 g, 17 mmol) and 3-(5-bromo-6-methoxypyridin-2-yl)-5-(4-chlorophenyl)-6-methyl-5,6-dihydro-4H-1,2,4-oxadiazine (1.7 g, 4 mmol) at room temperature. The reaction mixture was heated to reflux for 6 h.

After consumption of starting material (by TLC), the reaction mixture was filtered diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 20-30%EtOAc: Hexane to afford 3-(5-bromo-6-methoxypyridin-2-yl)-5-(4-chlorophenyl)-6-methyl-5,6-dihydro-4H-1,2,4-oxadiazine (150 mg, 9%) as an off-white solid.

Racemic compound of Example 48 was separated using a Chiralpak-IB column (250×20 mm, 5 μm) (20 mg loading; 0.1% DEA in n-Hexane: CH₂Cl₂:MeOH (50:50) (A:B: 90:10) as mobile phase; flow rate: 20 mL/min) to afford the compounds of Example 48A (Fraction I (−)) and Example 48B (Fraction II (+)).

Analytical conditions for Example 48A and Example 48B. HPLC (purity): (column; Zorbax SB C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 90:10); flow Rate: 1.0 mL/min).

### Example 48A

(−)-5-(4-chlorophenyl)-3-(2-methoxy-2′-methyl-[3,4′-bipyridin]-6-yl)-6-methyl-5,6-dihydro-4H-1,2,4-oxadiazine, fraction (I), (−): Mass (ESI): 409 [M+1]; HPLC (purity): 99.3%; RT 7.35 mm; Chiral HPLC: 100% RT=13.88 mm; Optical rotation [α]_{D}^{20.00}: −200.84 (c=0.25, CH₂Cl₂).

### Example 48B

(+)-5-(4-chlorophenyl)-3-(2-methoxy-2′-methyl-[3,4′-bipyridin]-6-yl)-6-methyl-5,6-dihydro-4H-1,2,4-oxadiazine, fraction (II), (+): ¹H NMR (CD₃OD, 400 MHz): δ 8.44 (d, 1H), 7.93 (d, 1H), 7.68 (d, 1H), 7.56 (s, 1H), 7.50 (d, 1H), 7.39-7.34 (m, 2H), 7.32-7.26 (m, 2H), 4.73 (d, 1H), 4.05 (dd, 1H), 4.03 (s, 3H), 2.59 (s, 3H), 0.99 (d, 3H); Mass (ESI): 409.1 [M+1]; HPLC (purity): 98.7%; RT 7.34 min; Chiral HPLC: 99.6% RT=19.37 min; Optical rotation [α]_{D}^{20.03}: +181.10 (c=0.25, CH₂Cl₂).

### Example 49

### Synthesis of 5-(3-chlorophenyl)-3-(2-methoxy-2′-methyl-[3,4′-bipyridin]-6-yl)-6-methyl-5,6-dihydro-4H-1,2,4-oxadiazine

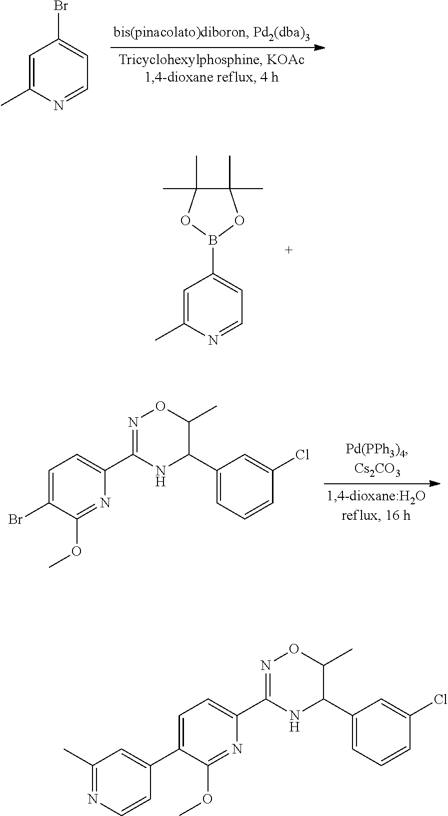

### Synthesis of 5-(3-chlorophenyl)-3-(2-methoxy-2′-methyl-[3,4′-bipyridin]-6-yl)-6-methyl-5,6-dihydro-4H-1,2,4-oxadiazine:

To a stirred solution of 4-bromo-2-methylpyridine (150 mg, 0.8 mmol) in 1,4-dioxane (5 mL) under argon atmosphere were added his (pinacolato) diboron (243 mg, 0.9 mmol), potassium phosphate (171 mg, 2 mmol), Pd₂(dba)₃ (39 mg, 0.04 mmol), tricyclohexyl phosphine (366 mg, 1 mmol) and degassed under argon atmosphere for 30 min at room temperature. The reaction mixture was stirred at reflux for 4 h.

To a stirred solution of the above reaction mixture were added 3-(5-bromo-6-methoxypyridin-2-yl)-5-(3-chlorophenyl)-6-methyl-5,6-dihydro-4H-1,2,4-oxadiazine (345 mg, 1 mmol) in 1,4-dioxane:water (2:1, 3 mL), cesium carbonate (1.13 g, 3 mmol) and Pd(PPh₃)₄ (1 g, 1 mmol) at room temperature. The reaction mixture was stirred at reflux for 16 h. After completion of starting material (by TLC), the reaction mixture was diluted with water (20 mL) and extracted with 5%MeOH:CH₂Cl₂ (2×20 mL). The combined organic extracts were dried over sodium sulphate, filtered and concentrated in vacuo. The crude material was purified by column chromatography using 4-5%MeOH:CH₂Cl₂ to afford 5-(3-chlorophenyl)-3-(2-methoxy-2′-methyl-[3,4′-bipyridin]-6-yl)-6-methyl-5,6-dihydro-4H-1,2,4-oxadiazine (140 mg, 39%) as an brown solid.

Racemic compound of Example 49 was separated using a Chiralpak-I column (250×20 mm, 5 μm) (35 mg loading; 0.1% DEA in n-Hexane: CH₂Cl₂:MeOH (50:50) (A:B: 70:30) as mobile phase; flow rate: 20 mL/min) to afford the compounds of Example 49A (Fraction I (−)) and Example 49B (Fraction II (+)).

Analytical conditions for Example 49A and Example 49B. HPLC (purity): (column; X-select CSH C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 70:30); flow Rate: 1.0 mL/min):

### Example 49A

(−)-5-(3-chlorophenyl)-3-(2-methoxy-2′-methyl-[3,4′-bipyridin]-6-yl)-6-methyl-5,6-dihydro-4H-1,2,4-oxadiazine, fraction (I), (−): Mass (ESI): 409 [M+1]; HPLC (purity): 99.7%; RT 7.02 mm; Chiral HPLC: 100% RT=10.43 mm; Optical rotation [α]_{D}^{20.00}: −185.21 (c=0.25, CH₂Cl₂).

### Example 49B

(+)-5-(3-chlorophenyl)-3-(2-methoxy-2′-methyl-[3,4′-bipyridin]-6-yl)-6-methyl-5,6-dihydro-4H-1,2,4-oxadiazine, fraction (II), (+): ¹H NMR (CD₃OD, 400 MHz): δ 8.44 (d, 1H), 7.93 (d, 1H), 7.69 (d, 1H), 7.56 (s, 1H), 7.50 (dd, 1H), 7.37-7.30 (m, 3H), 7.26-723 (m, 1H), 4.74-4.71 (m, 1H), 4.07-4.04 (m, 1H), 4.03 (s, 3H), 2.59 (s, 3H), 1.01 (d, 3H); Mass (ESI): 409 [M+1]; HPLC (purity): 98.0%; RT 7.02 mm; Chiral HPLC: 100% RT=13.98 mm; Optical rotation [α]_{D}^{20.00}: +173.93 (c=0.25, CH₂Cl₂).

### Example 50

### Synthesis of 8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole

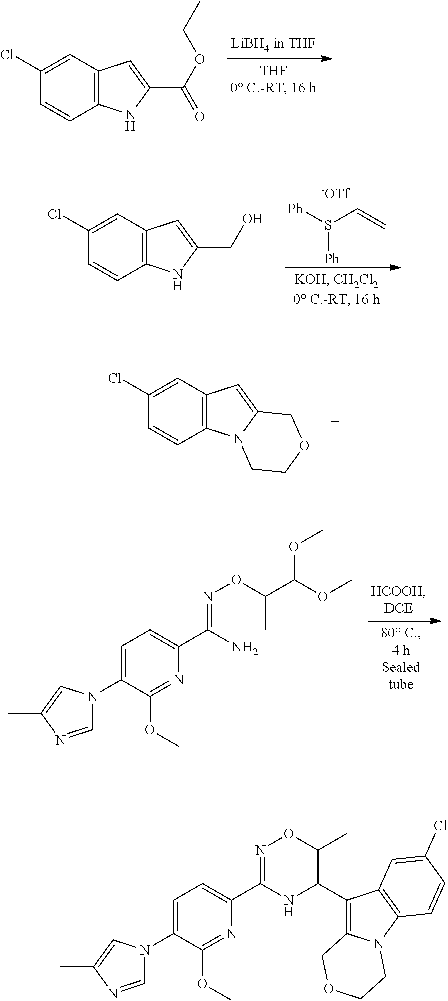

### Synthesis of (5-chloro-1H-indol-2-yl) methanol

To a stirred solution of ethyl 5-chloro-1H-indole-2-carboxylate (3 g, 13 mmol) in THF (60 mL) at 0° C. under an argon atmosphere was added 2M LiBH₄ in THF (582 mg, 27 mmol). The reaction mixture was warmed to room temperature and stirred for 16 h. After consumption of starting material (by TLC), the reaction mixture was diluted with saturated ammonium chloride solution (20 mL) and extracted with EtOAc (2×30 mL). The combined organic extracts were dried over sodium sulfate, filtered and concentrated in vacuo to obtain (5-chloro-1H-indo1-2-yl) methanol (1.75 g, 72%) as an off white solid used in the next step without further purification.

¹H NMR (DMSO-d₆, 500 MHz): δ 11.20 (brs, 1H), 7.49 (s, 1H), 7.32 (d, 1H), 7.01 (dd, 1H), 6.26 (s, 1H), 5.29 (t, 1H), 4.60 (d, 2H); TLC: 30% EtOAc/Hexane (R_{f}:03).

### Synthesis of 8-chloro-3,4-dihydro-1H-[1,4] oxazino [4,3-a] indole

To a stirred solution of 5-chloro-1H-indole-2-carboxylic acid (800 mg, 4 mmol) in CH₂Cl₂ (280 mL) at 0° C. under an argon atmosphere was added potassium hydroxide (618 mg, 11 mmol). Then ((trifluoromethyl) sulfonyl)-11-oxidane compound with diphenyl (vinyl)-13-sulfane (1.92 g, 5 mmol) in CH₂Cl₂(200 mL) was added to the reaction mixture at 0° C. The reaction mixture was warmed to room temperature and stirred for 16 h. After consumption of starting material (by TLC), the volatiles were concentrated in vacuo. The crude material was purified by column chromatography using 5% EtOAc: Hexane to afford 8-chloro-3,4-dihydro-1H-[1,4] oxazino [4,3-a] indole (530 mg, 58%) as an off-white solid. LCMS: 99.3%; 208 (M+1); (column; X-select CSH C-18 (50×3.0 mm, 2.5 μm); RT 3.38 min; mobile phase: 2.5 mM Aq NH₄OOCH in water +5% ACN:ACN+5% 2.5 mM NH₄OOCH in water; T/B %: 0.01/5, 0.5/5, 3.5/100, 6/100; flow rate: 0.8 mL/min) (Gradient); TLC: 20% EtOAc/Hexane (R_{f}:06).

### Synthesis of 8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole

To a stirred solution of (Z)-N′-((1,1-dimethoxypropan-2-yl) oxy)-6-methoxy-5-(4-methyl-1H-imidazol-1-yl) picolinimidamide (450 mg, 2 mmol) in 1, 2-dichloro ethane (9 mL) at room temperature under an argon atmosphere was added 8-chloro-3,4-dihydro-1H-[1,4] oxazino [4,3-a] indole (539 mg, 3 mmol) and formic acid (9 mL). The reaction mixture was stirred at 80° C. for 4 h in a sealed tube. After consumption of starting material (by TLC), the reaction mixture was diluted with sodium bicarbonate solution (20 mL) and extracted with EtOAc (2×20 mL). The combined organic extracts were dried over sodium sulfate, filtered and concentrated in vacuo. The crude material was purified by column chromatography using 4% MeOH:CH₂Cl₂ to afford 8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole (405 mg, 64%) as a pale brown solid.

### Separation of Diastereomers:

Racemic compound of Example 50 was separated using a Inertsil Diol column (250×20 mm, 5 μ) (30 mg loading; n-Hexane: CH₂Cl₂:MeOH (80:20) (A:B: 80:20) as mobile phase; flow rate: 20 mL/min) to provide the compounds of Example 50X and Example 50Y.

Analytical conditions for Example 50X and Example 50Y: HPLC: column; Zorbax SB-C-18 (150×4.6 mm, 3.5 μm); mobile Phase: Acetonitrile: 0.05% TFA; flow rate: 1.0 mL/min; Gradient program: T/B % 0.01/90, 10/10, 15/10: diluent: CH₃CN: Water.

### Example 50X

8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole: HPLC (purity): 99.3%; RT 7.34 min

### Example 50Y

8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole: HPLC (purity): 98.2%; RT 7.35 min

### Separation of Enantiomers:

Racemic compound of Example 50X was separated using a Chiralpak-IA column (250×20 mm, 5 μm) (50 mg loading; 0.1% DEA in n-Hexane: CH₂Cl₂:MeOH (50:50) (A:B: 75:25) as mobile phase; flow rate: 20 mL/min) to afford the compounds of Example 50A (Fraction I (+)) and Example 50B (Fraction II (−)).

Analytical conditions for Example 50A and Example 50B. HPLC (purity): (column; X-select CSH-C-18 150×4.6 mm, 3.5 μm); mobile Phase: ACN: 0.5% TFA; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) EtOH:MeOH (80:20) (A::B; 75:25); flow Rate: 1.0 mL/min).

### Example 50A

(+)-8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole, fraction (I), (+): ¹H NMR (CD₃OD, 400 MHz): δ 7.98 (s, 1H), 7.90 (d, 1H), 7.65 (d, 1H), 7.58 (s, 1H), 7.39 (d, 1H), 7.22 (s, 1H), 7.16 (dd, 1H), 5.10 (d, 2H), 4.62 (d, J=7.9 Hz, 1H), 4.25-4.06 (m, 4H), 3.95 (s, 3H), 3.85 (dd, J=6.2, 8.0 Hz, 1H), 2.26 (d, J=0.9 Hz, 3H), 1.24 (d, J=6.4 Hz, 3H); Mass (ESI): 493.2 [M+1]; HPLC (purity): 99.8%; RT 7.06 min; Chiral HPLC: 100% RT=9.81 mm; Optical rotation [α]_{D}^{19.99}: +182.33 (c=0.25, CH₂Cl₂).

### Example 50B

(−)-8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole, fraction (II), (−): Mass (ESI): 493.1 [M+1]; HPLC (purity): 99.8%; RT 7.06 min; Chiral HPLC: 99.6% RT=11.43 mm; Optical rotation [α]_{D}^{19.99}: −165.82 (c=0.25, CH₂Cl₂).

Racemic compound of Example 50Y was separated using a Chiralpak-IA column (250×20 mm, 5 μm) (50 mg loading; 0.1% DEA in n-Hexane: CH₂Cl₂:MeOH (50:50) (A:B: 75:25) as mobile phase; flow rate: 20 mL/min) to afford the compounds of Example 50C (Fraction III (−)) and Example 50D (Fraction IV (+)).

Analytical conditions for Example 50C and Example 50D. HPLC (purity): (column; X-select CSH-C-18 150×4.6 mm, 3 5 nm); mobile Phase: ACN: 0.5% TFA; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) EtOH:MeOH (80:20) (A::B; 75:25); flow Rate: 1.0 mL/min).

### Example 50C

(−)-8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole, fraction (III), (−): ¹H NMR (CD₃OD, 400 MHz): δ 7.98 (s, 1H), 7.90 (d, 1H), 7.65 (d, 1H), 7.58 (s, 1H), 7.39 (d, 1H), 7.22 (s, 1H), 7.16 (dd, 1H), 5.10 (d, 2H), 4.62 (d, J=7.9 Hz, 1H), 4.25-4.06 (m, 4H), 3.95 (s, 3H), 3.85 (dd, J=6.2, 8.0 Hz, 1H), 2.26 (d, J=0.9 Hz, 3H), 1.24 (d, J=6.4 Hz, 3H); Mass (ESI): 493.1 [M+1]; HPLC (purity): 98.7%; RT 7.08 min; Chiral HPLC: 100% RT=9.85 mm; Optical rotation [α]_{D}^{20.04}: −50.59 (c=0.25, CH₂Cl₂).

### Example 50D

(+)-8-chloro-10-(3-(6-methoxy-5-(4-methyl-1H-imidazol-1-yl) pyridin-2-yl)-6-methyl-5,6-dihydro-4H-1,2,4-oxadiazin-5-yl)-3,4-dihydro-1H-[1,4] oxazino [4,3-a] indole, fraction (IV), (+): Mass (ESI): 493.1 [M+1]; HPLC (purity): 98.9%; RT 7.08 min; Chiral HPLC: 99.7% RT=11.81 mm; Optical rotation [α]_{D}^{20.01}: +49.34 (c=0.25, CH₂Cl₂).

### Example 51

### Synthesis of 5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

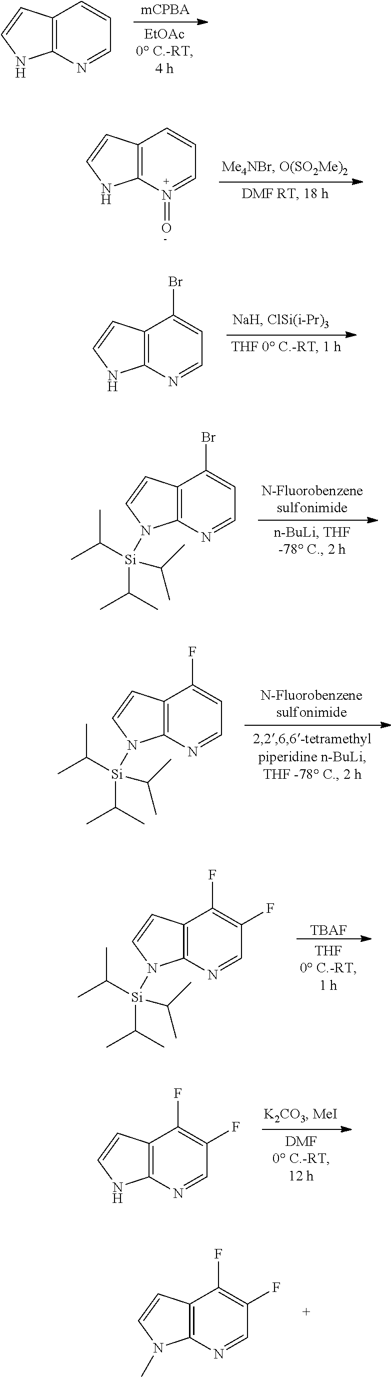

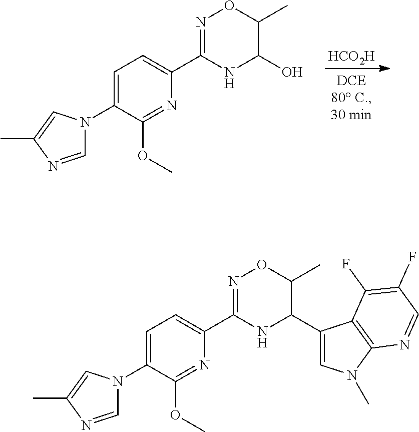

### Synthesis of 1H-pyrrolo [2,3-b] pyridine 7-oxide

To a stirred solution of m-chloro per benzoic acid (64 g, 372 mmol) in EtOAc (260 mL) at 0° C. under an argon atmosphere was added 1H-pyrrolo [2,3-b] pyridine (40 g, 338 mmol) in EtOAc (780 mL). The reaction mixture was warmed to room temperature and stirred for 4 h. After consumption of starting material (by TLC), the obtained solid was filtered and dried in vacuo to obtain 4-pyrrolo [2,3-6] pyridine N-oxide 3-chloro benzoic acid salt as white solid.

To a stirred solution of the above solid in water (334 mL) m-chloro per benzoic acid at room temperature was added 30% Aq potassium carbonate to pH above 9-10 and stirred for 1 h. Then the reaction mixture was cooled to 0° C., to obtain the solid, the solid was washed with water (50 mL) and dried in vacuo to obtain 1H-pyrrolo [2,3-b] pyridine 7-oxide (26 g, 57%) as an off-white solid. ¹H NMR (DMSO-d₆, 400 MHz): δ 12.45 (br s, 1H), 8.10 (d, 1H), 7.62 (d, 1H), 7.43 (s, 1H), 7.05 (dd, 1H), 6.57 (s, 1H); TLC: 5% MeOH/CH₂Cl₂ (R_{f}:0.2).

### Synthesis of 4-bromo-1H-pyrrolo [2,3-b] pyridine

To a stirred solution of tetra methyl ammonium bromide (45 g, 291 mmol) in DMF (182 mL) at 0° C. under an argon atmosphere were added 1H-pyrrolo [2,3-b] pyridine 7-oxide (26 g, 194 mmol) and methane sulfonic anhydride (67.5 g, 388 mmol). The reaction mixture was warmed to room temperature and stirred for 18 h. After consumption of starting material (by TLC), the reaction mixture was neutralised with 50% sodium hydroxide solution to pH above 8-9, diluted with water (400 mL) to obtain the solid. The solid was dissolved in 10%MeOH/CH₂Cl₂(50 mL), dried over sodium sulfate, filtered and concentrated in vacuo. The crude material was washed with n-pentane (2×50 mL) to afford 4-bromo-1H-pyrrolo [2,3-b] pyridine (15.2 g, 40%) as an off-white solid. ¹H NMR (DMSO-d₆, 500 MHz): δ 12.01 (br s, 1H), 8.07 (s, 1H), 7.60 (s, 1H), 7.30 (d, 1H), 6.40 (s, 1H), 6.57 (s, 1H); TLC: 5% MeOH/CH₂Cl₂ (R_{f}:07).

### Synthesis of 4-bromo-1-(triisopropylsilyl)-1H-pyrrolo [2,3-b] pyridine

To a stirred solution of 4-bromo-1H-pyrrolo [2,3-b] pyridine (19 g, 96 mmol) in THF (285 mL) at 0° C. under an argon atmosphere were added sodium hydride (7.7 g, 192 mmol). The reaction mixture was stirred for 15 min. Then chlorotriisopropylsilane (28 g, 144 mmol) was added to the reaction mixture at 0° C. The reaction mixture was warmed to room temperature and stirred for 1 h. After consumption of starting material (by TLC), the reaction mixture was quenched with saturated ammonium chloride solution (450 mL) and extracted with hexane (2×350 mL). The combined organic extracts were washed with water (300 mL), brine (200 mL), dried over sodium sulfate, filtered and concentrated in vacuo. The crude material was purified by column chromatography using 1-2%EtOAc: Hexane to afford 4-bromo-1-(triisopropylsilyl)-1H-pyrrolo [2,3-b] pyridine (27 g, 80%) as colorless liquid. ¹H NMR (CDCl₃, 500 MHz): δ 8.05 (d, 1H), 7.34 (d, 1H), 7.22 (d, 1H), 6.59 (d, 1H), 1.88-1.82 (m, 3H), 1.12 (d, 18H); TLC: 20% EtOAc/Hexane (R_{f}:07).

### Synthesis of 4-fluoro-1-(triisopropylsilyl)-1H-pyrrolo [2,3-b] pyridine

To a stirred solution of 4-bromo-1-(triisopropylsilyl)-1H-pyrrolo [2,3-b] pyridine (27 g, 76 mmol) in THF (772 mL) at -78° C. under an argon atmosphere was added n-BuLi (9.79 g, 153 mmol). The reaction mixture was stirred for 15 min. Then N-fluorobenzenesulfonimide (26.50 g, 84 mmol) in THF (416 mL) was added to the reaction mixture at −78° C. The reaction mixture was stirred for 2 h. After consumption of starting material (by TLC), the reaction mixture was quenched with saturated ammonium chloride solution (400 mL) and extracted with hexane (2×400 mL). The combined organic extracts were dried over sodium sulfate, filtered and concentrated in vacuo. The crude material was purified by column chromatography using Hexane to afford 4-fluoro-1-(triisopropylsilyl)-1H-pyrrolo [2,3-b] pyridine (10.5 g, 47%) as colorless liquid. ¹H NMR (CDCl₃, 500 MHz): δ 8.18 (dd, 1H), 7.25 (s, 1H), 6.75 (dd, 1H), 6.63 (d, 1H), 1.90-1.80 (m, 3H), 1.12 (d, 18H); TLC: Hexane (R_{f}:07).

### Synthesis of 4,5-difluoro-1-(triisopropylsilyl)-1H-pyrrolo [2,3-b] pyridine

To a stirred solution of 2,2′,6,6′-tetramethyl piperidine (9.65 g, 68 mmol) in THF (30 mL) at −78° C. under an argon atmosphere was added n-butyl lithium (27.3 mL, 68 mmol). The reaction mixture was stirred at 0° C. for 30 mm Then 4-fluoro-1-(triisopropylsilyl)-1H-pyrrolo [2,3-b] pyridine (5 g, 17 mmol) in THF (40 mL) was added and stirred for 15 mm, again N-fluorobenzene sulfonimide (13.5 g, 43 mmol) in THF (30 mL) was added to the reaction mixture at −78° C. The reaction mixture was stirred for 15 mm at −78° C. After consumption of starting material (by TLC), the reaction mixture was quenched with saturated ammonium chloride solution (50 mL) and extracted with EtOAc (2×100 mL). The combined organic extracts were dried over sodium sulfate, filtered and concentrated in vacuo. The crude material was purified by column chromatography using 20% Hexane to afford 4,5-difluoro-1-(triisopropylsilyl)-1H-pyrrolo [2,3-b] pyridine (1.7 g, 32%) as an off-white solid. ¹H NMR (CDCl₃, 500 MHz): δ 8.16 (dd, 1H), 7.29 (d, 1H), 6.64 (t, 1H), 1.87-1.73 (m, 3H), 1.10 (d, 18H); TLC: Hexane (R_{f}:07).

### Synthesis of 4,5-difluoro-1H-pyrrolo [2,3-b] pyridine

To a stirred solution of 4,5-difluoro-1-(triisopropylsilyl)-1H-pyrrolo [2,3-b] pyridine (3.5 g, 11 mmol) in THF (17.5 mL) at 0° C. under an argon atmosphere was added tertiary butyl ammonium fluoride (12.25 mL). The reaction mixture was warmed to room temperature and stirred for 1 h. After consumption of starting material (by TLC), the reaction mixture was diluted with water (50 mL) and extracted with EtOAc (2×100 mL). The combined organic extracts were dried over sodium sulfate, filtered and concentrated in vacuo. The crude material was purified by column chromatography using 20%EtOAc: Hexane to afford 4,5-difluoro-1H-pyrrolo [2,3-b] pyridine (1.6 g, 92%) as an off-white solid. ¹H NMR (CDCl₃, 400 MHz): δ 10.00 (br s, 1H), 8.28 (dd, 1H), 7.35 (s, 1H), 6.60 (s, 1H); TLC: 20% EtOAc/Hexane (R_{f}:02).

### Synthesis of 4,5-difluoro-1-methyl-1H-pyrrolo [2,3-b] pyridine

To a stirred solution of 4,5-difluoro-1H-pyrrolo [2,3-b] pyridine (1.6 g, 10 mmol) in DMF (48 mL) at 0° C. under an argon atmosphere were added potassium carbonate (2.15 g, 15 mmol) and methyl iodide (1.77 g, 12 mmol). The reaction mixture was warmed to room temperature and stirred for 12 h. After consumption of starting material (by TLC), the reaction mixture was diluted with water (50 mL) and extracted with EtOAc (2×50 mL). The combined organic extracts were dried over sodium sulfate, filtered and concentrated in vacuo. The crude material was purified by column chromatography using 15%EtOAc: Hexane to afford 4,5-difluoro-1-methyl-1H-pyrrolo [2,3-b] pyridine (1.3 g, 75%) as an off-white solid. ¹H NMR (CDCl₃, 400 MHz): δ 8.28 (dd, 1H), 7.18 (s, 1H), 6.52 (s, 1H), 3.88 (s, 3H); TLC: 20% EtOAc/Hexane (R_{f}:05).

### Synthesis of 5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

To a stirred solution of 3-(6-methoxy-5-(4-methyl-1H-imidazol-1-yl) pyridin-2-yl)-6-methyl-5,6-dihydro-4H-1,2,4-oxadiazin-5-ol (800 mg, 3 mmol) in 1,2-dichloro ethane (16 mL) at room temperature under an argon atmosphere were added formic acid (16 mL) and 4,5-difluoro-1-methyl-1H-pyrrolo [2,3-b] pyridine (887 mg, 5 mmol). The reaction mixture was stirred at 80° C. for 30 mm After consumption of starting material (by TLC), the reaction mixture was diluted with sodium bicarbonate solution (20 mL) and extracted with EtOAc (2×20 mL). The combined organic extracts were dried over sodium sulfate, filtered and concentrated in vacuo. The crude material was purified by column chromatography using 3% MeOH:CH₂Cl₂ to afford 5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine (260 mg, 22%) as an off-white solid.

### Seperation of Diastereomers:

Racemic compound of Example 51 was separated using a YMC silica column (250×20 mm, 5 μ) (50 mg loading; n-Hexane: CH₂Cl₂:MeOH (50:50) (A:B: 85:15) as mobile phase; flow rate: 20 mL/min) to provide the compounds of Example 51X and Example 51Y.

Analytical conditions for Example 51X and Example 51Y: HPLC: column; X-select CSH C-18 (150×4.6 mm, 3.5 μm); mobile Phase: Acetonitrile: 0.05% TFA (50:50) (A:B: 85:15); flow rate: 1.0 mL/min; Gradient program: T/B % 0.01/90, 10/10, 15/10: diluent: CH₃CN: Water.

### Example 51X

5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 98.8%; RT 6.48 min

### Example 51Y

5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine HPLC (purity): 91.8%; RT 6.27 min

### Seperation of Enantiomers:

Racemic compound of Example 51X was separated using a Chiralpak-IA column (250×20 mm, 5 μm) (25 mg loading; 0.1% DEA in n-Hexane: CH₂Cl₂:MeOH (50:50) (A:B: 85:15) as mobile phase; flow rate: 20 mL/min) to afford the compounds of Example 51A (Fraction I (+)) and Example 51B (Fraction II (−)).

Analytical conditions for Example 51A and Example 51B. HPLC (purity): (column; X-select CSH-C-18 (150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 51A

(+)-5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (I), (+): ¹H NMR (CD₃OD, 400 MHz): δ 8.32 (dd, 1H), 7.96 (s, 1H), 7.88 (d, 1H), 7.65 (s, 1H), 7.63 (s, 1H), 7.20 (s, 1H), 4.67 (dd, 1H), 3.95 (s, 3H), 3.89-3.86 (m, 4H), 2.24 (s, 3H), 1.27 (d, 3H); Mass (ESI): 454.1 [M+1]; HPLC (purity): 99.2%; RT 6.28 min; Chiral HPLC: 99.6% RT=8.74 min; Optical rotation [α]_{D}^{20.00}: +93.28 (c=0.25, CH₂Cl₂).

### Example 51B

(−)-5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (II), (−): Mass (ESI): 454.1 [M+1]; HPLC (purity): 96.8%; RT 6.28 min; Chiral HPLC: 97.5% RT=11.33 min; Optical rotation [α]_{D}^{20.00}: −87.05 (c=0.25, CH₂Cl₂).

Racemic compound of Example 51Y was separated using a Chiralpak-IC column (250×20 mm, 5 μm) (35 mg loading; 0.1% DEA in n-Hexane: CH₂Cl₂:MeOH (50:50) (A:B: 40:60) as mobile phase; flow rate: 20 mL/min) to afford the compounds of Example 51C (Fraction III (−)) and Example 51D (Fraction IV (+)).

Analytical conditions for Example 51C and Example 51D. HPLC (purity): (column; X-select CSH-C-18 (150×4.6 mm, 3.5 μ); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 40:60); flow Rate: 1.0 mL/min).

### Example 51C

(+)-5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (III), (−): Mass (ESI): 454.1 [M+1]; HPLC (purity): 99.8%; RT 6.49 min; Chiral HPLC: 100% RT=15.87 min; Optical rotation [α]_{D}^{19.98}: −71.23 (c=0.25, CH₂Cl₂).

### Example 51D

(+)-5-(4,5-difluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (IV), (+): ¹H NMR (CD₃OD, 400 MHz): δ 8.32 (dd, 1H), 7.96 (s, 1H), 7.88 (d, 1H), 7.65 (s, 1H), 7.63 (s, 1H), 7.20 (s, 1H), 4.67 (dd, 1H), 3.95 (s, 3H), 3.89-3.86 (m, 4H), 2.24 (s, 3H), 1.27 (d, 3H); Mass (ESI): 454.1 [M+1]; HPLC (purity): 99.1%; RT 6.49 mm; Chiral HPLC: 100% RT=22.82 mm; Optical rotation [α]_{D}^{20.00}: +79.36 (c=0.25, CH₂Cl₂).

### Example 52

### Synthesis of 5-(4,5-difluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine

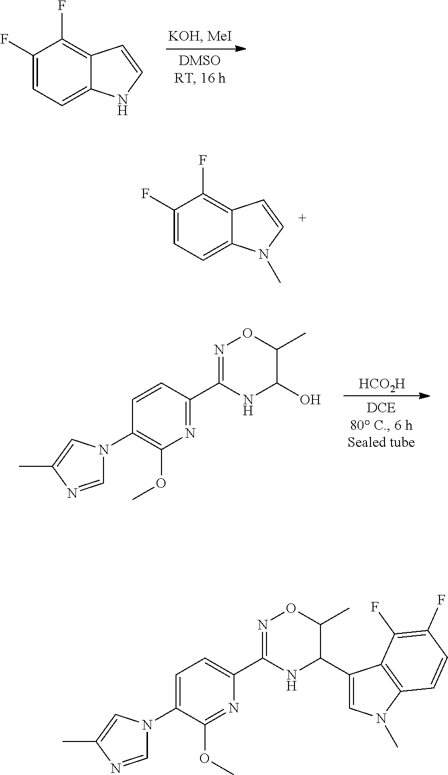

### Synthesis of 4,5-difluoro-1-methyl-1H-indole

To a stirred solution of 4,5-difluoro-1H-indole (1 g, 6 mmol) in DMSO (10 mL) at 0° C. under an argon atmosphere were added potassium hydroxide (732 mg, 13 mmol) and methyl iodide (1 g, 7 mmol). The reaction mixture was warmed to room temperature and stirred for 16 h. After consumption of starting material (by TLC), the reaction mixture was quenched with water (100 mL) and extracted with EtOAc (2×100 mL). The combined organic extracts were dried over sodium sulfate, filtered and concentrated in vacuo. The crude material was purified by column chromatography using 8% EtOAc: Hexane to afford 4,5-difluoro-1-methyl-1H-indole (900 mg, 90%) as a pale yellow solid. ¹H NMR (CDCl₃, 400 MHz): 7.36-7.30 (m, 1H), 7.09-7.01 (m, 2H), 6.40 (d, 1H), 3.71 (s, 3H); TLC: 20% EtOAc/Hexane (R_{f}:06).

### Synthesis of 5-(4,5-difluoro-1-methyl-1H-indo1-3-yl)-3-(6-methoxy-5-(4-methyl-1H-imidazol-1-yl) pyridin-2-yl)-6-methyl-5,6-dihydro-4H-1,2,4-oxadiazine

To a stirred solution of 3-(6-methoxy-5-(4-methyl-1H-imidazol-1-yl) pyridin-2-yl)-6-methyl-5,6-dihydro-4H-1,2,4-oxadiazin-5-ol (350 mg, 1 mmol) in 1, 2-dichloro ethane (3.5 mL) at room temperature under an argon atmosphere were added formic acid (3.5 mL) and 4,5-difluoro-1-methyl-1H-indole (385 mg, 2 mmol). The reaction mixture was stirred at 80° C. for 6 h a sealed tube. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 2% MeOH:CH₂Cl₂ to afford 5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine (420 mg, 80%) as an off-white solid.

### Separation of Diastereomers:

Racemic compound of Example 52 was separated using a Inertsil Diol column (250×20 mm, 5 μm) (50 mg loading; n-Hexane: CH₂Cl₂:MeOH (50:50) (A:B: 85:15) as mobile phase; flow rate: 20 mL/min) to provide the compounds of Example 52X and Example 52Y.

Analytical conditions for Example 52X and Example 52Y: HPLC: (column; X-select CSH-C-18 150×4.6 mm, 3.5 μ); mobile Phase: ACN+5%0.05%TFA: 0.05% Aq TFA+5%ACN; flow rate: 1.0 mL/min; Gradient program: T/B % 0.01/90, 10/10, 15/10: diluent: CH₃CN: Water.

### Example 52X

5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 99.0%; RT 7.00 min

### Example 52Y

5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 92.9%; RT 7.06 min

### Separation of Enantiomers:

Racemic compound of Example 52X was separated using a Chiralpak-IB column (250×20 mm, 5 μm) (25 mg loading; 0.1% DEA in n-Hexane: CH₂Cl₂:MeOH (50:50) (A:B: 85:15) as mobile phase; flow rate: 20 mL/min) to afford the compounds of Example 52A (Fraction I (−)) and Example 52B (Fraction II (+)).

Analytical conditions for Example 52A and Example 52B. HPLC (purity): (column; X-select CSH-C-18 150×4.6 mm, 3.5 μ); mobile Phase: ACN+5%0.05%TFA: 0.05% Aq TFA+5%ACN; flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (50:50) (A::B; 85:15); flow Rate: 1.0 mL/min).

### Example 52A

(−)-5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (I), (−): Mass (ESI): 453.1[M+1]; HPLC (purity): 96.5%; RT 6.84 min; Chiral HPLC: 98.6% RT=11.30 mm; Optical rotation [α]_{D}^{19.99}: −95.44 (c=0.25, CH₂Cl₂).

### Example 52B

(+)-5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (II), (+): ¹H NMR (CD₃OD, 400 MHz): δ 7.97 (s, 1H), 7.89 (d, 1H), 7.65 (d, 1H), 7.48 (dd, 1H), 7.39 (s, 1H), 7.35 (dd, 1H), 7.21 (s, 1H), 4.61 (d, 1H), 3.94 (s, 3H), 3.84 (dd, 1H), 3.79 (s, 3H), 2.25 (s, 3H), 1.25 (d, 3H); Mass (ESI): 453.1 [M+1]; HPLC (purity): 99.5%; RT 6.85 min; Chiral HPLC: 100% RT=16.82 mm; Optical rotation [α]_{D}^{19.99}: +99.16 (c=0.25, CH₂Cl₂).

Racemic compound of Example 52Y was separated using a Chiralpak-ODH column (250×20 mm, 5 μm) (25 mg loading; 0.1% DEA in n-Hexane: EtOH:MeOH (90:10) (A:B: 70:30) as mobile phase; flow rate: 20 mL/min) to afford the compounds of Example 52C (Fraction III (+)) and Example 52D (Fraction IV (−)).

Analytical conditions for Example 52C and Example 52D. HPLC (purity): (column; X-select CSH-C-18 150×4.6 mm, 3.5 μ); mobile Phase: ACN+5%0.05%TFA: 0.05% TFA+5%ACN; flow rate: 1.0 mL/min; Gradient program: T/B % 0.01/90, 10/10, 15/10: diluent: CH₃CN: Water; Chiral HPLC: (Chiralpak-ODH (250×4.6 mm, 5 μm; mobile phase (A) 0.1% DEA in n-Hexane (B) CH₂Cl₂:MeOH (50:50) (A::B; 70:30); flow Rate: 1.0 mL/min).

### Example 52C

(+)-5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (I), (+): ¹H NMR (CD₃OD, 400 MHz): δ 7.97 (s, 1H), 7.89 (d, 1H), 7.65 (d, 1H), 7.48 (dd, 1H), 7.39 (s, 1H), 7.35 (dd, 1H), 7.21 (s, 1H), 4.61 (d, 1H), 3.94 (s, 3H), 3.84 (dd, 1H), 3.79 (s, 3H), 2.25 (s, 3H), 1.25 (d, 3H); Mass (ESI): 453 [M+1]; HPLC (purity): 95.0%; RT 6.78 min; Chiral HPLC: 100% RT=14.20 mm; Optical rotation [α]_{D}^{19.98}: +54.06 (c=0.25, CH₂Cl₂).

### Example 52D

(−)-5-(4,5-difluoro-1-methyl-1H-indol-3-yl)-3-(6-methoxy-5-(4-methyl-1H-imidazol-1-yl) pyridin-2-yl)-6-methyl-5,6-dihydro-4H-1,2,4-oxadiazine, fraction (II), (−): Mass (ESI): 453.1 [M+1]; HPLC (purity): 98.4%; RT 6.79 mm; Chiral HPLC: 100% RT=21.46 mm; Optical rotation [α]_{D}^{19.99}: −51.20 (c=0.25, CH₂Cl₂).

### Example 53

### Synthesis of 5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine

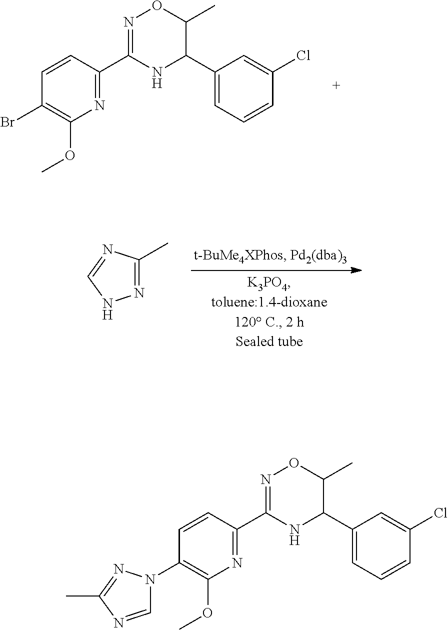

### Synthesis of 5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine

To a dry vial was added a suspension of Pd₂(dba)₃ (29 mg, 0.03 mmol) and tert-butyl tetramethyl Xphos (30 mg, 0.06 mmol) in toluene: 1,4-dioxane (2:1, 3.75 mL) at room temperature. The suspension was degassed with argon, heated to 120° C., and stirred at 120° C. for 3 min. A mixture of 3-(5-bromo-6-methoxypyridin-2-yl)-5-(3-chlorophenyl)-6-methyl-5,6-dihydro-4H-1,2,4-oxadiazine (250 mg, 0.6 mmol), 3-methyl-1H-1,2,4-triazole (63 mg, 0.8 mmol) and potassium phosphate (267 mg, 1 mmol) in toluene: 1,4-dioxane (2:1, 3.75 mL) was degassed and the catalyst premix was added. The resulting mixture was stirred at 120° C. for 2 h in a sealed tube. After consumption of the starting material (monitored by TLC and LCMS), the reaction mixture was diluted with water (20 mL) and extracted with EtOAc (2×20 mL). The combined organic extracts were dried over sodium sulfate, filtered and concentrated in vacuo. The crude material was purified by column chromatography using 1-5%MeOH:CH₂Cl₂ to afford 5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine (110 mg, 44%) as an off-white solid.

Racemic compound of Example 53 was separated using a Chiralpak-IB column (250×20 mm, 5 μm) (20 mg loading; 0.1% DEA in n-Hexane: CH₂Cl₂:MeOH (80:20) (A:B: 90:10) as mobile phase; flow rate: 20 mL/min) to afford the compounds of Example 53A (Fraction I (−)) and Example 53B (Fraction II (+)).

Analytical conditions for Example 53A and Example 53B. HPLC (purity): (column; X-select CSH C-18 150×4.6 mm, 3.5 μm); mobile Phase: ACN: 5% 0.05% TFA; 0.05% TFA+5% ACN flow rate: 1.0 mL/min; Gradient program: T/B % 0.01/90, 10/10, 15/10: diluent: CH₃CN: Water; Chiral HPLC: (Chiralpak-IB (250×4.6 mm, 5 μm; mobile phase (A) 0.1% DEA in n-Hexane (B) CH₂Cl₂:MeOH (80:20) (A::B; 90:10); flow Rate: 1.0 mL/min).

### Example 53A

(−)-5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine, fraction (I), (−): Mass (ESI): 399 [M+1]; HPLC (purity): 96.2%; RT 9.37 mm; Chiral HPLC: 99.3% RT=19.67 min; Optical rotation [α]_{D}^{19.98}: −139.31 (c=0.25, CH₂Cl₂).

### Example 53B

(+)-5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine, fraction (II), (+): ¹H NMR (CD₃OD, 400 MHz): δ 8.97 (s, 1H), 8.24 (d, 1H), 7.72 (dd, 1H), 7.34-7.30 (m, 3H), 7.22-7.20 (m, 1H), 4.70 (s, 1H), 4.15 (s, 3H), 4.05 (dd, 1H), 2.42(s, 3H), 1.00 (dd, 3H); Mass (ESI): 399 [M+1]; HPLC (purity): 99.1%; RT 9.37 mm; Chiral HPLC: 99.4% RT=24.90 min; Optical rotation [α]_{D}^{19.98}: +143.34 (c=0.25, CH₂Cl₂).

### Example 54

### Synthesis of 5-(3-chlorophenyl)-3-(6-methoxy-5-(3-methyl-1H-1,2,4-triazol-1-yl) pyridin-2-yl)-6-methyl-5,6-dihydro-4H-1,2,4-oxadiazine

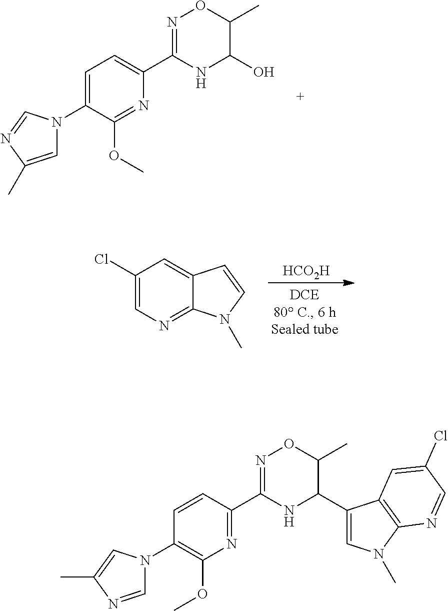 [SMILES: COc1nc(C2=NOC(C)C(O)N2)ccc1-n1cnc(C)c1 ; Cn1ccc2cc(Cl)cnc21 ; "HCO2H" ; COc1nc(C2=NOC(C)C(c3cn(C)c4ncc(Cl)cc34)N2)ccc1-n1cnc(C)c1]

### Synthesis of 5-(5-chloro-1-methyl-1H-pyrrolo [2,3-b]pyridin-3-yl)-3-(6-methoxy-5-(4-methyl-1H-imidazol-1-yl)pyridin-2-yl)-6-methyl-5,6-dihydro-4H-1,2,4-oxadiazine

To a stirred solution of 3-(6-methoxy-5-(4-methyl-1H-imidazol-1-yl) pyridin-2-yl)-6-methyl-5,6-dihydro-4H-1,2,4-oxadiazin-5-ol (300 mg, 1 mmol) in dichloro ethane (3 mL) at room temperature under an argon atmosphere were added 5-chloro-1-methyl-1H-pyrrolo [2,3-b] pyridine (325 mg, 2 mmol) and formic acid (3 mL). The reaction mixture was stirred at 80° C. for 16 h in a sealed tube. After consumption of starting material (by TLC), the reaction mixture was diluted with saturated sodium bicarbonate solution (20 mL) and extracted with EtOAc (2×20 mL). The combined organic extracts were dried over sodium sulfate, filtered and concentrated in vacuo. The crude material was purified by column chromatography using 1-5% MeOH:CH₂Cl₂ to afford 5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine (210 mg, 47%) as an off-white solid.

### Separation of Diastereomers:

Racemic compound of Example 54 was separated using a YMC silica column (250×20 mm, 5μ) (30 mg loading; n-Hexane: CH₂Cl₂:MeOH (50:50) (A:B: 85:15) as mobile phase; flow rate: 20 mL/min) to provide the compounds of Example 54X and Example 54Y.

Analytical conditions for Example 54X and Example 54Y: HPLC: column; X-select CSH-C18 (150×4.6 mm, 3.5 μ); mobile Phase: Acetonitrile: 5mM NH₄OAc; flow rate: 1.0 mL/min; Gradient program: T/B % 0.01/80, 3/80, 10/10, 20/10: diluent: CH₃CN: Water.

### Example 54X

5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine HPLC (purity): 98.1%; RT 10.24 min

### Example 54Y

5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 91.3%; RT 10.05 min

### Separation of Enantiomers:

Racemic compound of Example 54X was separated using a Chiralpak-IB column (250×20 mm, 5 μm) (50 mg loading; 0.1% DEA in n-Hexane: CH₂Cl₂:MeOH (50:50) (A:B: 80:20) as mobile phase; flow rate: 20 mL/min) to afford the compounds of Example 54A (Fraction I (−)) and Example 54B (Fraction II (+)).

Analytical conditions for Example 54A and Example 54B. HPLC (purity): (column; X-select CSH-C-18 (150×4.6 mm, 3.5 μm); mobile Phase: CH₃CN: 5 mM NH₄OAc; flow rate: 1.0 mL/min; Gradient program: T/B % 0.01/80, 3/80, 10/10, 20/10: Chiral HPLC: (Chiralpak IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 54A

(−)-5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (I) (−): Mass (ESI): 452.1 [M+1]; HPLC (purity): 98.9%; RT 10.31 mm; Chiral HPLC: 100% RT=10.97 min; Optical rotation [α]_{D}^{20.02}: −176.06 (c=0.25, CH₂Cl₂).

### Example 54B

(+)-5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (I) (+): ¹H NMR (CD₃OD, 400 MHz): δ 8.21 (s, 1H), 8.10 (s, 1H), 7.97 (s, 1H), 7.89 (dd, 1H), 7.65 (dd, 1H), 7.60 (s, 1H), 7.20 (s, 1H), 4.62 (d, 1H), 3.94 (s, 3H), 3.90-3.84 (m, 4H), 2.21 (s, 3H), 1.29 (d, 3H); Mass (ESI): 452 [M+1]; HPLC (purity): 99.8%; RT 10.31 min; Chiral HPLC: 100% RT=17.93 min; Optical rotation [α]_{D}^{20.00}: +185.44 (c=0.25, CH₂Cl₂).

Racemic compound of Example 54Y was separated using a Chiralpak-IB column (250×20 mm, 5 μm) (50 mg loading; 0.1% DEA in n-Hexane: CH₂Cl₂:MeOH (50:50) (A:B: 80:20) as mobile phase; flow rate: 20 mL/min) to afford the compounds of Example 54C (Fraction III (−)) and Example 54D (Fraction IV (+)).

Analytical conditions for Example 54C and Example 54D. HPLC (purity): (column; X-select CSH-C-18 (150×4.6 mm, 3.5 μm); mobile Phase: CH₃CN: 5 mM NH₄OAc; flow rate: 1.0 mL/min; Gradient program: T/B % 0.01/80, 3/80, 10/10, 20/10: Chiral HPLC: (Chiralpak IB (250×4.6 mm, 5 μm; mobile phase (A) 0.1% DEA in n-Hexane (B) CH₂Cl₂:MeOH (50:50) (A::B; 80:20); flow Rate: 1.0 mL/min).

### Example 54C

(−)-5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (III) (−): Mass (ESI): 452 [M+1]; HPLC (purity): 98.2%; RT 10.15 mm; Chiral HPLC: 99.7% RT=12.84 mm; Optical rotation [α]_{D}^{20.01}: −435 (c=0.25, CH₂Cl₂).

### Example 54D

(+)-5-(5-chloro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (IV) (+): ¹1-1 NMR (CD₃OD, 400 MHz): δ 8.21 (s, 1H), 8.10 (s, 1H), 7.97 (s, 1H), 7.89 (dd, 1H), 7.65 (dd, 1H), 7.60 (s, 1H), 7.20 (s, 1H), 4.62 (d, 1H), 3.94 (s, 3H), 3.90-3.84 (m, 4H), 2.21 (s, 3H), 1.29 (d, 3H); Mass (ESI): 452 [M+1]; HPLC (purity): 96.0%; RT 10.14 mm; Chiral HPLC: 100% RT=15.87 mm; Optical rotation [α]_{D}^{19.99}: +41.32 (c=0.25, CH₂Cl₂).

### Example 55

### Synthesis of 5-(4-chloro-3-(difluoromethyl) phenyl)-3-(6-methoxy-5-(4-methyl-1H-imidazol-1-yl) pyridin-2-yl)-6-methyl-5,6-dihydro-4H-1,2,4-oxadiazine

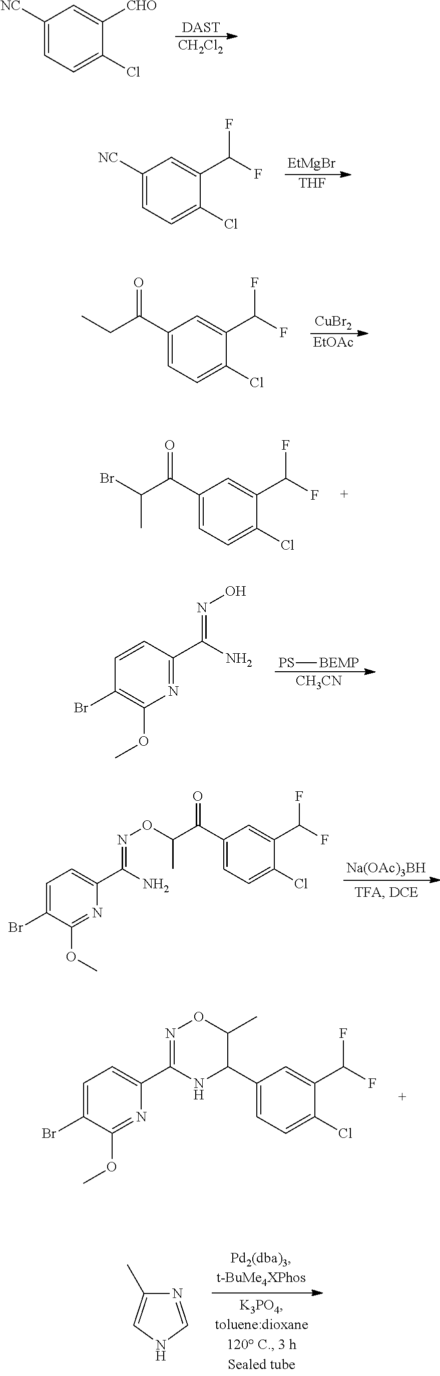

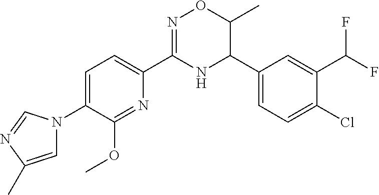 [SMILES: COc1nc(C2=NOC(C)C(c3ccc(Cl)c(C(F)F)c3)N2)ccc1-n1cnc(C)c1]

### Synthesis of 4-chloro-3-(difluoromethyl) benzonitrile

To a stirred solution of 4-chloro-3-formylbenzonitrile (2 g, 12 mmol) in CH₂Cl₂ (40 mL) at 0° C. under an argon atmosphere was added DAST (5.89 g, 36 mmol). The reaction mixture was warmed to room temperature and stirred for 16 h. After consumption of starting material (by TLC), the reaction mixture was diluted with ice cold water (100 mL) and extracted with CH₂Cl₂ (2×100 mL). The combined organic extracts were washed with sodium bicarbonate solution (100 mL), dried over sodium sulfate, filtered and concentrated in vacuo. The crude material was purified by column chromatography using 10-20% EtOAc: Hexane to afford 4-chloro-3-(difluoromethyl) benzonitrile (1.7 g, 75%) as an off-white solid. ¹H NMR (CDCl₃, 500 MHz): δ 7.96 (s, 1H), 7.71 (d, 1H), 7.58 (d, 1H), 6.94 (t, 1H); TLC: 20% EtOAc/Hexane (R_{f}:06).

### Synthesis of 1-(4-chloro-3-(difluoromethyl) phenyl) propan-1-one

To a stirred solution of 4-chloro-3-(difluoromethyl) benzonitrile (1.5 g, 9 mmol) in THF (33 mL) at 0° C. under an argon atmosphere was added ethyl magnesium bromide (17.6 mL, 18 mmol). The reaction mixture was warmed to room temperature and stirred for 2 h. After consumption of starting material (by TLC), the reaction mixture was quenched with saturated ammonium chloride solution (50 mL) and extracted with EtOAc (2×50 mL). The combined organic extracts were dried over sodium sulfate, filtered and concentrated in vacuo. The crude material was purified by column chromatography using 10-15% EtOAc: Hexane to afford 1-(4-chloro-3-(difluoromethyl) phenyl) propan-1-one (890 mg, 51%) as an off-white solid. ¹H NMR (CDCl₃, 400 MHz): δ 8.28 (s, 1H), 7.96 (s, 1H), 7.73-7.70 (m, 1H), 6.98 (t, 1H), 3.04-2.99 (m, 1H), 1.24 (t, 3H); TLC: 20% EtOAc/Hexane (R_{f}:05).

### Synthesis of 2-bromo-1-(4-chloro-3-(difluoromethyl) phenyl) propan-1-one

To a stirred solution of 1-(4-chloro-3-(difluoromethyl) phenyl) propan-1-one (100 mg, 0.4 mmol) in EtOAc (2 mL) at room temperature under an argon atmosphere was added copper bromide (208 mg, 0.9 mmol). The reaction mixture was stirred at 80° C. for 3 h. After consumption of starting material (by TLC), the reaction mixture was filtered and the filtrate was concentrated in vacuo. The crude material was purified by column chromatography using 2-5% EtOAc: Hexane to afford 2-bromo-1-(4-chloro-3-(difluoromethyl) phenyl) propan-1-one (80 mg, 58%) as a pale yellow solid. ¹H NMR (500MHz, CDCl₃): δ 8.31 (s, 1H), 8.08 (d, 1H), 7.60-7.53 (m, 2H), 6.98 (t, 2H), 5.26-5.22 (m, 1H), 1.92 (d, 3H); TLC: 20% EtOAc/Hexane (R_{f}:05).

### Synthesis of (Z)-5-bromo-N′-((1-(4-chloro-3-(difluoromethyl) phenyl)-1-oxopropan-2-yl) oxy)-6-methoxypicolinimidamide

To a stirred solution of (Z)-5-bromo-N′-hydroxy-6-methoxypicolinimidamide (50 mg, 0.2 mmol) in CH₃CN (1.25 mL) at room temperature under an argon atmosphere was added PS-BEMP (100 mg). The reaction mixture was stirred for 5 mm at room temperature. Then 2-bromo-1-(4-chloro-3-(difluoromethyl) phenyl) propan-1-one (91.7 mg, 0.3 mmol) in CH₃CN (1.25 mL) was added to the reaction mixture at room temperature. The reaction mixture was stirred for 3 h at room temperature. After consumption of starting material (by TLC), the volatiles were concentrated in vacuo to obtain (Z)-5-bromo-N′-((1-(4-chloro-3-(difluoromethyl) phenyl)-1-oxopropan-2-yl) oxy)-6-methoxypicolinimidamide (70 mg, crude) as brown semi solid used in the next step without further purification. LCMS: 47.6%; 463.8 (M+3); (column; Ascentis Express C-18 (50×3.0 mm, 2.7 μm); RT 3.07 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:05).

### Synthesis of 3-(5-bromo-6-methoxypyridin-2-yl)-5-(4-chloro-3-(difluoromethyl) phenyl)-6-methyl-5,6-dihydro-4H-1,2,4-oxadiazine

To a stirred solution of (Z)-5-bromo-N′-((1-(4-chloro-3-(difluoromethyl) phenyl)-1-oxopropan-2-yl) oxy)-6-methoxypicolinimidamide (72 mg, 0.2 mmol) in 1, 2-dichloro ethane (1.5 mL) at room temperature under an argon atmosphere were added trifluoroacetic acid (0.06 mL, 0.9 mmol) and sodium triacetoxyborohydride (111 mg, 0.5 mmol). The reaction mixture was stirred for 16 h at room temperature. After consumption of starting material (by TLC), the reaction mixture was diluted with 1N sodium hydroxide solution (10 mL) and extracted with CH₂Cl₂ (2×10 mL). The combined organic extracts were dried over sodium sulfate, filtered and concentrated in vacuo to obtain 3-(5-bromo-6-methoxypyridin-2-yl)-5-(4-chloro-3-(difluoromethyl) phenyl)-6-methyl-5,6-dihydro-4H-1,2,4-oxadiazine (61 mg, crude) as brown solid. ¹H NMR (CDCl₃, 400 MHz): δ 7.89 (d, 1H), 7.63 (d, 1H), 7.54 (s, 1H), 7.42-7.38 (m, 3H), 6.96 (t, 1H), 4.67-4.65 (m, 1H), 4.15-4.07 (m, 1H), 4.00 (s, 3H), 1.02 (d, 3H); LCMS: 51.3%; 447.8 (M+3); (column; Ascentis Express C-18 (50×3.0 mm, 2.7 μm); RT 2.88 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:06).

### Synthesis of 5-(4-chloro-3-(difluoromethyl) phenyl)-3-(6-methoxy-5-(4-methyl-1H-imidazol-1-yl) pyridin-2-yl)-6-methyl-5,6-dihydro-4H-1,2,4-oxadiazine

To a dry vial was added a suspension of Pd₂(dba)₃ (10.2 mg, 0.01 mmol) and tert-butyl tetramethyl Xphos (10.7 mg, 0.02 mmol) in toluene: 1,4-dioxane (2:1, 2 mL) at room temperature. The suspension was degassed with argon, heated to 120° C., and stirred at 120° C. for 3 min. A mixture of 3-(5-bromo-6-methoxypyridin-2-yl)-5-(4-chloro-3-(difluoromethyl) phenyl)-6-methyl-5,6-dihydro-4H-1,2,4-oxadiazine (100 mg, 0.2 mmol), 4-methyl-1H-imidazole (22 mg, 0.2 mmol) and potassium phosphate (95 mg, 0.4 mmol) in toluene: 1,4-dioxane (2:1, 2 mL) was degassed and the catalyst premix was added. The resulting mixture was stirred at 110° C. for 3 h in a sealed tube. After consumption of the starting material (monitored by TLC and LCMS), the reaction mixture was filtered and the filtrate was concentrated in vacuo. The crude material was purified by column chromatography using 1-3%MeOH:CH₂Cl₂ to afford 5-(4-chloro-3-(difluoromethyl) phenyl)-3-(6-methoxy-5-(4-methyl-1H-imidazol-1-yl) pyridin-2-yl)-6-methyl-5,6-dihydro-4H-1,2,4-oxadiazine (53 mg, 55%) as an off-white solid.

Racemic compound of Example 55 was separated using a Chiralpak-IA column (250×20 mm, 5 μm) (20 mg loading; 0.1% DEA in n-Hexane: CH₂Cl₂:MeOH (50:50) (A:B: 90:10) as mobile phase; flow rate: 20 mL/min) to afford the compounds of Example 55A (Fraction I (+)) and Example 55B (Fraction II (−)).

Analytical conditions for Example 55A and Example 55B. HPLC (purity): (column; Zorbax SBC-C-18 150×4.6 mm, 3.5 μm); mobile Phase: ACN: 0.05% TFA; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 90:10); flow Rate: 1.0 mL/min).

### Example 55A

(+)-5-(4-chloro-3-(difluoromethyl) phenyl)-3-(6-methoxy-5-(4-methyl-1H-imidazol-1-yl) pyridin-2-yl)-6-methyl-5,6-dihydro-4H-1,2,4-oxadiazine, fraction (I), (+): ¹H NMR (CD₃OD, 500 MHz): δ 7.97 (s, 1H), 7.89 (d, 1H), 7.66 (d, 1H), 7.60 (s, 1H), 7.50-7.40 (m, 2H), 7.20 (s, 1H), 7.02 (t, 1H), 4.81-4.79 (m, 1H), 4.08 (s, 3H), 4.07-4.05 (m, 1H), 2.22 (s, 3H), 1.00 (d, 3H); Mass (ESI): 448 [M+1]; HPLC (purity): 98.6%; RT 7.39 min; Chiral HPLC: 98.5% RT=13.30 min; Optical rotation [α]_{D}^{19.98}: +156.86 (c=0.25, CH₂Cl₂).

### Example 55B

(−)-5-(4-chloro-3-(difluoromethyl) phenyl)-3-(6-methoxy-5-(4-methyl-1H-imidazol-1-yl) pyridin-2-yl)-6-methyl-5,6-dihydro-4H-1,2,4-oxadiazine, fraction (II), (−): Mass (ESI): 447.9 [M+1]; HPLC (purity): 96.6%; RT 7.42 min; Chiral HPLC: 96.1% RT=14.96 min; Optical rotation [α]_{D}^{20.00}:−142.64 (c=0.25, CH₂Cl₂).

### Example 56

### Synthesis of 5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

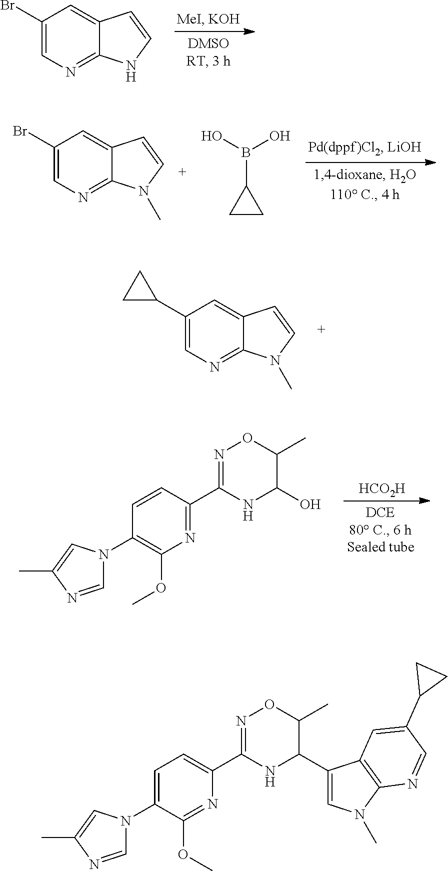

### Synthesis of 5-bromo-1-methyl-M-pyrrolo [2, 3-b] pyridine

To a stirred solution of 5-bromo-1H-pyrrolo [2, 3-b] pyridine (2 g, 10 mmol) in DMSO (20 mL) at room temperature under an argon atmosphere were added potassium hydroxide (852 mg, 15 mmol) and methyl iodide (1.95 mL, 30 mmol). The reaction mixture was warmed to room temperature and stirred for 4 h. After consumption of starting material (by TLC), the reaction mixture was diluted with ice cold water (50 mL) and extracted with EtOAc (2×50 mL). The combined organic extracts were dried over sodium sulfate, filtered and concentrated in vacuo to obtain 5-bromo-1-methyl-1H-pyrrolo [2,3-b] pyridine (2.1 g, crude) as colorless syrup used in the next step without further purification. LCMS: 98.9%; 212.7 (M+3); (column; Ascentis Express C-18 (50×3.0 mm, 2.7 μm); RT 2.49 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:06).

### Synthesis of 5-cyclopropyl-1-methyl-1H-pyrrolo [2,3-b] pyridine

To a stirred solution of 5-bromo-1-methyl-1H-pyrrolo [2,3-b] pyridine (350 mg, 3 mmol) in 1,4-dioxane: water (4:1, 2.5 mL) at room temperature under an argon atmosphere were added lithium hydroxide (445 mg, 10 mmol), cyclopropylboronic acid (455 mg, 5 mmol) and Pd(dppf)₂Cl₂ (193 mg, 0.26 mmol). The reaction mixture was stirred at 120° C. for 4 h in a sealed tube. After consumption of starting material (by TLC), the reaction mixture was filtered, the filtrate was diluted with water (20 mL) and extracted with EtOAc (2×20 mL). The combined organic extracts were dried over sodium sulfate, filtered and concentrated in vacuo. The crude material was purified by column chromatography using 10-20% EtOAc: Hexane to afford 5-cyclopropyl-1-methyl-1H-pyrrolo [2,3-b] pyridine (120 mg, 26%) as colorless liquid. ¹H NMR (CDCl₃, 400 MHz): δ 8.20 (s, 1H), 7.56 (s, 1H), 7.13 (d, 1H), 6.36 (d, 1H), 3.87 (s, 3H), 2.05-2.02 (m, 1H), 1.00-0.94 (m, 2H), 0.73-0.68 (m, 2H); LCMS: 62.8%; 172.8 (M+1); (column; Ascentis Express C-18 (50×3.0 mm, 2.7 μm); RT 1.97 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:05).

### Synthesis of 5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

To a stirred solution of 3-(6-methoxy-5-(4-methyl-1H-imidazol-1-yl) pyridin-2-yl)-6-methyl-5,6-dihydro-4H-1,2,4-oxadiazin-5-ol (650 mg, 2 mmol) in 1,2-dichloro ethane (13 mL) at room temperature under an argon atmosphere were added 5-cyclopropyl-1-methyl-1H-pyrrolo [2,3-b] pyridine (548 mg, 3 mmol) and formic acid (13 mL). The reaction mixture was stirred at 80° C. for 5 h in a sealed tube. After consumption of starting material (by TLC), the reaction mixture was diluted with saturated sodium bicarbonate solution (20 mL) and extracted with EtOAc (2×20 mL). The combined organic extracts were dried over sodium sulfate, filtered and concentrated in vacuo. The crude material was purified by column chromatography using 1-5% MeOH:CH₂Cl₂ to afford 5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine (400 mg, 41%) as an off-white solid.

### Separation of Diastereomers:

Racemic compound of Example 56 was separated using a Inertsil Diol column (250×20 mm, 5 μ) (30 mg loading; n-Hexane: CH₂Cl₂:MeOH (80:20) (A:B: 80:20) as mobile phase; flow rate: 20 mL/min) to provide the compounds of Example 56X and Example 56Y.

Analytical conditions for Example 56X and Example 56Y: HPLC: column; X-select CSH-C18 (150×4.6 mm, 3.5 μ); mobile Phase: Acetonitrile: 5mM NH₄OAc; flow rate: 1.0 mL/min; Gradient program: T/B % 0.01/80, 3/80, 10/10, 20/10: diluent: CH₃CN: Water.

### Example 56X

5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 97.8%; RT 5.02 min

### Example 56Y

5-(;5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine: HPLC (purity): 92.3%; RT 5.7 min

### Separation of Enantiomers:

Racemic compound of Example 56X was separated using a Chiralpak-IA column (250×20 mm, 5 μm) (25 mg loading; 0.1% DEA in n-Hexane: CH₂Cl₂:MeOH (50:50) (A:B: 85:15) as mobile phase; flow rate: 20 mL/min) to afford the compounds of Example 56A (Fraction I (+)) and Example 56B (Fraction II (−)).

Analytical conditions for Example 56A and Example 56B. HPLC (purity): (column; zorbax-SB-C-18 150×4.6 mm, 3.5 μ); mobile Phase: ACN: 5% 0.05% Aq TFA; 0.05% TFA: 5% ACN; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 85:15); flow Rate: 1.0 mL/min).

### Example 56A

(+)-5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (I), (+): ¹H NMR (CD₃OD, 400 MHz): δ 8.12 (s, 1H), 7.99 (s, 1H), 7.90 (d, 1H), 7.70 (s, 1H), 7.68 (s, 1H), 7.49 (s, 1H), 7.20 (s, 1H), 4.62 (d, 1H), 3.90 (s, 3H), 3.86-3.83 (m, 4H), 2.23 (s, 3H), 2.01-1.99 (m, 1H), 1.29 (d, 3H), 0.96-0.94 (m, 2H), 0.60-0.58 (m, 2H); Mass (ESI): 458.1 [M+1]; HPLC (purity): 98.1%; RT 5.03 min; Chiral HPLC: 99.4% RT=10.97 min; Optical rotation [α]_{D}^{19.99}: +40.35 (c=0.25, CH₂Cl₂).

### Example 56B

(−)-5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (II), (−): Mass (ESI): 458.5 [M+1]; HPLC (purity): 97.9%; RT 5.00 min; Chiral HPLC: 98.0% RT=12.61 min; Optical rotation [α]_{D}^{20.00}:−43.55 (c=0.25, CH₂Cl₂).

Racemic compound of Example 56Y was separated using a Chiralpak-IC column (250×20 mm, 5 μm) (70 mg loading; 0.1% DEA in n-Hexane: CH₂Cl₂:MeOH (50:50) (A:B: 30:70) as mobile phase; flow rate: 20 mL/min) to afford the compounds of Example 56C (Fraction III (−)) and Example 56D (Fraction IV (+)).

Analytical conditions for Example 56C and Example 56D. HPLC (purity): (column; X-select CSH-C-18 150×4.6 mm, 3.5 μ); mobile Phase: ACN: 5% 0.05% Aq TFA; 0.05% TFA in water: 5% ACN; flow rate: 1.0 mL/min; Gradient program: T/B % 0.01/90, 10/10, 15/10: diluent: CH₃CN: Water; Chiral HPLC: (Chiralpak-IC (250×4 6 mm, 5 μm; mobile phase (A) 0.1% DEA in n-Hexane (B) CH₂Cl₂:MeOH (50:50) (A::B; 30:70); flow Rate: 1.0 mL/min).

### Example 56C

(−)-5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (III) (−): Mass (ESI): 458.1 [M+1]; HPLC (purity): 96.7%; RT 5.90 min; Chiral HPLC: 100% RT=16.30 min; Optical rotation [α]_{D}^{20.00}: −148.92 (c=0.25, CH₂Cl₂).

### Example 56D

(+)-5-(5-cyclopropyl-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (IV) (+): NMR (CD₃OD, 400 MHz): δ 8.12 (s, 1H), 7.99 (s, 1H), 7.90 (d, 1H), 7.70 (s, 1H), 7.68 (s, 1H), 7.49 (s, 1H), 7.20 (s, 1H), 4.62 (d, 1H), 3.90 (s, 3H), 3.86-3.83 (m, 4H), 2.23 (s, 3H), 2.01-1.99 (m, 1H), 1.29 (d, 3H), 0.96-0.94 (m, 2H), 0.60-0.58 (m, 2H); Mass (ESI): 458.1 [M+1]; HPLC (purity): 98.2%; RT 5.90 min; Chiral HPLC: 100% RT=29.80 min; Optical rotation [α]_{D}^{20.01}: +131.34 (c=0.25, CH₂Cl₂).

### Example 57

### Synthesis of 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine

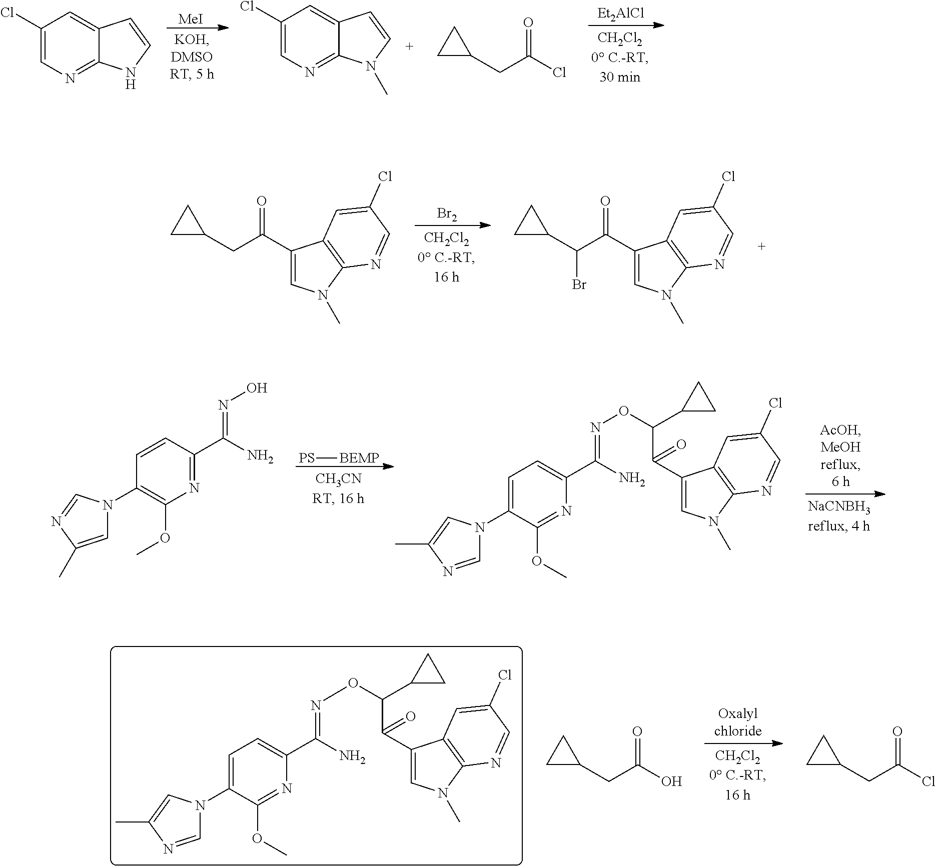

### Synthesis of 2-cyclopropylacetyl chloride

To a stirred solution of 2-cyclopropylacetic acid (500 mg, 5 mmol) in CH₂Cl₂ (2.5 mL) at 0° C. was added oxalyl chloride (0.64 mL, 7 mmol) and DMF (catalytic amount) under an argon atmosphere. The reaction mixture was warmed to room temperature and stirred for 16 h. After consumption of acid (by TLC), the volatiles were evaporated in vacuo to obtain 2-cyclopropylacetyl chloride (450 mg, crude) as colorless liquid used in the next step without further purification.

### Synthesis of 5-chloro-1-methyl-M-pyrrolo [2, 3-b] pyridine

To a stirred solution of 5-chloro-1H-pyrrolo [2, 3-b] pyridine (1 g, 7 mmol) in DMSO (10 mL) at room temperature under an argon atmosphere were added potassium hydroxide (736 mg, 13 mmol) and methyl iodide (1 g, 7 mmol). The reaction mixture was warmed to room temperature and stirred for 16 h. After consumption of starting material (by TLC), the reaction mixture was diluted with ice cold water (50 mL) and extracted with EtOAc (2×50 mL). The combined organic extracts were dried over sodium sulfate, filtered and concentrated in vacuo to obtain 5-chloro-1-methyl-1H-pyrrolo [2,3-b] pyridine (1 g, crude) as a pale yellow solid used in the next step without further purification.

¹H NMR (CDCl₃, 500 MHz): δ 8.28 (s, 1H), 7.88 (s, 1H), 7.22 (s, 1H), 6.41 (s, 1H), 3.89 (s, 3H); LCMS: 94.2%; 166.8 (M+1); (column; X-select CSH C-18 (50×3.0 mm, 2.5 μm); RT 3.13 min; mobile phase: 2.5mM NH₄OOCH in water+5% ACN:ACN+5% 2.5 mM NH₄OOCH in water; T/B %: 0.01/5, 0.5/5, 3.5/100, 6/100; flow rate: 0.8 mL/min) (Gradient); TLC: 20% EtOAc/Hexane (R_{f}:06).

### Synthesis of 1-(5-chloro-1-methyl-1H-pyrrolo [2,3-b] pyridin-3-yl)-2-cyclopropylethan-1-one

To a stirred solution of 5-chloro-1-methyl-1H-pyrrolo [2,3-b] pyridine (1.2 g, 7 mmol) in CH₂Cl₂ (7.5 mL) at 0° C. under an argon atmosphere were added diethyl aluminium chloride 1M in Hexane (12.2 mL, 12 mmol). The reaction mixture was stirred for 15 min Then 2-(2-chloro-2-oxoethyl) cyclopropan-1-ylium (2.56 g, 22 mmol) in CH₂Cl₂ (7.5 mL) was added to the reaction mixture at 0° C. The reaction mixture was warmed to room temperature and stirred for 30 min After consumption of starting material (by TLC), the reaction mixture was diluted with saturated sodium bicarbonate solution (20 mL) and extracted with EtOAc (2×20 mL). The combined organic extracts were washed with water (20 mL), dried over sodium sulfate, filtered and concentrated in vacuo. The crude material was purified by column chromatography using 20%EtOAc: Hexane to afford 1-(5-chloro-1-methyl-1H-pyrrolo pyridin-3-yl)-2-cyclopropylethan-1-one (1.4 g, 78%) as an off-white solid. ¹H NMR (CD₃OD, 400 MHz): δ 8.54 (s, 1H), 8.36 (s, 1H), 8.35 (d, 1H), 3.91 (s, 3H), 2.77 (d, 2H), 1.18-1.13 (m, 1H), 0.60-0.55 (m, 2H), 0.27-0.20 (m, 2H); TLC: 20% EtOAc/Hexane (R_{f}:05).

### Synthesis of 2-bromo-1-(5-chloro-1-methyl-1H-pyrrolo [2,3-b] pyridin-3-yl)-2-cyclopropylethan-1-one

To a stirred solution of 1-(5-chloro-1-methyl-1H-pyrrolo pyridin-3-yl)-2-cyclopropylethan-1-one (500 mg, 2 mmol) in CH₂Cl₂ (10 mL) at 0° C. under an argon atmosphere was added bromine (0.09 mL, 2 mmol) in CH₂Cl₂ (5 mL). The reaction mixture was stirred for 16 h at room temperature. After consumption of starting material (by TLC), the reaction mixture was diluted with saturated sodium bicarbonate solution (20 mL) and extracted with EtOAc (2×20 mL). The combined organic extracts were dried over sodium sulfate, filtered and concentrated in vacuo. The crude material was purified by column chromatography using 10-15%EtOAc: Hexane to afford 2-bromo-1-(5-chloro-1-methyl-1H-pyrrolo pyridin-3-yl)-2-cyclopropylethan-1-one (300 mg, 46%) as an off-white solid.

¹H NMR (DMSO-d₆, 400 MHz): δ 8.77 (s, 1H), 8.49 (d, 1H), 8.44 (d, 1H), 4.89 (d, 1H), 3.89 (s, 3H), 1.70-1.64 (m, 1H), 0.85-0.75 (m, 2H), 0.60-0.50 (m, 2H); TLC: 20% EtOAc/Hexane (R_{f}:06).

### Synthesis of (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (200 mg, 0.8 mmol) in CH₃CN (10 mL) at 0° C. under an argon atmosphere were added PS-BEMP (400 mg) and 2-bromo-1-(5-chloro-1-methyl-1H-pyrrolo [2,3-b] pyridin-3-yl)-2-cyclopropylethan-1-one (316 mg, 1 mmol). The reaction mixture was stirred at room temperature for 16 h. After consumption of starting material (by TLC), the reaction mixture was filtered. The filtrate was concentrated in vacuo to (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide (250 mg, crude) as brown solid used in the next step without further purification. LCMS: 79.5%; 494 (M+1); (column; Ascentis Express C-18 (50×3.0 mm, 2.7 μm); RT 2.09 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 10% MeOH/CH₂Cl₂ (R_{f}:05).

### Synthesis of 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine

To a stirred solution of (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide (260 mg, 0.5 mmol) in MeOH (8 mL) at room temperature under an argon atmosphere was added acetic acid (2 mL). The reaction mixture was stirred for 6 h at 60° C. Then sodium cyanoborohydride (39 mg, 0.6 mmol) was added to the reaction mixture at room temperature. The reaction mixture was stirred for 4 h at 60° C. After consumption of starting material (by TLC), the volatiles were evaporated in vacuo. The residue was diluted with saturated sodium bicarbonate solution (20 mL) and extracted with EtOAc (2×20 mL). The combined organic extracts were dried over sodium sulfate, filtered and concentrated in vacuo. The crude material was purified by column chromatography using 2-3%MeOH: CH₂Cl₂ to afford 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine (200 mg, 26%, over two steps) as white solid.

Racemic compound of Example 57 was separated using a Chiralpak-IC column (250×20 mm, 5 μm) (25 mg loading; 0.1% DEA in n-Hexane: CH₂Cl₂:MeOH (50:50) (A:B: 30:70) as mobile phase; flow rate: 20 mL/min) to afford the compounds of Example 57A (Fraction I (+)) and Example 57B (Fraction II (−)).

Analytical conditions for Example 57A and Example 57B. HPLC (purity): (column; X-select CSH-C-18 150×4.6 mm, 3.5 μm); mobile Phase: ACN: 5% 0.05% TFA; 0.05% TFA: 5% ACN; flow rate: 1.0 mL/min; Gradient program: T/B % 0.01/90, 10/10, 15/10: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 30:70); flow Rate: 1.0 mL/min).

### Example 57A

(+)-5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine, fraction (I) (+): ¹H NMR (CD₃OD, 400 MHz): δ 8.19 (s, 1H), 8.10 (s, 1H), 7.95 (s, 1H), 7.89 (d, 1H), 7.70 (d, 1H), 7.43 (s, 1H), 7.20 (s, 1H), 5.09 (s, 1H), 4.03 (s, 3H), 3.87 (s, 3H), 3.14 (dd, 1H), 2.20 (s, 3H), 0.51-0.32 (m, 5H); Mass (ESI): 478 [M+1]; HPLC (purity): 99.7%; RT 6.47 min; Chiral HPLC: 100% RT=7.94 min; Optical rotation [α]_{D}^{20.00}: +46.43 (c=0.25, CH₂Cl₂).

### Example 57B

(−)-5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine, fraction (II) (−): Mass (ESI): 478 [M+1]; HPLC (purity): 99.5%; RT 6.45 min; Chiral HPLC: 99.5% RT=10.61 min; Optical rotation [α]_{D}^{20.03}:−45.37 (c=0.25, CH₂Cl₂).

### Example 58

### Synthesis of 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine

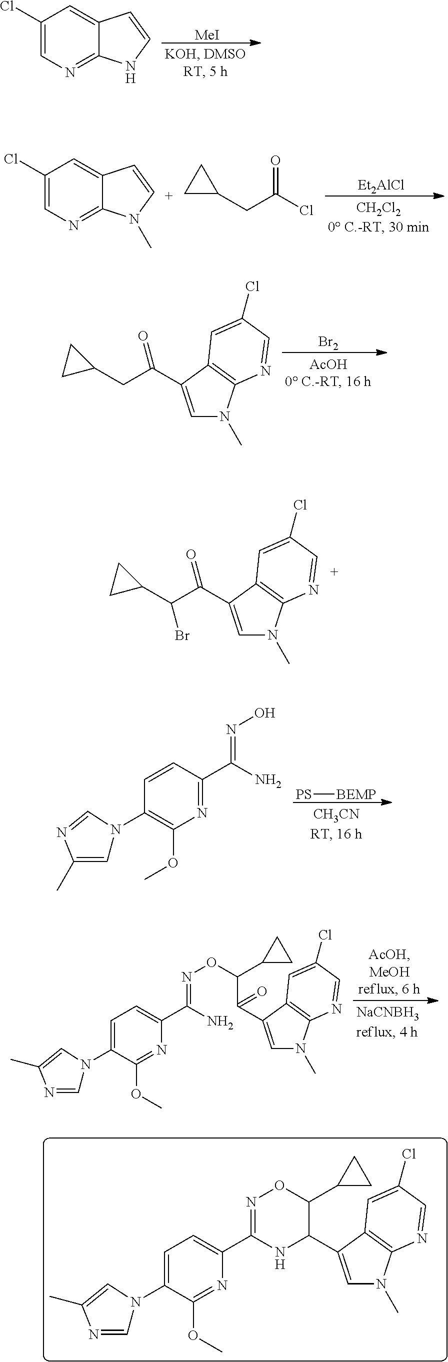

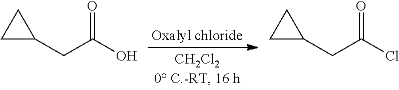 [SMILES: O=C(O)CC1CC1 ; O=C(Cl)CC1CC1]

### Synthesis of 5-chloro-1-methyl-M-pyrrolo [2,3-b] pyridine

To a stirred solution of 5-chloro-1H-pyrrolo [2,3-b] pyridine (25 g, 164 mmol) in DMSO (125 mL) at 0° C. under an argon atmosphere were added potassium hydroxide (14 g, 246 mmol) and methyl iodide (35 g, 246 mmol). The reaction mixture was warmed to room temperature and stirred for 16 h. After consumption of starting material (by TLC), the reaction mixture was diluted with ice cold water (500 mL) and extracted with EtOAc (2×200 mL). The combined organic extracts were dried over sodium sulfate, filtered and concentrated in vacuo to obtain crude. The crude material was purified by column chromatography using 10% EtOAc: Hexane to afford 5-chloro-1-methyl-1H-pyrrolo [2,3-b] pyridine (22.5 g, 83%) as a pale yellow solid. ¹H NMR (CDCl₃, 500 MHz): δ 8.28 (s, 1H), 7.88 (s, 1H), 7.22 (s, 1H), 6.41 (s, 1H), 3.89 (s, 3H); LCMS: 90.6%; 166.8 (M+1); (column; Kinetex EVO C-18 (50×3.0 mm, 2.6 μm); RT 2.70 min; mobile phase: 2.5mM NH₄OOCH in water+5% ACN:ACN+5% 2.5mM NH₄OOCH in water; T/B %: 0.01/5, 4/95, 5.5/95; flow rate: 0.8 mL/min) (Gradient); TLC: 20% EtOAc/Hexane (R_{f}:06).

### Synthesis of 2-cyclopropylacetyl chloride

To a stirred solution of 2-cyclopropylacetic acid (55 g, 550 mmol) in CH₂Cl₂ (275 mL) at 0° C. under an argon atmosphere was added oxalyl chloride (94 mL, 1100 mmol). The reaction mixture was stirred for 10 min Then DMF (2.2 mL) was added to the reaction mixture drop wise for 30 min at 0° C. The reaction mixture was warmed to room temperature, heated at 85° C. and stirred for 16 h. (Color changes: (Colorless to thick brown color)) After consumption of acid (by TLC), the volatiles were evaporated in vacuo to obtain 2-cyclopropylacetyl chloride (65 g, crude) as colorless liquid used in the next step without further purification.

### Synthesis of 1-(5-chloro-1-methyl-1H-pyrrolo [2,3-b] pyridin-3-yl)-2-cyclopropylethan-1-one

To a stirred solution of 5-chloro-1-methyl-1Hpyrrolo [2,3-b] pyridine (22.5 g, 135 mmol) in CH₂Cl₂ (225 mL) at 0° C. under an argon atmosphere were added diethyl aluminium chloride 1M in Hexane (271 mL, 270 mmol). The reaction mixture was stirred for 15 min Then 2-(2-chloro-2-oxoethyl) cyclopropan-1-ylium (65 g, crude) in CH₂Cl₂ (112 mL) was added to the reaction mixture at 0° C. for 1 h. The reaction mixture was warmed to room temperature and stirred for 1 h. After consumption of starting material (by TLC), the reaction mixture was poured in to ice cold water (750 mL) and extracted with CH₂Cl₂ (2×300 mL). The combined organic extracts were washed with saturated sodium bicarbonate solution (500 mL) and brine (500 mL). The organic extract was dried over sodium sulfate, filtered and concentrated in vacuo. The crude material was purified by column chromatography using 15% EtOAc: Hexane to afford 1-(5-chloro-1-methyl-1H-pyrrolo [2,3-b] pyridin-3-yl)-2-cyclopropylethan-1-one (15 g, 44%) as an off-white solid. ¹H NMR (CDCl₃, 500 MHz): δ 8.65 (s, 1H), 8.33 (s, 1H), 7.81 (s, 1H), 3.92 (s, 3H), 2.74 (d, 2H), 1.21-1.18 (m, 1H), 0.62-0.57 (m, 2H), 0.26-0.20 (m, 2H); LCMS: 91.0%; 249 (M+1); (column; Ascentis Express C-18 (50×3.0 mm, 2.7 μm); RT 2.48 min; mobile phase: 0.025% Aq TFA+5% ACN: ACN+5% 0.025% Aq TFA; T/B %: 0.01/5, 0.5/5, 3/100, 5/100; flow rate: 1.2 mL/min) (Gradient); TLC: 30% EtOAc/Hexane (R_{f}:05).

### Synthesis of 2-bromo-1-(5-chloro-1-methyl-1H-pyrrolo [2,3-b] pyridin-3-yl)-2-cyclopropylethan-1-one

To a stirred solution of 1-(5-chloro-1-methyl-1H-pyrrolo [2,3-b] pyridin-3-yl)-2-cyclopropylethan-1-one (15 g, 60 mmol) in AcOH (75 mL) at 0° C. under an argon atmosphere was added bromine (3.1 mL, 60 mmol) followed by Aq 48% HBr (30 mL). The reaction mixture was stirred for 16 h at room temperature. After consumption of starting material (by TLC), the reaction mixture was poured in cold saturated sodium bicarbonate solution (500 mL) and extracted with EtOAc (2×250 mL). The combined organic extracts were washed with brine (500 mL), dried over sodium sulfate, filtered and concentrated in vacuo. The crude material was purified by column chromatography using 5-10% EtOAc: Hexane to afford 2-bromo-1-(5-chloro-1-methyl-1H-pyrrolo [2,3-b] pyridin-3-yl)-2-cyclopropylethan-1-one (14 g, 70%) as an off-white solid. ¹H NMR (CDCl₃, 400 MHz): δ 8.67 (s, 1H), 8.38 (s, 1H), 7.99 (s, 1H), 4.21 (d, 1H), 3.99 (s, 3H), 1.90-1.80 (m, 1H), 0.99-0.90 (m, 2H), 0.58-0.48 (m, 2H); LCMS: 76.1%; 328 (M+1); (column; Ascentis Express C-18 (50×3.0 mm, 2.7 μm); RT 2.75 mm; mobile phase: 0.025% Aq TFA+5% ACN: ACN+5% 0.025% Aq TFA; T/B %: 0.01/5, 0.5/5, 3/100, 5/100; flow rate: 1.2 mL/min) (Gradient); TLC: 20% EtOAc/Hexane (R_{f}:06).

### Synthesis of (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (7 g, 28 mmol) in CH₃CN (250 mL) at 0° C. under an argon atmosphere were added PS-BEMP (15.4 g) and 2-bromo-1-(5-chloro-1-methyl-1H-pyrrolo [2,3-b] pyridin-3-yl)-2-cyclopropylethan-1-one (14 g, 42 mmol) in CH₃CN (100 mL). The reaction mixture was stirred at room temperature for 12 h. After consumption of starting material (by TLC), the reaction mixture was filtered. The filtrate was concentrated in vacuo to (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide (10.8 g, crude) as brown solid used in the next step without further purification. LCMS: 68.9%; 494.1 (M+1); (column; Ascentis Express C-18 (50×3.0 mm, 2.7 μm); RT 2.07 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:04).

### Synthesis of 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine

To a stirred solution of (Z)-N′-(2-(5-chloro-1-methyl-1H-pyrrolo [2,3-b] pyridin-3-yl)-1-cyclopropyl-2-oxoethoxy)-6-methoxy-5-(4-methyl-1H-imidazol-1-yl) picolinimidamide (10.8 g, 22 mmol) in MeOH (216 mL) at room temperature under an argon atmosphere was added acetic acid (54 mL). The reaction mixture was stirred for 12 h at 60° C. Then sodium cyanoborohydride (1.65 g, 26 mmol) was added to the reaction mixture at room temperature. The reaction mixture was stirred for 6 h at 60° C. After consumption of starting material (by TLC), the volatiles were evaporated in vacuo. The residue was poured in to cold saturated sodium bicarbonate solution (250 mL) and extracted with EtOAc (2×150 mL). The combined organic extracts were washed with brine (300 m), dried over sodium sulfate, filtered and concentrated in vacuo. The crude material was purified by column chromatography using 2% MeOH:CH₂Cl₂to afford 5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine (4.6 g, 34%, over two steps) as a pale yellow solid.

Racemic compound of Example 58 was separated using a Chiralpak-IC column (250×20 mm, 5 μm) (25 mg loading; 0.1% DEA in n-Hexane: CH₂Cl₂:MeOH (50:50) (A:B: 30:70) as mobile phase; flow rate: 20 mL/min) to afford the compounds of Example 58A (Fraction I (+)) and Example 58B (Fraction II (−)).

Analytical conditions for Example 58A and Example 58B. HPLC (purity): (column; X-select CSH-C-18 150×4.6 mm, 3.5 μm); mobile Phase: 0.05% TFA+5% ACN: ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 30:70); flow Rate: 1.0 mL/min).

### Example 58A

(+)-5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine, fraction (I) (+): ¹H NMR (CD₃OD, 400 MHz): δ 8.19 (s, 1H), 8.10 (s, 1H), 7.95 (s, 1H), 7.89 (d, 1H), 7.70 (d, 1H), 7.49 (s, 1H), 7.20 (s, 1H), 5.10-5.08 (m, 1H), 4.03 (s, 3H), 3.87 (s, 3H), 3.14 (dd, 1H), 2.22 (s, 3H), 0.51-0.32 (m, 5H); Mass (ESI): 478.1 [M+1]; HPLC (purity): 99.2%; RT 6.56 mm; Chiral HPLC: 100% RT=7.59 mm; Optical rotation [α]_{D}^{19.97}: +58.65 (c=0.25, CH₂Cl₂).

### Example 58B

(−)-5-(5-chloro-1-methyl-1H-pyrrolo [2,3-b] pyridin-3-yl)-6-cyclopropyl-3-(6-methoxy-5-(4-methyl-1H-imidazol-1-yl) pyridin-2-yl)-5,6-dihydro-4H-1,2,4-oxadiazine, fraction (II) (−): Mass (ESI): 478 [M+1]; HPLC (purity): 98.9%; RT 6.55 min; Chiral HPLC: 100% RT=10.54 mm; Optical rotation [α]_{D}^{20.01}: −61.56 (c=0.25, CH₂Cl₂).

### Example 59

### Synthesis of 6-cyclopropyl-5-(3,5-difluorophenyl)-3-(6-methoxy-5-(4-methyl-1H-imidazol-1-yl) pyridin-2-yl)-5,6-dihydro-4H-1,2,4-oxadiazine

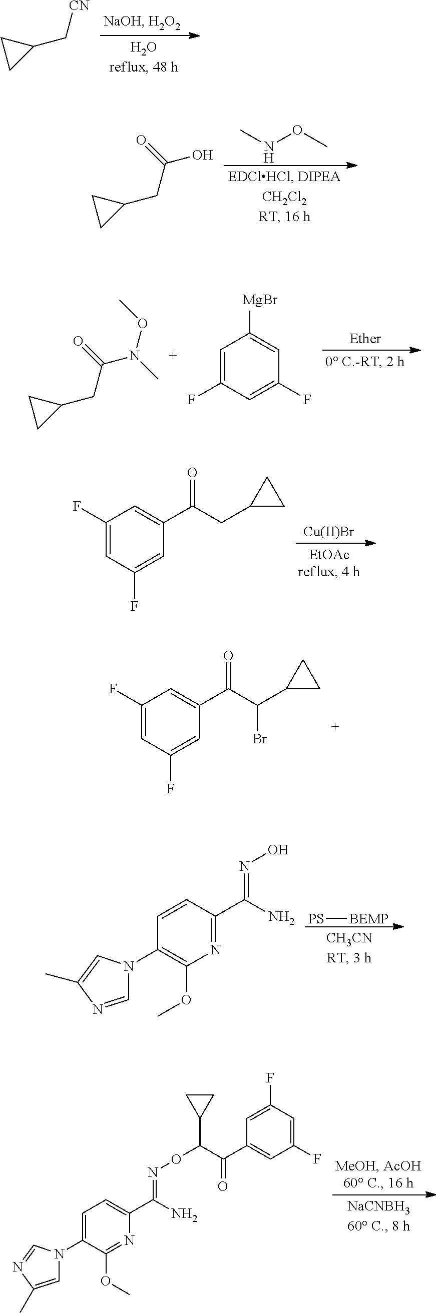

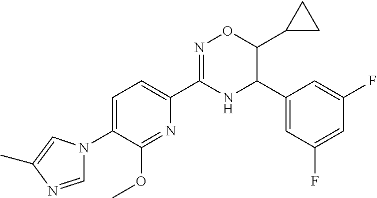 [SMILES: COc1nc(C2=NOC(C3CC3)C(c3cc(F)cc(F)c3)N2)ccc1-n1cnc(C)c1]

### Synthesis of 2-cyclopropylacetic acid

To a stirred solution of sodium hydroxide (23.95 g, 598 mmol) in H₂O (20 mL) at 0° C. under an argon atmosphere was added 30% H₂O₂ (20 mL). Then 2-cyclopropylacetonitrile (5 g, 61 mmol) was added to the reaction mixture at room temperature. The reaction mixture was reflux for 48 h. After consumption of starting material (by TLC), the reaction mixture was cooled to 0° C., acidified with HCl up to pH ˜1 and extracted with CH₂Cl₂ (2×100 mL). The combined organic extracts were dried over sodium sulfate, filtered and concentrated in vacuo to obtain 2-cyclopropylacetic acid (5 g, 83%) as a pale yellow liquid. ¹H NMR (CDCl₃, 500 MHz): δ 2.24 (d, 2H), 1.10-1.01 (m, 1H), 0.59-0.53 (m, 2H), 0.20-0.14 (m, 2H)

### Synthesis of 2-cyclopropyl-N-methoxy-N-methylacetamide

To a stirred solution of 2-cyclopropylacetic acid (2 g, 20 mmol) in CH₂Cl₂ (20 mL) at 0° C. under an argon atmosphere were added EDCI. HCl (2.13 g, 22 mmol), diisopropyl ethyl amine (4.84 g, 40 mmol) and N, O-dimethylhydroxylamine (2.13 g, 22 mmol). The reaction mixture was warmed to room temperature and stirred for 16 h. After consumption of starting material (by TLC), the reaction mixture was diluted with saturated sodium bicarbonate solution (50 mL) and extracted with CH₂Cl₂ (2×50mL). The combined organic extracts were dried over sodium sulfate, filtered and concentrated in vacuo to obtain 2-cyclopropyl-N-methoxy-N-methylacetamide (1.2 g, crude) as brown solid used in the next step without further purification. ¹H NMR (CDCl₃, 400 MHz): δ 3.67 (s, 3H), 3.18 (s, 3H), 2.34 (d, 2H), 1.10-1.02 (m, 1H), 0.58-0.50 (m, 2H), 0.20-0.13 (m, 2H); TLC: 30% EtOAc/Hexane (R_{f}:03).

### Synthesis of 2-cyclopropyl-1-(3,5-difluorophenyl) ethan-1-one

To a stirred solution of 2-cyclopropyl-N-methoxy-N-methylacetamide (2 g, 14 mmol) in ether (20 mL) at 0° C. under an argon atmosphere was added (3,5-difluorophenyl) magnesium bromide (3 g, 14 mmol). The reaction mixture was warmed to room temperature and stirred for 2 h. After consumption of starting material (by TLC), the reaction mixture was quenched with saturated ammonium chloride solution (50 mL) and extracted with EtOAc (2×50mL). The combined organic extracts were dried over sodium sulfate, filtered and concentrated in vacuo. The crude material was purified by column chromatography using 2-3%EtOAc: Hexane to afford 2-cyclopropyl-1-(3,5-difluorophenyl) ethan-1-one (1.5 g, 55%) as colorless liquid. ¹H NMR (500 MHz, DMSO-d₆): δ 7.62 (d, 2H), 7.58-7.54 (m, 1H), 2.97 (d, 2H), 1.07-0.98 (m, 1H), 0.53-0.46 (m, 2H), 0.15-0.13 (m, 2H); TLC: 5% EtOAc/Hexane (R_{f}:05).

### Synthesis of 2-bromo-2-cyclopropyl-1-(3,5-difluorophenyl) ethan-1-one

To a stirred solution of 2-cyclopropyl-1-(3,5-difluorophenyl) ethan-1-one (1.5 g, 7 mmol) in EtOAc (20 mL) at room temperature under an argon atmosphere was added copper bromide (3.4 g, 15 mmol). The reaction mixture was stirred for 4 h at 90° C. After consumption of starting material (by TLC), the reaction mixture was filtered. The filtrate was concentrated in vacuo. The crude material was purified by column chromatography using 2-3%EtOAc: Hexane to afford 2-bromo-2-cyclopropyl-1-(3,5-difluorophenyl) ethan-1-one (1.6 g, 80%) as a pale yellow liquid. ¹H NMR (400 MHz, DMSO-d₆): δ 7.73-7.60 (m, 3H), 5.29 (d, 1H), 1.67-1.60 (m, 1H), 0.83-0.80 (m, 2H), 0.57-0.51 (m, 2H); TLC: 5% EtOAc/Hexane (R_{f}:05).

### Synthesis of (Z)-N′-(1-cyclopropyl-2-(3,5-difluorophenyl)-2-oxoethoxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (500 mg, 2 mmol) in CH₃CN (15 mL) at room temperature under an argon atmosphere were added PS-BEMP (1 g) and 2-bromo-2-cyclopropyl-1-(3,5-difluorophenyl) ethan-1-one (822 mg, 3 mmol). The reaction mixture was stirred at room temperature for 3 h. After consumption of starting material (by TLC), the reaction mixture was filtered. The filtrate was concentrated in vacuo to obtain (Z)-N′-(1-cyclopropyl-2-(3,5-difluorophenyl)-2-oxoethoxy)-6-methoxy-5-(4-methyl-1H-imidazol-1-yl) picolinimidamide (900 mg, crude) as brown solid used in the next step without further purification. LCMS: 16.7%; 442 (M+1); (column; Ascentis Express C-18 (50×3.0 mm, 2.7 μm); RT 2.01 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 10% MeOH/CH₂Cl₂ (R_{f}:05).

### Synthesis of 6-cyclopropyl-5-(3,5-difluorophenyl)-3-(6-methoxy-5-(4-methyl-1H-imidazol-1-yl) pyridin-2-yl)-5,6-dihydro-4H-1,2,4-oxadiazine

To a stirred solution of (Z)-N′-(1-cyclopropyl-2-(3,5-difluorophenyl)-2-oxoethoxy)-6-methoxy-5-(4-methyl-1H-imidazol-1-yl) picolinimidamide (800 mg, 2 mmol) in MeOH (8 mL) at room temperature under an argon atmosphere was added acetic acid (2 mL). The reaction mixture was stirred for 16 h at 60° C. Then sodium cyanoborohydride (228 mg, 4 mmol) was added to the reaction mixture at room temperature. The reaction mixture was stirred for 8 h at 60° C. After consumption of starting material (by TLC), the volatiles were evaporated in vacuo. The residue was diluted with saturated sodium bicarbonate solution (20 mL) and extracted with EtOAc (2×20 mL). The combined organic extracts were dried over sodium sulfate, filtered and concentrated in vacuo. The crude material was purified by column chromatography using 2-3%MeOH:CH₂Cl₂ to afford 6-cyclopropyl-5-(3,5-difluorophenyl)-3-(6-methoxy-5-(4-methyl-1H-imidazol-1-yl) pyridin-2-yl)-5,6-dihydro-4H-1,2,4-oxadiazine (100 mg, 13%) as colorless solid.

Racemic compound of Example 59 was separated using a Chiralpak-IA column (250×20 mm, 5 μm) (50 mg loading; 0.1% DEA in n-Hexane:CH₂Cl₂:MeOH (50:50) (A:B: 85:15) as mobile phase; flow rate: 20 mL/min) to afford the compounds of Example 59A (Fraction I (+)) and Example 59B (Fraction II (−)).

Analytical conditions for Example 59A and Example 59B. HPLC (purity): (column; X-select CSH-C-18 150×4.6 mm, 3.5 μm); mobile Phase: ACN: 5% 0.05% TFA; 0.05% TFA: 5% ACN; flow rate: 1.0 mL/min; Gradient program: T/B % 0.01/90, 10/10, 15/10: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (80:20) (A::B; 85:15); flow Rate: 1.0 mL/min).

### Example 59A

(+)-6-cyclopropyl-5-(3,5-difluorophenyl)-3-(6-methoxy-5-(4-methyl-1H-imidazol-1-yl) pyridin-2-yl)-5,6-dihydro-4H-1,2,4-oxadiazine, fraction (I), (+): ¹H NMR (DMSO-d₆, 400 MHz): δ 8.00 (d, 1H), 7.93 (s, 1H), 7.91 (s, 1H), 7.64 (d, 1H), 7.26 (s, 1H), 7.21-7.13 (m, 1H), 6.96 (d, 2H), 4.84-4.78 (m, 1H), 4.02 (s, 3H), 3.08 (dd, 1H), 2.16 (s, 3H), 0.52-0.43 (m, 3H), 0.40-0.34 (m, 1H), 0.26-0.17 (m, 1H); Mass (ESI): 426 [M+1]; HPLC (purity): 98.1%; RT 7.13 min; Chiral HPLC: 95.7% RT=13.04 min; Optical rotation [α]_{D}^{19.99}: +67.07 (c=0.25, CH₂Cl₂).

### Example 59B

(−)-6-cyclopropyl-5-(3,5-difluorophenyl)-3-(6-methoxy-5-(4-methyl-1H-imidazol-1-yl) pyridin-2-yl)-5,6-dihydro-4H-1,2,4-oxadiazine, fraction (II), (−): Mass (ESI): 426 [M+1]; HPLC (purity): 95.6%; RT 7.13 min; Chiral HPLC: 96.6% RT=15.88 min; Optical rotation [α]_{D}^{19.97}: −65.92 (c=0.25, CH₂Cl₂).

### Example 60

### Synthesis of 6-cyclopropyl-5-(4,5-difluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine

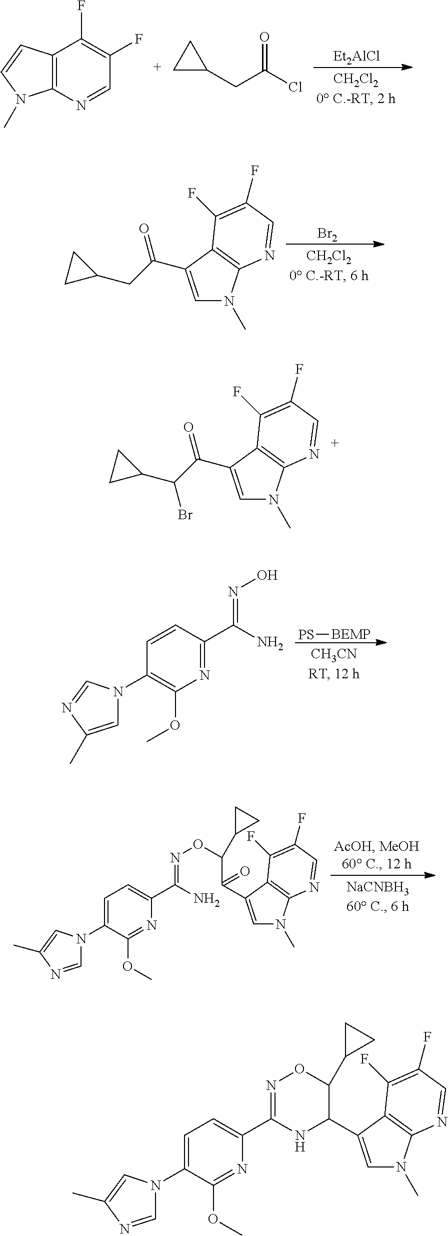

### Synthesis of 2-cyclopropyl-1-(4,5-difluoro-1-methyl-1H-pyrrolo [2,3-b] pyridin-3-yl) ethan-1-one

To a stirred solution of 4,5-difluoro-1-methyl-1H-pyrrolo [2,3-b] pyridine (1 g, 6 mmol) in CH₂Cl₂ (10 mL) at 0° C. under an argon atmosphere were added diethyl aluminium chloride 1M in Hexane (11.5 mL, 12 mmol). The reaction mixture was stirred for 15 min Then 2-(2-chloro-2-oxoethyl) cyclopropan-1-ylium (2.8 g, 24 mmol) in CH₂Cl₂(5 mL) was added to the reaction mixture at 0° C. The reaction mixture was warmed to room temperature and stirred for 2 h. After consumption of starting material (by TLC), the reaction mixture was diluted with cold water (50 mL) and extracted with EtOAc (2×50 mL). The combined organic extracts were washed with saturated sodium bicarbonate solution (50 mL), dried over sodium sulfate, filtered and concentrated in vacuo. The crude material was purified by column chromatography using 20%EtOAc: Hexane to afford 2-cyclopropyl-1-(4,5-difluoro-1-methyl-1H-pyrrolo [2,3-b] pyridin-3-yl) ethan-1-one (818 mg, 55%) as an off-white solid. ¹H NMR (CDCl₃, 400 MHz): δ 8.32 (dd, 1H), 7.90 (s, 1H), 3.93 (s, 3H), 2.81 (dd, 2H), 1.24-1.14 (m, 1H), 0.63-0.57 (m, 2H), 0.24-0.17 (m, 2H); TLC: 30% EtOAc/Hexane (R_{f}:02).

### Synthesis of 2-bromo-2-cyclopropyl-1-(4,5-difluoro-1-methyl-1H-pyrrolo [2,3-b] pyridin-3-yl) ethan-1-one

To a stirred solution of 2-cyclopropyl-1-(4,5-difluoro-1-methyl-1H-pyrrolo [2,3-b] pyridin-3-yl) ethan-1-one (850 mg, 3 mmol) in CH₂Cl₂ (12 mL) at 0° C. under an argon atmosphere was added bromine (490 mg, 3 mmol) in CH₂Cl₂ (5 mL). The reaction mixture was stirred for 6 h at room temperature. After consumption of starting material (by TLC), the reaction mixture was diluted with saturated sodium bicarbonate solution (100 mL) and extracted with EtOAc (2×100 mL). The combined organic extracts were dried over sodium sulfate, filtered and concentrated in vacuo. The crude material was purified by column chromatography using 20%EtOAc: Hexane to afford 2-bromo-2-cyclopropyl-1-(4,5-difluoro-1-methyl-1H-pyrrolo [2,3-b] pyridin-3-yl) ethan-1-one (800 mg, 68%) as an off-white solid. ¹H NMR (CDCl₃, 400 MHz): δ 8.36 (dd, 1H), 8.04 (s, 1H), 4.36 (dd, 1H), 3.94 (s, 3H), 1.92-1.81 (m, 1H), 0.98-0.86 (m, 2H), 0.61-0.45 (m, 2H); TLC: CH₂Cl₂ (R_{f}:06).

### Synthesis of (Z)-N′-(1-cyclopropyl-2-(4,5-difluoro-1-methyl-1H-pyrrolo [2,3-b] pyridin-3-yl)-2-oxoethoxy)-6-methoxy-5-(4-methyl-1H-imidazol-1-yl) picolinimidamide

To a stirred solution of (Z)-5-bromo-N′-hydroxy-6-methoxypicolinimidamide (300 mg, 1 mmol) in CH₃CN (10 mL) at room temperature under an argon atmosphere was added PS-BEMP (660 mg). The reaction mixture was stirred for 10 min at room temperature. Then 2-bromo-2-cyclopropyl-1-(4,5-difluoro-1-methyl-1H-pyrrolo [2,3-b] pyridin-3-yl) ethan-1-one (600 mg, 2 mmol) in CH₃CN (5 mL) was added to the reaction mixture at room temperature. The reaction mixture was stirred for 12 h at room temperature. After consumption of starting material (by TLC), the reaction mixture was filtered and the filtrate was concentrated in vacuo to obtain (Z)-N′-(1-cyclopropyl-2-(4,5-difluoro-1-methyl-1H-pyrrolo [2,3-b] pyridin-3-yl)-2-oxoethoxy)-6-methoxy-5-(4-methyl-1H-imidazol-1-yl) picolinimidamide (800 mg, crude) as brown semi solid used in the next step without further purification. LCMS: 67.2%; 496.1 (M+1); (column; Ascentis Express C-18 (50×3.0 mm, 2.7 μm); RT 1.96 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:03).

### Synthesis of 6-cyclopropyl-5-(4,5-difluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine

To a stirred solution of (Z)-N′-(1-cyclopropyl-2-(4,5-difluoro-1-methyl-1H-pyrrolo [2,3-b] pyridin-3-yl)-2-oxoethoxy)-6-methoxy-5-(4-methyl-1H-imidazol-1-yl) picolinimidamide (800 mg, 2 mmol) in MeOH (32 mL) at room temperature under an argon atmosphere was added acetic acid (4 mL). The reaction mixture was stirred for 12 h at 60° C. Then sodium cyanoborohydride (130 mg, 2 mmol) was added to the reaction mixture at room temperature. The reaction mixture was stirred for 6 h at 60° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 2%MeOH:CH₂Cl₂ to afford 6-cyclopropyl-5-(4,5-difluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine (250 mg, 43% over two steps) as an off-white solid.

Racemic compound of Example 60 was separated using a Chiralpak-IC column (250×20 mm, 5 μm) (50 mg loading; 0.1% DEA in n-Hexane: CH₂Cl₂:MeOH (50:50) (A:B: 60:40) as mobile phase; flow rate: 20 mL/min) to afford the compounds of Example 60A (Fraction I (+)) and Example 60B (Fraction II (−)). Analytical conditions for Example 60A and Example 60B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 30:70); flow Rate: 1.0 mL/min).

### Example 60A

(+)-6-cyclopropyl-5-(4,5-difluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine, fraction (I) (+):¹H NMR (CD₃OD, 400 MHz): δ 8.28 (dd, 1H), 7.97 (s, 1H), 7.88 (d, 1H), 7.67 (d, 1H), 7.36 (s, 1H), 7.21 (s, 1H), 5.34 (d, 1H), 4.07 (s, 3H), 3.84 (s, 3H), 3.17 (dd, 1H), 2.25 (s, 3H), 0.63-0.33 (m, 5H); Mass (ESI): 480.1 [M+1]; HPLC (purity): 98.2%; RT 6.35 min; Chiral HPLC: 97.5% RT =9.92 min; Optical rotation [α]_{D}^{19.99}: +13.18 (c=0.25, CH₂Cl₂).

### Example 60B

(−)-6-cyclopropyl-5-(4,5-difluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine, fraction (II) (−): Mass (ESI): 480.1 [M+1]; HPLC (purity): 99.6%; RT 6.48 min; Chiral HPLC: 99.5% RT =12.08 min; Optical rotation [α]_{D}^{20.01}: −9.85 (c=0.25, CH₂Cl₂).

### Example 61

### Synthesis of 5-(benzofuran-2-yl)-6-cyclopropyl-3-(6-methoxy-5-(4-methyl-1H-imidazol-1-yl) pyridin-2-yl)-5,6-dihydro-4H-1,2,4-oxadiazine

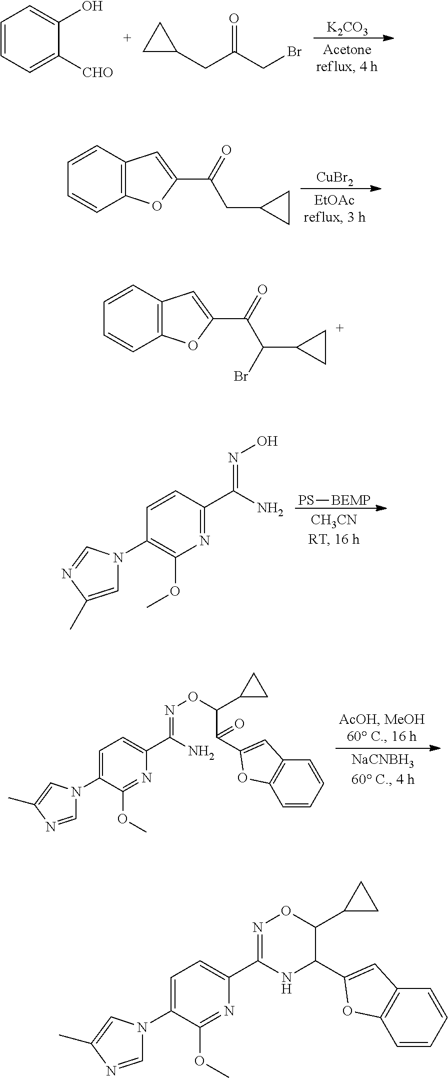

### Synthesis of 1-(benzofuran-2-yl)-2-cyclopropylethan-1-one

To a stirred solution of 2-hydroxybenzaldehyde (100 mg, 1 mmol) in acetone (2 mL) at room temperature under an argon atmosphere were added potassium carbonate (169 mg, 1 mmol) and 1-bromo-3-cyclopropylpropan-2-one (174 mg, 1 mmol). The reaction mixture was stirred at reflux for 4 h. After consumption of starting material (by TLC), the reaction mixture was diluted with water (20 mL) and extracted with CH₂Cl₂ (2×20 mL). The combined organic extracts were dried over sodium sulfate, filtered and concentrated in vacuo to obtain 1-(benzofuran-2-yl)-2-cyclopropylethan-1-one (105 mg, crude) as brown solid used in the next step without further purification. ¹H NMR (CDCl₃, 500 MHz): δ 7.71 (d, 1H), 7.58 (d, 1H), 7.51-7.45 (m, 2H), 7.31 (t, 1H), 2.86 (d, 1H), 1.33-1.14 (m, 1H), 0.65-0.58 (m, 2H), 0.28-0.23 (m, 2H); TLC: 5% EtOAc/Hexane (R_{f}:05).

### Synthesis of 1-(benzofuran-2-yl)-2-bromo-2-cyclopropylethan-1-one

To a stirred solution of 1-(benzofuran-2-yl)-2-cyclopropylethan-1-one (500 mg, 2.5 mmol) in EtOAc (15 mL) at room temperature under an argon atmosphere was added copper bromide (1.11 g, 5 mmol). The reaction mixture was stirred for 3 h at reflux. After consumption of starting material (by TLC), the reaction mixture was filtered and the filtrate was concentrated in vacuo. The crude material was purified by column chromatography using 10-20%EtOAc: Hexane to afford 1-(benzofuran-2-yl)-2-bromo-2-cyclopropylethan-1-one (501 mg, 70%) as an off-white solid. ¹H NMR (CDCl₃, 500 MHz): δ 7.73 (d, 1H), 7.66 (s, 1H), 7.59 (d, 1H), 7.51 (t, 1H), 7.33 (t, 1H), 4.48 (d, 1H), 1.87-1.77 (m, 1H), 0.97-0.88 (m, 2H), 0.63-0.52 (m, 2H)

### Synthesis of (Z)-N′-(2-(benzofuran-2-yl)-1-cyclopropyl-2-oxoethoxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (300 mg, 1 mmol) in CH₃CN (15 mL) at room temperature under an argon atmosphere were added PS-BEMP (600 mg) and 1-(benzofuran-2-yl)-2-bromo-2-cyclopropylethan-1-one (508 mg, 2 mmol). The reaction mixture was stirred at room temperature for 16 h. After consumption of starting material (by TLC), the reaction mixture was filtered. The filtrate was concentrated in vacuo to obtain (Z)-N′-(2-(benzofuran-2-yl)-1-cyclopropyl-2-oxoethoxy)-6-methoxy-5-(4-methyl-1H-imidazol-1-yl) picolinimidamide (440 mg, crude) as brown solid used in the next step without further purification. LCMS: 26.3%; 446 (M+1); (column; Ascentis Express C-18 (50×3.0 mm, 2.7 μm); RT 2.16 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:05). Synthesis of 5-(benzofuran-2-yl)-6-cyclopropyl-3-(6-methoxy-5-(4-methyl-1H-imidazol-1-yl) pyridin-2-yl)-5,6-dihydro-4H-1,2,4-oxadiazine

To a stirred solution of (Z)-N′-(2-(benzofuran-2-yl)-1-cyclopropyl-2-oxoethoxy)-6-methoxy-5-(4-methyl-1H-imidazol-1-yl) picolinimidamide (440 mg, 1 mmol) in MeOH (6 mL) at room temperature under an argon atmosphere was added acetic acid (1.5 mL). The reaction mixture was stirred for 16 h at 60° C. Then sodium cyanoborohydride (100 mg) was added to the reaction mixture at room temperature. The reaction mixture was stirred for 4 h at 60° C. After consumption of starting material (by TLC), the reaction mixture was diluted with saturated sodium bicarbonate solution (20 mL) and extracted with CH₂Cl₂ (2×20 mL). The combined organic extracts were dried over sodium sulfate, filtered and concentrated in vacuo. The crude material was purified by column chromatography using 2-3%MeOH:CH₂Cl₂ to afford 5-(benzofuran-2-yl)-6-cyclopropyl-3-(6-methoxy-5-(4-methyl-1H-imidazol-1-yl) pyridin-2-yl)-5,6-dihydro-4H-1,2,4-oxadiazine (180 mg, 42%) as an off-white solid.

Racemic compound of Example 61 was separated using a Chiralpak-IA column (250×20 mm, 5 μm) (28 mg loading; 0.1% DEA in n-Hexane: CH₂Cl₂:MeOH (50:50) (A:B: 70:30) as mobile phase; flow rate: 20 mL/min) to afford the compounds of Example 61A (Fraction I (+)) and Example 61B (Fraction II (−)).

Analytical conditions for Example 61A and Example 61B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 70:30); flow Rate: 1.0 mL/min).

### Example 61A

(+)-5-(benzofuran-2-yl)-6-cyclopropyl-3-(6-methoxy-5-(4-methyl-1H-imidazol-1-yl) pyridin-2-yl)-5,6-dihydro-4H-1,2,4-oxadiazine, fraction (I) (+): ¹H NMR (CD₃OD, 400 MHz): δ 7.97 (s, 1H), 7.88 (d, 1H), 7.68 (d, 1H), 7.57-7.54 (m, 1H), 7.48-7.44 (m, 1H), 7.28-7.17 (m, 3H), 6.73 (s, 1H), 5.06 (d, 1H), 4.08 (s, 3H), 3.23 (dd, 1H), 2.25 (s, 3H), 0.70-0.56 (m, 3H), 0.51-0.46 (m, 2H); Mass (ESI): 430 [M+1]; HPLC (purity): 97.6%; RT 6.69 min; Chiral HPLC: 98.7% RT=4.94 min; Optical rotation [α]_{D}^{19.98}: +105.50 (c=0.25, CH₂Cl₂).

### Example 61B

(−)-5-(benzofuran-2-yl)-6-cyclopropyl-3-(6-methoxy-5-(4-methyl-1H-imidazol-1-yl) pyridin-2-yl)-5,6-dihydro-4H-1,2,4-oxadiazine, fraction (II) (−): Mass (ESI): 430 [M+1]; HPLC (purity): 99.4%; RT 6.62 min; Chiral HPLC: 100% RT=6.26 min; Optical rotation [α]_{D}^{19.99}: −116.11 (c=0.25, CH₂Cl₂).

### Example 62

### Synthesis of 5-(5-chloro-4-fluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

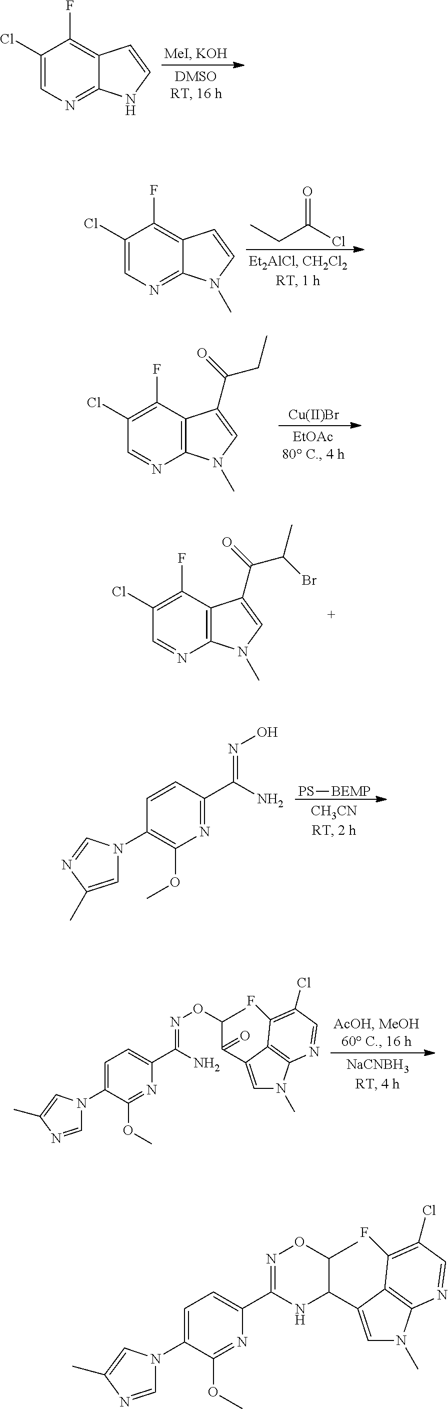

### Synthesis of 5-chloro-4-fluoro-1-methyl-M-pyrrolo [2,3-b] pyridine

To a stirred solution of 5-chloro-4-fluoro-1H-pyrrolo [2,3-b] pyridine (400 mg, 2 mmol) in DMSO (8 mL) at room temperature under an argon atmosphere was added potassium hydroxide (198 mg, 4 mmol) and methyl iodide (501 mg, 4 mmol). The reaction mixture was stirred at room temperature for 16 h. After consumption of starting material (by TLC), the reaction mixture was diluted with water (50 mL) and extracted with EtOAc (2×50 mL). The combined organic extracts were dried over sodium sulfate, filtered and concentrated in vacuo to obtain 5-chloro-4-fluoro-1-methyl-1H-pyrrolo [2,3-b] pyridine (350 mg, crude) as colorless liquid. ¹H NMR (CDCl₃, 500 MHz): δ 8.25 (d, 1H), 7.17 (s, 1H), 6.52 (s, 1H), 3.87 (s, 3H); TLC: 20% EtOAc/Hexane (R_{f}:06).

### Synthesis of 1-(5-chloro-4-fluoro-1-methyl-1H-pyrrolo [2,3-b] pyridin-3-yl) propan-1-one

To a stirred solution of 5-chloro-4-fluoro-1-methyl-1H-pyrrolo [2,3-b] pyridine (200 mg, 1 mmol) in CH₂Cl₂ (4 mL) at 0° C. under an argon atmosphere were added diethyl aluminium chloride 1M in Hexane (1.6 mL, 1.63 mmol). The reaction mixture was stirred for 10 mm Then propionyl chloride (120 mg, 1 mmol) in CH₂Cl₂ (1 mL) was added to the reaction mixture at 0° C. The reaction mixture was warmed to room temperature and stirred for 2 h. After consumption of starting material (by TLC), the reaction mixture was diluted with saturated ammonium chloride solution (20 mL) and extracted with CH₂Cl₂ (2×20 mL). The combined organic extracts were dried over sodium sulfate, filtered and concentrated in vacuo. The crude material was washed with hexane (2×20 mL) to afford 1-(5-chloro-4-fluoro-1-methyl-1H-pyrrolo [2,3-b] pyridin-3-yl) propan-1-one (200 mg, crude) as an off-white solid. ¹H NMR (CDCl₃, 500 MHz): δ 8.33 (d, 1H), 7.90 (s, 1H), 3.93 (s, 3H), 2.99-2.96 (m, 2H), 1.23 (t, 3H); TLC: 30% EtOAc/Hexane (R_{f}:02).

### Synthesis of 2-bromo-1-(5-chloro-4-fluoro-1-methyl-1H-pyrrolo [2,3-b] pyridin-3-yl) propan-1-one

To a stirred solution of 1-(5-chloro-4-fluoro-1-methyl-1H-pyrrolo [2,3-b] pyridin-3-yl) propan-1-one (300 mg, 1 mmol) in EtOAc (10 mL) at room temperature under an argon atmosphere was added copper bromide (613 mg, 3 mmol). The reaction mixture was stirred for 4 h at 80° C. After consumption of starting material (by TLC), the reaction mixture was filtered. The filtrate was concentrated in vacuo to obtain 2-bromo-1-(5-chloro-4-fluoro-1-methyl-1H-pyrrolo [2,3-b] pyridin-3-yl) propan-1-one (280 mg, crude) as brown syrup used in the next step without further purification. ¹H NMR (CDCl₃, 500 MHz): δ 8.40 (s, 1H), 8.07 (s, 1H), 5.23-5.20 (m, 1H), 3.98 (s, 3H), 1.90 (d, 3H); TLC: 20% EtOAc/Hexane (R_{f}: 0.5).

### Synthesis of (Z)-N′-((1-(5-chloro-4-fluoro-1-methyl-1H-pyrrolo [2,3-b] pyridin-3-yl)-1-oxopropan-2-yl) oxy)-6-methoxy-5-(4-methyl-1H-imidazol-1-yl) picolinimidamide

To a stirred solution of (Z)-N′-hydroxy-6-methoxy-5-(4-methyl-1H-imidazol-1-yl) picolinimidamide (150 mg, 0.6 mmol) in CH₃CN (3.75 mL) at room temperature under an argon atmosphere was added PS-BEMP (350 mg). The reaction mixture was stirred for 10 min at room temperature. Then 2-bromo-1-(5-chloro-4-fluoro-1-methyl-1H-pyrrolo [2,3-b] pyridin-3-yl) propan-1-one (290 mg, 0.9 mmol) in CH₃CN (3.75 mL) was added to the reaction mixture at room temperature. The reaction mixture was stirred for 2 h at room temperature. After consumption of starting material (by TLC), the reaction mixture was filtered and the filtrate was concentrated in vacuo to obtain (Z)-N′-((1-cyclopropyl-2-(4-fluoro-1-methyl-1H-pyrrolo [2,3-b] pyridin-3-yl)-2-oxoethoxy)-6-methoxy-5-(4-methyl-1H-imidazol-1-yl) picolinimidamide (250 mg, crude) as a pale yellow solid used in the next step without further purification. LCMS: 46.5%; 486 (M+1); (column; Ascentis Express C-18 (50×3.0 mm, 2.7 μm); RT 1.95 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:05).

### Synthesis of 5-(5-chloro-4-fluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine

To a stirred solution of (Z)-N′-(1-cyclopropyl-2-(4-fluoro-1-methyl-1H-pyrrolo [2,3-b] pyridin-3-yl)-2-oxoethoxy)-6-methoxy-5-(4-methyl-1H-imidazol-1-yl) picolinimidamide (200 mg, 0.4 mmol) in MeOH (6 mL) at room temperature under an argon atmosphere was added acetic acid (1 mL). The reaction mixture was stirred for 16 h at 60° C. Then sodium cyanoborohydride (31 mg, 0.5 mmol) was added to the reaction mixture at room temperature. The reaction mixture was warmed to room temperature and stirred for 4 h. After consumption of starting material (by TLC), the reaction mixture was diluted with water (20 mL) and extracted with EtOAc (2×20 mL). The combined organic extracts were dried over sodium sulfate, filtered and concentrated in vacuo. The crude material was purified by column chromatography using 5%MeOH:CH₂Cl₂ to afford 5-(5-chloro-4-fluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine (40 mg, 21% over two steps) as an off-white solid.

Racemic compound of Example 62 was separated using a Chiralpak-IA column (250×20 mm, 5 μm) (30 mg loading; 0.1% DEA in n-Hexane: CH₂Cl₂:MeOH (50:50) (A:B: 85:15) as mobile phase; flow rate: 20 mL/min) to afford the compounds of Example 62A (Fraction I (+)) and Example 62B (Fraction II (−)).

Analytical conditions for Example 62A and Example 62B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 85:15); flow Rate: 1.0 mL/min).

### Example 62A

(+)-5-(5-chloro-4-fluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (I) (+): ¹H NMR (CD₃OD, 400 MHz): δ 8.28 (d, 1H), 7.97 (s 1H), 7.89 (d, 1H), 7.68 (d, 1H), 7.30 (s, 1H), 7.22 (s, 1H), 5.22 (d, 1H), 4.11 (dd, 1H), 4.07 (s, 3H), 3.85 (s, 3H), 2.25 (s, 3H), 1.08 (d, 3H); Mass (ESI): 470 1M+11; HPLC (purity): 99.3%; RT 6.28 mm; Chiral HPLC: 100% RT =12.21 mm; Optical rotation [α]_{D}^{19.99}: +8.81 (c=0.25, CH₂Cl₂).

### Example 62B

(−)-5-(5-chloro-4-fluoro-1-methyl-1H-pyrrolo [2,3-b] pyridin-3-yl)-3-(6-methoxy-5-(4-methyl-1H-imidazol-1-yl) pyridin-2-yl)-6-methyl-5,6-dihydro-4H-1,2,4-oxadiazine, fraction (II) (−): Mass (ESI): 470 [M+1]; HPLC (purity): 99.0%; RT 6.26 min; Chiral HPLC: 97.2% RT =14.45 mm; Optical rotation [α]_{D}^{20.00}: −8.73 (C =0. 25, CH₂Cl₂).

### Example 63

### Synthesis of 6-cyclopropyl-5-(4-fluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine

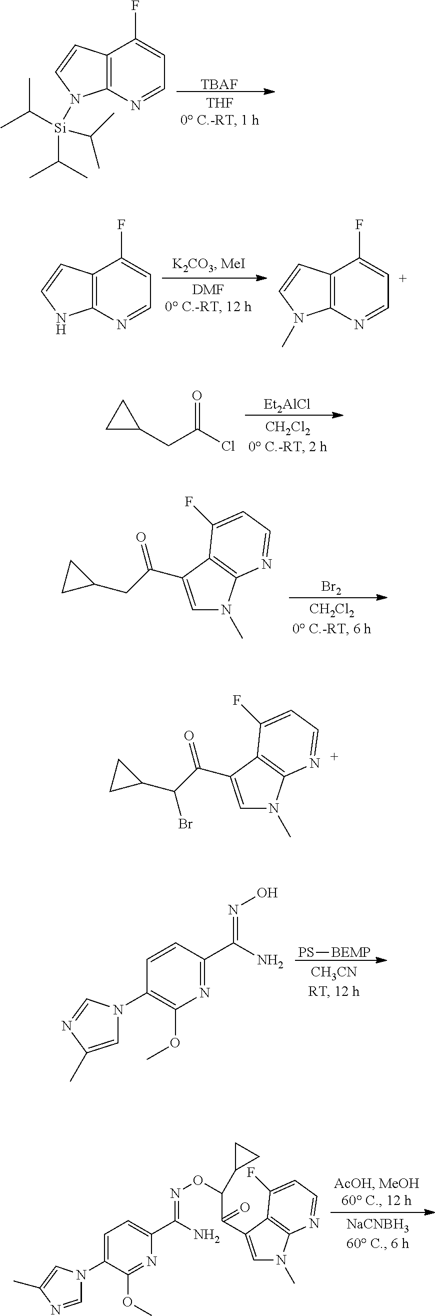

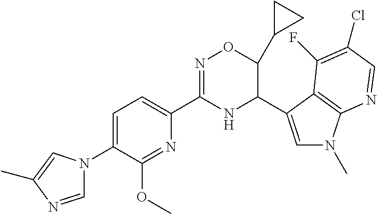 [SMILES: COc1nc(C2=NOC(C3CC3)C(c3cn(C)c4ncc(Cl)c(F)c34)N2)ccc1-n1cnc(C)c1]

### Synthesis of 4-fluoro-M-pyrrolo [2,3-b] pyridine

To a stirred solution of 4-fluoro-1-(triisopropylsilyl)-1H-pyrrolo [2,3-b] pyridine (3 g, 10 mmol) in THF (15 mL) at 0° C. under an argon atmosphere was added tetra butyl ammonium fluoride 1M in THF (10.5 mL). The reaction mixture was warmed to room temperature and stirred for 1 h. After consumption of starting material (by TLC), the reaction mixture was diluted with water (50 mL) and extracted with EtOAc (2×100 mL). The combined organic extracts were dried over sodium sulfate, filtered and concentrated in vacuo. The crude material was purified by column chromatography using 20%EtOAc: Hexane to afford 4-fluoro-1H-pyrrolo [2,3-b] pyridine (1.3 g, 94%) as an off-white solid. ¹H NMR (CDCl₃, 400 MHz): δ 10.52 (brs, 1H), 8.30-8.25 (m, 1H), 7.32-7.30 (m, 1H), 6.81 (dd, 1H), 6.59 (s, 1H); TLC: 20% EtOAc/Hexane (R_{f}:02).

### Synthesis of 4-fluoro-1-methyl-M-pyrrolo [2,3-b] pyridine

To a stirred solution of 4-fluoro-1H-pyrrolo [2,3-b] pyridine (1.4 g, 10 mmol) in DMF (42 mL) at 0° C. under an argon atmosphere were added potassium carbonate (2.1 g, 15 mmol) and methyl iodide (1.75 g, 12 mmol). The reaction mixture was warmed to room temperature and stirred for 12 h. After consumption of starting material (by TLC), the reaction mixture was diluted with water (100 mL) and extracted with EtOAc (2×50 mL). The combined organic extracts were dried over sodium sulfate, filtered and concentrated in vacuo. The crude material was purified by column chromatography using 15%EtOAc: Hexane to afford 4-fluoro-1-methyl-1H-pyrrolo [2,3-b] pyridine (700 mg, 45%) as pale brown liquid. ¹H NMR (CDCl₃, 400 MHz): δ 8.26 (dd, 1H), 7.12 (d, 1H), 6.78 (dd, 1H), 6.51 (d, 1H), 3.90 (s, 3H); TLC: 20% EtOAc/Hexane (R_{f}:05).

### Synthesis of 2-cyclopropyl-1-(4-fluoro-1-methyl-M-pyrrolo [2,3-b] pyridin-3-yl) ethan-1-one

To a stirred solution of 4-fluoro-1-methyl-1H-pyrrolo [2,3-b] pyridine (450 mg, 3 mmol) in CH₂Cl₂ (5 mL) at 0° C. under an argon atmosphere were added diethyl aluminium chloride 1M in Hexane (728 mg, 6 mmol). The reaction mixture was stirred for 15 min. Then 2-cyclopropylacetyl chloride (1.42 g, 12 mmol) in CH₂Cl₂(1.75 mL) was added to the reaction mixture at 0° C. The reaction mixture was warmed to room temperature and stirred for 2 h. After consumption of starting material (by TLC), the reaction mixture was diluted with cold water (50 mL) and extracted with EtOAc (2×100 mL). The combined organic extracts were washed with saturated sodium bicarbonate solution (50 mL), dried over sodium sulfate, filtered and concentrated in vacuo. The crude material was purified by column chromatography using 25%EtOAc: Hexane to afford 2-cyclopropyl-1-(4-fluoro-1-methyl-1H-pyrrolo [2,3-b] pyridin-3-yl) ethan-1-one (300 mg, 43%) as an off-white solid.

¹H NMR (CDCl₃, 400 MHz): δ 8.34 (dd, 1H), 7.88 (s, 1H), 6.96 (dd, 1H), 3.95 (s, 3H), 2.85 (d, 2H), 0.91-0.84 (m, 1H), 0.63-0.56 (m, 2H), 0.24-0.18 (m, 2H); TLC: 30% EtOAc/Hexane (R_{f}:02).

### Synthesis of 2-bromo-2-cyclopropyl-1-(4-fluoro-1-methyl-1H-pyrrolo [2,3-b] pyridin-3-yl) ethan-1-one

To a stirred solution of 2-cyclopropyl-1-(4-fluoro-1-methyl-1H-pyrrolo [2,3-b] pyridin-3-yl) ethan-1-one (400 mg, 2 mmol) in CH₂Cl₂ (5 mL) at 0° C. under an argon atmosphere was added bromine (248 mg, 1 mmol) in CH₂Cl₂ (3 mL). The reaction mixture was warmed to room temperature and stirred for 6 h. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 20%EtOAc: Hexane to afford 2-bromo-2-cyclopropyl-1-(4-fluoro-1-methyl-1H-pyrrolo [2,3-b] pyridin-3-yl) ethan-1-one (300 mg, 56%) as yellow liquid. ¹H NMR (CDCl₃, 400 MHz): δ 8.37 (dd, 1H), 8.02 (s, 1H), 7.01 (dd, 1H), 4.44 (dd, 1H), 3.99 (s, 3H), 1.95-1.78 (m, 1H), 0.98-0.82 (m, 2H), 0.62-0.45 (m, 2H); TLC: 50% EtOAc/Hexane (R_{f}:05).

### Synthesis of (Z)-N′-(1-cyclopropyl-2-(4-fluoro-1-methyl-1H-pyrrolo [2,3-b] pyridin-3-yl)-2-oxoethoxy)-6-methoxy-5-(4-methyl-1H-imidazol-1-yl) picolinimidamide

To a stirred solution of (Z)-5-bromo-N′-hydroxy-6-methoxypicolinimidamide (200 mg, 0.8 mmol) in CH₃CN (7 mL) at room temperature under an argon atmosphere was added PS-BEMP (440 mg). The reaction mixture was stirred for 5 min at room temperature. Then 2-bromo-2-cyclopropyl-1-(4-fluoro-1-methyl-1H-pyrrolo [2,3-b] pyridin-3-yl) ethan-1-one (376 mg, 1 mmol) in CH₃CN (3 mL) was added to the reaction mixture at room temperature. The reaction mixture was stirred for 12 h at room temperature. After consumption of starting material (by TLC), the reaction mixture was filtered and the filtrate was concentrated in vacuo to obtain (Z)-N′-(1-cyclopropyl-2-(4-fluoro-1-methyl-1H-pyrrolo [2,3-b] pyridin-3-yl)-2-oxoethoxy)-6-methoxy-5-(4-methyl-1H-imidazol-1-yl) picolinimidamide (450 mg, crude) as brown semi solid used in the next step without further purification. LCMS: 36.4%; 478 (M+1); (column; Ascentis Express C-18 (50×3.0 mm, 2.7 μm); RT 1.88 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:03).

### Synthesis of 6-cyclopropyl-5-(4-fluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine

To a stirred solution of (Z)-N′ -(1-cyclopropyl-2-(4-fluoro-1-methyl-1H-pyrrolo [2,3-b] pyridin-3-yl)-2-oxoethoxy)-6-methoxy-5-(4-methyl-1H-imidazol-1-yl) picolinimidamide (450 mg, 1 mmol) in MeOH (18 mL) at room temperature under an argon atmosphere was added acetic acid (2 mL). The reaction mixture was stirred for 12 h at 60° C. Then sodium cyanoborohydride (71 mg, 1 mmol) was added to the reaction mixture at room temperature. The reaction mixture was stirred for 6 h at 60° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo. The crude material was purified by column chromatography using 2%MeOH:CH₂Cl₂ to afford 6-cyclopropyl-5-(4-fluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine (80 mg, 18% over two steps) as an off-white solid.

Racemic compound of Example 63 was separated using a Chiralpak-IC column (250×20 mm, 5 μm) (25 mg loading; 0.1% DEA in n-Hexane: CH₂Cl₂:MeOH (50:50) (A:B: 30:70) as mobile phase; flow rate: 20 mL/min) to afford the compounds of Example 63A (Fraction I (+)) and Example 63B (Fraction II (−)).

Analytical conditions for Example 63A and Example 63B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 30:70); flow Rate: 1.0 mL/min).

### Example 63A

(+)-6-cyclopropyl-5-(4-fluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine, fraction (I) (+): ¹H NMR (400 MHz, CD₃OD): δ 8.24 (dd, 1H), 8.12 (brs, 1H), 7.90 (d, 1H), 7.68 (d, 1H), 7.29 (s, 2H), 6.92 (dd, 1H), 5.39 (d, 1H), 4.07 (s, 3H), 3.86 (s, 3H), 3.18 (dd, 1H), 2.27 (s, 3H), 0.49-0.41 (m, 5H); Mass (ESI): 462.1 [M+1]; HPLC (purity): 99.0%; RT 5.75 mm; Chiral HPLC: 100% RT =10.27 mm; Optical rotation [α]_{D}^{19.96}: +15.55 (c=0.25, CH₂Cl₂).

### Example 63B

(−)-6-cyclopropyl-5-(4-fluoro-1-methyl-1H-pyrrolo [2,3-b] pyridin-3-yl)-3-(6-methoxy-5-(4-methyl-1H-imidazol-1-yl) pyridin-2-yl)-5,6-dihydro-4H-1,2,4-oxadiazine, fraction (II) (−): Mass (ESI): 462.2 [M+1]; HPLC (purity): 98.7%; RT 5.73 min; Chiral HPLC: 99.7% RT =13.73 mm; Optical rotation [α]_{D}^{19.99}: −16.12 (c=0.25, CH₂Cl₂).

### Example 64

### Synthesis of 5-chloro-2-(6-cyclopropyl-3-(6-methoxy-5-(4-methyl-1H-imidazol-1-yl) pyridin-2-yl)-5,6-dihydro-4H-1,2,4-oxadiazin-5-yl) furo [2,3-b] pyridine

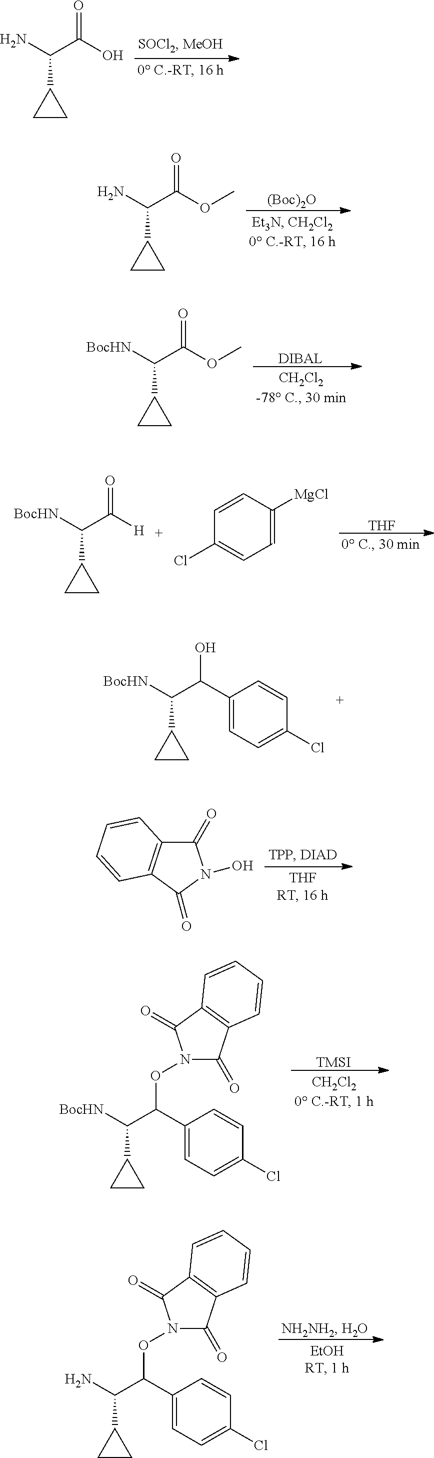

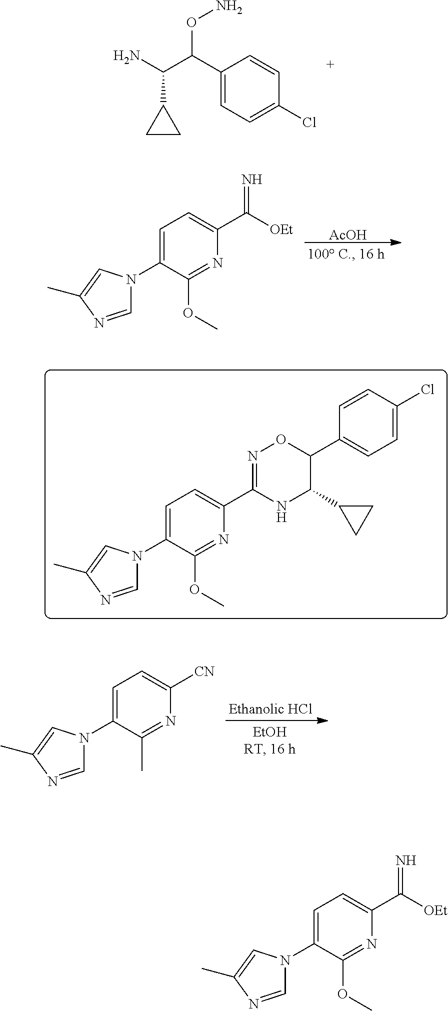 [SMILES: "AcOH" ; NOC(c1ccc(Cl)cc1)[C@@H](N)C1CC1 ; CCOC(=N)c1ccc(-n2cnc(C)c2)c(OC)n1 ; COc1nc(C2=NOC(c3ccc(Cl)cc3)[C@H](C3CC3)N2)ccc1-n1cnc(C)c1 ; Cc1cn(-c2ccc(C#N)nc2C)cn1 ; CCOC(=N)c1ccc(-n2cnc(C)c2)c(OC)n1]

### Synthesis of methyl (S)-2-amino-2-cyclopropylacetate

To a stirred solution of (S)-2-amino-2-cyclopropylacetic acid (750 mg, 6 mmol) in MeOH (7.5 mL) at 0° C. under an argon atmosphere was added thionyl chloride (2.36 mL, 32 mmol). The reaction mixture was warmed to room temperature and stirred for 16 h. After consumption of starting material (by TLC), the volatiles were evaporated in vacuo to obtain methyl (S)-2-amino-2-cyclopropylacetate (800 mg, crude) as brown syrup used in the next step without further purification. ¹H NMR (CD₃OD, 400 MHz): δ 3.83 (s, 3H), 3.36-3.32 (m, 1H), 1.20-1.10 (m, 1H), 0.81-0.65 (m, 3H), 0.57-0.50 (m, 1H); TLC: 10% EtOAc/Hexane (R_{f}:07).

### Synthesis of methyl (S)-2-((tert-butoxycarbonyl) amino)-2-cyclopropylacetate

To a stirred solution of methyl (S)-2-amino-2-cyclopropylacetate (800 mg, 6 mmol) in CH₂Cl₂ (15 mL) at 0° C. under an argon atmosphere were added triethyl amine (4.31mL) and boc anhydride (1.7 mL, 7 mmol). The reaction mixture was warmed to room temperature and stirred for 16 h. After consumption of starting material (by TLC), the volatiles were evaporated in vacuo. The residue was washed with water (30 mL) and extracted with CH₂Cl₂ (2×30 mL). The combined organic extracts were dried over sodium sulfate, filtered and concentrated in vacuo to obtain methyl (S)-2-((tert-butoxycarbonyl) amino)-2-cyclopropylacetate (1 g, crude) as an off-white solid. ¹H NMR (CDCl₃, 500 MHz): 6 5.03 (br s, 1H), 3.78(s, 3H), 1.42 (s, 9H), 1.10-1.03 (m, 1H), 0.60-0.45 (m, 3H), 0.41-0.39 (m, 1H); TLC: 20% EtOAc/Hexane (R_{f}:07).

### Synthesis of tert-butyl (S)-(1-cyclopropyl-2-oxoethyl) carbamate

To a stirred solution of methyl (S)-2-((tert-butoxycarbonyl) amino)-2-cyclopropylacetate (300 mg, 1 mmol) in CH₂Cl₂ (3 mL) at −78° C. under an argon atmosphere was added DIBAL-H 1M in Hexane (1.4 mL, 1.44 mmol). The reaction mixture was stirred at −78° C. for 30 mm After consumption of starting material (by TLC), the reaction mixture was diluted with ammonium chloride solution (30 mL) and extracted with EtOAc (2×30 mL). The combined organic extracts were washed with water (10 mL), brine (10 mL) and dried over sodium sulfate, filtered and concentrated in vacuo to obtain tert-butyl (S)-(1-cyclopropyl-2-oxoethyl) carbamate (220 mg, crude) as an off-white solid. ¹H NMR (DMSO-d₆, 400 MHz): δ 9.49 (s, 1H), 7.35-7.30 (m, 1H), 3.31-3.25 (m, 1H), 1.40 (s, 9H), 1.00-0.91 (m, 1H), 0.56-0.50 (m, 1H), 0.45-0.38 (m, 2H), 0.32-0.25 (m, 1H); TLC: 20% EtOAc/Hexane (R_{f}:03).

### Synthesis of tert-butyl ((1 S)-2-(4-chlorophenyl)-1-cyclopropyl-2-hydroxyethyl) carbamate

To a stirred solution of tert-butyl (S)-(1-cyclopropyl-2-oxoethyl) carbamate (220 mg, 1 mmol) in THF (3 mL) at 0° C. under an argon atmosphere was added (4-chlorophenyl) magnesium chloride (1.2 mL, 1 mmol). The reaction mixture was stirred at 0° C. for 30 min After consumption of starting material (by TLC), the reaction mixture was diluted with saturated ammonium chloride solution (10 mL) and extracted with EtOAc (2×10 mL). The combined organic extracts were washed with water (10 mL), brine (10 mL), dried over sodium sulfate, filtered and concentrated in vacuo. The crude material was purified by column chromatography using 10%EtOAc: Hexane to afford tert-butyl ((1S)-2-(4-chlorophenyl)-1-cyclopropyl-2-hydroxyethyl) carbamate (110 mg, 32%) as white solid. ¹H NMR (DMSO-d₆, 500 MHz): δ 7.32 (s, 4H), 6.32 (d, 1H), 5.38-5.35 (m, 1H), 4.63-4.60 (m, 1H), 3.00 (br s, 1H), 1.30 (s, 9H), 0.83-0.80 (m, 1H), 0.30-0.20 (m, 3H), 0.11-0.08 (m, 1H); TLC: 10% EtOAc/Hexane (R_{f}:01).

### Synthesis of tert-butyl ((1 S)-2-(4-chlorophenyl)-1-cyclopropyl-2-((1,3-dioxoisoindolin-2-yl) oxy) ethyl) carbamate

To a stirred solution of tert-butyl 41S)-2-(4-chlorophenyl)-1-cyclopropyl-2-hydroxyethyl) carbamate (100 mg, 0.3 mmol) in CH₂Cl₂ (7 mL) at 0° C. under an argon atmosphere were added triphenyl phosphine (253 mg, 0.9 mmol), diisopropyl azodicarboxylate (195 mg, 0.9 mmol) and N′-hydroxy phthalamide (52 mg, 0.32 mmol). The reaction mixture was stirred at room temperature for 1 h. After consumption of starting material (by TLC), the volatiles were evaporated in vacuo. The crude material was purified by column chromatography using 10-15%EtOAc: Hexane to afford tert-butyl ((1S)-2-(4-chlorophenyl)-1-cyclopropyl-2-hydroxyethyl) carbamate (40 mg, 27%) as white solid. LCMS: 34.7%; 227.2 (M+1); (column; X select CSH C-18 (50×3.0 mm, 2.5 μm); RT 3.17 min; mobile phase: 2.5 mM Aq NH₄OAc; ACN; T/B: 0.01/10, 0.5/10, 3.5/90, 7/90; flow rate: 0.8 mL/min); (Gradient) TLC: 30% EtOAc/Hexane (R_{f}:06).

### Synthesis of 2-((2S)-2-amino-1-(4-chlorophenyl)-2-cyclopropylethoxy) isoindoline-1,3-dione

To a stirred solution of tert-butyl ((1S)-2-(4-chlorophenyl)-1-cyclopropyl-2-hydroxyethyl) carbamate (30 mg, 0.6 mmol) in CH₂Cl₂ (3 mL) at 0° C. under an argon atmosphere was added TMSI (0.1 mL, 0.08 mmol). The reaction mixture was stirred at room temperature for 1 h. After consumption of starting material (by TLC), the volatiles were evaporated in vacuo. The residue was diluted with n-hexane (2×2 mL), stirred for 5 min, filtered to obtain 2-((2S)-2-amino-1-(4-chlorophenyl)-2-cyclopropylethoxy) isoindoline-1,3-dione (15 mg, 48%) as yellow solid used in the next step without further purification.

¹H NMR (DMSO-d₆, 400 MHz): δ 8.20 (br s, 2H), 7.88 (s, 4H), 7.60 (d, 2H), 7.51 (d, 2H), 5.46-5.43 (m, 1H), 3.06-3.00 (m, 1H), 0.75-0.70 (m, 1H), 0.58-0.51 (m, 2H), 0.47-0.40 (m, 1H), 0.37-0.30 (m, 1H); TLC: 10% MeOH/CH₂Cl₂ (R_{f}:01).

### Synthesis of (5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine

To a stirred solution of 6-methyl-5-(4-methyl-1H-imidazol-1-yl) picolinonitrile (100 mg, 0.2 mmol) in EtOH (2.1 mL) at room temperature under an argon atmosphere was added hydrazine hydrate (362 mg, 0.7 mmol). The reaction mixture was stirred at room temperature for 1 h. After consumption of starting material (by TLC), the reaction mixture was filtered, the filtrate was concentrated in vacuo to obtain (5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine (45 mg, 97%) as yellow solid used in the next step without further purification. TLC: 5% MeOH/CH₂Cl₂ (R_{f}:01).

### Synthesis of ethyl 6-methoxy-5-(4-methyl-1H-imidazol-1-yl) picolinimidate

To a stirred solution of 6-methyl-5-(4-methyl-1H-imidazol-1-yl) picolinonitrile (150 mg, 0.7 mmol) in EtOH (2 mL) at room temperature under an argon atmosphere was added 4M ethanolic HCl (3 mL). The reaction mixture was stirred at room temperature for 16 h. After consumption of starting material (by TLC), the volatiles were evaporated in vacuo. The residue was diluted with EtOAc (10 mL), to obtain the solid. The solid was filtered, washed with 50% EtOH: EtOAc and dried in vacuo to afford ethyl 6-methoxy-5-(4-methyl-1H-imidazol-1-yl) picolinimidate (130 mg, 71%) as yellow solid used in the next step without further purification. LCMS: 78.9%; 261.9 (M+1); (column; Ascentis Express C-18 (50×3.0 mm, 2.7 μm); RT 1.60 mm; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:05).

### Synthesis of (5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine

To a stirred solution of ethyl 6-methoxy-5-(4-methyl-1H-imidazol-1-yl) picolinimidate (100 mg, 0.4 mmol) in AcOH (3 mL) at room temperature under an argon atmosphere was added (5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine (87 mg, 0.4 mmol). The reaction mixture was stirred at 100° C. for 16 h. After consumption of starting material (by TLC), the volatiles were evaporated in vacuo. The crude material was purified by column chromatography using 1%MeOH:CH₂Cl₂ to afford (5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine (30 mg, 18%) as white solid. TLC: 5% MeOH/CH₂Cl₂ (R_{f}:02).

Racemic compound of Example 64 was separated using a Chiralpak-IA column (250×20 mm, 5 μm) (40 mg loading; 0.1% DEA in n-Hexane: CH₂Cl₂:MeOH (50:50) (A:B: 70:30) as mobile phase; flow rate: 20 mL/min) to afford the compounds of Example 64A (Fraction (I) (−)) and Example 64B (Fraction (II) (+)).

Analytical conditions for Example 64A and Example 64B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 70:30); flow Rate: 1.0 mL/min).

### Example 64A

(−)-(5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine, fraction (I) (−); Mass (ESI): 424.1 [M+1]; HPLC (purity): 99.0%; RT 7.74 min; Chiral HPLC: 100% RT =6.12 min; Optical rotation [α]_{D}^{20.00}: −36.20 (c=0.25, CH₂Cl₂).

### Example 64B

(+)-(5S)-6-(4-chlorophenyl)-5-cyclopropyl-3-(6-methoxy-5-(4-methyl-1H-imidazol-1-yl) pyridin-2-yl)-5,6-dihydro-4H-1,2,4-oxadiazine, fraction (II) (+); ¹H NMR (CD₃OD, 400 MHz): δ 8.09 (s, 1H), 7.89 (d, 1H), 7.64 (d, 1H), 7.49-7.45 (m, 2H), 7.42-7.37 (m, 2H), 7.26 (s, 1H), 4.91 (d, 1H), 4.14 (s, 3H), 3.39 (dd, 1H), 2.30-2.25 (m, 3H), 0.94-0.84 (m, 1H), 0.48-0.33 (m, 2H), 0.17-0.11 (m, 1H), −0.09-0.17 (m, 1H); Mass (ESI): 424.1 [M+1]; HPLC (purity): 99.1%; RT 7.74 min; Chiral HPLC: 100% RT =9.71 min; Optical rotation [α]_{D}^{20.00}: +38.68 (c=0.25, CH₂Cl₂).

### Example 65

### Synthesis of 5-(4-chlorophenyl)-3-(5-methoxy-6-(4-methyl-1H-imidazol-1-yl) pyridin-3-yl)-6-methyl-5,6-dihydro-4H-1,2,4-oxadiazine

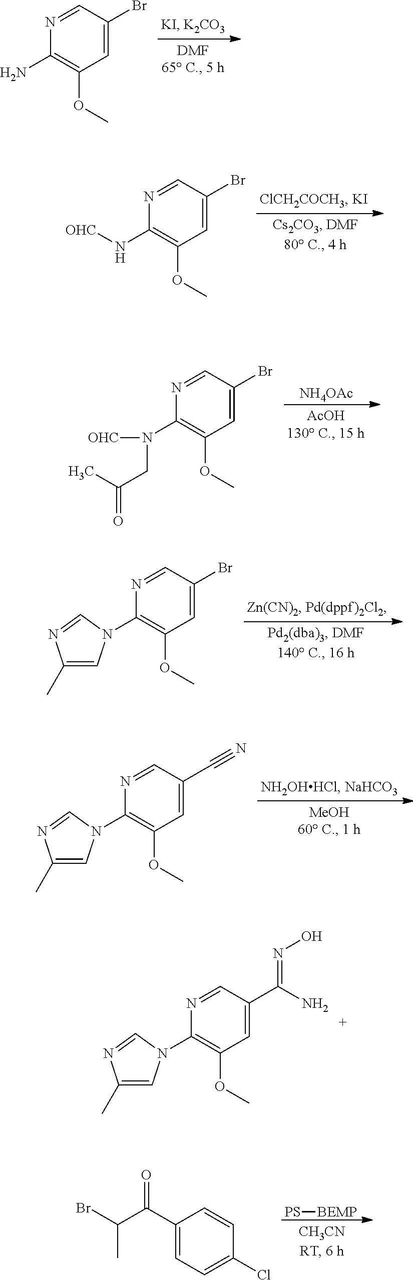

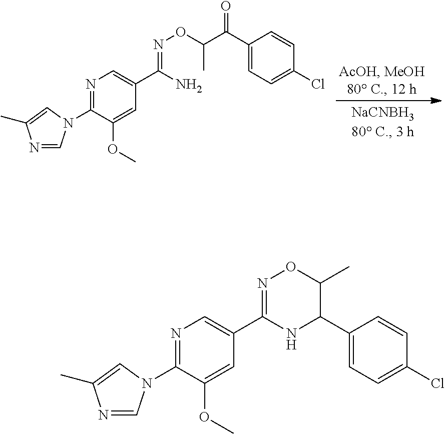 [SMILES: COc1cc(/C(N)=N/OC(C)C(=O)c2ccc(Cl)cc2)cnc1-n1cnc(C)c1 ; COc1cc(C2=NOC(C)C(c3ccc(Cl)cc3)N2)cnc1-n1cnc(C)c1]

### Synthesis of N-(5-bromo-3-methoxypyridin-2-yl) formamide

To a stirred solution of 5-bromo-3-methoxypyridin-2-amine (1 g, 5 mmol) in THF (2 mL) at 0° C. under an argon atmosphere were added formic acid (16.25 mL, 4 mmol) and acetic anhydride (16.2 mL, 2 mmol). The reaction mixture was stirred at room temperature for 1 h. After consumption of starting material (by TLC), the reaction mixture was diluted with saturated sodium bicarbonate solution (50 mL) and extracted with CH₂Cl₂ (2×50 mL). The combined organic extracts were dried over sodium sulfate, filtered and concentrated in vacuo to obtain N-(5-bromo-3-methoxypyridin-2-yl) formamide (930 mg, 81%) as a pale yellow solid. ¹H NMR (DMSO-d₆, 500 MHz): δ 10.24 (d, 1H), 9.12 (hr s, 1H), 7.96 (s, 1H), 7.68 (s, 1H), 3.89 (s, 3H); TLC: 10% MeOH/CH₂Cl₂ (R_{f}:06).

### Synthesis of N-(5-bromo-3-methoxypyridin-2-yl)-N-(2-oxopropyl) formamide

To a stirred solution of N-(5-bromo-3-methoxypyridin-2-yl) formamide (500 mg, 2 mmol) in DMF (3 mL) at room temperature under an argon atmosphere were added cesium carbonate (1.4 g, 4 mmol), chloro acetone (0.26 mL, 3 mmol), potassium iodide (35 mg, 0.2 mmol). The reaction mixture was stirred at 80° C. for 4 h. After consumption of starting material (by TLC), the reaction mixture was diluted with water (20 mL) and extracted with CH₂Cl₂ (2×20 mL). The combined organic extracts were dried over sodium sulfate, filtered and concentrated in vacuo to obtain N-(5-bromo-3-methoxypyridin-2-yl)-N-(2-oxopropyl) formamide (150 mg, 26%) as a pale yellow solid. LCMS: 80.6%; 288.8 (M+3); (column; Kinetex EVO C-18 (50×3.0 mm, 2.6 μm); RT 1.96 min; mobile phase: 2.5 mM NH₄OOCH in water+5% ACN:ACN+5% 2.5 mM NH₄OOCH in water; T/B %: 0.01/5, 4/95, 5.5/95; flow rate: 0.8 mL/min) (Gradient); TLC: 10% MeOH/CH₂Cl₂ (R_{f}:05).

### Synthesis of 5-bromo-3-methoxy-2-(4-methyl-M-imidazol-1-yl) pyridine

To a stirred solution of N-(5-bromo-3-methoxypyridin-2-yl)-N-(2-oxopropyl) formamide (1.1 g, 4 mmol) in acetic acid (10 mL) at room temperature under an argon atmosphere was added ammonium acetate (1.5 g, 20 mmol). The reaction mixture was stirred at 130° C. for 15 h. After consumption of starting material (by TLC), the reaction mixture was diluted with water (50 mL) and extracted with EtOAc (2×50 mL). The combined organic extracts were dried over sodium sulfate, filtered and concentrated in vacuo. The crude material was purified by column chromatography using 50-70% EtOAc: Hexane to afford 5-bromo-3-methoxy-2-(4-methyl-1H-imidazol-1-yl) pyridine (180 mg, 17%) as an off-white solid. ¹H NMR (CDCl₃, 400 MHz): δ 8.30 (s, 1H), 8.10 (s, 1H), 7.45 (s, 2H), 3.92 (s, 3H), 2.28 (s, 3H); TLC: 70% EtOAc/Hexane (R_{f}:02).

### Synthesis of 5-methoxy-6-(4-methyl-1H-imidazol-1-yl) nicotinonitrile

To a stirred solution of 5-bromo-3-methoxy-2-(4-methyl-1H-imidazol-1-yl) pyridine (170 mg, 0.6 mmol) in DMF (2 mL) at room temperature under an argon atmosphere were added Pd₂(dba)₃ (6 g, 0.06 mmol). Pd(dppf)₂Cl₂ (8.5 mg, 0.007 mmol) and zinc cyanide (44 mg, 0.4 mmol). The reaction mixture was stirred at 140° C. for 16 h. After consumption of starting material (by TLC), the reaction mixture was diluted with water (50 mL) and extracted with EtOAc (2×50 mL). The combined organic extracts were dried over sodium sulfate, filtered and concentrated in vacuo to obtain 5-methoxy-6-(4-methyl-1H-imidazol-1-yl) nicotinonitrile (150 mg, crude) as an off-white solid. TLC: 5% MeOH/CH₂Cl₂ (R_{f}:05).

### Synthesis of (Z)-N′-hydroxy-5-methoxy-6-(4-methyl-1H-imidazol-1-yl) nicotinimidamide

To a stirred solution of 5-methoxy-6-(4-methyl-1H-imidazol-1-yl) nicotinonitrile (300 mg, 1 mmol) in MeOH (7.5 mL) at room temperature under an argon atmosphere were added sodium bicarbonate (182 mg, 2 mmol) and hydroxyl amine hydrochloride (125 mg, 2 mmol). The reaction mixture was stirred at 60° C. for 1 h. After consumption of starting material (by TLC), the reaction mixture was diluted with water (50 mL) and extracted with EtOAc (2×50 mL). The combined organic extracts were dried over sodium sulfate, filtered and concentrated in vacua The crude material was purified by column chromatography using 15-25% EtOAc: Hexane to afford (Z)-N′-hydroxy-5-methoxy-6-(4-methyl-1H-imidazol-1-yl) nicotinimidamide (200 mg, 58%) as an off-white solid. ¹H NMR (DMSO-d₆, 400 MHz): δ 9.90 (s, 1H), 8.34 (s, 1H), 8.30 (s, 1H), 7.85 (s, 1H), 7.54 (s, 1H), 6.08 (s, 2H), 3.98 (s, 3H), 2.17 (s, 3H); TLC: 10% MeOH/CH₂Cl₂ (R_{f}:05).

### Synthesis of (Z)-N′-((1-(4-chlorophenyl)-1-oxopropan-2-yl) oxy)-5-methoxy-6-(4-methyl-1H-imidazol-1-yl) nicotinimidamide

To a stirred solution of (Z)-N′-hydroxy-5-methoxy-6-(4-methyl-1H-imidazol-1-yl) nicotinimidamide (200 mg, 0.8 mmol) in CH₃CN (5 mL) at room temperature under an argon atmosphere were added PS-BEMP (266 mg). The reaction mixture was stirred for 15 min at room temperature. Then 2-bromo-1-(4-chlorophenyl) propan-1-one (301 mg, 1 mmol) in CH₃CN (5 mL) was added to the reaction mixture at room temperature. The reaction mixture was stirred for 6 h at room temperature. After consumption of starting material (by TLC), the reaction mixture was filtered, washed with EtOAc (50 mL). The filtrate was concentrated in vacuo to obtain (Z)-N4(1-(4-chlorophenyl)-1-oxopropan-2-yl) oxy)-5-methoxy-6-(4-methyl-1H-imidazol-1-yl) nicotinimidamide (350 mg, crude) as a brown syrup used in the next step without further purification. LCMS: 16.2%; 414.1 (M+1); (column; Ascentis Express C-18 (50×3.0 mm, 3.5 μm); RT 2.03 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 10% MeOH/CH₂Cl₂ (R_{f}:04).

### Synthesis of 5-(4-chlorophenyl)-3-(5-methoxy-6-(4-methyl-1H-imidazol-1-yl) pyridin-3-yl)-6-methyl-5,6-dihydro-4H-1,2,4-oxadiazine

To a stirred solution of (Z)-N4(1-(4-chlorophenyl)-1-oxopropan-2-yl) oxy)-5-methoxy-6-(4-methyl-1H-imidazol-1-yl) nicotinimidamide (350 mg, 0.7 mmol) in MeOH (2 mL) at room temperature under an argon atmosphere was added acetic acid (1.75 mL). The reaction mixture was stirred for 12 h at 80° C. Then sodium cyanoborohydride (54 mg, 1 mmol) was added to the reaction mixture. The reaction mixture was stirred for 3 h at 80° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo to obtain the crude. The crude material was purified by column chromatography using 1% MeOH:CH₂Cl₂ to afford 5-(4-chlorophenyl)-3-(5-methoxy-6-(4-methyl-1H-imidazol-1-yl) pyridin-3-yl)-6-methyl-5,6-dihydro-4H-1,2,4-oxadiazine (24 mg, 7%, over two steps) as an off-white solid. TLC: 15% MeOH/CH₂Cl₂ (R_{f}:06).

Racemic compound of Example 65 was separated using a Chiralpak-IA column (250×20 mm, 5 μm) (40 mg loading; 0.1% DEA in n-Hexane: CH₂Cl₂:MeOH (50:50) (A:B: 85:15) as mobile phase; flow rate: 20 mL/min) to afford the compounds of Example 65A (Fraction (I) and Example 65B (Fraction (II).

Analytical conditions for Example 65A and Example 65B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IA (250×4.6 mm, 5 μm; mobile phase (A) 0.1% DEA in n-Hexane (B) CH₂Cl₂:MeOH (50:50) (A::B; 85:15); flow Rate: 1.0 mL/min).

### Example 65A

5-(4-chlorophenyl)-3-(5-methoxy-6-(4-methyl-1H-imidazol-1-yl) pyridin-3-yl)-6-methyl-5,6-dihydro-4H-1,2,4-oxadiazine, fraction (I); Mass (ESI): 398 [M+1]; HPLC (purity): 97.0%; RT 6.62 mm; Chiral HPLC: 97.0% RT =14.64 min

### Example 65B

5-(4-chlorophenyl)-3-(5-methoxy-6-(4-methyl-1H-imidazol-1-yl) pyridin-3-yl)-6-methyl-5,6-dihydro-4H-1,2,4-oxadiazine, fraction (II); ¹H NMR (CD₃OD, 400 MHz): δ 8.40 (s, 1H), 8.38 (s, 1H), 7.87 (s, 1H), 7.63 (s, 1H), 7.38 (d, 2H), 7.30 (d, 2H), 4.66-4.64 (m, 1H), 4.11-4.08 (m, 1H), 4.05 (s, 3H), 2.22 (s, 3H), 0.99 (d, 3H); Mass (ESI): 398 [M+1]; HPLC (purity): 96.7%; RT 6.61 mm; Chiral HPLC: 97.1% RT =16.32 mm.

### Example 66

### Synthesis of (1R, 7R, 8aS)-1-(4-chlorophenyl)-7-methoxy-4-(6-methoxy-5-(4-methyl-1H-imidazol-1-yl) pyridin-2-yl)-6,7,8,8a-tetrahydro-1H-pyrrolo [1,2-d] [1,2,4] oxadiazine

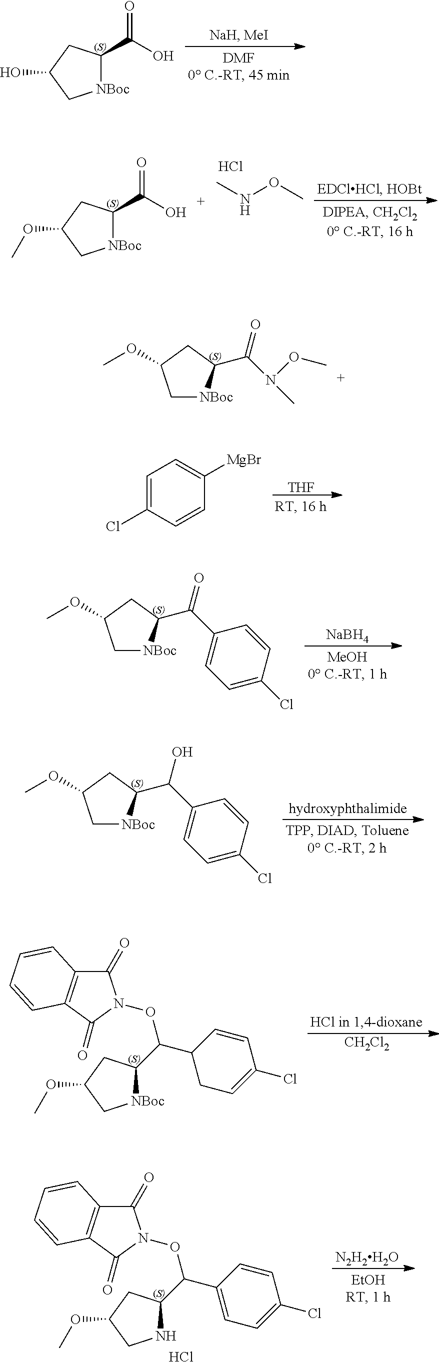

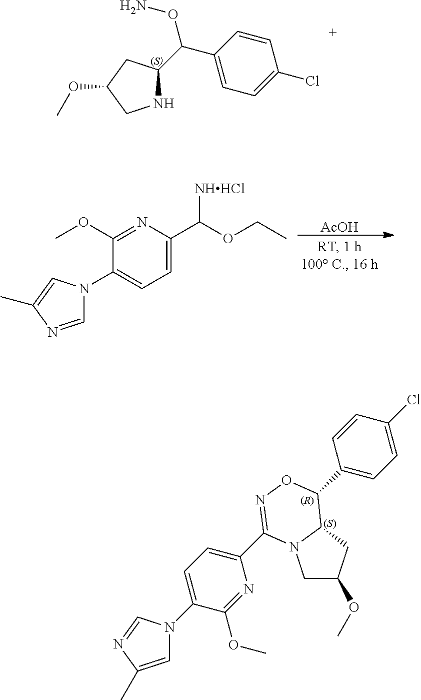

### Synthesis of (2S, 4R)-1-(tert-butoxycarbonyl)-4-methoxypyrrolidine-2-carboxylic acid

To a stirred solution of sodium hydride (4.3 g, 108 mmol) in DMF (74 mL) at 0° C. under an argon atmosphere was added (2S, 4R)-1-(tert-butoxycarbonyl)-4-hydroxypyrrolidine-2-carboxylic acid (10 g, 43 mmol) in DMF (93 mL). The reaction mixture was stirred at RT for 30 mm Then methyl iodide (2.7 mL) was added to the reaction mixture at 0° C. The reaction mixture was stirred at room temperature for 1 h. After consumption of starting material (by TLC), the reaction mixture was quenched with 1N HCl solution (100 mL) and extracted with EtOAc (2×500 mL). The combined organic extracts were washed with water (100 mL), dried over sodium sulfate, filtered and concentrated in vacuo. The crude material was purified by column chromatography using 15-25% EtOAc: Hexane to afford (2S,4R)-1-(tert-butoxycarbonyl)-4-methoxypyrrolidine-2-carboxylic acid (6.5 g, 61%) as an off-white solid. ¹H NMR (DMSO-d₆, 400 MHz): δ 12.52 (br s, 1H), 4.10-4.00 (m, 1H), 3.94 (d, 1H), 3.44-3.36 (m, 2H), 3.20 (s, 3H), 2.34-2.22 (m, 1H), 1.99-1.86 (m, 1H), 1.39 (s, 9H); TLC: 10% MeOH/CH₂Cl₂ (R_{f}:05).

### Synthesis of tert-butyl (2S, 4R)-4-methoxy-2-(methoxy (methyl) carbamoyl) pyrrolidine-1-carboxy late

To a stirred solution of (28,4R)-1-(tert-butoxycarbonyl)-4-methoxypyrrolidine-2-carboxylic acid (200 g, 0.8 mmol) in CH₂Cl₂ (0.43 mL) at 0° C. under an argon atmosphere were added EDCI. HCl (235 mg, 1 mmol), HOBt (150 mg, 0.7 mmol), diisopropyl ethyl amine (5 mL, 2 mmol) and N,O-dimethylhydroxylamine hydrochloride (95 mg, 1 mmol). The reaction mixture was stirred at room temperature for 16 h. After consumption of starting material (by TLC), the reaction mixture was quenched with saturated sodium bicarbonate solution (50 mL) and extracted with CH₂Cl₂ (2×50 mL). The combined organic extracts were dried over sodium sulfate, filtered and concentrated in vacuo. The crude material was purified by column chromatography using 15-10% MeOH:CH₂Cl₂ to afford tert-butyl (2S, 4R)-4-methoxy-2-(methoxy (methyl) carbamoyl) pyrrolidine-1-carboxylate (100 mg, 43%) as colorless liquid. ¹H NMR (DMSO-d₆, 400 MHz): δ 4.70-4.51 (m, 1H), 3.98-3.84 (m, 1H), 3.69 (s, 3H), 3.46-3.32 (m, 2H), 3.23 (s, 3H), 3.11 (s, 3H), 2.34-2.17 (m, 1H), 1.91-1.80 (m, 1H), 1.37 (s, 9H); TLC: 15% MeOH/CH₂Cl₂ (R_{f}:05).

### Synthesis of tert-butyl (2S, 4R)-2-(4-chlorobenzoyl)-4-methoxypyrrolidine-1-carboxylate

To a stirred solution of tert-butyl (2S, 4R)-4-methoxy-2-(methoxy (methyl) carbamoyl) pyrrolidine-1-carboxylate (4 g, 14 mmol) in THF (80 mL) at 0° C. under an argon atmosphere was added (4-chlorophenyl) magnesium bromide (8.98 g, 41 mmol). The reaction mixture was stirred at room temperature for 16 h. After consumption of starting material (by TLC), the reaction mixture was quenched with saturated sodium bicarbonate solution (200 mL) and extracted with EtOAc (2×100 mL). The combined organic extracts were dried over sodium sulfate, filtered and concentrated in vacuo. The crude material was purified by column chromatography using 2-3% MeOH:CH₂Cl₂ to afford tert-butyl (2S, 4R)-2-(4-chlorobenzoyl)-4-methoxypyrrolidine-1-carboxylate (3 g, 64%) as an off-white solid.

¹H NMR (DMSO-d₆, 500 MHz): δ 8.02 (d, 2H), 7.61 (d, 2H), 5.29-5.22 (m, 1H), 3.96 (brs, 1H), 3.54-3.44 (m, 2H), 3.24 (s, 3H), 2.47-2.34 (m, 1H), 1.91-1.81 (m, 1H), 1.37 (s, 9H); TLC: 5% MeOH/CH₂Cl₂ (R_{f}:06).

### Synthesis of tert-butyl (2S, 4R)-2-((4-chlorophenyl) (hydroxy) methyl)-4-methoxypyrrolidine-1-carboxylate

To a stirred solution of tert-butyl (2S, 4R)-2-(4-chlorobenzoyl)-4-methoxypyrrolidine-1-carboxylate (3 g, 9 mmol) in MeOH (30 mL) at 0° C. under an argon atmosphere was added sodium borohydride (840 mg, 22 mmol). The reaction mixture was stirred at room temperature for 1 h. After consumption of starting material (by TLC), the reaction mixture was quenched with saturated ice cold water (100 mL) and extracted with EtOAc (2×100 mL). The combined organic extracts were dried over sodium sulfate, filtered and concentrated in vacuo to obtain tert-butyl (2S, 4R)-2-(4-chlorobenzoyl)-4-methoxypyrrolidine-1-carboxylate (2.7 g, 89%) as a pale yellow syrup. ¹H NMR (DMSO-d₆, 400 MHz): δ 7.38 (d, 2H), 7.22 (d, 2H), 5.64-5.52 (m, 1H), 5.09-4.88 (m, 1H), 4.14-4.00 (m, 1H), 3.97-3.84 (m, 1H), 3.42-3.32 (m, 1H), 3.13 (s, 3H), 2.68-2.56 (m, 1H), 1.93-1.73 (m, 2H), 1.43 (d, 9H); TLC: 30% EtOAc/Hexane (R_{f}:06).

### Synthesis of tert-butyl (25, 4R)-2-((4-chlorocyclohexa-2,4-dien-1-yl) ((1,3-dioxoisoindolin-2-yl)oxy)methyl)-4-methoxypyrrolidine-1-carboxylate

To a stirred solution of N-hydroxy phthalimide (210 mg, 1 mmol) in toluene (16 mL) at 0° C. under an argon atmosphere were added diisopropylazodicarboxylate (591.4 mg, 3 mmol) and triphenyl phosphine (619.6 mg, 3 mmol). The reaction mixture was stirred for 5 min. Then (tert-butyl (2S, 4R)-2-(4-chlorobenzoyl)-4-methoxypyrrolidine-1-carboxylate (400 mg, 1 mmol) in toluene (24 mL) was added to the reaction mixture. The reaction mixture was stirred at room temperature for 2 h. After consumption of starting material (by TLC), the volatiles were evaporated in vacuo. The crude material was purified by column chromatography using 10-15% EtOAc: Hexane to afford tert-butyl (2S, 4R)-2-((4-chlorocyclohexa-2,4-dien-1-yl) ((1,3-dioxoisoindolin-2-yl) oxy) methyl)-4-methoxypyrrolidine-1-carboxylate (450 mg, 79%) as an off-white solid. LCMS: 86.2%; 389.1 (M-Boc); (column; Kinetex EVO C-18 (50×3.0 mm, 2.6 μm); RT 3.96 min; mobile phase: 2.5 mM NH₄OOCH in water+5% ACN:ACN+5% 2.5 mM NH₄OOCH in water; T/B %: 0.01/5, 4/95, 5.5/95; flow rate: 0.8 mL/min) (Gradient); TLC: 30% EtOAc/Hexane (R_{f}:06).

### Synthesis of 2-((4-chlorophenyl) ((2S,4R)-4-methoxypyrrolidin-2-yl) methoxy) isoindoline-1,3-dione

To a stirred solution of tert-butyl (2S, 4R)-2-((4-chlorocyclohexa-2, 4-dien-1-yl) ((1, 3-dioxoisoindolin-2-yl) oxy) methyl)-4-methoxypyrrolidine-1-carboxylate (300 mg, 0.6 mmol) in CH₂Cl₂ (4.5 mL) at 0° C. under an argon atmosphere was added HCl in 1,4-dioxane (0.75 mL). The reaction mixture was stirred at room temperature for 2 h. After consumption of starting material (by TLC), the reaction mixture was quenched with saturated ice cold water (50 mL) and extracted with CH₂Cl₂ (2×50 mL). The combined organic extracts were dried over sodium sulfate, filtered and concentrated in vacuo. The crude material was purified by column chromatography using 2-3% MeOH:CH₂Cl₂ to afford 2-((4-chlorophenyl) ((2S,4R)-4-methoxypyrrolidin-2-yl) methoxy) isoindoline-1,3-dione (120 mg, crude) as an off-white solid. LCMS: 48.6%; 387 (M+1); (column; Kinetex EVO C-18 (50×3.0 mm, 2.6 μm); RT 2.71 min; mobile phase: 2.5 mM NH₄OOCH in water+5% ACN:ACN+5% 2.5 mM NH₄OOCH in water; T/B %: 0.01/5, 4/95, 5.5/95; flow rate: 0.8 mL/min) (Gradient); TLC: 5% MeOH/CH₂Cl₂ (R_{f}:03).

### Synthesis of O-((4-chlorophenyl) ((2S,4R)-4-methoxypyrrolidin-2-yl) methyl) hydroxylamine

To a stirred solution of 2-((4-chlorophenyl) ((2S,4R)-4-methoxypyrrolidin-2-yl) methoxy) isoindoline-1,3-dione (150 mg, 0.4 mmol) in EtOH (4 mL) at 0° C. under an argon atmosphere was added hydrazine hydrate (68 mg, 1 mmol). The reaction mixture was stirred at room temperature for 1 h. After consumption of starting material (by TLC), the reaction mixture was quenched with saturated ice cold water (50 mL) and extracted with EtOAc (2×50 mL). The combined organic extracts were dried over sodium sulfate, filtered and concentrated in vacuo to obtain O-((4-chlorophenyl) ((2S,4R)-4-methoxypyrrolidin-2-yl) methyl) hydroxylamine (130 mg, crude) as brown solid used in the next step without further purification. LCMS: 19.6%; 256.9 (M+1); (column; Kinetex EVO C-18 (50×3.0 mm, 2.6 μm); RT 1.65 min; mobile phase: 2.5 mM NH₄OOCH in water+5% ACN:ACN+5% 2.5 mM NH₄OOCH in water; T/B %: 0.01/5, 4/95, 5.5/95; flow rate: 0.8 mL/min) (Gradient); TLC: 10% MeOH/CH₂Cl₂ (R_{f}:04).

### Synthesis of (1R,7R,8aS)-1-(4-chlorophenyl)-7-methoxy-4-(6-methoxy-5-(4-methyl-1H-imidazol-1-yl) pyridin-2-yl)-6,7,8,8a-tetrahydro-1H-pyrrolo [1,2-d] [1,2,4] oxadiazine

To a stirred solution of O-((4-chlorophenyl) ((2S,4R)-4-methoxypyrrolidin-2-yl) methyl) hydroxylamine (130 mg, 0.5 mmol) in AcOH (7 mL) at room temperature under an argon atmosphere was added ethyl 6-methoxy-5-(4-methyl-1H-imidazol-1-yl) picolinimidate hydrochloride (170 mg, 0.5 mmol). The reaction mixture was stirred at room temperature for 1 h. The reaction mixture was stirred at 100° C. for 16 h. After consumption of starting material (by TLC), the reaction mixture was quenched with saturated ice cold water (50 mL) and extracted with CH₂Cl₂ (2×50 mL). The combined organic extracts were dried over sodium sulfate, filtered and concentrated in vacuo. The crude material was purified by column chromatography using 2-3% MeOH:CH₂Cl₂ to afford (1R,7R,8a5)-1-(4-chlorophenyl)-7-methoxy-4-(6-methoxy-5-(4-methyl-1H-imidazol-1-yl) pyridin-2-yl)-6,7,8,8a-tetrahydro-1H-pyrrolo [1,2-d] [1,2,4] oxadiazine (15 mg, 7%) as an off-white solid. ¹H NMR (CD₃OD, 400 MHz): δ 8.00 (s, 1H), 7.91 (d, 1H), 7.45 (d, 1H), 7.38 (s, 4H), 7.24 (s, 1H), 5.45 (d, 1H), 4.24-4.17 (m, 1H), 4.09 (s, 3H), 4.00 (d, 1H), 3.77 (t, 1H), 3.52 (dd, 1H), 3.32 (s, 3H), 2.26 (s, 3H), 2.24-2.21 (m, 1H), 1.49-1.42 (m, 1H); LCMS: 11.4%; 454.1 (M+1); (column; Kinetex EVO C-18 (50×3.0 mm, 2.6 μm); RT 2.87 min; mobile phase: 2.5 mM NH₄OOCH in water+5% ACN:ACN+5% 2.5 mM NH₄OOCH in water; T/B %: 0.01/5, 4/95, 5.5/95; flow rate: 0.8 mL/min) (Gradient); TLC: 5% MeOH/CH₂Cl₂ (R_{f}:05).

### Example 67

### Synthesis of 2-(aminooxy)-1-(5-chloro-1-methyl-1H-indazol-3-yl)propan-1-one

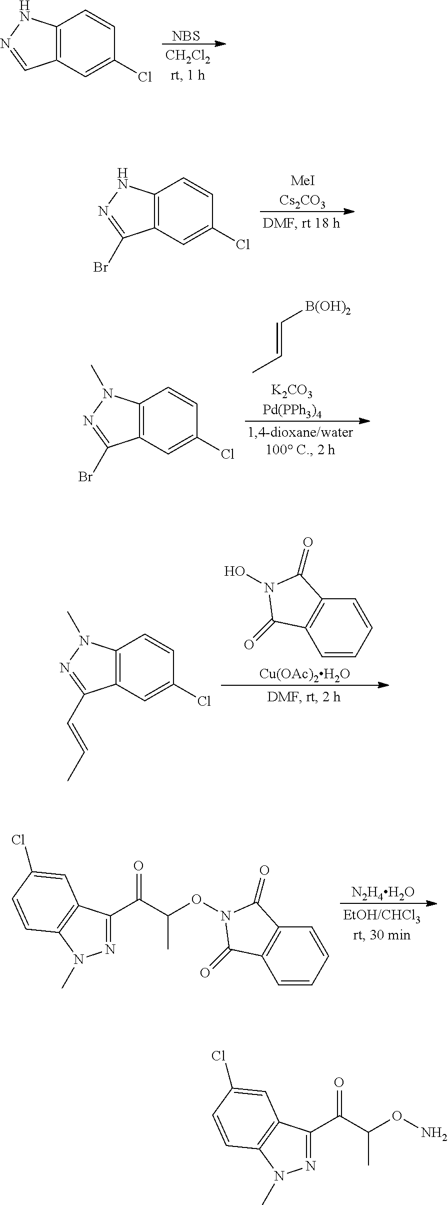

3-bromo-5-chloro-1H-indazole: To a solution of 5-chloro-1H-indazole (2.5 g, 16.1 mmol) in dichloromethane (150 mL) was added N-bromosuccinimide (2.9 g, 16.6 mmol) and the mixture was stirred at room temperature for 1 hour. The mixture was concentrated under reduced pressure and the residue was dissolved in ethyl acetate (100 mL), washed with water (50 mL) and brine (50 mL), dried over sodium sulfate and concentrated under reduced pressure to afford 3-bromo-5-chloro-1H-indazole (3.7 g, 94%) as a brown solid. ¹H NMR (DMSO-d6, 400 MHz) δ 13.63 (s, 1H), 7.67-7.59 (m, 2H), 7.49-7.44 (m, 1H). TLC: 50% ethyl acetate/heptane (R_{f}:043).

3-bromo-5-chloro-1-methyl-M-indazole: To a solution of 3-bromo-5-chloro-1H-indazole (3.7 g, 15.1 mmol) in N,N-dimethylformamide (30 mL) was added cesium carbonate (6.4 g, 19.6 mmol) followed by iodomethane (1.1 mL, 18.1 mmol) and the mixture was stirred at room temperature for 18 hours. The mixture was diluted with water (200 mL) and extracted with ethyl acetate (3×100 mL). The combined organic extracts were washed with brine (3×75 mL), dried over sodium sulfate and concentrated under reduced pressure to afford an oil that was purified by silica flash chromatography [15% to 15% ethyl acetate in heptane] to obtain 3-bromo-5-chloro-1-methyl-1H-indazole (2.5 g, 67%) as a dark yellow solid. 1H NMR (CDCl₃, 400 MHz) δ 7.59 (dd, J=1.9, 0.6 Hz, 1H), 7.39 (dd, J=8.9, 1.9 Hz, 1H), 7.31 (dd, J=8.9, 0.5 Hz, 1H), 4.04 (s, 3H). LCMS: 100%; 244.9 (M+1); RT 2.14 min (method C). TLC: 20% ethyl acetate/heptane (R_{f}:040).

(E)-5-chloro-1-methyl-3-(prop-1-en-1-yl)-1H-indazole: A suspension of 3-bromo-5-chloro-1-methyl-1H-indazole (3.1 g, 12.5 mmol) and potassium carbonate (5.2 g, 37.4 mmol) in 1,4-dioxane (75 mL) and water (20 mL) was degassed with argon. Trans-1-propen-1-ylboronic acid (2.1 g, 24.5 mmol) and tetrakis(triphenylphosphine)palladium (720 mg, 0.6 mmol) were added and the mixture was heated at 100° C. for 2 hours. The mixture was diluted with water (150 mL) and extracted with ethyl acetate (2×150 mL). The combined organic extracts were washed with brine, dried over sodium sulfate and concentrated under reduced pressure to afford a brown solid that was purified by silica flash chromatography [15% to 15% ethyl acetate in heptane] to afford (E)-5-chloro-1-methyl-3-(prop-1-en-1-yl)-1H-indazole (2.2 g, 84%) as an orange solid. 1H NMR (CDCl₃, 400 MHz) δ 7.83 (dd, J=1.8, 0.6 Hz, 1H), 7.32 (dd, J=8.9, 1.8 Hz, 1H), 7.29-7.23 (m, 1H), 6.66 (m, 1H), 6.55 (m, 1H), 4.01 (s, 3H), 1.97 (dd, J=6.3, 1.4 Hz, 3H). LCMS: 96.7%; 207.0 (M+1); RT 2.14 mm (method C). TLC: 20% ethyl acetate/heptane (R_{f}:028).

2-((1-(5-chloro-1-methyl-1H-indazol-3-yl)-1-oxopropan-2-yl)oxy)isoindoline-1,3-dione: To a solution of 2-hydroxyisoindoline-1,3-dione (750 mg, 4.6 mmol) and (E)-5-chloro-1-methyl-3-(prop-1-en-1-yl)-1H-indazole (1.0 g, 4.8 mmol) in N,N-dimethylformamide (20 mL) was added copper(II) acetate monohydrate (68 mg, 0.5 mmol) and the mixture was stirred in an open flask at room temperature for 2 hours. The mixture was diluted with water (100 mL) and extracted with ethyl acetate (2×100 mL). The combined organic extracts were washed with brine (2×50 mL), dried over sodium sulfate and concentrated under reduced pressure. The residue was purified twice by silica flash chromatography [30% to 50% ethyl acetate in heptane] followed by [0% to 2% methanol in dichloromethane] to afford 2-((1-(5-chloro-1-methyl-1H-indazol-3-yl)-1-oxopropan-2-yl)oxy)isoindoline-1,3-dione (55 mg, 3%) as a white solid. 1H NMR (CDCl₃, 400 MHz) δ 8.40 (dd, J=1.9, 0.7 Hz, 1H), 7.82-7.77 (m, 2H), 7.76-7.66 (m, 2H), 7.41 (dd, J=8.9, 1.9 Hz, 1H), 7.34 (dd, J=8.9, 0.7 Hz, 1H), 6.08 (m, 1H), 4.03 (s, 3H), 1.78 (d, J=6.9 Hz, 3H). LCMS: 98.8%; 384.0 (M+1); RT 2.23 mm (method C). TLC: 50% ethyl acetate/heptane (R_{f}: 0.33).

2-(aminooxy)-1-(5-chloro-1-methyl-1H-indazol-3-yl)propan-1-one: To a solution of 2-((1-(5-chloro-1-methyl-1H-indazol-3-yl)-1-oxopropan-2-yl)oxy)isoindoline-1,3-dione (652 mg, 1.6 mmol) in ethanol (15 mL) and chloroform (20 mL) was added hydrazine monohydrate (0.08 mL, 1.7 mmol) and the mixture was stirred at room temperature for 30 minutes. The solids were filtered off to afford 2-(aminooxy)-1-(5-chloro-1-methyl-1H-indazol-3-yl)propan-1-one as a solution in ethanol and chloroform. LCMS: 254.0 (M+1); RT 1.96 min (method A).

### Example 68

### N′-((1-(5-chloro-1-methyl-1H-indazol-3-yl)-1-oxopropan-2-yl)oxy)-6-methoxy-5-(4-methyl-1H-imidazol-1-yl)picolinimidamide

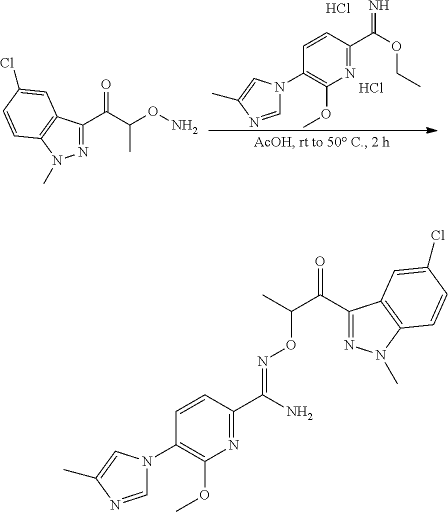 [SMILES: CC(ON)C(=O)c1nn(C)c2ccc(Cl)cc12 ; CCOC(=N)c1ccc(-n2cnc(C)c2)c(OC)n1 ; Cl ; Cl ; COc1nc(/C(N)=N/OC(C)C(=O)c2nn(C)c3ccc(Cl)cc23)ccc1-n1cnc(C)c1]

N′-((1-(5-chloro-1-methyl-1H-indazol-3-yl)-1-oxopropan-2-yl)oxy)-6-methoxy-5-(4-methyl-1H-imidazol-1-yl)picolinimidamide : Ethyl 6-methoxy-5-(4-methyl-1H-imidazol-1-yl)-picolin-imidate dihydrochloride (632 mg, 1.6 mmol) was added to a solution of 2-(aminooxy)-1-(5-chloro-1-methyl-1H-indazol-3-yl)propan-1-one in ethanol (35 mL) and chloroform (20 mL). The mixture was stirred at room temperature for 2 hours and then heated at 50° C. for 30 minutes. Saturated aqueous sodium hydrogencarbonate (100 mL) was added and the mixture was extracted with chloroform (2×75 mL). The combined organic extracts were dried over sodium sulfate and concentrated under reduced pressure. The residue was purified by silica flash chromatography [1% to 8% methanol in dichloromethane] followed by acidic reversed phase MPLC [Linear gradient: t=0 min 5% A, t=1 min 5% A, t=13 min 100% A, t=16 min 100% A; detection: DAD (254 nm)] to afford N′-((1-(5-chloro-1-methyl-1H-indazol-3-yl)-1-oxopropan-2-yl)oxy)-6-methoxy-5-(4-methyl-1H-imidazol-1-yl)picolinimidamide (80 mg, 0.17 mmol, 11%) as a white solid. 1H NMR (CDCl₃, 400 MHz) δ 8.39 (m, 1H), 7.91-7.31 (m, 5H), 6.93 (s, 1H), 5.93 (m, 1H), 5.68 (s, 2H), 4.17 (s, 3H), 4.05 (s, 3H), 2.28 (s, 3H), 1.68 (d, J=7.0 Hz, 3H). TLC: 5% methanol/dichloromethane (R_{f}: 0.20).

### Example 69

### (+/−)-(cis)-5-(5-chloro-1-methyl-1H-indazol-3-yl)-3-(6-methoxy-5-(4-methyl-1H-imidazol-1-yl)pyridin-2-yl)-6-methyl-5,6-dihydro-4H-1,2,4-oxadiazine

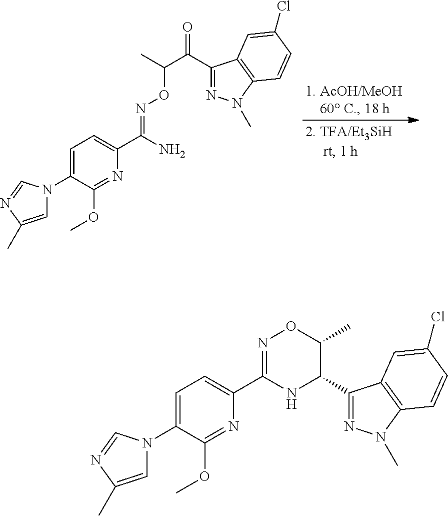 [SMILES: COc1nc(/C(N)=N/OC(C)C(=O)c2nn(C)c3ccc(Cl)cc23)ccc1-n1cnc(C)c1 ; COc1nc(C2=NO[C@H](C)[C@H](c3nn(C)c4ccc(Cl)cc34)N2)ccc1-n1cnc(C)c1]

(+/−)-(c is)-54 5-chloro-1-methyl-1H-indazol-3-yl)-3-(6-methoxy-5-(4-methyl-1H-imidazol-1-yl)-pyridin-2-yl)-6-methyl-5,6-dihydro-4H-1,2,4-oxadiazine: A solution of N -((1-(5-chloro-1-methyl-1H-indazol-3-yl)-1-oxopropan-2-yl)oxy)-6-methoxy-5-(4-methyl-1H-imidazol-1-yl)-picolinimidamide (110 mg, 0.2 mmol) in methanol (7 mL) and acetic acid (1 mL) was heated at 60° C. for 18 hours. The mixture was concentrated under reduced pressure and the residue was dissolved in trifluoroacetic acid (1 mL). Triethylsilane (1 mL, 6.2 mmol) was added and the mixture was stirred at room temperature for 1 hour. The mixture was concentrated under reduced pressure and the residue was partitioned between dichloromethane (10 mL) and saturated aqueous sodium hydrogencarbonate (10 mL). The layers were separated using a phase separation filter and the organic layer was concentrated under reduced pressure to afford a solid that was purified by silica flash chromatography [2% to 7% methanol in dichloromethane] to afford (+/−)-(cis)-5-(5-chloro-1-methyl-1H-indazol-3-yl)-3-(6-methoxy-5-(4-methyl-1H-imidazol-1-yl)pyri-din-2-yl)-6-methyl-5,6-dihydro-4H-1,2,4-oxadiazine (80 mg, 75%) as a white solid.

Racemic compound Example 69 was separated using a Chiralpak OD-H column (250×20 mm, 10 μm) (12 mg loading; heptane:ethanol (80:20) as mobile phase; flow rate: 18 mL/min) to afford the compounds of Example 69A (Fraction (I) (+)) and Example 69B (Fraction (II) (−)).

### Example 69A

(+)-(cis)-5-(5-chloro-1-methyl-1H-indazol-3-yl)-3-(6-methoxy-5-(4-methyl-1H-imidazol-1-yl)pyridin-2-yl)-6-methyl-5,6-dihydro-4H-1,2,4-oxadiazine, Fraction (I) (+): 1H NMR (CDCl₃, 400 MHz) δ 7.93 (d, J=8.0 Hz, 1H), 7.84-7.80 (m, 1H), 7.80-7.76 (m, 1H), 7.65 (d, J=8.0 Hz, 1H), 7.37-7.27 (m, 2H), 6.98 (s, 1H), 6.87 (d, J=4.8 Hz, 1H), 5.14 (dd, J=4.8, 3.3 Hz, 1H), 4.26 (m, 1H), 4.05 (s, 3H), 3.99 (s, 3H), 2.30 (d, J=0.8 Hz, 3H), 1.08 (d, J=6.4 Hz, 3H). LCMS: 100%; 452.0 (M+1); RT 3.34 min (method D); Chiral HPLC: 100%; RT =17.09 min (Chiralcel OD-H (250×4 6 mm, 5 μm); mobile phase heptane:ethanol (80:20); flow Rate: 1.0 mL/min); Optical rotation [α]_{D}^{21.0}: 150.9 (c=0.023, dichloromethane).

### Example 69B

(−)-(cis)-5-(5-chloro-1-methyl-1H-indazol-3-yl)-3-(6-methoxy-5-(4-methyl-1H-imidazol-1-yl)pyridin-2-yl)-6-methyl-5,6-dihydro-4H-1,2,4-oxadiazine, Fraction (II) (−): LCMS: 100%; 452.0 (M+1); RT 3.35 mm (method D); Chiral HPLC: 100%; RT=24.49 mm (Chiralcel OD-H (250×4.6 mm, 5 μm); mobile phase heptane:ethanol (80:20); flow rate: 1.0 mL/min); Optical rotation [α]_{D}^{21.0}: −137.3 (c=0.021, dichloromethane).

### Example 70

### Synthesis of (1R, 2S)-1-(aminooxy)-1-(4-chlorophenyl)propan-2-amine

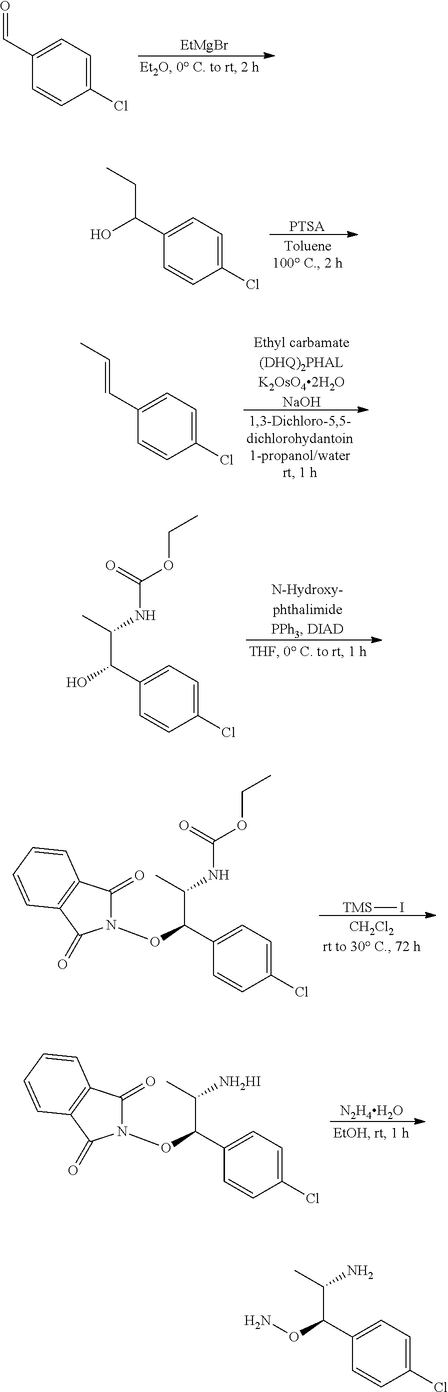

1-(4-chlorophenyl)propan-1-ol: To an ice bath cooled solution of ethylmagnesium bromide in diethyl ether (3M, 25 mL, 75.0 mmol) under a nitrogen atmosphere was added dropwise a solution of 4-chlorobenzaldehyde (10.0 g, 71.1 mmol) in diethyl ether (40 mL). After complete addition, the mixture was allowed to warm up to room temperature and stirred for 2 hours. The mixture was quenched with saturated aqueous ammonium chloride (250 mL) and extracted with dichloromethane (2×200 mL). The combined organic extracts were washed with water (100 mL) and brine (100 mL), dried over sodium sulfate and concentrated under reduced pressure to afford 1-(4-chlorophenyl)propan-1-ol (11.4 g, 89%) as a turbid oil. 1H NMR (CDCl₃, 400 MHz) δ 7.39-7.21 (m, 4H), 4.59 (m, 1H), 1.90-1.65 (m, 3H), 0.91 (t, J=7.4 Hz, 3H). GCMS: 98.1%; 170.0 (M); RT 3.17 mm (method A). TLC: 25% ethyl acetate in heptane (R_{f}:035).

(E)-1-chloro-4-(prop-1-en-1-yl)benzene: To a solution of 1-(4-chlorophenyl)propan-1-ol (1.1 g, 6.3 mmol) in toluene (10 mL) was added p-toluenesulfonic acid monohydrate (90 mg, 0.5 mmol) and the mixture was heated at reflux temperature for 2 hours. Saturated aqueous sodium hydrogencarbonate (100 mL) was added and the mixture was extracted with diethyl ether (2x75 mL). The combined organic extracts were dried over sodium sulfate and concentrated under reduced pressure (30 mbar) to afford (E)-1-chloro-4-(prop-1-en-1-yl)benzene (958 mg, 89%) as a yellow oil. ¹H NMR (CDCl₃, 400 MHz) δ 7.25-7.23 (m, 4H), 6.35 (m, 1H), 6.21 (m, 1H), 1.87 (dd, J=6.5, 1.6 Hz, 3H). GCMS: 92.3%; 152.1 (M); RT 2.73 mm (method A). TLC: 25% ethyl acetate in heptane (R_{f}:083).

Ethyl ((1S,2S)-1-(4-chlorophenyl)-1-hydroxypropan-2-yl)carbamate: To a solution of ethyl carbamate (1.5 g, 17.0 mmol) in 1-propanol (20 mL) and aqueous sodium hydroxide (0.5M, 30 mL) was added 1,3-dichloro-5,5-dimethylhydantoin (1.7 g, 8.5 mmol) and the mixture was stirred at room temperature for 10 minutes. A solution of hydroquinidine 1,4-phthalazinediyldiether (110 mg, 0.14 mmol) and (E)-1-chloro-4-(prop-1-en-1-yl)benzene (958 mg, 5.7 mmol) in 1-propanol (15 mL) was added, followed by a solution of potassium osmate(VI) dihydrate (52 mg, 0.14 mmol) in aqueous sodium hydroxide (0.5M, 1.5 mL). The resulting mixture was stirred at room temperature for 1 hour. The mixture was diluted with water (150 mL) and extracted with ethyl acetate (2×150 mL). The combined organic extracts were washed with brine (100 mL), dried over sodium sulfate and concentrated under reduced pressure to afford a brown oil that was purified by silica flash chromatography 120% to 40% ethyl acetate in heptanel to afford ethyl ((1S,2S)-1-(4-chlorophenyl)-1-hydroxypropan-2-yl)carbamate (100 mg, 7%, minor regioisomer, e.e. not determined) as a white solid. ¹H NMR (DMSO-d6, 400 MHz) δ 7.40-7.32 (m, 2H), 7.32-7.25 (m, 2H), 6.66 (d, J=7.8 Hz, 1H), 5.42 (d, J=4.8 Hz, 1H), 4.58-4.48 (m, 1H), 3.92 (m, 2H), 3.76-3.63 (m, 1H), 1.11 (t, J=6.9 Hz, 3H), 0.91 (d, J=6.7 Hz, 3H). LCMS: 98.3%; 258.0 (M+1); RT 1.90 min (method C). TLC: 50% ethyl acetate in heptane (R_{f}:055).

Ethyl ((1 R,2S)-1-(4-chlorophenyl)-1-((1,3-dioxoisoindolin-2-yl)oxy)propan-2-yl)carbamate: To an ice bath cooled solution of ethyl ((1S,2S)-1-(4-chlorophenyl)-1-hydroxypropan-2-yl)carbamate (195 mg, 0.8 mmol), triphenylphosphine (218 mg, 0.8 mmol) and N-hydroxyphthalimide (136 mg, 0.8 mmol) in dry tetrahydrofuran (5 mL) was added diisopropyl azodicarboxylate (0.16 mL, 0.8 mmol) and the mixture was stirred at room temperature for 45 minutes. After this time, additional triphenylphosphine (109 mg, 0.4 mmol) and diisopropyl azodicarboxylate (0.08 mL, 0.04 mmol) were added and the mixture was stirred at room temperature for 1 hour. The mixture was concentrated under reduced pressure and the residue was purified by silica flash chromatography [10% to 40% ethyl acetate in heptane] to afford ethyl ((1R,2S)-1-(4-chlorophenyl)-1-((1,3-dioxoisoindolin-2-yl)oxy)propan-2-yl)carbamate (215 mg, 67%) as an off-white solid. ¹H NMR (DMSO-d6, 400 MHz) δ 7.87-7.71 (m, 4H), 7.47-7.40 (m, 2H), 7.40-7.32 (m, 2H), 7.13 (d, J=8.9 Hz, 1H), 5.19 (d, J=8.0 Hz, 1H), 4.04-3.93 (m, 1H), 3.84 (m, 2H), 1.36 (d, J=6.7 Hz, 3H), 1.01 (t, J=7.1 Hz, 3H). LCMS: 99.3%; 403.1 (M+1); RT 2.14 mm (method C). TLC: 50% ethyl acetate in heptane (R_{f}:053).

2-((1R,2S)-2-amino-1-(4-chlorophenyl)propoxy)isoindoline-1, 3-dione hydroiodide: Under an argon atmosphere, iodotrimethylsilane (0.15 mL, 1.0 mmol) was added to a solution of ethyl ((1R,2S)-1-(4-chlorophenyl)-1-((1,3-dioxoisoindolin-2-yl)oxy)propan-2-yl)carbamate (215 mg, 0.5 mmol) in dichloromethane (5 mL). The mixture was stirred at room temperature for 18 hours. Additional iodotrimethylsilane (0.07 mL, 0.5 mmol) was added and the mixture was stirred at room temperature for 48 hours. The mixture was concentrated under reduced pressure and the residue was triturated from dichloromethane (0.5 mL) and heptane (3 mL) to afford 2-((1R,2S)-2-amino-1-(4-chlorophenyl)propoxy)isoindoline-1,3-dione hydroiodide (128 mg, 55%) as a pale yellow solid. ¹H NMR (DMSO-d6, 400 MHz) δ 7.95 (s, 2H), 7.90-7.83 (m, 4H), 7.61-7.49 (m, 4H), 5.36 (d, J=4.7 Hz, 1H), 3.88-3.76 (m, 1H), 1.23 (d, J=6.7 Hz, 3H). LCMS: 100%; 331.1 (M+1); RT 1.64 mm (method C).

(1R,2S)-1-(aminooxy)-1-(4-chlorophenyl)propan-2-amine: To a suspension of 2-((1R,2S)-2-amino-1-(4-chlorophenyl)propoxy)isoindoline-1,3-dione hydroiodide (149 mg, 0.3 mmol) in ethanol (4 mL) was added hydrazine monohydrate (0.05 mL, 0.9 mmol) and the mixture was stirred at room temperature for 1 hour. The solids were filtered off and the residue was washed with ethanol (2 mL). The filtrate was concentrated under reduced pressure and the residue was purified over an ion-exchange column (SCX-2: column rinsed with two column volumes of methanol, product eluted with three column volumes of 2M ammonia in methanol). The basic product fraction was concentrated under reduced pressure to afford (1R,2S)-1-(aminooxy)-1-(4-chlorophenyl)propan-2-amine (64 mg, 98%) as an oil. ¹H NMR (CDCl₃, 400 MHz) δ 7.39-7.33 (m, 2H), 7.30-7.26 (m, 2H), 4.41 (d, J=5.2 Hz, 1H), 3.31-3.15 (m, 1H), 1.09 (d, J=6.6 Hz, 3H). LCMS: 97.6%; 201.0 (M+1); RT 1.66 min (method A).

### Example 71

### (5S,6R)-6-(4-chlorophenyl)-3-(6-methoxy-5-(4-methyl-1H-imidazol-1-yl)pyridin-2-yl)-5-methyl-5,6-dihydro-4H-1,2,4-oxadiazine

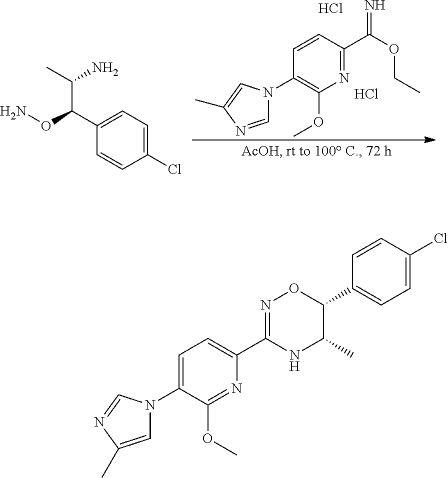 [SMILES: C[C@H](N)[C@H](ON)c1ccc(Cl)cc1 ; CCOC(=N)c1ccc(-n2cnc(C)c2)c(OC)n1 ; Cl ; Cl ; COc1nc(C2=NO[C@H](c3ccc(Cl)cc3)[C@H](C)N2)ccc1-n1cnc(C)c1]

(5S,6R)-6-(4-chlorophenyl)-3-(6-methoxy-5-(4-methyl-1H-imidazol-1-yl)pyridin-2-yl)-5-methyl-5,6-dihydro-4H-1,2,4-oxadiazine:

A solution of ethyl 6-methoxy-5-(4-methyl-1H-imidazol-1-yl)picolinimidate dihydrochloride (150 mg, 0.36 mmol) and (1R,2S)-1-(aminooxy)-1-(4-chlorophenyl)propan-2-amine (63 mg, 0.31 mmol) in acetic acid (4 mL) was stirred at room temperature for 1 hour and at 100° C. for 6 hours. The mixture was concentrated under reduced pressure and the residue was purified over na ion-exchange column (SCX-2: column rinsed with two column volumes of methanol, product eluted with two column volumes of 2M ammonia in methanol). The basic product fraction was concentrated under reduced pressure to afford a solid that was purified by silica flash chromatography [0% to 5% methanol in dichloromethane] to afford (5S,6R)-6-(4-chlorophenyl)-3-(6-methoxy-5-(4-methyl-1H-imidazol-1-yl)pyridin-2-yl)-5-methyl-5,6-dihydro-4H-1,2,4-oxadiazine (80 mg, 64%, e.e. 32%) as a white solid.

Enantio-enriched compound Example 71 was separated using a Chiralpak OD-H column (250×20 mm, 10 μm) (15 mg loading; heptane:ethanol (70:30) as mobile phase; flow rate: 18 mL/min) to afford the compounds of Example 71A (Fraction (I) (−)) and Example 71B (Fraction (II) (+)).

### Example 71A

(5S,6R)-6-(4-chlorophenyl)-3-(6-methoxy-5-(4-methyl-1H-imidazol-1-yl)pyridin-2-yl)-5-methyl-5,6-dihydro-4H-1,2,4-oxadiazine, Fraction (I) (−): 1H NMR (CDCl₃, 400 MHz) δ 7.84-7.82 (m, 1H), 7.81 (d, J=8.0 Hz, 1H), 7.63 (d, J=8.0 Hz, 1H), 7.42-7.32 (m, 4H), 7.04-6.94 (m, 1H), 6.53 (d, J=4.8 Hz, 1H), 4.92 (d, J=2.6 Hz, 1H), 4.08 (s, 3H), 4.06-3.98 (m, 1H), 2.31 (d, J=0.8 Hz, 3H), 1.08 (d, J=6.5 Hz, 3H). LCMS: 100%; 398.2 (M+1); RT 2.79 min (method E); Chiral HPLC: 100%; RT =10.8 min (Chiralpak OD-H (250×4.6 mm, 5 μm); mobile phase heptane:ethanol (70:30); flow rate: 1.0 mL/min); Optical rotation [α]_{D}^{21.0}: −10.3 (c=0.021, dichloromethane).

### Example 71B

(5R,65)-6-(4-chlorophenyl)-3-(6-methoxy-5-(4-methyl-1H-imidazol-1-yl)pyridin-2-yl)-5-methyl-5,6-dihydro-4H-1,2,4-oxadiazine, Fraction (II) (+): LCMS: 99.5%; 398.2 (M+1); RT 2.79 min (method E); Chiral HPLC: 99.4%; RT =19.2 min (Chiralpak OD-H (250×4.6 mm, 5 μm); mobile phase heptane:ethanol (70:30); flow rate: 1.0 mL/min); Optical rotation [α]_{D}^{21.0}: +7.0 (c=0.018, dichloromethane).

### Example 72

### Synthesis of 6-cyclopropyl-3-(6-methoxy-5-(4-methyl-1H-imidazol-1-yl) pyridin-2-yl)-5-(2-methylbenzo [d] thiazol-5-yl)-5, 6-dihydro-4H-1,2,4-oxadiazine

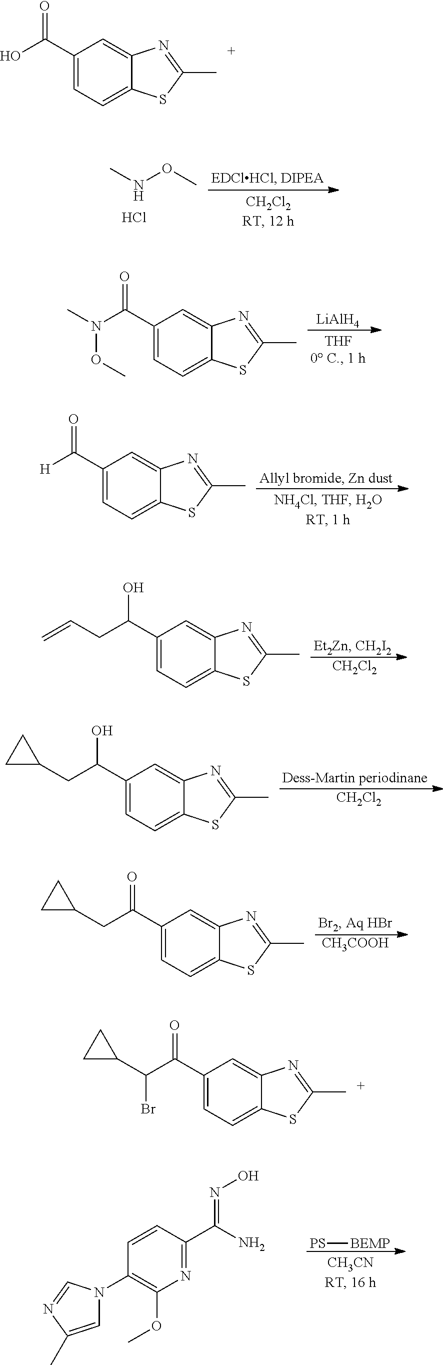

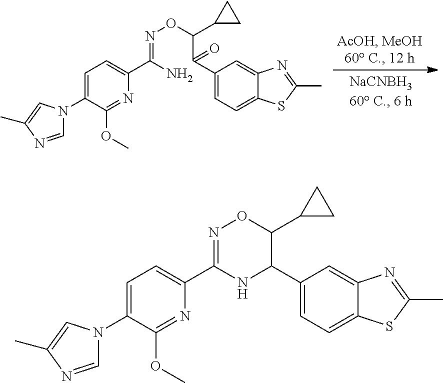

### Synthesis of N-methoxy-N,2-dimethylbenzo [d]thiazole-5-carboxamide

To a stirred solution of 2-methylbenzo [d] thiazole-5-carboxylic acid (6.5 g, 34 mmol) in CH₂Cl₂ (65 mL) at 0° C. under an argon atmosphere were added N,O-dimethylhydroxylamine hydrochloride (3.6 g, 37 mmol), EDCI. HCl (7.72 g, 40 mmol) and bromine (20.4 mg, 0.1 mmol). The reaction mixture was warmed to room temperature and stirred for 12 h. After consumption of starting material (by TLC), the reaction mixture was diluted with saturated sodium bicarbonate solution (20 mL) and extracted with EtOAc (2×20 mL). The combined organic extracts were dried over sodium sulfate, filtered and concentrated in vacuo to obtain N-methoxy-N,2-dimethylbenzo [d] thiazole-5-carboxamide (6.2 g, crude) as a pale yellow solid. LCMS: 52.9%; 236.9 (M+1); (column; Ascentis Express C-18 (50×3.0 mm, 2.7 μm); RT 1.81 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient);TLC: 5% MeOH/CH₂Cl₂ (R_{f}:03).

### Synthesis of 2-methylbenzo [d] thiazole-5-carbaldehyde

To a stirred solution of 2-bromo-1-(6-chlorofuro [3,2-b] pyridin-2-yl)-2-cyclopropylethan-1-one (6.2 g, 26 mmol) in THF (62 mL) at 0° C. under an argon atmosphere was added lithium aluminium hydride (3 g, 79 mmol). The reaction mixture was stirred at 0° C. for 1.5 h. After consumption of starting material (by TLC), the reaction mixture was diluted with saturated ammonium chloride solution (200 mL) and extracted with EtOAc (2×150 mL). The combined organic extracts were dried over sodium sulfate, filtered and concentrated in vacuo to obtain the crude. The crude material was purified by preparative TLC using 15-20% EtOAc: Hexane to afford 2-methylbenzo [d] thiazole-5-carbaldehyde (3.2 g, 69%) as a pale yellow solid. ¹ H-NMR (CDCl₃, 400 MHz): δ 10.11 (s, 1H), 8.40 (s, 1H), 7.94 (d, 1H), 7.90 (d, 1H), 2.88 (s, 3H); TLC: 5% MeOH/CH₂Cl₂ (R_{f}:06).

### Synthesis of 1-(2-methylbenzo [d]thiazol-5-yl)but-3-en-1-ol

To a stirred solution of 2-methylbenzo [d] thiazole-5-carbaldehyde (3.2 g, 18 mmol) in THF (70 mL) at 0° C. under an argon atmosphere were added allyl bromide (6.56 g, 54 mmol) in THF (58 mL), Zn dust (11.75 g, 180 mmol) and ammonium chloride (9.67 g, 18 mmol) in water (32 mL). The reaction mixture was warmed to room temperature for 1 h. After consumption of starting material (by TLC), the reaction mixture was filtered, washed with EtOAc (50 mL). The organic layer was dried over sodium sulfate, filtered and concentrated in vacuo to obtain the crude. The crude material was purified by column chromatography using 30% EtOAc: Hexane to afford 1-(2-methylbenzo [d] thiazol-5-yl)but-3-en-1-ol (3.2 g, 81%) as brown liquid. ¹H-NMR (CDCl₃, 400 MHz): δ 7.91 (s, 1H), 7.80 (d, 1H), 7.39 (d, 1H), 5.87-5.79 (m, 1H), 5.20-5.11 (m, 2H), 4.90-4.84 (m, 1H), 2.85 (s, 3H), 2.60-2.52 (m, 2H), 2.12 (s, 1H); TLC: 30% EtOAc/Hexane (R_{f}:03).

### Synthesis of 2-cyclopropyl-1-(2-methylbenzo [d] thiazol-5-yl) ethan-1-ol

To a stirred solution of diethyl zinc (15% solution in hexane, 120 mL, 146 mmol) in CH₂Cl₂ (100 mL) at 0° C. under an argon atmosphere was added diiodo methane (19 g, 73 mmol) dropwise. Then 1-(2-methylbenzo [d] thiazol-5-yl)but-3-en-1-ol (3.2 g, 14 mmol) in CH₂Cl₂ (28 mL) was added to the reaction mixture at room temperature. The reaction mixture was stirred at room temperature for 24 h. After consumption of starting material (by TLC), the reaction mixture was diluted with saturated ammonium chloride solution (100 mL) and extracted with CH₂Cl₂ (2×100 mL). The combined organic extracts were dried over sodium sulfate, filtered and concentrated in vacuo to obtain the crude. The crude material was purified by column chromatography using 25% EtOAc: Hexane to afford 2-cyclopropyl-1-(2-methylbenzo [d] thiazol-5-yl) ethan-1-ol (680 mg, 20%) as an off-white solid.

¹H-NMR (CDCl₃, 400 MHz): δ 7.91 (s, 1H), 7.79 (d, 1H), 7.40 (d, 1H), 4.90 (t, 1H), 2.81 (s, 3H), 1.75-1.70 (m, 2H), 0.75-0.69 (m, 1H), 0.51-0.40 (m, 2H), 0.17-0.10 (m, 1H), 0.08-0.01 (m, 1H); TLC: 40% EtOAc/Hexane (R_{f}:05).

### Synthesis of 2-cyclopropyl-1-(2-methylbenzo [d] thiazol-5-yl) ethan-1-one

To a stirred solution of 2-cyclopropyl-1-(2-methylbenzo [d] thiazol-5-yl) ethan-1-ol (680 mg, 3 mmol) in CH₂Cl₂ (6.8 mL) at 0° C. under an argon atmosphere was added dessmartin periodinane (1.48 g, 3.5 mmol). The reaction mixture was stirred at room temperature for 1 h. After consumption of starting material (by TLC), the reaction mixture was diluted with water (50 mL) and extracted with CH₂Cl₂ (2×50 mL). The combined organic extracts were washed with saturated sodium bicarbonate solution (50 mL), dried over sodium sulfate, filtered and concentrated in vacuo to obtain the crude. The crude material was purified by column chromatography using 15% EtOAc: Hexane to afford 2-cyclopropyl-1-(2-methylbenzo [d] thiazol-5-yl) ethan-1-one (600 mg, 88%) as an off-white solid. ¹H-NMR (CDCl₃, 400 MHz): δ 8.50 (s, 1H), 7.99 (d, 1H), 7.89 (s, 1H), 2.95 (d, 2H), 2.88 (s, 3H), 1.21-1.18 (m, 1H), 0.64-0.60 (m, 2H), 0.24-0.20 (m, 2H); TLC: 30% EtOAc/Hexane (R_{f}:06).

### Synthesis of 2-bromo-2-cyclopropyl-1-(2-methylbenzo [d] thiazol-5-yl) ethan-1-one

To a stirred solution of 2-cyclopropyl-1-(2-methylbenzo [d] thiazol-5-yl) ethan-1-one (550 mg, 2 mmol) in acetic acid (22 mL) at 0° C. under an argon atmosphere were added bromine (381 mg, 2 mmol) and 48% aq. HBr (5.5 mL). The reaction mixture was stirred at room temperature for 16 h. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo to obtain the crude. The crude material was purified by column chromatography using 10% EtOAc: Hexane to afford 2-bromo-2-cyclopropyl-1-(2-methylbenzo [d] thiazol-5-yl) ethan-1-one (660 mg, 89%) as an off-white solid.

¹H-NMR (CDCl₃, 400 MHz): δ 8.52 (s, 1H), 8.01 (d, 1H), 7.90 (d, 1H), 4.51 (d, 1H), 2.88 (s, 3H), 1.90-1.80 (m, 1H), 0.97-0.90 (m, 2H), 0.01-0.58 (m, 1H), 0.50-0.43 (m, 1H); TLC: 20% EtOAc/Hexane (R_{f}:03).

### Synthesis of (Z)-N′-(1-cyclopropyl-2-(2-methylbenzo [d] thiazol-5-yl)-2-oxoethoxy)-6-methoxy-5-(4-methyl-1 H-imidazol-1-yl) picolinimidamide

To a stirred solution of (Z)-N′-hydroxy-6-methoxy-5-(4-methyl-1H-imidazol-1-yl) picolinimidamide (300 mg, 1 mmol) in CH₃CN (10 mL) at room temperature under an argon atmosphere was added PS-BEMP (660 mg). The reaction mixture was stirred for 5 min at room temperature. Then 2-bromo-2-cyclopropyl-1-(2-methylbenzo [d] thiazol-5-yl)ethan-1-one (562 mg, 2 mmol) in CH₃CN (5 mL) was added to the reaction mixture at room temperature. The reaction mixture was stirred for 16 h at room temperature. After consumption of starting material (by TLC), the reaction mixture was filtered, washed with EtOAc (50 mL). The filtrate was concentrated in vacuo to obtain (Z)-N′-(1-cyclopropyl-2-(2-methylbenzo [d] thiazol-5-yl)-2-oxoethoxy)-6-methoxy-5-(4-methyl-1H-imidazol-1-yl) picolinimidamide (600 mg, crude) as a brown syrup used in the next step without further purification. LCMS: 61.5%; 477.1 (M+1); (column; Ascentis Express C-18 (50×3.0 mm, 3.5 μm); RT 2.01 min; mobile phase: 0.025% Aq TFA+5% ACN:ACN+5% 0.025% Aq TFA; T/B %: 0.01/5, 0.5/5, 3/100, 5/100; flow rate: 1.2 mL/min) (Gradient); TLC: 5% MeOH/CH₂Cl₂ (R_{f}:04).

### Synthesis of 6-cyclopropyl-3-(6-methoxy-5-(4-methyl-M-imidazol-1-yl) pyridin-2-yl)-5-(2-methylbenzo [d] thiazol-5-yl)-5,6-dihydro-4H-1,2,4-oxadiazine

To a stirred solution of (Z)-N′-(1-cyclopropyl-2-(2-methylbenzo [d] thiazol-5-yl)-2-oxoethoxy)-6-methoxy-5-(4-methyl-1H-imidazol-1-yl) picolinimidamide (600 mg, 1 mmol) in MeOH (24 mL) at room temperature under an argon atmosphere was added acetic acid (3 mL, 6 mmol). The reaction mixture was stirred for 12 h at 60° C. Then sodium cyanoborohydride (95 mg, 1.5 mmol) was added to the reaction mixture at room temperature. The reaction mixture was stirred for 6 h at 60° C. After consumption of starting material (by TLC), the reaction mixture was diluted with saturated sodium bicarbonate solution (50 mL) and extracted with EtOAc (2×50 mL). The combined organic extracts were dried over sodium sulfate, filtered and concentrated in vacuo to obtain the crude. The crude material was purified by column chromatography using 2% MeOH:CH₂Cl₂ to afford 6-cyclopropyl-3-(6-methoxy-5-(4-methyl-1H-imidazol-1-yl) pyridin-2-yl)-5-(2-methylbenzo [d] thiazol-5-yl)-5,6-dihydro-4H-1,2,4-oxadiazine (200 mg, 36%, over two steps) as an off-white solid. TLC: 5% MeOH/CH₂Cl₂ (R_{f}:05).

Racemic compound of Example 72: CH₂Cl₂:MeOH (50:50) (A:B: 75:25) as mobile phase; flow rate: 20 mL/min) to afford the compounds of Example 72A (Fraction (I) (−)) and Example 72B (Fraction (II) (+)).

Analytical conditions for Example 72A and Example 72B. HPLC (purity): (column; X-select CSH C-18 150×4.6 mm, 3.5 μm); mobile Phase: 0.05% TFA+5% ACN; ACN: 5% 0.05% TFA; flow rate: 1.0 mL/min; Gradient program: T/B % 0.01/10, 10/90, 15/90: diluent: CH₃CN: Water; Chiral HPLC: (Chiralpak-IC (250×4.6 mm, 5 μm; mobile phase (A) 0.1% DEA in n-Hexane (B) CH₂Cl₂:MeOH (50:50) (A::B; 75:25); flow Rate: 1.0 mL/min).

### Example 72A

(−)-6-cyclopropyl-3-(6-methoxy-5-(4-methyl-1H-imidazol-1-yl) pyridin-2-yl)-5-(2-methylbenzo [d] thiazol-5-yl)-5, 6-dihydro-4H-1,2,4-oxadiazine, fraction (I) (−); Mass (ESI): 461.1 [M+1]; HPLC (purity): 97.7%; RT 6.07 min; Chiral HPLC: 100% RT =6.46 min; Optical rotation [α]_{D}^{19.97}: −114.48 (c=0.25, CH₂Cl₂).

### Example 72B

(+)-6-cyclopropyl-3-(6-methoxy-5-(4-methyl-1H-imidazol-1-yl) pyridin-2-yl)-5-(2-methylbenzo [d] thiazol-5-yl)-5, 6-dihydro-4H-1,2,4-oxadiazine, fraction (II) (+); ¹H NMR (CD₃OD, 400 MHz): δ 7.97 (s, 1H), 7.90-7.85 (m, 3H), 7.65 (d, 1H), 7.43 (dd, 1H), 7.20 (s, 1H), 5.00-4.98 (m, 1H), 4.03 (s, 3H), 3.20 (dd, 1H), 2.80 (s, 3H), 2.21 (s, 3H), 0.55-0.43 (m, 4H), 0.30-0.23 (m, 1H); Mass (ESI): 461.1 [M+1]; HPLC (purity): 99.2%; RT 6.09 min; Chiral HPLC: 100% RT =8.27 min; Optical rotation [α]_{D}^{19.93}: +119.95 (c=0.25, CH₂Cl₂).

1. A compound of Formula (I) 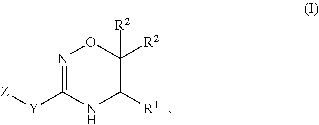
or a pharmaceutically acceptable salt thereof,
wherein:
R¹ is phenyl, 5- to 6-membered aromatic heterocycle, 8- to 10-membered bicyclic heterocycle or 11- to 14-membered tricyclic heterocycle, each of which is unsubstituted or substituted with one or more substituents independently selected from the group consisting of -halo, —C1-C4 alkyl, —C3-C8 monocyclic cycloalkyl, halo-substituted C1-C4 alkyl, —CN, —OH, —C1-C4 alkoxy, —O—C3-C8 monocyclic cycloalkyl, halo-substituted C1-C4 alkoxy and 3- to 7-membered monocyclic heterocycle;
each R² is independently hydrogen, —C1-C4 alkyl or —C3-C6 monocyclic cycloalkyl with the proviso that both R² are not hydrogen, or both R² together with the carbon atom they are attached to form a C3-C6 monocyclic cycloalkyl, wherein each —C1-C4 alkyl and —C3-C6 monocyclic cycloalkyl is unsubstituted or substituted with one or more substituents independently selected from the group consisting of -halo, —OH, —C1-C4 alkoxy, —O—C3-C8 monocyclic cycloalkyl which is unsubstituted or substituted with halo, halo-substituted C1-C4 alkyl or halo-substituted C1-C4 alkoxy;
Y is pyridinyl or phenyl, each of which is unsubstituted or substituted with one or more substituents independently selected from the group consisting of -halo, —C1-C4 alkoxy, halo-substituted C1-C4 alkoxy, —C1-C4 alkyl, halo-substituted C1-C4 alkyl, —CN and —OH; and
Z is nitrogen-containing 3- to 7-membered monocyclic heterocycle which is unsubstituted or substituted with one or more substituents independently selected from the group consisting of -halo, —C1-C4 alkyl, halo-substituted C1-C4 alkyl, —C1-C4 alkoxy and —OCF₃.

2. (canceled)

3. A compound of claim 1, wherein R¹ is phenyl which is unsubstituted or substituted with one or more substituents independently selected from the group consisting of -halo, —C1-C4 alkyl, —C3-C8 monocyclic cycloalkyl and halo-substituted C1-C4 alkyl; or a pharmaceutically acceptable salt thereof.

4-6. (canceled)

7. A compound of claim 1, wherein R¹ is 5- to 6-membered aromatic heterocycle which is unsubstituted or substituted with one or more substituents independently selected from the group consisting of -halo, —C1-C4 alkyl, —C3-C8 monocyclic cycloalkyl and halo-substituted C1-C4 alkyl; or a pharmaceutically acceptable salt thereof.

8-10. (canceled)

11. A compound of claim 1, wherein R¹ is 8-to 10-membered bicyclic heterocycle which is unsubstituted or substituted with one or more substituents independently selected from the group consisting of -halo, —C1-C4 alkyl, —C3-C8 monocyclic cycloalkyl and halo-substituted C1-C4 alkyl; or a pharmaceutically acceptable salt thereof.

12-21. (canceled)

22. A compound of claim 21, wherein one R² is hydrogen and the other R² is —C1-C4 alkyl or cyclopropyl; or a pharmaceutically acceptable salt thereof.

23-29. (canceled)

30. A compound of claim 1, wherein Y is pyridinyl or phenyl, each of which is unsubstituted or substituted with one or more substituents independently selected from the group consisting of -halo, —C1-C4 alkoxy, halo-substituted C1-C4 alkoxy, —C1-C4 alkyl, halo-substituted C1-C4 alkyl, —CN and —OH; or a pharmaceutically acceptable salt thereof.

31-33. (canceled)

34. The compound of claim 1, wherein Y is 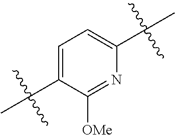 [SMILES: *c1ccc(*)c(OC)n1]
wherein the left most radical is connected to the Z group in Formula (I); or a pharmaceutically acceptable salt thereof.

35-39. (canceled)

40. A compound of claim 1, wherein Z is 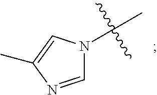 [SMILES: *n1cnc(C)c1]
or a pharmaceutically acceptable salt thereof.

41. A compound selected from the group consisting of:
(+)-(5S,6R)-5-(4-chlorophenyl)-3-[6-methoxy-5-(4-methyl-1H-imidazol-1-yl)pyridin-2-yl]-6-methyl-5,6-dihydro-4H-1,2,4-oxadiazine;
(+)-cis-5-(3,4-dichlorophenyl)-3-[6-methoxy-5-(4-methyl-1H-imidazol-1-yl)pyridin-2-yl]-6-methyl-5,6-dihydro-4H-1,2,4-oxadiazine;
(+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazin;
(−)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-3-[6-methoxy-5-(4-methyl-1H-imidazol-1-yl)pyridin-2-yl]-6,6-dimethyl-5-phenyl-5,6-dihydro-4H-1,2,4-oxadiazin;
(+)-5-(4-chlorophenyl)-3-[6-methoxy-5-(4-m ethyl-1H-imidazol-1-yl)pyridin-2-yl]-6,6-dimethyl-5,6-dihydro-4H-1,2,4-oxadiazine;
(+)-trans-5-(4-chlorophenyl)-3-[6-methoxy-5-(4-methyl-1H-imidazol-1-yl)pyridin-2-yl]-6-methyl-5,6-dihydro-4H-1,2,4-oxadiazine; and
(+)-8-(4-chlorophenyl)-6-[6-methoxy-5-(4-methyl-1H-imidazol-1-yl)pyridin-2-yl]-4-oxa-5,7-diazaspiro[2.5]oct-5-ene;
or a pharmaceutically acceptable salt thereof.

42. The compound of claim 41, selected from the group consisting of:
(+)-(5 S,6R)-5-(4-chlorophenyl)-3-[6-methoxy-5-(4-methyl-1H-imidazol -1-yl)pyridin-2-yl]-6-methyl-5,6-dihydro-4H-1,2,4-oxadiazine;
(+)-cis-5-(3,4-dichlorophenyl)-3-[6-methoxy-5-(4-methyl-1H-imidazol -1-yl)pyridin-2-yl]-6-methyl -5,6-dihydro-4H-1,2,4-oxadiazine;
(+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazin;
(−)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole;
(+)-5-(4-chlorophenyl)-3-[6-methoxy-5-(4-methyl-1H-imidazol-1-yl)pyridin-2-yl]-6,6-dimethyl-5,6-dihydro-4H-1,2,4-oxadiazine;
(+)-trans-5-(4-chlorophenyl)-3-[6-methoxy-5-(4-methyl-1H-imidazol-1-yl)pyridin-2-yl]-6-methyl-5,6-dihydro-4H-1,2,4-oxadiazine; and
(+)-8-(4-chlorophenyl)-6-[6-methoxy-5-(4-methyl-1H-imidazol-1-yl)pyridin-2-yl]-4-oxa-5,7-diazaspiro [2.5]oct-5-ene;
or a pharmaceutically acceptable salt thereof

43. The compound of claim 42, wherein the compound is (+)-(5S,6R)-5-(4-chlorophenyl)-3-[6-methoxy-5-(4-methyl-1H-imidazol-1-yl)pyridin-2-yl]-6-methyl-5,6-dihydro-4H-1,2,4-oxadiazine; or a pharmaceutically acceptable salt thereof.

44. The compound of claim 42, wherein the compound is (+)-5-chloro-6-fluoro-3-[3-[6-methoxy-5-(4-methyl-1H-imidazol-1-yl)pyridin-2-yl]-6-methyl-5,6-dihydro-4H-1,2,4-oxadiazin-5-yl]-1-methyl-1H-indole; or a pharmaceutically acceptable salt thereof.

45. The compound of claim 42, wherein the compound is (+)-cis-5-(4-chlorophenyl)-6-cyclopropyl-3-[6-methoxy-5-(4-methyl-1H-imidazol-1-yl)pyridin-2-yl]-5,6-dihydro-4H-1,2,4-oxadiazine; or a pharmaceutically acceptable salt thereof.

46. A compound of Formula (II) 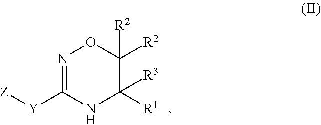
or a pharmaceutically acceptable salt thereof,
wherein:
R¹ is phenyl, 5- to 6-membered aromatic heterocycle, 8- to 10-membered bicyclic heterocycle or 11- to 14-membered tricyclic heterocycle, each of which is unsubstituted or substituted with one or more substituents independently selected from the group consisting of -halo, —C1-C4 alkyl, —C3-C8 monocyclic cycloalkyl, halo-substituted C1-C4 alkyl, —CN, —OH, —C1-C4alkoxy, —O—C3-C8 monocyclic cycloalkyl, halo-substituted C1-C4alkoxy and 3- to 7-membered monocyclic heterocycle;
each R² is independently hydrogen, —C1-C4 alkyl or —C3-C6 monocyclic cycloalkyl, or both R² together with the carbon atom they are attached to form a C3-C6 monocyclic cycloalkyl, wherein each —C1-C4 alkyl and —C3-C6 monocyclic cycloalkyl is unsubstituted or substituted with one or more substituents independently selected from the group consisting of -halo, —OH, —C1-C4alkoxy, —O—C₃-C8 monocyclic cycloalkyl which is unsubstituted or substituted with halo, halo-substituted C1-C4 alkyl or halo-substituted C1-C4alkoxy;
R³ is —C1-C4 alkyl or —C3-C6 monocyclic cycloalkyl, each of which is unsubstituted or substituted with one or more substituents independently selected from the group consisting of -halo, —OH, —C1-C4 alkoxy, —O—C3-C8 monocyclic cycloalkyl which is unsubstituted or substituted with halo, halo-substituted C1-C4 alkyl or halo-substituted C1-C4alkoxy;
Y is pyridinyl or phenyl, each of which is unsubstituted or substituted with one or more substituents independently selected from the group consisting of -halo, —C1-C4 alkoxy, halo-substituted C1-C4 alkoxy, —C1-C4 alkyl, halo-substituted C1-C4 alkyl, —CN and —OH; and
Z is nitrogen-containing 3- to 7-membered monocyclic heterocycle which is unsubstituted or substituted with one or more substituents independently selected from the group consisting of -halo, —C1-C4 alkyl, halo-substituted C1-C4 alkyl, —C1-C4 alkoxy and —OCF₃.

47-88. (canceled)

89. The compound of claim 46, wherein the compound is (+)-3-[3-[6-methoxy-5-(4-methyl-1H-imidazol-1-yl)pyridin-2-yl]-5-methyl-5,6-dihydro-4H-1,2,4-oxadiazin-5-yl]-1-methyl-1H-indole; or a pharmaceutically acceptable salt thereof.

90. The compound of claim 46, wherein the compound is (−)-3-[3-[6-methoxy-5-(4-methyl-1H-imidazol-1-yl)pyridin-2-yl]-5-methyl-5,6-dihydro-4H-1,2,4-oxadiazin-5-yl]-1-methyl-1H-indole; or a pharmaceutically acceptable salt thereof.

91-92. (canceled)

93. A pharmaceutical composition comprising a pharmaceutically acceptable carrier or vehicle and an effective amount of a compound of claim 1 or a pharmaceutically acceptable salt thereof.

94. A method for treating a neurodegenerative disease, comprising administering to a subject in need thereof an effective amount of a compound of claim 1 or a pharmaceutically acceptable salt thereof.

95-104. (canceled)

105. A pharmaceutical composition comprising a pharmaceutically acceptable carrier or vehicle and an effective amount of a compound of claim 46 or a pharmaceutically acceptable salt thereof.

106. A method for treating a neurodegenerative disease, comprising administering to a subject in need thereof an effective amount of a compound of claim 46 or a pharmaceutically acceptable salt thereof.

107-108. (canceled)
